(19) United States
(12) Patent Application Publication
(10) Pub. No.: US 2023/0257347 A1
(43) Pub. Date: Aug. 17, 2023

(54) ADVANTAGEOUS TRYPTAMINE COMPOSITIONS FOR MENTAL DISORDERS OR ENHANCEMENT

(71) Applicant: Tactogen Inc, Palo Alto, CA (US)

(72) Inventor: Matthew Baggott, Redwood City, CA (US)

(73) Assignee: Tactogen Inc, Palo Alto, CA (US)

(21) Appl. No.: 18/123,812

(22) Filed: Mar. 20, 2023

Related U.S. Application Data
(63) Continuation of application No. PCT/US2021/051129, filed on Sep. 20, 2021.
(60) Provisional application No. 63/080,791, filed on Sep. 20, 2020.
(60) Provisional application No. 63/120,198, filed on Dec. 1, 2020.
(60) Provisional application No. 63/149,091, filed on Feb. 12, 2021.

(51) Int. Cl. C07D 209/16

(52) U.S. Cl. CPC C07D 209/16; C07B 2200/07

(57) ABSTRACT

Pharmaceutically active advantageous tryptamine compounds and their pharmaceutically acceptable salts, salt mixtures and pharmaceutically acceptable compositions are disclosed for the treatment of mental disorders or for mental enhancement, including for entactogenic therapy, and generally for modulating central nervous system activity.

### CROSS-REFERENCE TO RELATED APPLICATIONS

This application is a continuation of International Patent Application No. PCT/US2021/051129, filed in the U.S. Receiving Office on Sep. 20, 2021, which claims the benefit of U.S. Provisional Application No. 63/080,791, filed Sep. 20, 2020; U.S. Provisional Application No. 63/120,198, filed Dec. 1, 2020; and U.S. Provisional Application No. 63/149,091, filed Feb. 12, 2021. The entirety of each of these applications is hereby incorporated by reference herein for all purposes.

### FIELD OF THE INVENTION

The present invention is in the area of pharmaceutically active tryptamine compounds and compositions for the treatment of mental disorders or for mental enhancement, including for entactogenic therapy. The present invention also includes tryptamine compounds, compositions, and methods for modulating central nervous system activity and treating central nervous system disorders.

### BACKGROUND

Mental disorders, including Post-Traumatic Stress Disorder (PTSD), are more common in society than most recognize, as they can be silent or hidden. The U.S. National Institute of Mental Health (NIMH) reports that 70% of all adults have experienced at least one traumatic event in their lives, and 20% of these people will develop PTSD. NIMH estimates that about 3.6% of U.S. adults have PTSD in a one-year period. PTSD can significantly impair a person's ability to function at work, at home, and socially. While many people associate PTSD with veterans and combat, in fact, it is prevalent in all aspects of society.

The World Health Organization reports that depression is a serious medical disorder affecting at least 264 million people globally of all ages. When long lasting and with even moderate intensity or severe intensity, depression can become a serious health condition. It is a leading cause of disability and if not treated can lead to suicidal thoughts and ideation which can progress to suicide as well as addiction. According to WHO, suicide is the second leading cause of death globally in 15-29 year olds.

Other mental disorders that can profoundly affect a person's ability to function normally in society include anxiety disorders such as generalized anxiety disorder, phobia, panic disorder, separation anxiety disorder, stress-related disorders, adjustment disorder, dissociative disorder, eating disorders (for example, bulimia, anorexia, etc.), attention deficit disorder, sleep disorders, disruptive disorders, neurocognitive disorders, obsessive compulsive disorders, and personality disorders, among others.

While medications are available or in clinical testing for a range of mental disorders, these disorders remain a large burden of disease globally and are insufficiently treated. Further, many of the medications have a long ramp-up time of weeks or more, during which period some patients needing therapy stop the medication out of impatience or the belief the medication does not work.

Many mental disorders are caused by, affected by and/or may be treated by altered levels of neurotransmitters, which are chemicals that transmit a signal from a neuron across the synapse to another neuron. Brain neurotransmitter systems include the serotonin system, the noradrenaline (norepinephrine) system, the dopamine system and the cholinergic system. Dopamine, serotonin, and noradrenaline (norepinephrine) are classed as phenylethylamines, and noradrenaline is also a catecholamine. Drugs that prevent a neurotransmitter from binding to its receptor are called receptor antagonists. Drugs that bind to a receptor and mimic the normal neurotransmitter are receptor agonists. Other drugs interfere with the deactivation of a neurotransmitter after it has been released, which prolongs its action. This can be accomplished by blocking the re-uptake of the transmitter (reuptake inhibitor) or by inhibiting enzymes that degrade the transmitter. A direct agonist binds directly to its associated receptor site. An indirect agonist increases the binding of a neurotransmitter at the target receptor by stimulating the release or preventing the reuptake of the neurotransmitter.

Dopamine receptors are involved in many neurological processes such as motivation, pleasure, cognition, memory, learning, and fine motor control. It is the primary neurotransmitter involved in the reward pathway. Drugs that increase dopamine may produce euphoria. Some widely used drugs such as methamphetamines alter the functioning of the dopamine transporter (DAT), which is responsible for removing dopamine from the neural synapse.

Norepinephrine, also called noradrenaline, mobilizes the body for activity, and is at a high level during stress or danger. It focuses attention and increases arousal and alertness.

Serotonin (5-hydroxytryptamine or “5-HT”) receptors influence various neurological functions such as aggression, anxiety, appetite, cognition, learning, memory, mood, and sleep. 5-HT receptors are the target of FDA approved drugs and unapproved drugs, including antidepressants, antipsychotics, hallucinogens (psychedelics), and entactogens (empathogens). There are seven families of 5-HT receptors, and each has subtypes, creating a highly complex signaling system. For example, when 5-HT_{2A} is agonized it often induces hallucinogenic effects (for example, perceptual distortions, delusions, depersonalization, derealization, and labile mood), whereas 5-HT_{2B}, which is more predominantly in the periphery than in the brain, when chronically agonized, can cause toxicity such as valvulopathy. In contrast, 5-HT_{1B} when agonized regulates neurons in the ventral striatum and likely contributes to the social effects of entactogens.

Current treatments for a range of mental disorders typically involve the use of selective serotonin reuptake inhibitors (SSRIs), such as citalopram (Celexa), escitalopram (Lexapro), fluoxetine (Prozac), paroxetine (Paxil) and sertraline (Zoloft). SSRIs block the reabsorption (i.e., reuptake) of serotonin into neurons, thereby increasing levels of serotonin in the brain. However, SSRIs are generally slow to achieve clinically meaningful benefit, requiring weeks to produce therapeutic effects. Moreover, many patients are nonresponders and show no benefit at all (Masand et al., Harv. Rev. Psychiatry, 1999, 4: 69-84; Rosen et al., J. Clin. Psychopharmacol., 1999, 19: 67-85).

Bupropion (Wellbutrin), in contrast, is an anti-depressant that is a norepinephrine-dopamine reuptake inhibitor, which provides more stimulant effects, including weight loss.

Another class of drugs for treatment of CNS mental disorders is monoamine releasers. Monoamine releasers induce the release of one or more monoamine neurotransmitters (for example, dopamine, serotonin, or epinephrine) from neurons in the brain. Monoamine releasers rapidly modulate the brain systems that are more slowly affected by SSRIs. However, their stimulant and euphoric effects frequently lead them to have high abuse liability. Hence, although the monoamine releasers based on the phenethylamine structure, such as amphetamine (Benzedrine, Dexedrine) and methamphetamine (Obetrol, Pervitin), were widely employed as antidepressants in the mid-20th century, such agents are now used much more cautiously, and primarily treat attention deficit hyperactivity disorder (ADHD).

In the search for alternatives to the flawed existing CNS mental disorder therapies, new classes of pharmacological agent have been investigated. Entactogens (empathogens) have received recent attention as promising agents to solve some of these serious health problems. Entactogens increase feelings of authenticity and emotional openness while decreasing social anxiety (Baggott et al., Journal of Psychopharmacology 2016, 30.4: 378-87). Entactogens are typically monoamine releasers that appear to produce their effects in part by releasing serotonin, which stimulates serotonergic receptors in the hypothalamus and nucleus accumbens areas of the brain (Ramos et al., Neuropsychopharmacology 2013, 38(11):2249-59; Heifets et al., Science translational medicine. 2019, 11:522). Entactogens are distinguished from drugs that are primarily hallucinogenic or psychedelic, and from stimulants, such as amphetamine. The most well-known entactogen is MDMA (3,4-methylenedioxymethamphetamine). Other examples of entactogens are MDA, MBDB, MDOH, and MDEA, however, these drugs do have varying and complex effects that result in part from binding to a range of 5-HT receptors.

MDMA is currently in human clinical trials in the United States (clinicaltrials.gov; NCT03537014) and Europe for approval for use in psychotherapy sessions for severe PTSD and has been suggested as useful for aiding social cognition (Preller & Vollenweider, Frontiers in Psychiatry, 2019, 10; Hysek et al., Social cognitive and affective neuroscience, 2015, 9.11, 1645-52). The FDA granted breakthrough therapy designation for the trial and has also agreed to an expanded access program, both indicative of promising results (Feduccia et al., Frontiers in Psychiatry, 2019, 10: 650; Sessa et al., Frontiers in Psychiatry, 2019, 10: 138). While MDMA has significant therapeutic potential, it has a number of features that potentially make it contraindicated for some patients. This includes its ability to produce acute euphoria, acute hypertensive effects, risk of hyponatremia, and oxidative and metabolic stress.

It is an object of the present invention to provide advantageous compositions and their use and manufacture for the treatment of mental disorders and enhancement. Additional objects are to provide drugs with a more rapid onset to be used in a clinical setting such as counseling or a home setting, which open the patient to empathy, sympathy and acceptance. A further object is to provide effective treatments for a range of CNS disorders.

### SUMMARY OF THE INVENTION

The present invention provides selected advantageous tryptamine compounds and their pharmaceutically acceptable salts and salt mixtures thereof, pharmaceutical compositions, and methods to treat mental disorders and more generally central nervous disorders. A tryptamine compound of the present invention can be used for mental enhancement comprising administering an effective amount of the compound to a host, typically a human, as further described herein.

Tryptamine is an indolamine metabolite of the amino acid tryptophan. A number of tryptamine compounds are known in the field of psychotropic drugs. Some are considered to act as serotonergic hallucinogens and act primarily on the 5-H_{2A} receptor. Known hallucinogenic tryptamines include psilocybin (found in the so-called “magic mushrooms” of the genus Psilocybe), N,N-dimethyltryptamine (DMT), α-methyltryptamine (“AMT”), 5-methoxy-N,N-dimethyltryptamine (5-MeO-DMT), 5-methoxy-N,N-diisopropyltryptamine (sometimes referred to as the street drug “Foxy methoxy” or “Foxy”) and lysergic acid diethylamide (“LSD”). These drugs are known to have a powerful effect on perception and spatial orientation as well as on thoughts, emotions and consciousness. They are listed on controlled substance registers. Two tryptamines—AMT and α-ethyltryptamine were previously marketed as antidepressants and have lessened effects at 5-HT_{2A} while still increasing extracellular serotonin. The drugs discussed in this paragraph are referred to herein as the “classic tryptamines.”

Certain embodiments of the present invention provide tryptamine compounds that have milder effects than the classical tryptamine hallucinogens and may be selected to minimize unwanted properties in a manner that makes them more useful for conventional mental health therapy and/or entactogenic therapy, including periodic or consistent use when administered in an effective amount to a human in need thereof.

Specifically, in certain embodiments, the tryptamines of the present invention are fast acting and/or have fewer properties that decrease the patient experience, are counterproductive to the therapy, or are undesirably toxic. These milder therapeutics can be used in an effective amount to assist persons with mental disorders or who suffer from other CNS disorders. One goal of the invention is to provide therapeutic compositions that increase empathy, sympathy, openness and acceptance of oneself and others, which can be taken, if necessary, as part of therapeutic counseling sessions, or when necessary, episodically, or even consistently, as prescribed by a healthcare provider.

Toxicity of classic tryptamines is often due to monoamine oxidase (MAO) inhibition. In contrast, in certain aspects, selected compounds of the present invention have less or minimal monoamine oxidase inhibitory activity than classic tryptamines, which represents a significant advance for clinical use.

In addition to decreased MAO inhibition activity, selected compounds of the present invention may have additional advantages when compared to traditional CNS disorder therapeutics. For example, selected compounds of the present invention activate the 5-HT_{2A} receptor significantly less than the classic tryptamines noted above. Activation of the 5-HT_{2A} receptor can cause hallucinations and feelings of fear, both of which complicate and can be detrimental to therapy and patient experience. In comparison, selected compounds and compositions of the present invention does not display any measurable 5-HT_{2A} activator properties, as demonstrated in Example 3 and Example 5.

The mood-altering effects of the selected tryptamines makes guided or assisted therapy sessions with a medical professional valuable. Selected compounds of the present invention also in some embodiments have a shorter duration of action, including when taken orally. When the duration of action of a compound is longer than a typical therapy session, for example more than a few hours, there is an increased burden on the medical professional's time and resources as well as on the patient's cost and convenience. It is therefore a beneficial aspect of the present invention that selected compounds or compositions described herein can have a short half-life and reduced duration of effect compared to previous tryptamine compounds. For example, in certain embodiments, the effect may last less than three hours, less than two hours, less than 90 minutes, less than one hour, less than 30 minutes, or less than 15 minutes.

The entactogenic properties of the present tryptamine compounds can be assessed by multiple published methods, including but not limited to those described in Example 10 (Evaluation of Entactogenic Effect of Decreased Neuroticism) and Example 11 (Evaluation of Entactogenic Effect of Authenticity).

In one aspect, the invention provides a tryptamine compound selected from BK-5F-NM-AMT, BK-5Cl-NM-AMT, or BK-5Br-NM-AMT, which may be a racemate, a pure R- or S-enantiomer thereof, or an enantiomerically enriched mixture of the R- or S-enantiomers, including any mixture of enantiomers, or a pharmaceutically acceptable salt or salt mixture thereof for any of the uses described herein. In certain aspects, a pharmaceutical composition is provided that comprises BK-5F-NM-AMT, BK-5Cl-NM-AMT, or BK-5Br-NM-AMT or a pure R- or S-enantiomer or enantiomerically enriched mixture thereof:

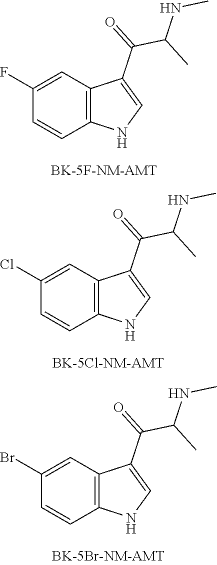 [SMILES: CNC(C)C(=O)c1c[nH]c2ccc(F)cc12 ; CNC(C)C(=O)c1c[nH]c2ccc(Cl)cc12 ; CNC(C)C(=O)c1c[nH]c2ccc(Br)cc12]

or a pharmaceutically acceptable salt thereof.

In another aspect, the invention provides a compound, that is a racemate, pure R- or S-enantiomer, or enantiomerically enriched mixture of Formulas I-XII:

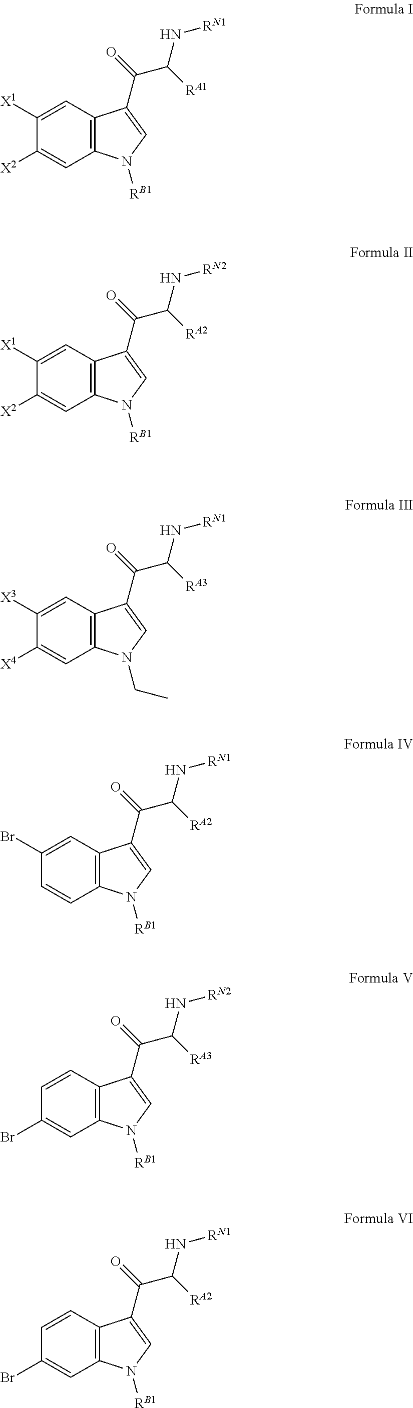

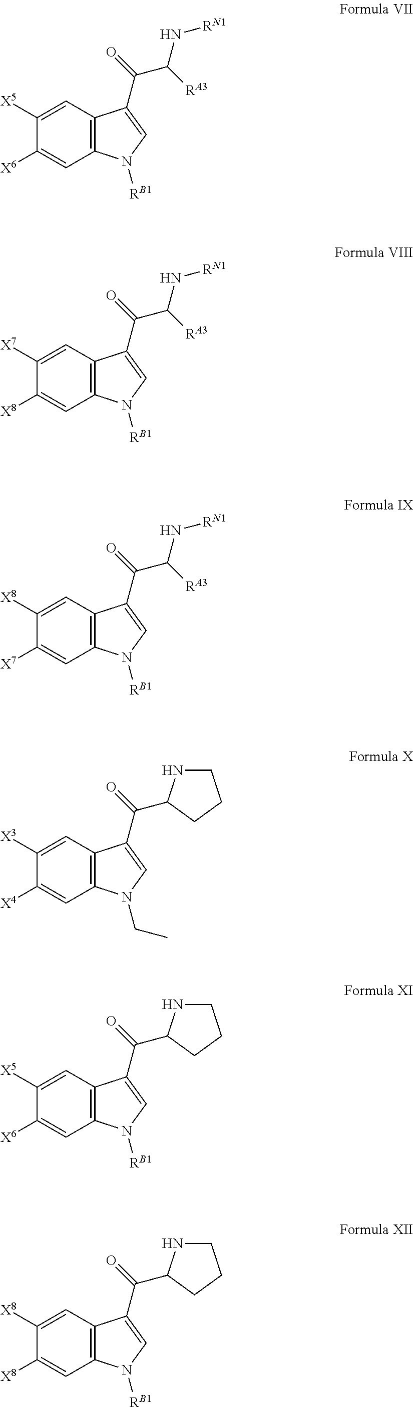

or a pharmaceutically acceptable salt or salt mixture thereof;

wherein:

R^{N1} is selected from —H, —CH₃, and —CH₂CH₃;

R^{N2} is selected from —CH₃, and —CH₂CH₃;

R^{A1} is selected from —CH₂CH₂X, —CH₂CHX₂, —CH₂CX₃, —CH₂OH and —CH₂CH₂OH;

R^{A2} is selected from —CH₃, —CH₂CH₃, —CH₂CH₂X, —CH₂CHX₂, —CH₂CX₃, —CH₂OH and —CH₂CH₂OH;

R^{A3} is selected from —H, —CH₃, —CH₂CH₃, —CH₂CH₂X, —CH₂CHX₂, —CH₂CX₃, —CH₂OH and —CH₂CH₂OH;

R^{B1} is selected from —H, —CH₃, and —CH₂CH₃;

X¹ is independently selected in each instance from —H, —F and —Cl;

X² is selected from —H, —F and —Cl, wherein X¹ and X² must be different;

X³ is selected from —H, —F, —Cl, and —Br;

X⁴ is selected from —H, —F, —Cl, and —Br, wherein X³ and X⁴ must be different;

X⁵ is selected from —H and —I;

X⁶ is selected from —H and —I, wherein X⁵ and X⁶ must be different;

X⁷ is selected from —F, —Br, and —I;

X⁸ is independently selected in each instance from —F, —Cl, —Br, and —I; and

X is independently selected in each instance from —F, —Cl, —Br, and —I.

A compound of Formulas I-XII can be used as a racemic mixture, enantiomerically enriched or pure isomer, as desired to achieve the goal of therapy.

In another aspect, the invention provides the racemate, pure R- or S-enantiomer or enantiomerically enriched mixtures of any of Formulas XIII-XXV or a pharmaceutically acceptable salt or mixed salt thereof. In certain aspects, a pharmaceutical composition is provided that comprises a pure R- or S-enantiomer or enantiomerically enriched mixture of any of Formulas XIII-XXV:

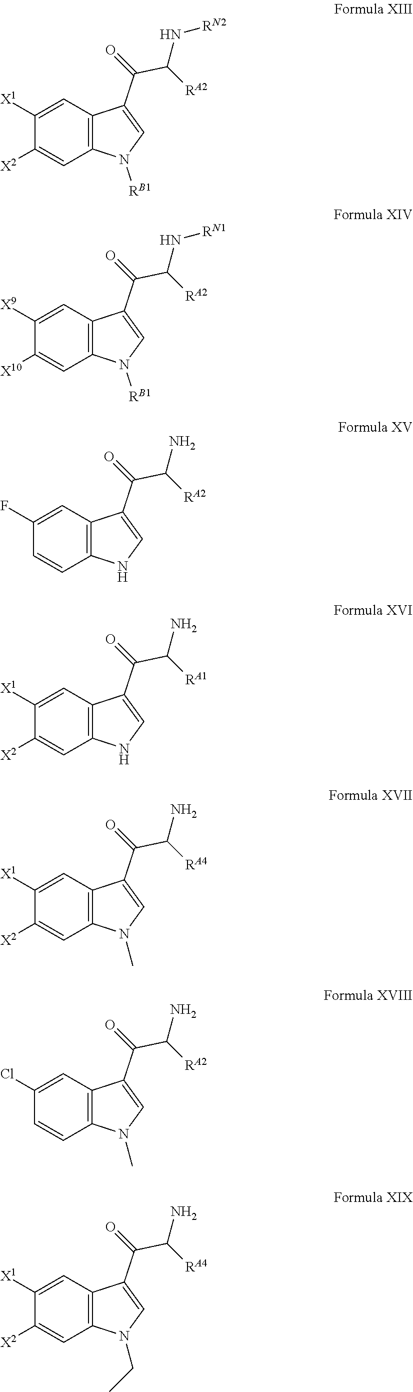

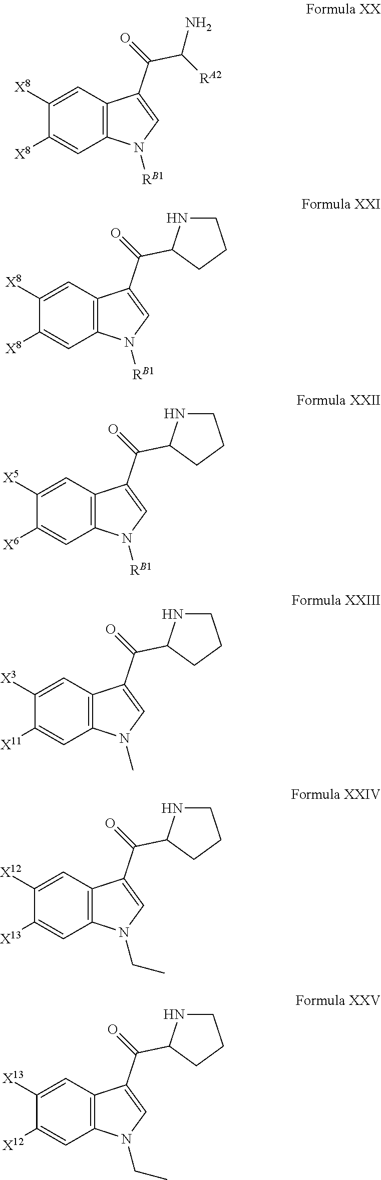

wherein:

R^{A4} is selected from —CH₂CH₃, —CH₂CH₂X, —CH₂CHX₂, —CH₂CX₃, —CH₂OH and —CH₂CH₂OH;
X⁹ is selected from —H, —Br, and —I;
X¹⁰ is selected from —H, —Br, and —I, wherein X⁹ and X¹⁰ must be different;
X¹¹ is selected from —H, —F, and —Br, wherein X³ and X¹¹ must be different;
X¹² is selected from —H, —F, —Cl, —Br, and —I;
X¹³ is selected from —F, —Cl, —Br, and —I; and
all other variables are as defined herein.

In another aspect, the invention provides the racemate, or pure R or S enantiomer or an enantiomerically enriched mixture of any of Formulas XXVI-XXIX or a pharmaceutically acceptable salt or mixed salt thereof. In certain aspects, a pharmaceutical composition is provided that comprises an enantiomerically enriched mixture of any of Formulas XXVI-XXIX:

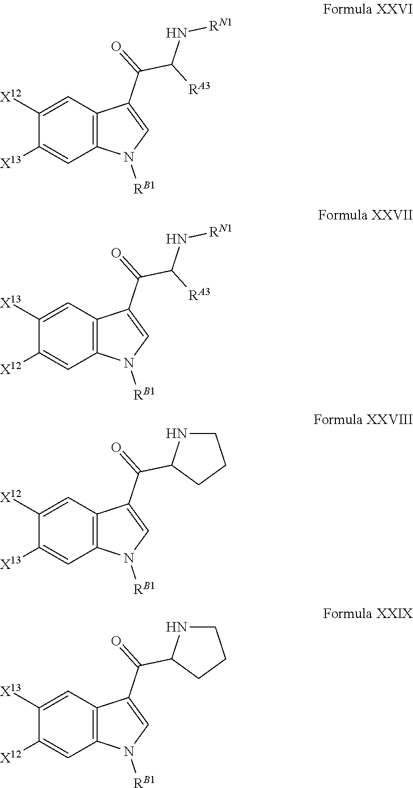

wherein all variables are as defined herein

An enantiomerically enriched mixture is a mixture that contains one enantiomer in a greater amount than the other. The term enantiomerically enriched mixture includes either the mixture enriched with the R-enantiomer or enriched with the S-enantiomer. Unless context clearly indicates otherwise, the term “enantiomerically enriched mixture” can be understood to mean “enantiomerically enriched mixture of the R- or S-enantiomer.” An enantiomerically enriched mixture of an S-enantiomer contains at least 55% of the S-enantiomer, and, typically at least about 60%, 65%, 70%, 75%, 80%, 85%, 90%, 95% of the S-enantiomer. An enantiomerically enriched mixture of an R-enantiomer contains at least 55% of the R-enantiomer, and typically at least about 60%, 65%, 70%, 75%, 80%, 85%, 90%, 95% of the R-enantiomer. The specific ratio of S or R enantiomer can be selected for the need of the patient according to the health care specialist to balance the desired effect.

The term enantiomerically enriched mixture as used herein typically does not include either a racemic mixture or a pure enantiomer.

In certain embodiments, isolated enantiomers of the compounds of the present invention show improved binding at a desired receptor or transporter relevant to the goal of treatment for the mental disorder or for mental enhancement. In certain embodiments, a tuned enantiomerically enriched mixture containing both R- and S-enantiomers in unequal amounts shows improved binding at the desired receptors and transporters relevant to the goal of treatment for the mental disorder or for mental enhancement

In some embodiments, an enantiomerically enriched mixture of the S-enantiomer or pure enantiomer of a compound described herein increases the serotonin-receptor-dependent therapeutic effects and minimizes unwanted dopaminergic effects when administered to a host in need thereof, for example a mammal, including a human, relative to the racemic form.

In other embodiments, an enantiomerically enriched mixture of the R-enantiomer or pure enantiomer of a compound described herein increases the serotonin-receptor-dependent therapeutic effects and minimizes unwanted dopaminergic effects when administered to a host in need thereof, for example a mammal, including a human, relative to the racemic form.

In certain embodiments an enantiomerically enriched mixture of the present invention that is non-racemic has a relatively greater amount of some therapeutic effects (such as emotional openness) while having lesser effects associated with abuse liability (such as perceptible ‘good drug effects’). Additionally, abuse liability can be attenuated to the extent that the substance also increases extracellular serotonin (see, for example, Wee et al., Journal of Pharmacology and Experimental Therapeutics, 2005, 313(2), 848-854). Therefore, one aspect of the present invention is a balanced enantiomerically enriched mixture of the S- and R-enantiomers of a compound of the present invention, optionally as a salt or salt mixture, that achieves a predetermined combination of emotional therapeutic effects and perceptible mood effects. The effect can be modulated as desired for optimal therapeutic effect.

In certain embodiments, an enantiomerically enriched mixture of the S-enantiomer or pure enantiomer of a compound of the present invention or a pharmaceutically acceptable salt or salt mixture thereof balances emotional openness and perceptible mood effects when administered to a host in need thereof, for example a mammal, including a human.

In certain aspects, the S-enantiomer of a compound of the present invention balances emotional openness and perceptible mood effects when administered to a host in need thereof, for example a mammal, including a human.

Additional non-limiting examples of unwanted effects that can be minimized by carefully selecting the balance of enantiomers in an enantiomerically enriched mixture include hallucinogenic effects, psychoactive effects (such as excess stimulation or sedation), physiological effects (such as transient hypertension or appetite suppression), toxic effects (such as to the brain or liver), effects contributing to abuse liability (such as euphoria or dopamine release), and/or other side effects.

The present invention includes a compound, pure enantiomer, or enantiomerically enriched mixture with a beneficial selectivity profile for neurotransmitter transporters. The balance of weakly activating NET (to reduce acute cardiovascular toxicity risk) and decreasing the DAT to SERT ratio over the racemate (to increase therapeutic effect relative to addictive liability) is a desirable feature of an entactogenic therapy displayed by the compounds and compositions of the present invention.

The general pharmacology of entactogen enantiomers and enantiomeric compositions has been poorly understood to date. They have been difficult to separate, and it is not currently easily predicted what the therapeutic effects of individual enantiomers or enantiomerically enriched compositions might be based on individual complex receptor binding. Further, trends in the contribution of individual enantiomers often do not translate to other members of the same class of compounds. For example, the S-(+)-enantiomer of MDMA is more psychoactive than the R-(−)-enantiomer, but in 3,4-methylenedioxyamphetamine (MDA, differing from MDMA only by the absence of an N-methyl group), the S-(+)-enantiomer is less active than its corresponding R-(−)-enantiomer (Anderson et al., NIDA Res Monogr, 1978, 22: 8-15; Nichols. J. Psychoactive Drugs, 1986, 18: 305-13).

In the case of amphetamine, a non-entactogenic stimulant, it has been observed that an enantiomerically enriched mixture of enantiomers displays properties superior to the racemic mix or either enantiomer alone (Joyce et al., Psychopharmacology, 2007, 191: 669-677). The drug Adderall is a paradigm example of a mixture of enantiomers of amphetamine. The mixture has equal parts racemic amphetamine and dextroamphetamine salt mixtures (sulfate, aspartate, and saccharate) which results in an approximately 3:1 ratio between the dextroamphetamine and levoamphetamine. The two enantiomers are different enough to give Adderall an effect profile different from the racemate or the d-enantiomer. However, to date, it has not been reported or predictable what properties a mixture of enantiomers of the entactogenic compounds described herein would produce or how to use the mixture in therapy.

Understanding the pharmacology of the entactogen enantiomers is further complicated by the fact that the therapeutic effects of entactogens are not identical to the more readily identifiable psychoactive effects. Moreover, different enantiomers may differ in potency and activity in dissimilar and unpredictable ways. For instance, when the enantiomers of 3,4-methylenedioxy-N-ethylamphetamine (MDE) were compared in humans, it was concluded that the therapeutic effects of MDE were due to the S-(+)-enantiomer while the R-(−)-enantiomer primarily contributed to unwanted and toxic effects (Spitzer et al., Neuropharmacology, 2001, 41.2: 263-271). In contrast, it has been argued that the R-(−)-enantiomer of MDMA may maintain the therapeutic effects of (±)-MDMA with a reduced side effect profile (Pitts et al., Psychopharmacology, 2018, 235.2: 377-392). Thus, it is not possible to predict which enantiomers will best retain or provide therapeutic activity.

Further, a compound of the present invention retains antagonism of the serotonin transporter (SERT), which is believed to be the principal mechanism of action for SSRIs. In this way the present invention provides a compound, composition, or method that acts in a similar way to the current standard of care for many CNS disorders, including mental disorders, but does not present the crucial drawback of delayed onset.

In other embodiments, a compound or composition of the present invention is provided in an effective amount to treat a host, typically a human, with a CNS disorder that can be either a neurological condition (one that is typically treated by a neurologist) or a psychiatric condition (one that is typically treated by a psychiatrist). Neurological disorders are typically those affecting the structure, biochemistry, or normal electrical functions of the brain, spinal cord or other nerves. Psychiatric conditions are more typically thought of as mental disorders, which are primarily abnormalities of thought, feeling or behavior that cause significant distress or impairment of personal functioning.

Thus, a disclosed compound can be used in an effective amount to improve neurological or psychiatric functioning in a patient in need thereof. Neurological indications include, but are not limited to, improved neuroplasticity, including treatment of stroke, brain trauma, dementia, and neurodegenerative diseases. MDMA has an EC₅₀ of 7.41 nM for promoting neuritogenesis and an Eₘₐₓ approximately twice that of ketamine, which has fast-acting psychiatric benefits that are thought to be mediated by its ability to promote neuroplasticity, including the growth of dendritic spines, increased synthesis of synaptic proteins, and strengthening synaptic responses (Ly et al. Cell reports 23, no. 11 (2018): 3170-3182; Figure S3). The compounds of the current invention can similarly be considered psychoplastogens, that is, small molecules that are able to induce rapid neuroplasticity (Olson, 2018, Journal of experimental neuroscience, 12, 1179069518800508). For example, in certain embodiments, the disclosed compounds and compositions can be used to improve stuttering and other dyspraxias or to treat Parkinson's disease or schizophrenia.

In other embodiments, a tryptamine composition or compound of the present invention may be used in an effective amount to treat a host, typically a human, to modulate an immune or inflammatory response. The compounds disclosed herein alter extracellular serotonin, which is known to alter immune functioning. MDMA produces acute time-dependent increases and decreases in immune response (for example, Pacifici et al. 2004. Journal of Pharmacology and Experimental Therapeutics, 309(1), 285-292).

In certain embodiments, a tryptamine compound of Formulas I-XXIX is used as described herein in enantiomerically enriched form of the R- or S-enantiomer to achieve the goals of the invention. In other embodiments, the compound is used as a racemate or a pure enantiomer.

The invention additionally includes methods to treat a neurological or psychiatric central nervous system disorder as further described herein, including a mental disorder, or to provide a mental enhancement, with a compound of Formula I-XXIX or a pharmaceutically acceptable salt or salt mixture thereof.

The present invention also provides new medical uses for the described compounds, including but not limited to, administration in an effective amount to a host in need thereof such as a human for post-traumatic stress disorder, depression, dysthymia, anxiety, generalized anxiety, social anxiety, panic, adjustment disorders, feeding and eating disorders, binge behaviors, body dysmorphic syndromes, addiction, drug abuse or dependence disorders, substance use disorders, disruptive behavior disorders, impulse control disorders, gaming disorders, gambling disorders, memory loss, dementia of aging, attention deficit hyperactivity disorder, personality disorders, attachment disorders, autism or dissociative disorders or any other disorder described herein, including in the Background. One particular treatment is for adjustment disorder, which is highly prevalent in society and currently insufficiently addressed. In nonlimiting aspects, the compound used in the treatment includes, for example, a racemic, enantiomerically pure or enriched composition of R- or S-enantiomer of BK-5F-NM-AMT, BK-5Cl-NM-AMT, or BK-5Br-NM-AMT, or a combination thereof.

The term “improving psychiatric function” is intended to include mental health and life conditions that are not traditionally treated by neurologists but sometimes treated by psychiatrists and can also be treated by psychotherapists, life coaches, personal fitness trainers, meditation teachers, counselors, and the like. For example, it is contemplated that the disclosed compounds will allow individuals to effectively contemplate actual or possible experiences that would normally be upsetting or even overwhelming. This includes individuals with fatal illnesses planning their last days and the disposition of their estate. This also includes couples discussing difficulties in their relationship and how to address them. This also includes individuals who wish to more effectively plan their career.

In some embodiments, methods of the present invention include treatment of a CNS disorder that has been linked to inadequate functioning of serotonergic neurotransmission, including adjustment disorder, anxiety, and depression, in mammals, particularly humans, using a selected tryptamine compound or a racemate or pure enantiomer or enantiomerically enriched mixtures of the present invention.

In certain embodiments, any of the selected compounds or mixtures of the present invention are administered to a human patient in an effective amount in conjunction with psychotherapy, cognitive enhancement, or life coaching (pharmacotherapy), or as part of routine medical therapy.

Any of the tryptamine compounds, including the enantiomerically enriched compounds, can be used in the form of a pharmaceutically acceptable salt or a salt mixture. Nonlimiting examples include those wherein the pharmaceutically acceptable salt(s) is selected from HCl, sulfate, aspartate, saccharate, phosphate, oxalate, acetate, amino acid anion, gluconate, maleate, malate, citrate, mesylate, nitrate or tartrate, or a mixture thereof.

In some embodiments, the present invention includes pharmaceutical compositions which comprise a tryptamine compound of any one of Formulas I-XXIX, either racemic, as pure enantiomers, or as an enantiomerically enriched mixture, and which may be in association with another active agent, as well as with a pharmaceutically acceptable carrier, diluent, or excipient.

The present invention thus includes at least the following aspects:
(i) A tryptamine compound of BK-5F-NM-AMT, BK-5Cl-NM-AMT, BK-5Br-NM-AMT, or Formula I-XXII or a pharmaceutically acceptable salt or salt mixture, isotopic derivative, or prodrug thereof,
(ii) An enantiomerically enriched or pure tryptamine compound of BK-5F-NM-AMT, BK-5Cl-NM-AMT, BK-5Br-NM-AMT, or Formula I-XXV, or a pharmaceutically acceptable salt, or salt mixture, an isotopic derivative, or prodrug thereof, as relevant;
(iii) An enantiomerically enriched mixture of BK-5F-NM-AMT, BK-5Cl-NM-AMT, BK-5Br-NM-AMT, or Formula I-XXIX, or a pharmaceutically acceptable salt, or salt mixture, an isotopic derivative, or prodrug thereof, as relevant;
(iv) A pharmaceutical composition comprising an effective patient-treating amount of a tryptamine compound of (i), (ii) or (iii) or a pharmaceutically acceptable salt or salt mixture, isotopic derivative, or prodrug thereof, optionally with a pharmaceutically acceptable carrier or diluent or any of the uses described herein;
(v) The pharmaceutically acceptable composition of (iv) in a solid or liquid, systemic, oral, topical or parenteral dosage form;
(vi) A method for treating a patient with any neurological or psychological CNS disorder as described herein that includes administering an effective amount of a compound of (i), (ii) or (iii) to a patient such as a human in need thereof,
(vii) A method for treating any neurological or psychological CNS disorder comprising administering an effective amount of a tryptamine compound of (i), (ii) or (iii) or a pharmaceutically acceptable salt, isotopic derivative, or prodrug thereof, as described herein, to a patient, typically a human, in need thereof;
(viii) A tryptamine compound of (i), (ii) or (iii) or a pharmaceutically acceptable salt, salt mixture, isotopic derivative, or prodrug thereof, for use to treat any disorder as described herein in an effective amount as further described herein;
(ix) A tryptamine compound of (i), (ii) or (iii) for use in the manufacture of a medicament for the treatment of any of the disorders described herein;
(x) Use of a tryptamine compound of (i), (ii) or (iii) or a pharmaceutically acceptable salt, salt mixture, isotopic derivative, or prodrug thereof, to treat any disorder as described herein in an effective amount as further described herein;
(xi) Processes for the preparation of therapeutic products that contain an effective amount of a tryptamine compound of (i), (ii) or (iii) or a pharmaceutically acceptable salt or salt mixture, isotopic derivatives, or prodrugs thereof, as described herein.

### DETAILED DESCRIPTION OF THE INVENTION

The present invention provides multiple embodiments of the described tryptamine compounds, compositions, and methods to treat mental disorders, and more generally central nervous disorders, as well as for mental enhancement. The tryptamine compounds of the present invention provide advantageous pharmacological properties that are highly desirable as therapeutics for the treatment of mental disorders, particularly as psychotherapeutics and neurotherapeutics.

The embodiments of the invention are presented to meet the goal of assisting persons with mental disorders, who desire mental enhancement, or who suffer from other CNS disorders by providing milder therapeutics that reduce the properties that decrease the patient experience, are counterproductive to the therapy, or are undesirably toxic. One goal of the invention is to provide therapeutic compositions that increase empathy, sympathy, openness and acceptance of oneself and others, which can be taken if necessary as part of therapeutic counseling sessions, when necessary episodically or even consistently, as prescribed by a healthcare provider.

### I. Definitions

When introducing elements of the present invention or the typical embodiments thereof, the articles “a,” “an,” “the,” and “said” are intended to mean that there are one or more of the elements. The terms “comprising,” “including,” “such as,” and “having” are intended to be inclusive and not exclusive (i.e., there may be other elements in addition to the recited elements). Thus, the terms “including,” “may include,” and “include,” as used herein mean, and are used interchangeably with, the phrase “including but not limited to.”

Where a range of values is provided, it is understood that the upper and lower limit, and each intervening value between the upper and lower limit of the range is encompassed within the embodiments.

Unless defined otherwise, all technical and scientific terms herein have the meaning as commonly understood by one of ordinary skill in the art to which this invention belongs. In the event there is a plurality of definitions for a term herein, those in this section prevail unless stated otherwise. Further definitions that may assist the reader to understand the disclosed embodiments are as follows, and such definitions may be used to interpret the defined terms, when those terms are used herein. However, the examples given in the definitions are generally non-exhaustive and must not be construed as limiting the invention. It also will be understood that a substituent should comply with chemical bonding rules and steric compatibility constraints in relation to the particular molecule to which it is attached.

“Compounds” refers to compounds encompassed by structural formulas disclosed herein (for example, Formula I), and includes any specific compounds within these formulas whose structure is disclosed herein. Although sometimes referred to using different terms, and sometimes used interchangeably with “structures,” compounds will be understood to include the conjugates, codrugs, and prodrugs of the invention. The compounds of the invention may be identified either by their chemical structure and/or chemical name. When the chemical structure and chemical name conflict, the chemical structure is determinative of the identity of the compound. The compounds of the invention may contain one or more chiral centers and/or double bonds and therefore, may exist as stereoisomers, such as double-bond isomers (i.e., geometric isomers), enantiomers, or diastereomers. The chemical structures depicted herein encompass all possible enantiomers and stereoisomers of the illustrated compounds including the stereoisomerically pure form (for example, geometrically pure, enantiomerically pure, or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. Further, it should be understood when partial structures of the compounds of the invention are illustrated, that brackets, dashes, or perpendicular rippled lines indicate the point of attachment of the partial structure to the rest of the molecule. In any compound described herein having one or more chiral centers, if an absolute stereochemistry is not expressly indicated, then each center may independently be of R-configuration or S-configuration or a mixture thereof. Thus, the compounds provided herein may be pure enantiomers, enantiomerically enriched mixtures, racemic mixtures, pure diastereomers, diastereomerically enriched, or a stereoisomeric mixture. In addition, it is understood that in any compound described herein having one or more double bond(s) generating geometrical isomers that can be defined as E or Z, each double bond may independently be E or Z a mixture thereof.

It will be understood that the compounds described herein include crystalline forms (also known as “polymorphs,” which include the different crystal packing arrangements of the same elemental composition of a compound), amorphous phases, salts, solvates, and hydrates. Furthermore, compounds disclosed herein may exist in one or more crystalline or amorphous forms. In addition, some of the compounds disclosed herein may form solvates with water (i.e., hydrates) or common organic solvents. Unless otherwise indicated, such solvates, crystalline, and amorphous forms are included in the scope of the present invention.

In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

“Composition of the invention” refers to at least one compound of the invention and a pharmaceutically acceptable vehicle with which the compound is administered to a patient. When administered to a patient, the compounds of the invention are administered in isolated form, which means separated from a synthetic organic reaction mixture.

An enantiomerically enriched mixture is a mixture that contains one enantiomer in a greater amount than the other. An enantiomerically enriched mixture of an S-enantiomer contains at least 55% of the S-enantiomer, and, typically at least about 60%, 65%, 70%, 75%, 80%, 85%, 90%, or 95% or more of the S-enantiomer. An enantiomerically enriched mixture of an R-enantiomer contains at least 55% of the R-enantiomer, and typically at least about 60%, 65%, 70%, 75%, 80%, 85%, 90% or 95% of the R-enantiomer. The specific ratio of S or R enantiomer can be selected for the need of the patient according to the health care specialist to balance the desired effect.

The term enantiomerically enriched mixture as used in this application does not include a racemic mixture and does not include a pure isomer. Notwithstanding, it should be understood that any compound described herein in enantiomerically enriched form can be used as a pure isomer if it achieves the goal of any of the specifically itemized methods of treatment described herein, including but not limited to BK-5F-NM-AMT, BK-5Cl-NM-AMT, or BK-5Br-NM-AMT.

The term “CNS disorder” as used herein refers to either a neurological condition (one that is typically treated by a neurologist) or a psychiatric condition (one that is typically treated by a psychiatrist). Neurological disorders are typically those affecting the structure, biochemistry or normal electrical functioning of the brain, spinal cord or other nerves. Psychiatric conditions are more typically thought of as mental disorders, which are primarily abnormalities of thought, feeling or behavior that cause significant distress or impairment of personal functioning. Thus, the disclosed compounds can be used in an effective amount to improve neurological or psychiatric functioning in a patient in need thereof. Neurological indications include, but are not limited to improved neuroplasticity, including treatment of stroke, brain trauma, dementia, and neurodegenerative diseases. Compounds of the current invention can be considered psychoplastogens, that is, small molecules that are able to induce rapid neuroplasticity. For example, in certain embodiments, the disclosed compounds and compositions can be used to improve stuttering and other dyspraxias or to treat Parkinson's disease or schizophrenia.

The term “improving psychiatric function” is intended to include mental health and life conditions that are not traditionally treated by neurologists but sometimes treated by psychiatrists and can also be treated by psychotherapists, life coaches, personal fitness trainers, meditation teachers, counselors, and the like. For example, it is contemplated that the disclosed compounds will allow individuals to effectively contemplate actual or possible experiences that would normally be upsetting or even overwhelming. This includes individuals with fatal illness planning their last days and the disposition of their estate. This also includes couples discussing difficulties in their relationship and how to address them. This also includes individuals who wish to more effectively plan their career.

The term “inadequate functioning of neurotransmission” is used synonymously with a CNS disorder that adversely affects normal healthy neurotransmission.

The present invention also includes compounds, including enantiomerically enriched compounds and their use, such as BK-5F-NM-AMT, BK-5Cl-NM-AMT, BK-5Br-NM-AMT or a compound of any of Formulas I-XXIX, with at least one desired isotopic substitution of an atom, at an amount above the natural abundance of the isotope, i.e., isotopically enriched. Isotopes are atoms having the same atomic number but different mass numbers, i.e., the same number of protons but a different number of neutrons.

Examples of isotopes that can be incorporated into compounds of the invention include isotopes of hydrogen, carbon, nitrogen, oxygen, fluorine and chlorine such as ²H, ³H, ¹¹C, ¹³C, ¹⁴C, ¹³N, ¹⁵N, ¹⁷O, ¹⁸O, ¹⁸F, ³⁶Cl, and respectively. In one non-limiting embodiment, isotopically labelled compounds can be used in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F labeled compound may be particularly desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

By way of general example and without limitation, isotopes of hydrogen, for example, deuterium (²H) and tritium (³H) may be used anywhere in described structures that achieves the desired result. Alternatively, or in addition, isotopes of carbon, for example, ¹³C and ¹⁴C, may be used.

Isotopic substitutions, for example deuterium substitutions, can be partial or complete. Partial deuterium substitution means that at least one hydrogen is substituted with deuterium. In certain embodiments, the isotope is at least 60, 70, 80, 90, 95 or 99% or more enriched in an isotope at any location of interest. In one non-limiting embodiment, deuterium is 90, 95 or 99% enriched at a desired location. Substitution with isotopes such as deuterium may afford certain therapeutic advantages resulting from greater metabolic stability, such as, for example, increased in vivo half-life or reduced dosage requirements. In a compound structure a hydrogen atom may be explicitly disclosed or should be understood to be optionally present in the compound. At any position of the compound that a hydrogen atom may be present, the hydrogen atom can be an isotope of hydrogen, including but not limited to protium and deuterium. Thus, reference herein to a compound encompasses all potential isotopic forms unless the context clearly dictates otherwise.

In one non-limiting embodiment, the substitution of a hydrogen atom for a deuterium atom can be provided in a compounds or compositions described herein. In one non-limiting embodiment, the substitution of a hydrogen atom for a deuterium atom occurs within a group selected from any R¹⁴, R¹⁵, R¹⁶, R¹⁷, R¹⁸, R¹⁹, R^{A}, R^{A1}, R^{A2}, R^{A3}, R^{A4}, R^{B1}, R^{BA}, R^{C}, R^{D}, R^{E}, R^{N1}, R^{N2}, R^{NA}, R^{NB}, R^{NC}, R^{P}, R^{X1}, R^{X2}, Q¹, Q², Q³, Q⁴, and Y.

For example, when any of the groups are, or contain for example through substitution, methyl, ethyl, or methoxy, the alkyl residue may be deuterated (in non-limiting embodiments, CDH₂, CD₂H, CD₃, CH₂CD₃, CD₂CD₃, CHDCH₂D, CH₂CD₃, CHDCHD₂, OCDH₂, OCD₂H, or OCD₃ etc.). The compounds of the invention also include isotopically labeled compounds where one or more atoms have an atomic mass different from the atomic mass most abundant in nature. Examples of isotopes that may be incorporated into the compounds of the invention include ²H, ³H, ¹³C, ¹⁴C, ¹³N, ¹⁵N, ¹⁸O, ¹⁷O, ³¹P, ³²P, ³⁵S, ¹⁸F, and ³⁶Cl.

For example, the methyl group on the nitrogen of BK-5F-NM-AMT, BK-5Cl-NM-AMT, and BK-5Br-NM-AMT is subject to metabolic removal, which produces pharmacologically active metabolites. In some embodiments, BK-5F-NM-AMT, BK-5Cl-NM-AMT, or BK-5Br-NM-AMT is prepared with deuterium replacing some or all of the three hydrogens on the N-methyl group. This creates a higher activation energy for bond cleavage and a slower formation of the methyl metabolites. Analogously, the two hydrogens on the indole ring may be replaced with one or two deuteriums to decrease metabolic opening of the indole ring and formation of hydroxyl-substituted metabolites.

Similarly, the methyl or ethyl group optionally on the nitrogen of Formula I-IX, XIII-XIV, or XXVI-XXVII of the invention is subject to metabolic removal, which produces pharmacologically active metabolites. In one embodiment, a compound of Formula I-IX, XIII-XIV, or XXVI-XXVII is prepared with deuterium replacing some or all of the three, four, or five hydrogens on the N-methyl or N-ethyl group. The primary amines of Formula I-IX, XIII-XIV, or XXVI-XXVII of the invention retain therapeutic effects while presenting a different profile of pharmacological effects.

The term “isotopically-labeled” analog refers to an analog that is a “deuterated analog”, a “¹³C-labeled analog,” or a “deuterated/¹³C-labeled analog.” The term “deuterated analog” means a compound described herein, whereby a H-isotope, i.e., hydrogen/protium (H), is substituted by a H-isotope, for example, deuterium (²H). Deuterium substitution can be partial or complete. Partial deuterium substitution means that at least one hydrogen is substituted by at least one deuterium. In certain embodiments, the isotope is at least 60, 70, 80 90, 95 or 99% or more enriched in an isotope at any location of interest. In some embodiments it is deuterium that is 90, 95 or 99% enriched at a desired location. Unless indicated to the contrary, the deuteration is at least 80% at the selected location. Deuteration of the nucleoside can occur at any replaceable hydrogen that provides the desired results.

“Alkyl” refers to a saturated or unsaturated, branched, straight-chain, or cyclic monovalent hydrocarbon radical derived by the removal of one hydrogen atom from a single carbon atom of a parent alkane, alkene or alkyne. Typical alkyl groups include methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like. Alkyl will be understood to include cyclic alkyl radicals such as cyclopropyl, cyclobutyl, and cyclopentyl.

“Alkyl” includes radicals having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. When the group contains a double bond, it may be in either the cis or trans conformation. In certain embodiments, an alkyl group comprises from 1 to 26 carbon atoms, typically from 1 to 10 carbon atoms.

“Halogen” or “halo” means fluoro (F), chloro (Cl), bromo (Br), or iodo (I). For groups containing two or more halogens, such as —CHY₂ or —CY₃, and for example “where Y is halogen,” it will be understood that each Y independently will be selected from the group of halogens.

“Alkoxy” refers to a radical OR where R represents an alkyl or cycloalkyl group as defined herein. Representative examples include methoxy, ethoxy, propoxy, butoxy, cyclohexyloxy, and the like.

“Hydroxy” means the radical OH.

“Oxo” means the divalent radical ═O.

“Aryl” refers to a monovalent aromatic hydrocarbon radical derived by the removal of one hydrogen atom from a single carbon atom of a parent aromatic ring system. Typical aryl groups include groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. Typically, an aryl group comprises from 6 to 20 carbon atoms, more typically, between 6 and 12 carbon atoms.

“Arylalkyl” refers to an acyclic alkyl radical in which one of the hydrogen atoms bonded to a carbon atom, typically a terminal or sp³ carbon atom, is replaced with an aryl group. Typical arylalkyl groups include benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. Preferably, an arylalkyl group is (C₆-C₃₀) arylalkyl, for example, the alkanyl, alkenyl or alkynyl moiety of the arylalkyl group is (C₁-C₁₀) and the aryl moiety is (C₆-C₂₀), more preferably, an arylalkyl group is (C₆-C₂M) arylalkyl, for example, the alkanyl, alkenyl or alkynyl moiety of the arylalkyl group is (C₁-C₈) and the aryl moiety is (C₆-C₁₂).

“Cycloalkyl” refers to a saturated or unsaturated cyclic alkyl radical. Where a specific level of saturation is intended, the nomenclature “cycloalkanyl” or “cycloalkenyl” is used. Typical cycloalkyl groups include groups derived from cyclopropane, cyclobutane, cyclopentane, cyclohexane, and the like. In a preferred embodiment, the cycloalkyl group is (C₃-C₁₀) cycloalkyl, more preferably (C₃-C₇) cycloalkyl.

“Cycloheteroalkyl” refers to a saturated or unsaturated cyclic alkyl radical in which one or more carbon atoms (and any associated hydrogen atoms) are independently replaced with the same or different heteroatom. Typical heteroatoms to replace the carbon atom(s) include N, P, O, S, Si, etc. Where a specific level of saturation is intended, the nomenclature “cycloheteroalkanyl” or “cycloheteroalkenyl” is used. Typical cycloheteroalkyl groups include groups derived from epoxides, imidazolidine, morpholine, piperazine, piperidine, pyrazolidine, pyrrolidine, quinuclidine, and the like.

“Heteroalkyl, Heteroalkanyl, Heteroalkenyl, Heteroalkynyl” refer to alkyl, alkanyl, alkenyl and alkynyl groups, respectively, in which one or more of the carbon atoms (and any associated hydrogen atoms) are each independently replaced with the same or different heteroatomic groups. Typical heteroatomic groups include —O—, —S—, —O—O—, —S—S—, —OS—, —NR′—, ═N—N═, —N═N—, —N═N—NR′—, —PH—, —P(O)₂—, —O—P(O)—, —S(O)—, —S(O)₂—, —SnH₂— and the like, wherein R′ is hydrogen, alkyl, substituted alkyl, cycloalkyl, substituted cycloalkyl, aryl or substituted aryl.

“Heteroaryl” refers to a monovalent heteroaromatic radical derived by the removal of one hydrogen atom from a single atom of a parent heteroaromatic ring system. Typical heteroaryl groups include groups derived from acridine, arsindole, carbazole, carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. Preferably, the heteroaryl group is between 5-20 membered heteroaryl, with 5-10 membered heteroaryl being particularly preferred. Preferred heteroaryl groups are those derived from thiophene, pyrrole, benzothiophene, benzofuran, indole, pyridine, quinoline, imidazole, oxazole, and pyrazine.

“Heteroarylalkyl” refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heterorylalkynyl is used. Preferably, the heteroarylalkyl radical is a 6-30 membered heteroarylalkyl, for example, the alkanyl, alkenyl or alkynyl moiety of the heteroarylalkyl is 1-10 membered and the heteroaryl moiety is a 5-20 membered heteroaryl, more preferably, a 6-20 membered heteroarylalkyl, for example, the alkanyl, alkenyl or alkynyl moiety of the heteroarylalkyl is 1-8 membered and the heteroaryl moiety is a 5-12 membered heteroaryl.

“Amino acid” as used herein can refer to the twenty amino acids encoded by the genetic code, selenocysteine, pyrrolysine or “non-standard amino acids” that are not incorporated into proteins. Non-standard amino acids include the sulfur-containing taurine and the neurotransmitter Gamma-aminobutyric acid (GABA). Other examples are Lanthionine, 2-Aminoisobutyric acid, Dehydroalanine, Carnitine, Ornithine, and Citrulline. As used herein, the amino acids can be in the “D,” “L,” or racemic form. Unless otherwise specified, discussion of any amino acid is intended to refer to all isomers. The amino acids described herein may optionally be modified to mask hydrogen bond donors and improve absorption, following the approach of Barlow et al. (2020, ACS Chemical Biology, 15(8), 2070-2078), wherein polarity is approximately preserved by adding structures with hydrogen bond acceptors to mask the donors. For example, —N—H groups may be masked with —C(O)OCH₂CH₃.

TABLE 1
Amino Acids and Abbreviations

| Abbreviation | Amino acid name |
|---|---|
| 3Hyp | 3-Hydroxyproline |
| 4Abu | 4-Aminobutyric acid, |
| | piperidinic acid |
| 4Hyp | 4-Hydroxyproline |
| Aad | 2-Aminoadipic acid |
| Abu | 2-Aminobutyric acid |
| Acp | 6-Aminocaproic acid |
| Ahe | 2-Aminoheptanoic |
| | acid |
| aHyl | allo-Hydroxylysine |
| Aib | 2-Aminoisobutyric |
| | acid |
| aIle | allo-Isoleucine |
| Ala | Alanine |
| Apm | 2-Aminopimelic acid |
| Arg | Arginine |
| Asn | Asparagine |
| Asp | Aspartic acid |
| Asx | Aspartic acid or |
| | Asparagine |
| bAad | 3-Aminoadipic acid |
| bAib | 3-Aminoisobutyric |
| | acid |
| bAla | beta-Alanine, beta- |
| | Aminoproprionic acid |
| Cys | Cysteine |
| Dbu | 2,4-Diaminobutyric |
| | acid |
| Des | Desmosine |
| Dpm | 2,2′-Diaminopimelic |
| | acid |
| Dpr | 2,3- |
| | Diaminoproprionic |
| | acid |
| EtAsn | N-Ethylasparagine |
| EtGly | N-Ethylglycine |
| Gln | Glutamine |
| Glu | Glutamic acid |
| Glx | Glutamic acid or Glutamine |
| Gly | Glycine |
| His | Histidine |
| Hyl | Hydroxylysine |
| Ide | Isodesmosine |
| Ile | Isoleucine |
| Leu | Leucine |
| Lys | Lysine |
| MeGly | N-Methylglycine, sarcosine |
| MeIle | N-Methylisoleucine |
| MeLys | 6-N-Methyllysine |
| Met | Methionine |
| MeVal | N-Methylvaline |
| Nle | Norleucine |
| Nva | Norvaline |
| Orn | Ornithine |
| Phe | Phenylalanine |
| Pro | Proline |
| Pyl | Pyrrolysine |
| Sec | Selenocysteine |
| Ser | Serine |
| Thr | Threonine |
| Trp | Tryptophan |
| Tyr | Tyrosine |
| Val | Valine |
| Xaa | Any amino acid |
| Xle | Leucine or Isoleucine |

“Dipeptide,” “tripeptide,” and “tetrapeptide,” as used herein refer to groups containing 2, 3, or 4 amino acids, respectively, wherein the amino acids are bonded together by amide bonds at their N- or C-terminus.

“Stereoisomers” includes enantiomers, diastereomers, the components of racemic mixtures, and combinations thereof. Stereoisomers can be prepared or separated as described herein or by using other methods.

“Isomers” includes stereo and geometric isomers, as well as diastereomers. Examples of geometric isomers include cis isomers or trans isomers across a double bond. Other isomers are contemplated among the compounds of the present disclosure. The isomers may be used either in pure form or in admixture with other isomers of the compounds described herein.

“Substituted” refers to a group in which one or more hydrogen atoms are each independently replaced with the same or different substituent(s). Typical substituents include —X, —R¹⁴, —O⁻, ═O, —OR¹⁴, —SR¹⁴, —S⁻, ═S, —NR¹⁴R¹⁵, ═NR¹⁴, —CX₃, —CF₃, —CN, —OCN, —SCN, —NO, —NO₂, ═N₂, —N₃, —S(O)₂O⁻, —S(O)₂OH, —S(O)₂R¹⁴, —OS(O₂)O⁻, —OS(O)₂R₁₄, —P(O)(O⁻)₂, —P(O)(OR¹⁴)(O⁻), —OP(O)(OR¹⁴)(OR¹⁵), —C(O)R¹⁴, —C(S)R¹⁴, —C(O)OR¹⁴, —C(O)NR¹⁴R¹⁵, —C(O)O⁻, —C(S)OR¹⁴, —NR¹⁶C(O)NR¹⁴R¹⁵, —NR¹⁶C(S)NR¹⁴R¹⁵, —NR¹⁷C(NR¹⁶)NR¹⁴R¹⁵ and —C(NR¹⁶)NR¹⁴R¹⁵, where each X is independently selected from —F, —Cl, —Br, and —I; each R¹⁴, R¹⁵, R¹⁶, and R¹⁷ are independently hydrogen, alkyl, aryl, arylalkyl, cycloalkyl, cycloheteroalkyl, heteroalkyl, heteroaryl, heteroarylalkyl, —NR¹⁸R¹⁹, —C(O)R¹⁸ or —S(O)₂R¹⁸; and R¹⁸ and R¹⁹ are independently hydrogen, alkyl, aryl, arylalkyl, cycloalkyl, cycloheteroalkyl, heteroalkyl, heteroaryl, or heteroarylalkyl; or optionally R¹⁸ and R¹⁹ together with the atom to which they are both attached form a cycloheteroalkyl.

“Pharmaceutically acceptable vehicle,” “pharmaceutically acceptable carrier,” refers to a diluent, adjuvant, excipient or carrier with which a compound of the invention is administered. Such diluents, adjuvants, excipients, or carriers result in pharmaceutical compositions that are generally safe, non-toxic, and neither biologically nor otherwise undesirable for veterinary and/or human pharmaceutical use. This term includes a 0.01-0.1M and preferably 0.05M phosphate buffer, or in another embodiment 0.8% saline. Additionally, pharmaceutically acceptable carriers may be in other embodiments aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In some embodiments, the carrier can be a) 10% PEG 400 (v/v)+30% (v/v) HPβCD, 50% w/v+60% (v/v) Sterile Water for Injection or b) 0.1% (v/v) Tween 80+0.5% (w/v) Carboxymethylcellulose in water.

“Subject,” as used herein, refers to a mammal, such as humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, and cats, avian species, such as chickens, turkeys, and songbirds. The subject can be, for example, a child, such as an adolescent, or an adult.

“Agonism” refers to the activation of a receptor or enzyme by a modulator, or agonist, to produce a biological response.

“Agonist” refers to a modulator that binds to a receptor or enzyme and activates the receptor to produce a biological response. As a nonlimiting example, “5HT_{1B} agonist” can be used to refer to a compound that exhibits an EC₅₀ with respect to 5HT_{1B} activity of no more than about 10, 25 or even 50 μM. In some embodiments, “agonist” includes full agonists or partial agonists. “Full agonist” refers to a modulator that binds to and activates a receptor with the maximum response that an agonist can elicit at the receptor. “Partial agonist” refers to a modulator that binds to and activates a given receptor, but has partial efficacy, that is, less than the maximal response, at the receptor relative to a full agonist.

“Antagonism” refers to the inactivation of a receptor or enzyme by a modulator, or antagonist. Antagonism of a receptor, for example, is when a molecule binds to the receptor and does not allow activity to occur.

“Antagonist” or “neutral antagonist” refers to a modulator that binds to a receptor or enzyme and blocks a biological response. An antagonist has no activity in the absence of an agonist or inverse agonist but can block the activity of either, causing no change in the biological response.

“DAT to SERT ratio” refers to the tendency of a substance (for example, a compound or a drug) to increase extracellular dopamine versus increasing extracellular 5-HT concentrations. Higher numbers of this ratio indicate a greater increase of dopamine than serotonin, while lower number indicate an increasing 5-HT more than dopamine. The exact numbers depend on the assay used. The ratio is calculated herein as (DAT EC₅₀)⁻¹/(SERT EC₅₀)⁻¹. Some publications use IC₅₀s for inhibiting uptake instead of EC₅₀s for causing release to calculate this ratio, which will often yield different results for substances that are monoamine releasers. Thus, it is important to review the numbers in view of the assay and measurement used.

“IC₅₀” refers to the concentration of a substance (for example, a compound or a drug) that is required for 50% inhibition of a biological process. For example, IC₅₀ refers to the half maximal (50%) inhibitory concentration (IC) of a substance as determined in a suitable assay. Similarly, EC₅₀ refers to the concentration of a substance that provokes a response halfway between the baseline activity and maximum response. In some instances, an IC₅₀ or EC₅₀ is determined in an in vitro assay system. In some embodiments as used herein, IC₅₀ (or EC₅₀) refers to the concentration of a modulator that is required for 50% inhibition (or excitation) of a receptor, for example, 5HT_{1B}.

“Modulate” or “modulating” or “modulation” refers to an increase or decrease in the amount, quality, or effect of a particular activity, function or molecule. By way of illustration and not limitation, agonists, partial agonists, antagonists, and allosteric modulators (for example, positive allosteric modulator) of a G protein-coupled receptor (for example, 5-HT_{2A}) are modulators of the receptor.

“Neuroplasticity” refers to the ability of the brain to change its structure and/or function throughout a subject's life. Examples of the changes to the brain include, but are not limited to, the ability to adapt or respond to internal and/or external stimuli, such as due to an injury, and the ability to produce new neurites, dendritic spines, and synapses.

“Treating” or “treatment” of a disease, as used in context, includes (i) inhibiting the disease, i.e., arresting or reducing the development or progression of the disease or its clinical symptoms; or (ii) relieving the disease, i.e., causing regression of the disease or its clinical symptoms. Inhibiting the disease, for example, would include prophylaxis. Hence, one of skill in the art will understand that a therapeutic amount necessary to effect treatment for purposes of this invention will, for example, be an amount that provides for objective indicia of improvement in patients having clinically diagnosable symptoms. Other such measurements, benefits, and surrogate or clinical endpoints, whether alone or in combination, would be understood to those of ordinary skill.

“Therapeutic effect” means the responses(s) in a mammal after treatment that are judged to be desirable and beneficial. Hence, depending on the CNS disorder to be treated, or improvement in CNS functioning sought, those responses shall differ, but would be readily understood by those of ordinary skill.

The term “hallucinations” or “hallucinogenic effects” includes but is not limited to perceptual distortions, delusions, depersonalization, derealization and/or labile mood. These effects can include dysphoria of intensities ranging from controllable anxiety to uncontrollable panic.

### II. Compounds of the Present Invention

Tryptamines are known to have beneficial effects in CNS disorders, with melatonin (for sleep disorders) and triptans (for migraines) being common examples. Because of the similarity of the tryptamine structure to serotonin, the tryptamine class of compounds often readily crosses the blood-brain barrier and imparts psychological effects. Looking to take advantage of this property, one tryptamine known as alpha-ethyltryptamine (AET, 1-(1H-indol-3-yl)butan-2-amine) was marketed as an antidepressants in the 1960's under the brand name Monase by Upjohn. The antidepressant effect was presumed to have resulted from monoamine oxidase (MAO) inhibition. It has since been found that alpha-ethyltryptamine also stimulates serotonin release, which likely affects mood changes, also. A similar compound, alpha-methyltryptamine (AMT, 1-(1H-indol-3-yl)propan-2-amine) was investigated and marketed as an antidepressant in the U.S.S.R. Both alpha-ethyltryptamine and alpha-methyltryptamine have since been discontinued, and alpha-ethyltryptamine was linked to idiosyncratic agranulocytosis when used daily. Additionally, a PCT publication from Gilgamesh Pharmaceuticals (WO2021168082) recently described N,N-dialkyltryptamines for the treatment mood disorders.

The mood-altering effects of certain tryptamines have led to their recreational use. Alpha-ethyltryptamine was found to produce entactogenic effects at higher doses, for example, and was subsequently listed as a controlled substance. Alpha-methyltryptamine has also been shown to stimulate 5-HT_{2A} receptors, which at high levels of stimulation can induce feelings of fear (ranging from mild anxiety to panic), hallucinations, feelings of labile mood, anxiety, derealization, and depersonalization.

Despite their potent entactogenic properties, there are several challenges to applying the tryptamine scaffold to therapeutics. The 5-HT_{2A} agonism often seen in tryptamines results in hallucinogen-like effects that render them impractical for use outside of guided settings (Blough et al. Bioorg. Med. Chem. Lett. 2014, 24(19), 4754-4758). Tryptamines also tend to have long-lived effects, lasting several hours. This long duration of action requires long therapy sessions, which can be inconvenient and costly for both the patient and the session administrator.

The mood-altering effects of the selected tryptamines makes guided or assisted therapy sessions with a medical professional valuable. Selected compounds of the present invention also in some embodiments have a shorter duration of action, including when taken orally. When the duration of action of a compound is longer than a typical therapy session, for example more than a few hours, there is an increased burden on the medical professional's time and resources as well as on the patient's cost and convenience. It is therefore a beneficial aspect of the present invention that selected compounds or compositions described herein can have a short half-life and reduced duration of effect compared to previous tryptamine compounds. For example, in certain embodiments, the effect may last less than three hours, less than two hours, less than 90 minutes, less than one hour, less than 30 minutes, or less than 15 minutes.

The MAO inhibition activity that was the suspected antidepressant mechanism of action in alpha-methyl- and alpha-ethyltryptamine also poses problems related to toxicity (Wagmann et al., Toxicology Letters, 2017, 272, 84-93). Compounds that release monoamines while also acting as MAOIs increase the likelihood of toxic syndromes such as agitation, mydriasis, vomiting, tachycardia, and even death. Many derivatives of alpha-methyl- and alpha-ethyltryptamine have been synthesized and shown to inhibit MAO, some even with sub-micromolar potency (Wagmann et al., Toxicology Letters, 2017, 272, 84-93; Blough et al., Bioorg. Med. Chem. Lett., 2014, 24(19), 4754-4758; Blough et al., Psychopharmacology, 2014, 231, 4135-4144; and Reyes-Parada et al. Front. Pharmacol., 2020, 10(1590)). Looking to better understand these risks, many have studied tryptamine-containing compounds to assess their toxicity risk and therapeutic potential (Blough et al., Bioorg. Med. Chem. Lett., 2014, 24(19), 4754-4758; Blough et al., Psychopharmacology, 2014, 231, 4135-4144; Blough et al., Psychopharmacology, 2019, 236, 915-924; Gerasimov et al., J. Med. Chem., 1999, 42(20), 4257-4263; and Reyes-Parada et al. Front. Pharmacol., 2020, 10(1590)). Despite this research, however, there remains a need for molecules that have entactogenic effects free of MAO inhibition.

The present invention provides entactogenic tryptamine compounds that do not inhibit MAO for treatment of mental disorders and enhancement. The combination of features common to all the embodiments of the invention can create unexpectedly beneficial properties for therapeutics, including rapid onset, short duration of effects, low toxicity, no 5-HT_{2A} agonism, and no MAO inhibition. To the inventor's knowledge, the tryptamines disclosed herein have not been proposed as entactogens and most have not been contemplated or synthesized.

In one aspect, the invention provides the compounds BK-5F-NM-AMT, BK-5Cl-NM-AMT, and BK-5Br-NM-AMT, pure R- or S-enantiomers thereof, or enantiomerically enriched mixtures of the R- or S-enantiomers, or a pharmaceutically acceptable salt or salt mixture thereof for any of the uses thereof as described herein. In certain aspects, a pharmaceutical composition is provided that comprises BK-5F-NM-AMT, BK-5Cl-NM-AMT, or BK-5Br-NM-AMT or a pure R- or S-enantiomer or enantiomerically enriched mixture thereof:

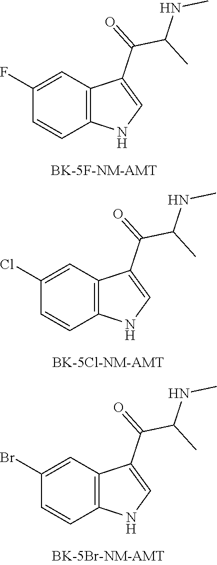 [SMILES: CNC(C)C(=O)c1c[nH]c2ccc(F)cc12 ; CNC(C)C(=O)c1c[nH]c2ccc(Cl)cc12 ; CNC(C)C(=O)c1c[nH]c2ccc(Br)cc12]

In another aspect, the invention provides the compounds, pure R- or S-enantiomers, or enantiomerically enriched mixtures of any of Formulas I-XII. In certain aspects, a pharmaceutical composition is provided that comprises a compound, pure R- or S-enantiomer, or enantiomerically enriched mixture of any of Formulas I-XII:

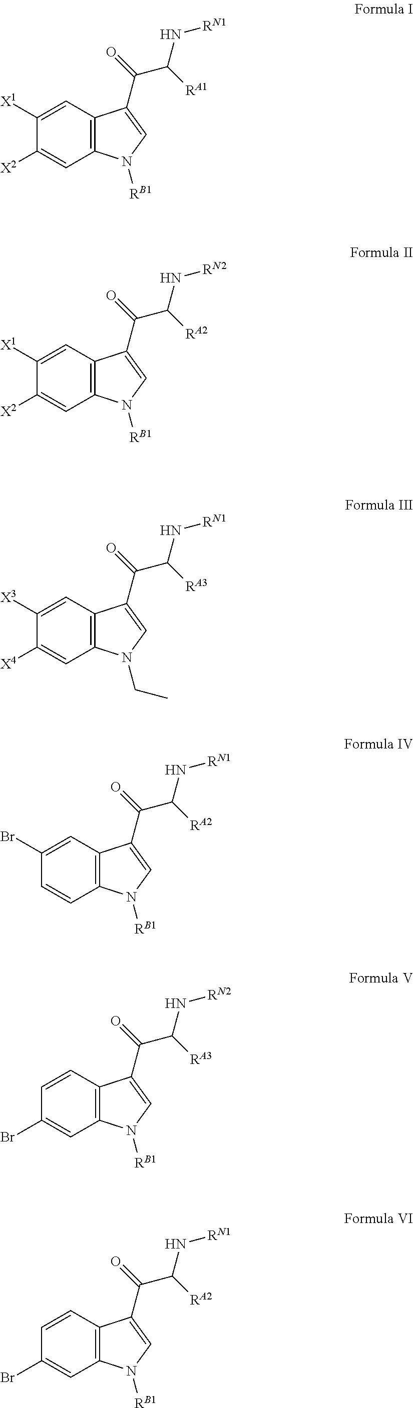

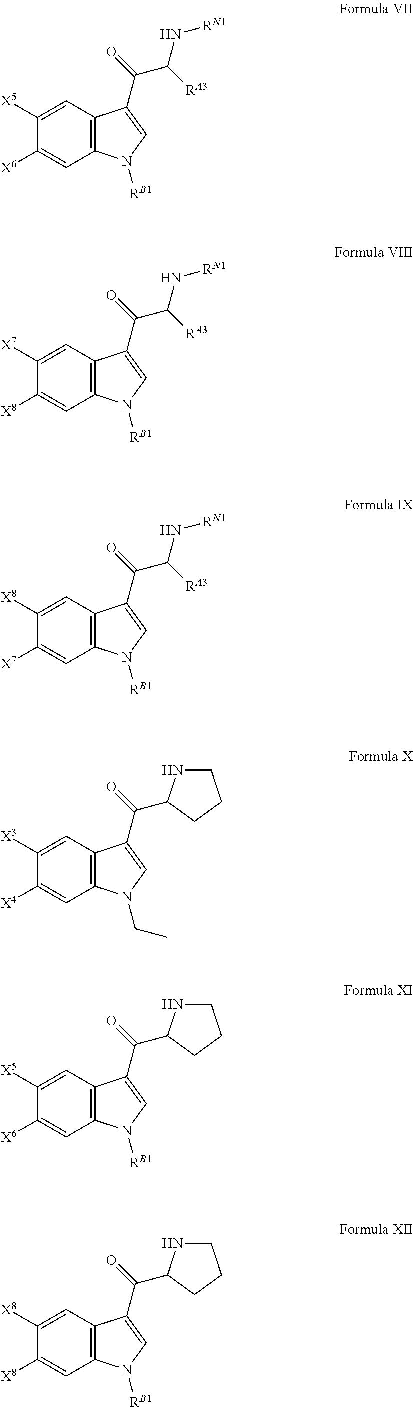

wherein:

R^{N1} is selected from —H, —CH₃, and —CH₂CH₃;

R^{N2} is selected from —CH₃, and —CH₂CH₃;

R^{A1} is selected from —CH₂CH₂X, —CH₂CHX₂, —CH₂CX₃, —CH₂OH and —CH₂CH₂OH;

R^{A2} is selected from —CH₃, —CH₂CH₃, —CH₂CH₂X, —CH₂CHX₂, —CH₂CX₃, —CH₂OH and —CH₂CH₂OH;

R^{A3} is selected from —H, —CH₃, —CH₂CH₃, —CH₂CH₂X, —CH₂CHX₂, —CH₂CX₃, —CH₂OH and —CH₂CH₂OH;

R^{B1} is selected from —H, —CH₃, and —CH₂CH₃;

X¹ is independently selected in each instance from —H, —F and —Cl;

X² is selected from —H, —F and —Cl, wherein X¹ and X² must be different;

X³ is selected from —H, —F, —Cl, and —Br;

X⁴ is selected from —H, —F, —Cl, and —Br, wherein X³ and X⁴ must be different;

X⁵ is selected from —H and —I;

X⁶ is selected from —H and —I, wherein X⁵ and X⁶ must be different;

X⁷ is selected from —F, —Br, and —I;

X⁸ is independently selected in each instance from —F, —Cl, —Br, and —I; and

X is independently selected in each instance from —F, —Cl, —Br, and —I.

In another aspect, the invention provides the pure R- or S-enantiomers or enantiomerically enriched mixtures of any of Formulas XIII-XXV. In certain aspects, a pharmaceutical composition is provided that comprises a pure R- or S-enantiomer or enantiomerically enriched mixture of any of Formulas XIII-XXV:

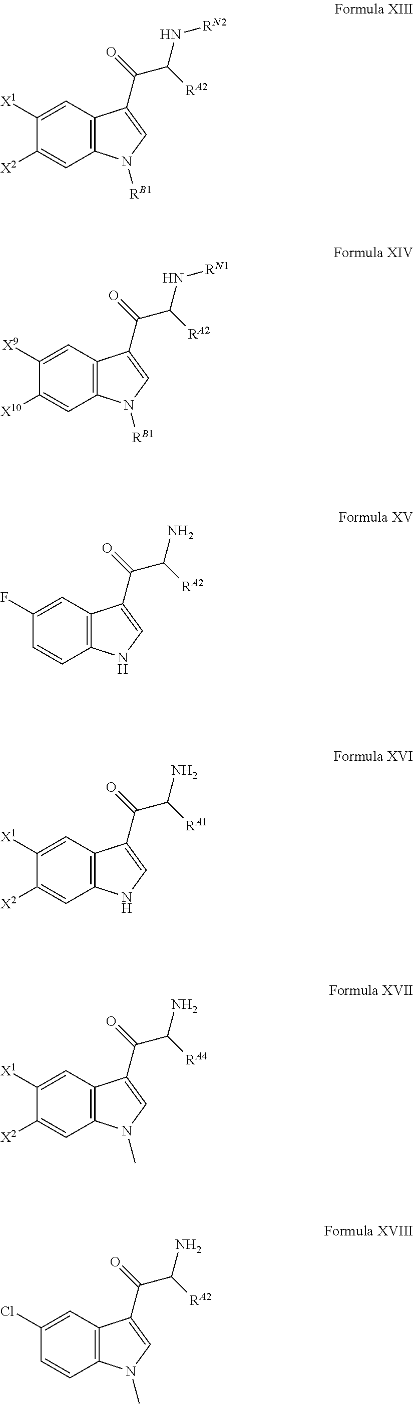

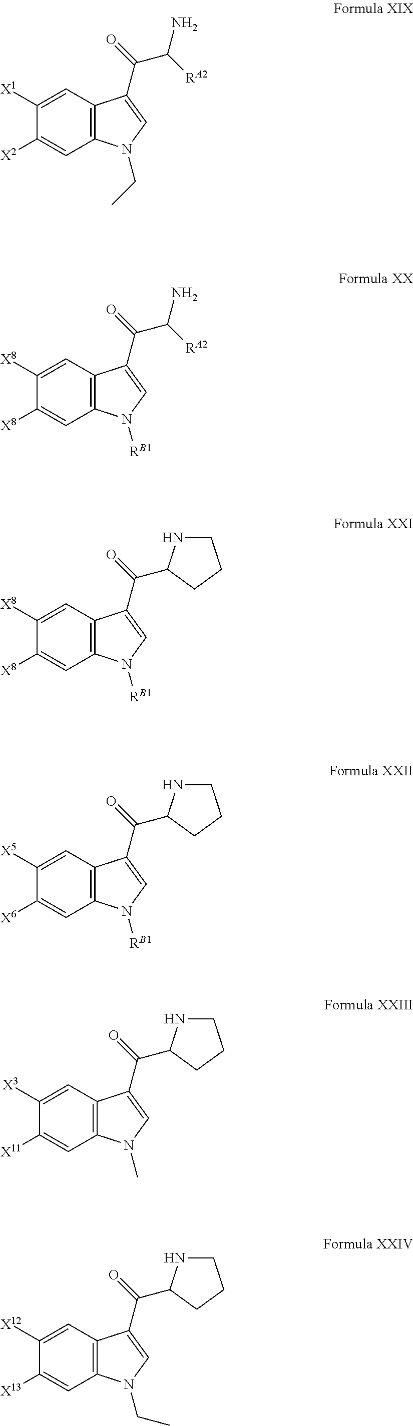

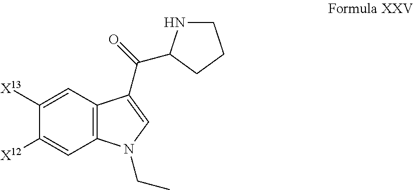

wherein:

R^{A4} is selected from —CH₂CH₃, —CH₂CH₂X, —CH₂CHX₂, —CH₂CX₃, —CH₂OH and —CH₂CH₂OH;
X⁹ is selected from —H, —Br, and —I;
X¹⁰ is selected from —H, —Br, and —I, wherein X⁹ and X¹⁰ must be different;
X¹¹ is selected from —H, —F, and —Br, wherein X³ and X¹¹ must be different;
X¹² is selected from —H, —F, —Cl, —Br, and —I;
X¹³ is selected from —F, —Cl, —Br, and —I; and
all other variables are as defined herein.

In another aspect, the invention provides enantiomerically enriched mixtures of any of Formulas XXVI-XXIX. In certain aspects, a pharmaceutical composition is provided that comprises an enantiomerically enriched mixture of any of Formulas XXVI-XXIX:

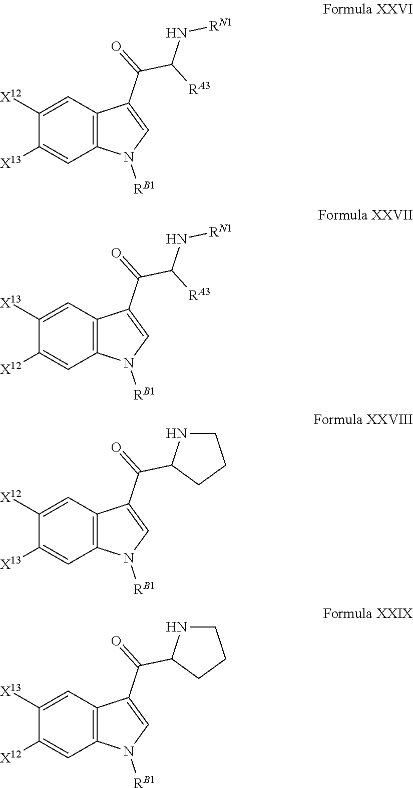

wherein all variables are as defined herein.

In some embodiments, a racemic compound, pure R- or S-enantiomer, or enantiomerically enriched mixture of the present invention is selected from:

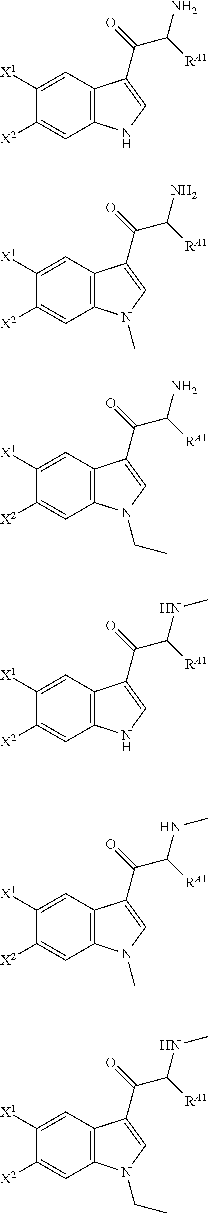

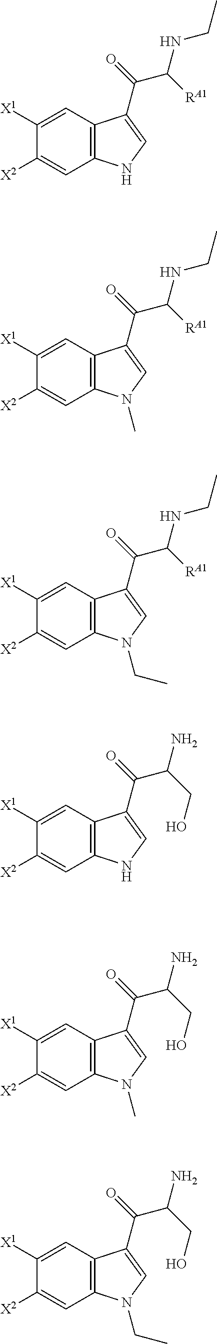

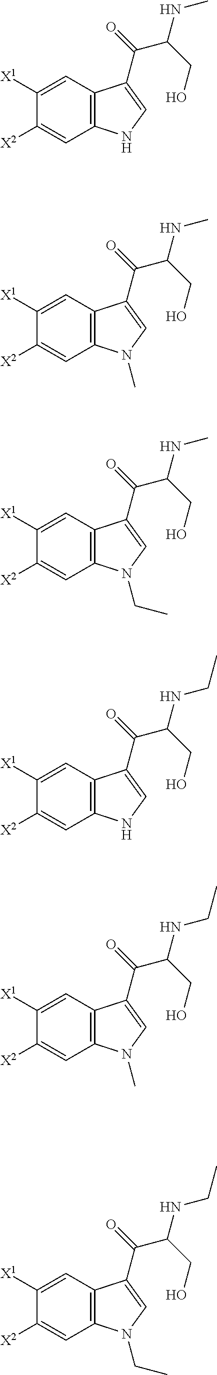

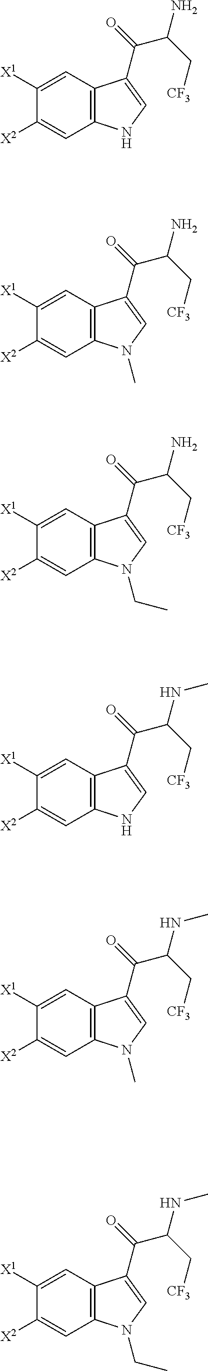

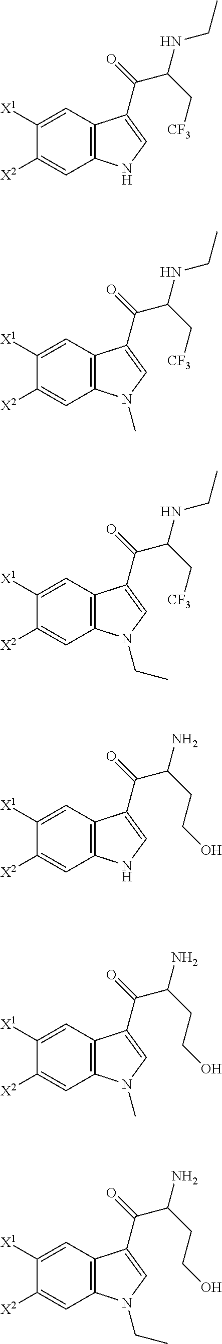

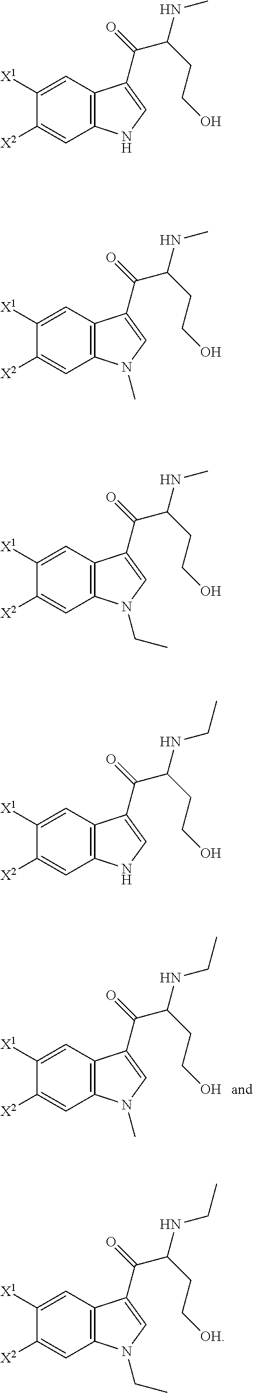

In other embodiments, a racemic compound, pure R- or S-enantiomer, or enantiomerically enriched mixture of the present invention is selected from:

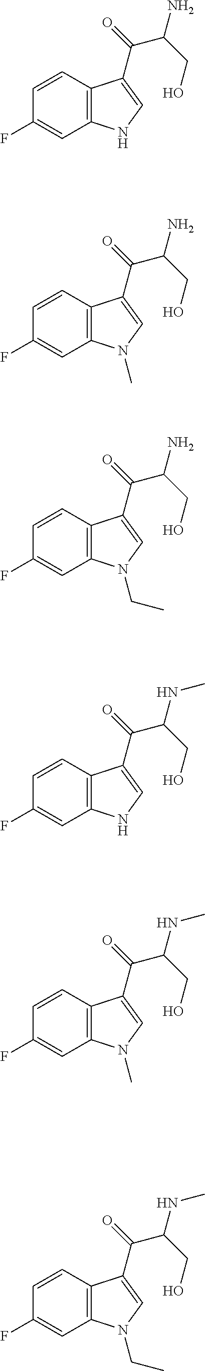 [SMILES: NC(CO)C(=O)c1c[nH]c2cc(F)ccc12 ; Cn1cc(C(=O)C(N)CO)c2ccc(F)cc21 ; CCn1cc(C(=O)C(N)CO)c2ccc(F)cc21 ; CNC(CO)C(=O)c1c[nH]c2cc(F)ccc12 ; CNC(CO)C(=O)c1cn(C)c2cc(F)ccc12 ; CCn1cc(C(=O)C(CO)NC)c2ccc(F)cc21]

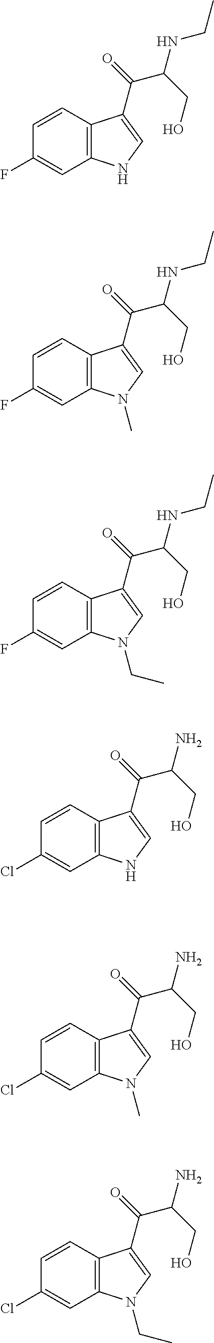 [SMILES: CCNC(CO)C(=O)c1c[nH]c2cc(F)ccc12 ; CCNC(CO)C(=O)c1cn(C)c2cc(F)ccc12 ; CCNC(CO)C(=O)c1cn(CC)c2cc(F)ccc12 ; NC(CO)C(=O)c1c[nH]c2cc(Cl)ccc12 ; Cn1cc(C(=O)C(N)CO)c2ccc(Cl)cc21 ; CCn1cc(C(=O)C(N)CO)c2ccc(Cl)cc21]

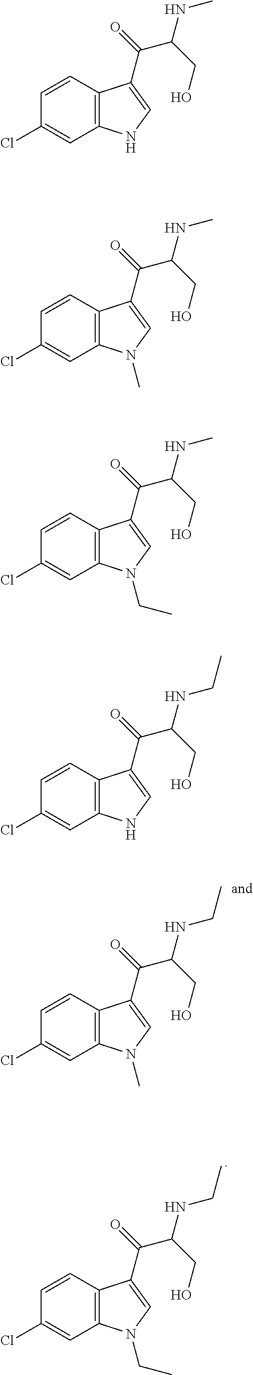 [SMILES: CNC(CO)C(=O)c1c[nH]c2cc(Cl)ccc12 ; CNC(CO)C(=O)c1cn(C)c2cc(Cl)ccc12 ; CCn1cc(C(=O)C(CO)NC)c2ccc(Cl)cc21 ; CCNC(CO)C(=O)c1c[nH]c2cc(Cl)ccc12 ; CCNC(CO)C(=O)c1cn(C)c2cc(Cl)ccc12 ; CCNC(CO)C(=O)c1cn(CC)c2cc(Cl)ccc12]

In certain other embodiments, a racemic compound, pure R- or S-enantiomer, or enantiomerically enriched mixture of the present invention is selected from:

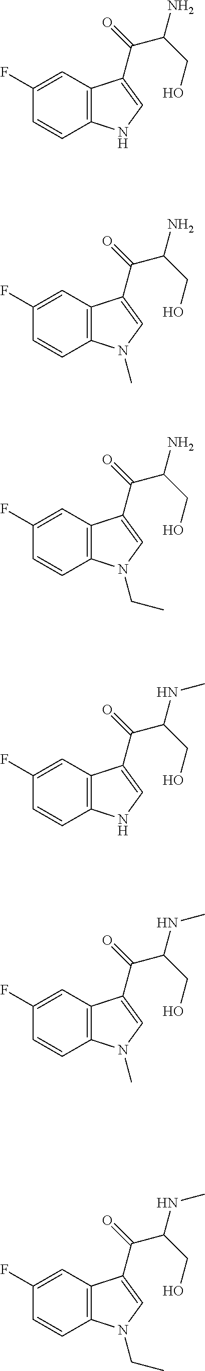 [SMILES: NC(CO)C(=O)c1c[nH]c2ccc(F)cc12 ; Cn1cc(C(=O)C(N)CO)c2cc(F)ccc21 ; CCn1cc(C(=O)C(N)CO)c2cc(F)ccc21 ; CNC(CO)C(=O)c1c[nH]c2ccc(F)cc12 ; CNC(CO)C(=O)c1cn(C)c2ccc(F)cc12 ; CCn1cc(C(=O)C(CO)NC)c2cc(F)ccc21]

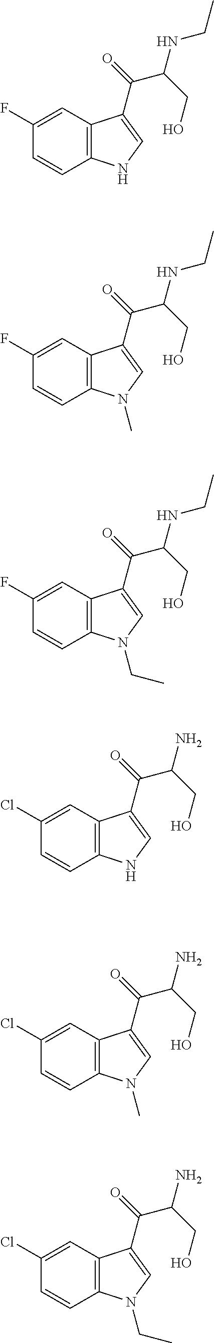 [SMILES: CCNC(CO)C(=O)c1c[nH]c2ccc(F)cc12 ; CCNC(CO)C(=O)c1cn(C)c2ccc(F)cc12 ; CCNC(CO)C(=O)c1cn(CC)c2ccc(F)cc12 ; NC(CO)C(=O)c1c[nH]c2ccc(Cl)cc12 ; Cn1cc(C(=O)C(N)CO)c2cc(Cl)ccc21 ; CCn1cc(C(=O)C(N)CO)c2cc(Cl)ccc21]

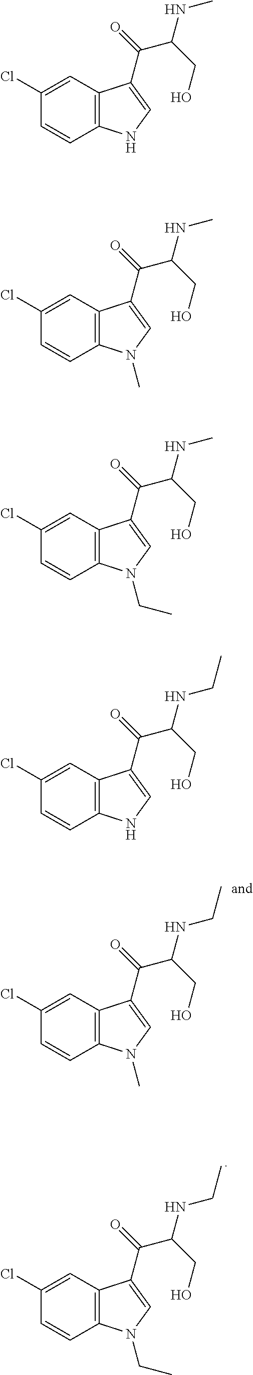 [SMILES: CNC(CO)C(=O)c1c[nH]c2ccc(Cl)cc12 ; CNC(CO)C(=O)c1cn(C)c2ccc(Cl)cc12 ; CCn1cc(C(=O)C(CO)NC)c2cc(Cl)ccc21 ; CCNC(CO)C(=O)c1c[nH]c2ccc(Cl)cc12 ; CCNC(CO)C(=O)c1cn(C)c2ccc(Cl)cc12 ; CCNC(CO)C(=O)c1cn(CC)c2ccc(Cl)cc12]

Additional embodiments include a racemic compound, pure R- or S-enantiomer, or enantiomerically enriched mixture of the present invention is selected from:

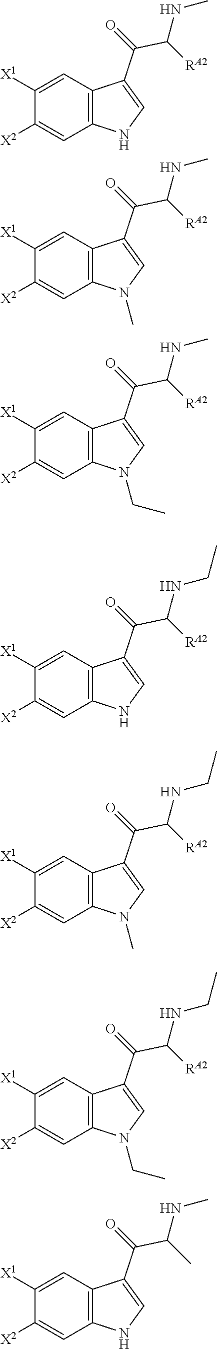

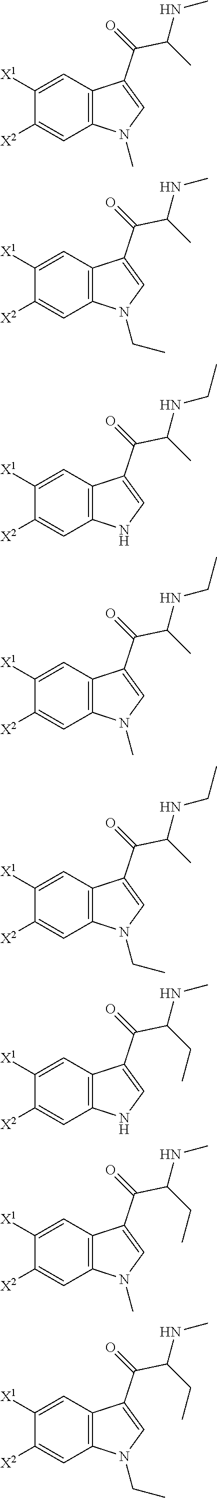

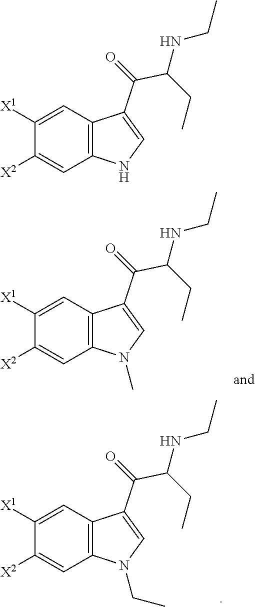

Further embodiments include a racemic compound, pure R- or S-enantiomer, or enantiomerically enriched mixture of the present invention is selected from:

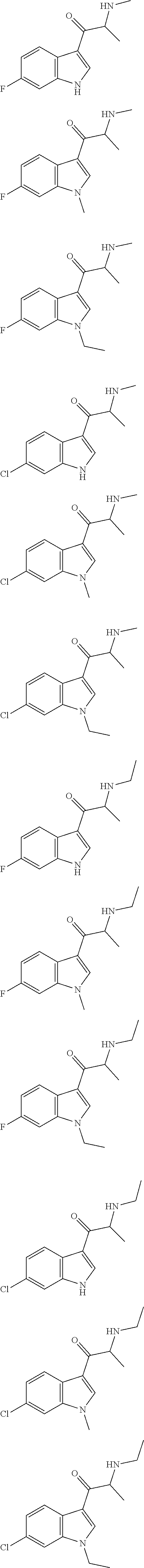

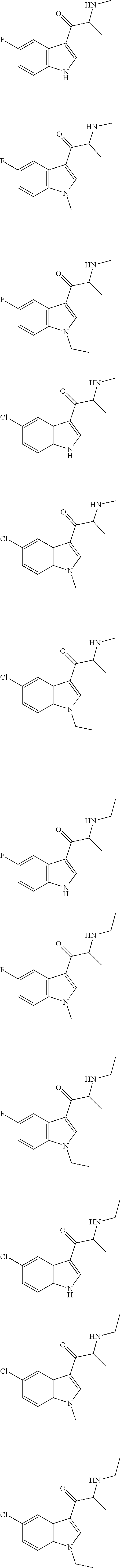

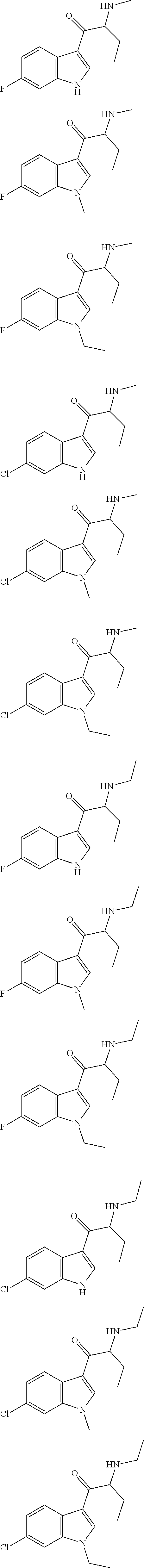

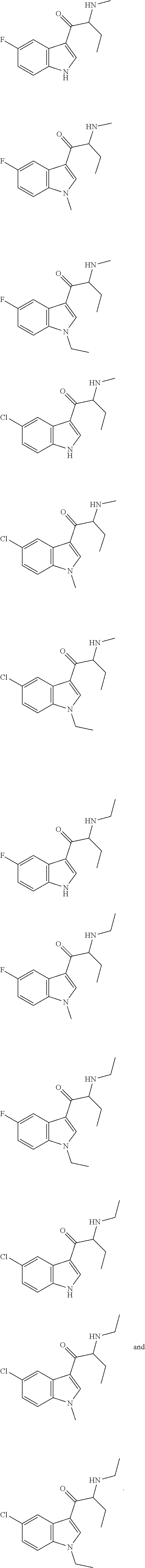

Other embodiments include a racemic compound, pure R- or S-enantiomer, or enantiomerically enriched mixture of the present invention is selected from:

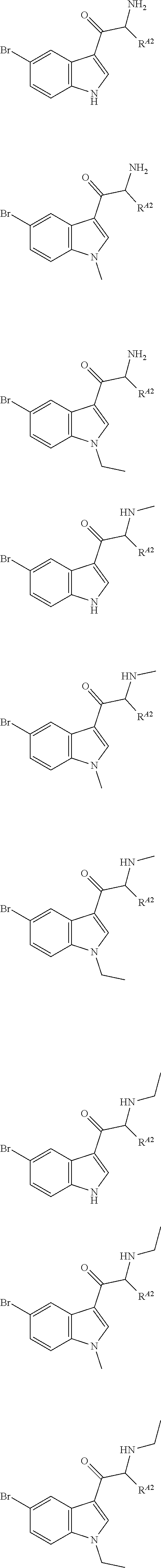

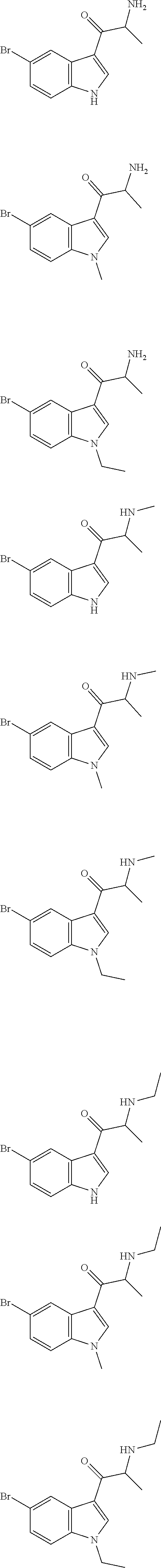

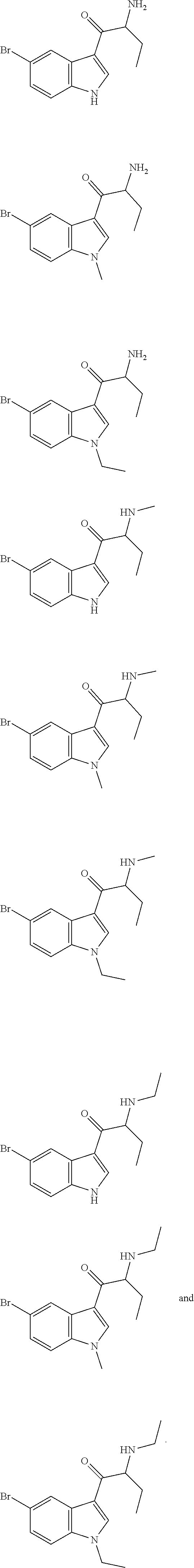

In other embodiments, a racemic compound, pure R- or S-enantiomer, or enantiomerically enriched mixture of the present invention is selected from:

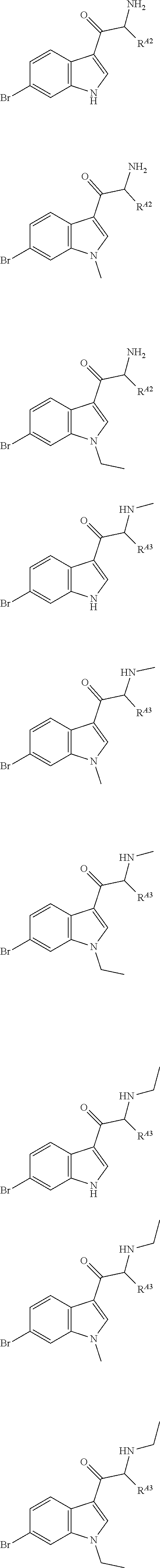

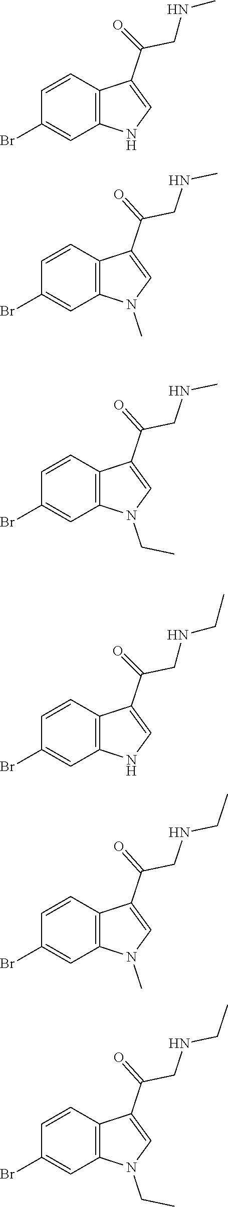 [SMILES: CNCC(=O)c1c[nH]c2cc(Br)ccc12 ; CNCC(=O)c1cn(C)c2cc(Br)ccc12 ; CCn1cc(C(=O)CNC)c2ccc(Br)cc21 ; CCNCC(=O)c1c[nH]c2cc(Br)ccc12 ; CCNCC(=O)c1cn(C)c2cc(Br)ccc12 ; CCNCC(=O)c1cn(CC)c2cc(Br)ccc12]

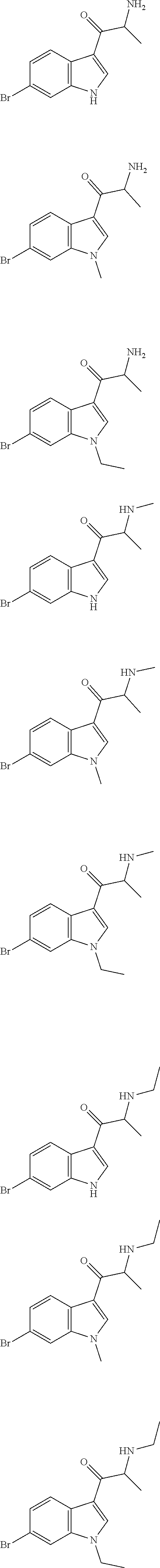

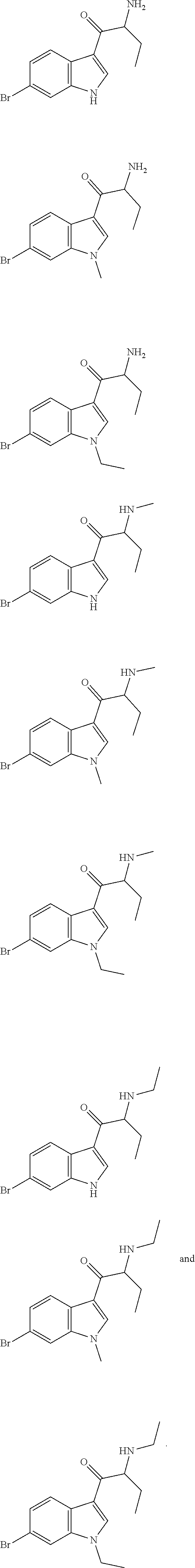

Further embodiments include a racemic compound, pure R- or S-enantiomer, or enantiomerically enriched mixture of the present invention is selected from:

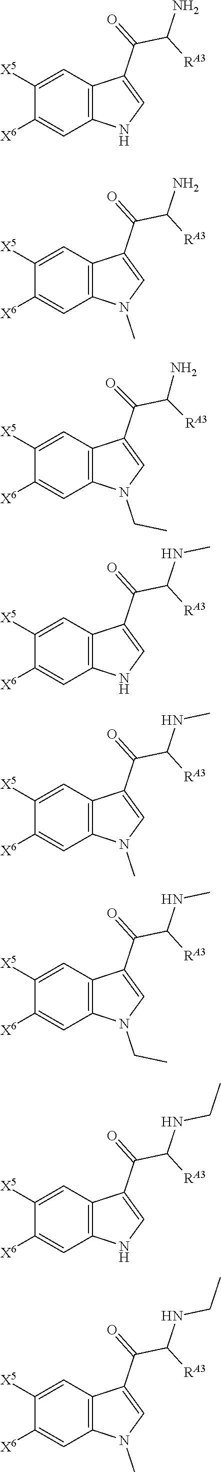

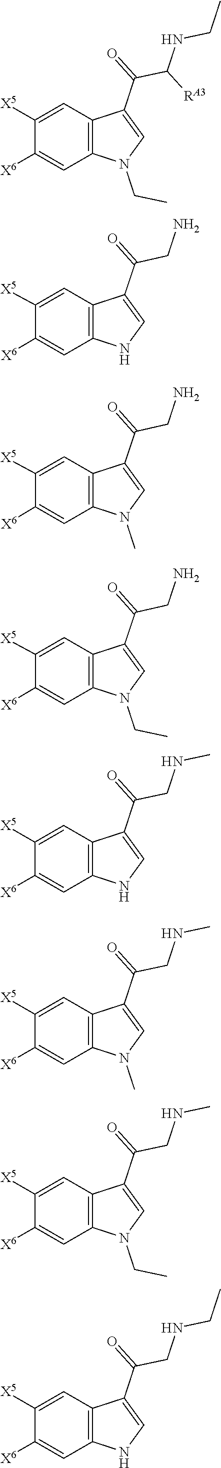

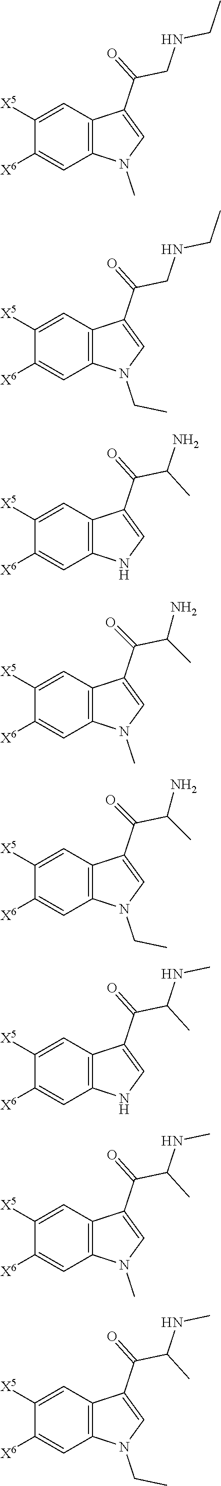

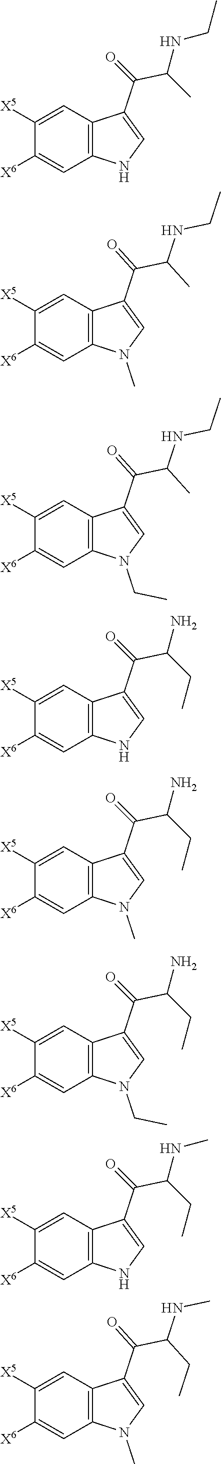

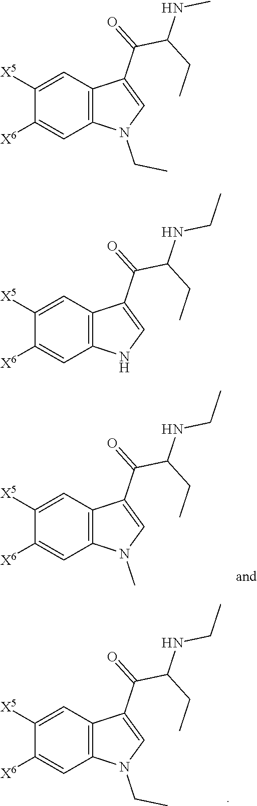

In certain embodiments, a racemic compound, pure R- or S-enantiomer, or enantiomerically enriched mixture of the present invention is selected from:

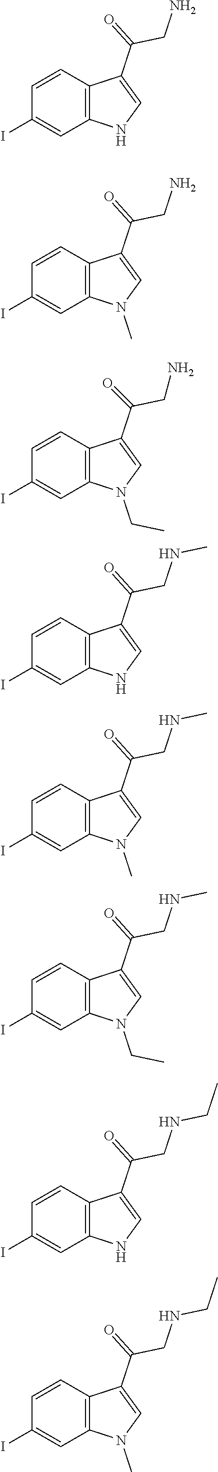

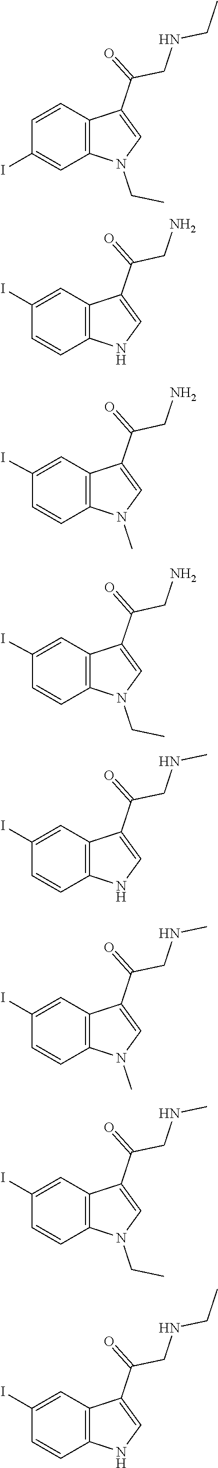

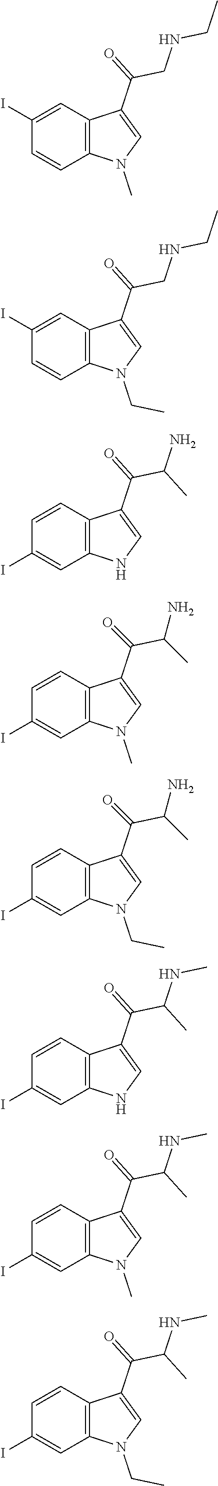

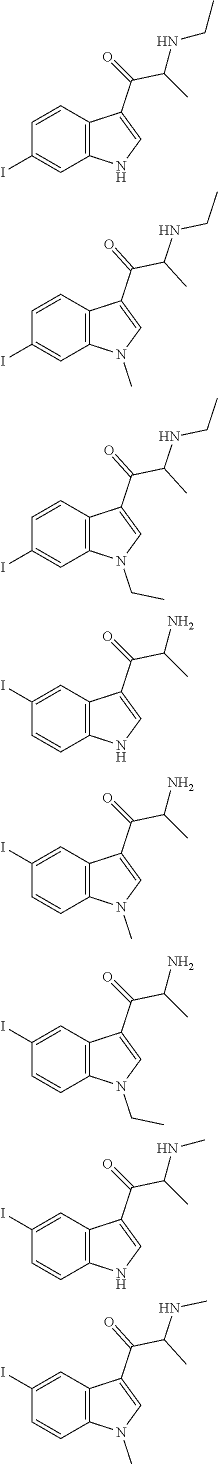

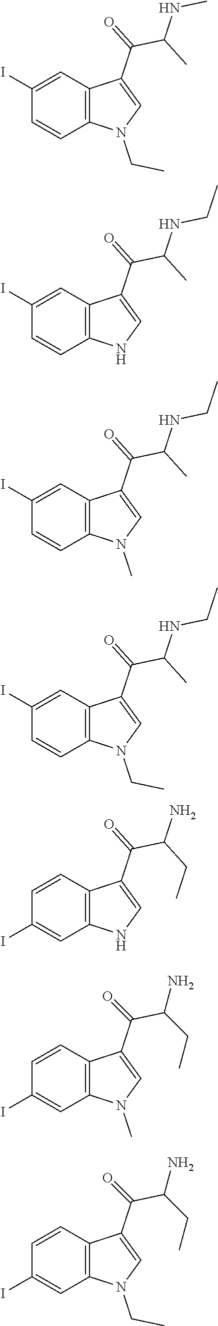

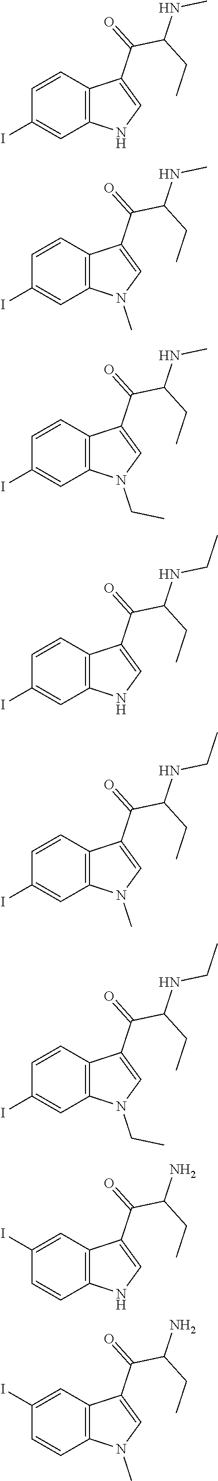

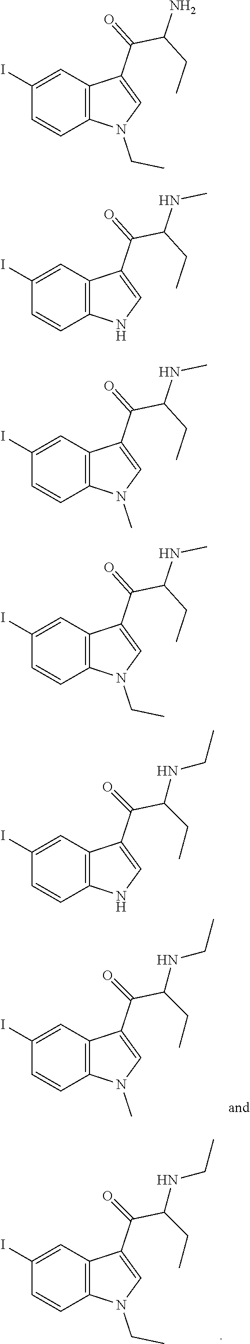

In some embodiments, a racemic compound, pure R- or S-enantiomer, or enantiomerically enriched mixture of the present invention is selected from:

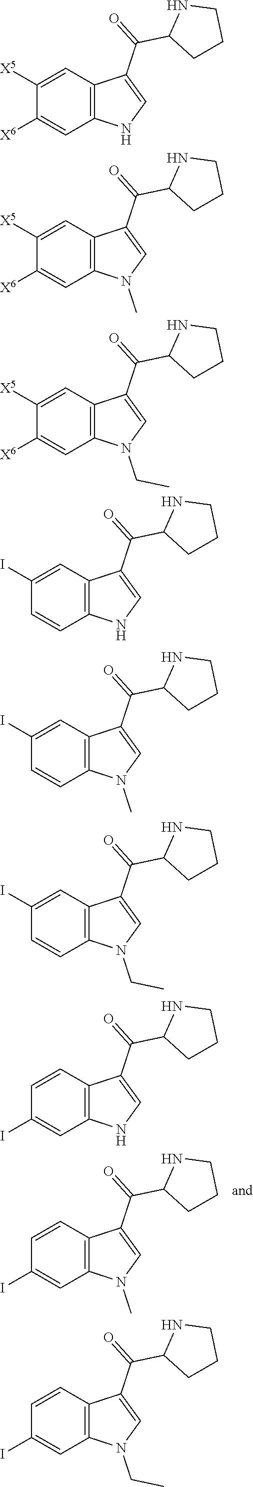

In certain embodiments, a racemic compound, pure R- or S-enantiomer, or enantiomerically enriched mixture of the present invention is selected from:

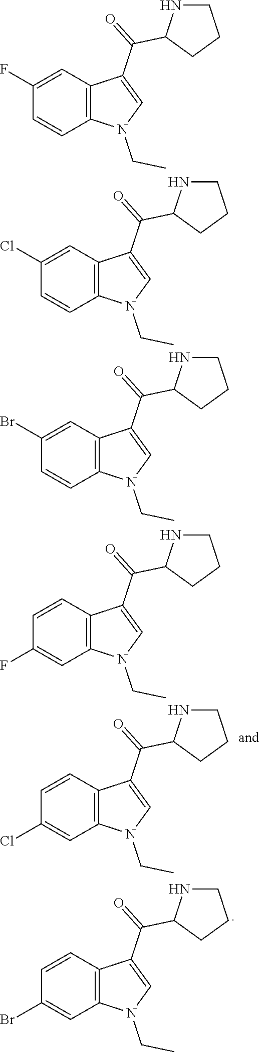

In further embodiments, a racemic compound, pure R- or S-enantiomer, or enantiomerically enriched mixture of the present invention is selected from:

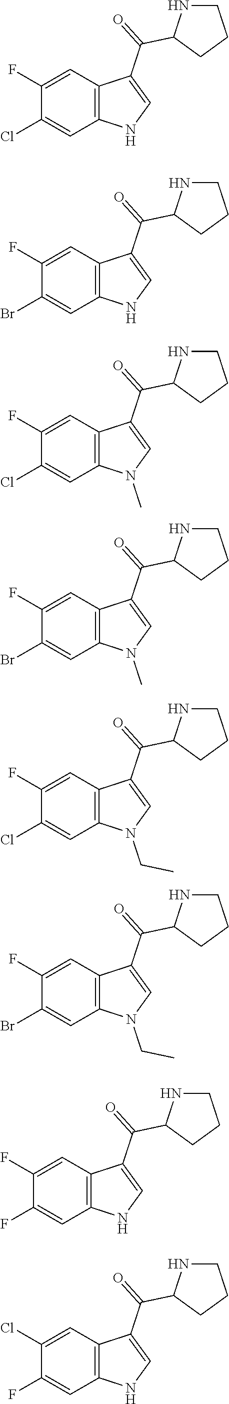

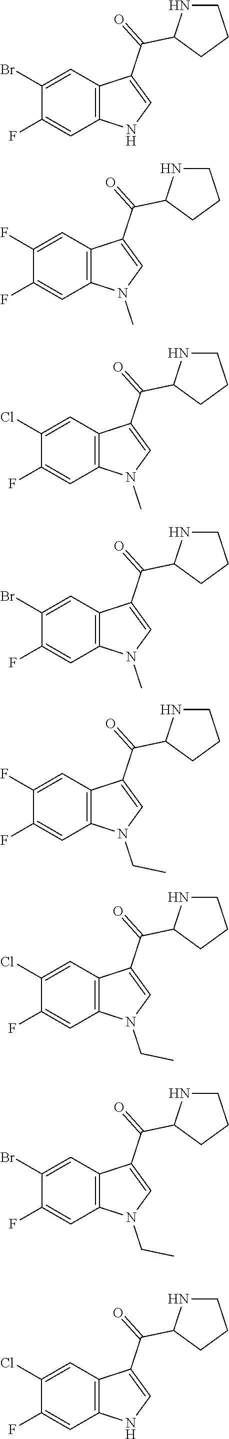

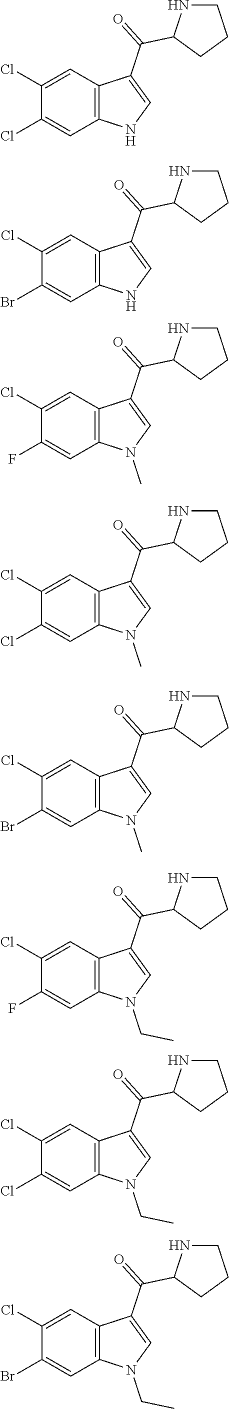

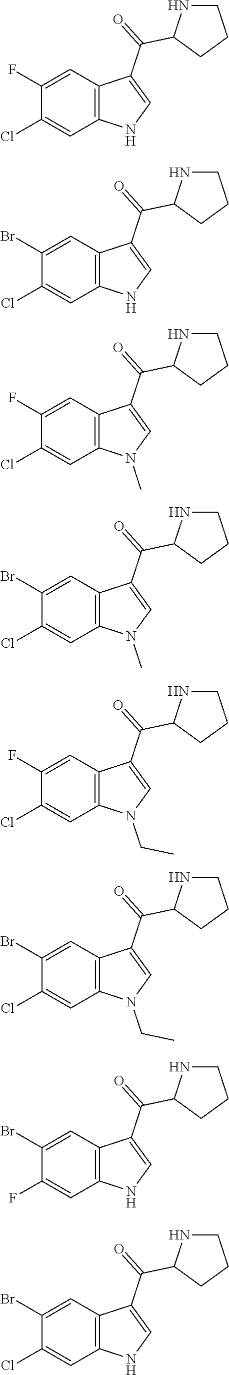

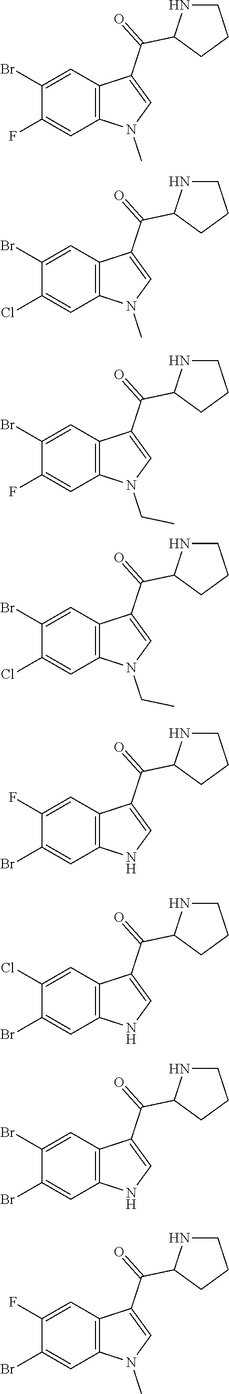

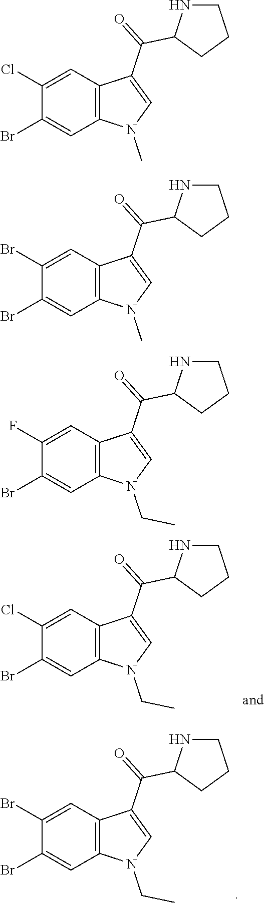

The chiral carbon typically referred to in this application is the carbon alpha to the amine in the heteroarylethylamine motif. Of course, the compounds can have additional chiral centers that result in diastereomers. Notwithstanding, in the present application, the primary chiral carbon referred to in the term “enantiomerically enriched” is that carbon alpha to the amine in the provided structures.

In certain embodiments, isolated enantiomers of the compounds of the present invention show improved binding at the desired receptors and transporters relevant to the goal of treatment for the mental disorder or for mental enhancement.

An enantiomerically enriched mixture is a mixture that contains one enantiomer in a greater amount than the other. An enantiomerically enriched mixture of an S-enantiomer contains at least 55% of the S-enantiomer, and, typically at least about 60%, 65%, 70%, 75%, 80%, 85%, 90%, or 95% or more of the S-enantiomer. An enantiomerically enriched mixture of an R-enantiomer contains at least 55% of the R-enantiomer, and typically at least about 60%, 65%, 70%, 75%, 80%, 85%, 90% or 95% of the R-enantiomer. The specific ratio of S or R enantiomer can be selected for the need of the patient according to the health care specialist to balance the desired effect.

The term enantiomerically enriched mixture as used in this application does not include a racemic mixture and does not include a pure isomer. Notwithstanding, it should be understood that any compound described herein in enantiomerically enriched form can be used as a pure isomer (or a racemic form) if it achieves the goal of any of the specifically itemized methods of treatment described herein, including but not limited to BK-5F-NM-AMT, BK-5Cl-NM-AMT, or BK-5Br-NM-AMT.

In certain embodiments, it is useful to have an S- or R-enantiomerically enriched mixture of these entactogenic compounds that is not a racemic mixture. Enantiomerically enriched mixtures that have a greater amount of the one enantiomer of BK-5F-NM-AMT, BK-5Cl-NM-AMT, or BK-5Br-NM-AMT potentially maximize serotonin-receptor-dependent therapeutic effects, whereas the enantiomerically enriched opposite enantiomer of BK-5F-NM-AMT, BK-5Cl-NM-AMT, or BK-5Br-NM-AMT potentially increases dopaminergic-receptor-dependent therapeutic effects relative to the racemic mixture. Therefore, one aspect of the present invention is a balanced mixture of S-BK-5F-NM-AMT and R-BK-5F-NM-AMT, a balanced mixture of S-BK-5Cl-NM-AMT and R-BK-5Cl-NM-AMT, and a balanced mixture of S-BK-5Br-NM-AMT and R-BK-5Br-NM-AMT that achieves a predetermined combination of serotonin-receptor-dependent therapeutic effects and dopaminergic therapeutic effects. The effect can be modulated as desired for optimal therapeutic effect.

Non-limiting examples of unwanted effects that can be minimized by carefully selecting the balance of enantiomers include hallucinogenic effects (for example, perceptual distortions, delusions, depersonalization, derealization, and labile mood), psychoactive effects (including excess stimulation or sedation), physiological effects (including transient hypertension or appetite suppression), toxic effects (including to the brain or liver), effects contributing to abuse liability (including euphoria or dopamine release), and/or other side effects.

In some aspects, an enantiomerically enriched mixture of the S-enantiomer or pure enantiomer of S-BK-5F-NM-AMT or an enantiomerically enriched mixture of the S-enantiomer or pure enantiomer of S-BK-5Cl-NM-AMT, or an enantiomerically enriched mixture of the S-enantiomer or pure enantiomer of S-BK-5Br-NM-AMT balances therapeutic effects (such as emotional openness and perceptible mood effects) while having lesser effects associated with abuse liability (such as perceptible ‘good drug effects’ or desire for more drug, which can lead to abuse; Pool et al. 2016. Neuroscience & Biobehavioral Reviews, 63, pp. 124-142) when administered to a host in need thereof, for example a mammal, including a human. The enantiomerically enriched mixture or pure enantiomer achieves a predetermined combination of emotional therapeutic effects and perceptible mood effects. The effect can be modulated as desired for optimal therapeutic effect.

In other aspects, an enantiomerically enriched mixture of the R-enantiomer or pure enantiomer of R-BK-5F-NM-AMT or an enantiomerically enriched mixture of the R-enantiomer or pure enantiomer of R-BK-5Cl-NM-AMT, or an enantiomerically enriched mixture of the R-enantiomer or pure enantiomer of R-BK-5Br-NM-AMT balances therapeutic effects (such as emotional openness and perceptible mood effects) while having lesser effects associated with abuse liability (such as perceptible ‘good drug effects’ or desire for more drug, which can lead to abuse; Pool et al. 2016. Neuroscience & Biobehavioral Reviews, 63, pp. 124-142) when administered to a host in need thereof, for example a mammal, including a human. The enantiomerically enriched mixture or pure enantiomer achieves a predetermined combination of emotional therapeutic effects and perceptible mood effects. The effect can be modulated as desired for optimal therapeutic effect.

The present invention also provides new medical uses for the compounds, pure R- or S-enantiomers or enantiomerically enriched mixtures of Formulas I-XXIX, BK-5F-NM-AMT, BK-5Cl-NM-AMT, and BK-5Br-NM-AMT, by administering an effective amount to a patient such as a human to treat a CNS disorder including but not limited to, the treatment of post-traumatic stress disorder, depression, dysthymia, anxiety, generalized anxiety, social anxiety, panic, post-traumatic stress disorder, adjustment disorders, feeding and eating disorders, binge behaviors, body dysmorphic syndromes, addiction, drug abuse or dependence disorders, substance use disorders, disruptive behavior disorders, impulse control disorders, gaming disorders, gambling disorders, memory loss, dementia of aging, attention deficit hyperactivity disorder, personality disorders, attachment disorders, autism or dissociative disorders or any other disorder described herein, including in the Background.

The present compounds are not significant agonists of 5-HT_{2A}. For example, as described in the non-limiting illustrative Example 5, all the tested compounds show minimal 5-HT_{2A} agonist activity. For all tested compounds, the 5-HT_{2A} agonist activity was too weak to detect an EC₅₀ below 10 μM. Additionally, as shown in Example 3, the compounds of the present invention do not significantly alter 5-HT_{2A} activity when measured by a calcium flux assay.

The present invention also includes compounds with beneficial selectivity profiles for neurotransmitter transporters. The balance of weakly activating NET (to reduce cardiovascular toxicity risk) and having a relatively low DAT to SERT ratio (to increase therapeutic effect relative to addictive liability) is a desirable feature of an entactogenic therapy displayed by the compounds and compositions of the present invention. Every tested compound displayed a DAT to SERT ratio less than one, indicating each compound is more selective for SERT (therapeutic effect) than DAT (addictive liability) as described in the non-limiting illustrative Example 7.

In certain aspects of these embodiments, one or more selected compounds of Formulas I-XXIX can be improved or “tuned” by administering an effective amount to a host such as a human, in need thereof, in a composition of a pure enantiomer (or diastereomer, where relevant), or alternatively, an enantiomerically enriched composition that has an abundance of one enantiomer over the other. In this way, as described above, the enantiomeric forms act differently from each other on various 5-HT receptors, dopamine receptors, and norepinephrine receptors, producing variable effects, and that those effects can be selected for based on desired outcome for the patient.

In certain embodiments, any of the selected compounds or mixtures of the present invention is administered to a patient in an effective amount in conjunction with psychotherapy, cognitive enhancement, or life coaching (pharmacotherapy), or as part of routine medical therapy.

The present invention also provides compounds that in certain embodiments can be used in methods for the modulation of CNS activity and/or a method for treatment of CNS disorders, including, but not limited to post-traumatic stress and adjustment disorders, comprising administering a compound of Formula XXVI, Formula XXVII, Formula XXVIII or Formula XXIX or a pharmaceutically acceptable salt thereof:

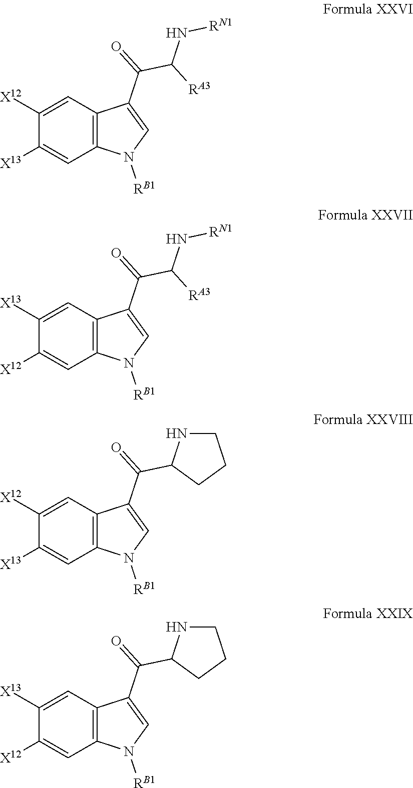

wherein all variables are as defined herein.

The compounds may be provided in a composition that is enantiomerically enriched, such as a mixture of enantiomers in which one enantiomer is present in excess, in particular to the extent of 60% or more, 70% or more, 75% or more, 80% or more, 90% or more, 95% or more, or 97% or more, or alternatively, as a pure isomer.

Where diastereomers exist, the compounds can be used in any diastereomeric form or mixture of forms that provides the appropriate therapeutic effect for the patient, as taught herein. Therefore, in one embodiment, the compounds of the present invention can be administered in a racemic mixture, as the R-enantiomer, as the S-enantiomer, or as an enantiomerically enriched mixture, or a diastereomeric form.

The following compound illustrations indicate where primary stereocenters exist when the designated R group (for example, R^{A1}, R^{A2}, or R^{A3}) is not hydrogen. In certain embodiments, the enantiomers of the present invention include compounds of Formula I:

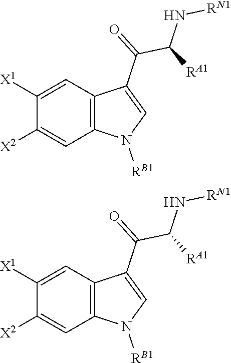

In some embodiments, the enantiomers of the present invention include compounds of Formula II:

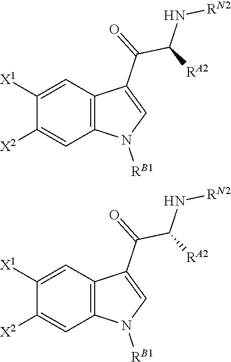

In other embodiments, the enantiomers of the present invention include compounds of Formula III:

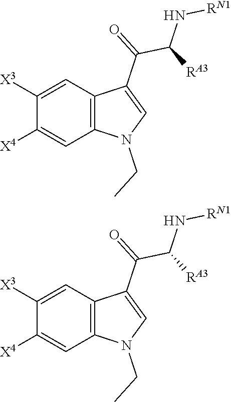

wherein R^{A3} is not —H.

In certain embodiments, the enantiomers of the present invention include compounds of Formula IV:

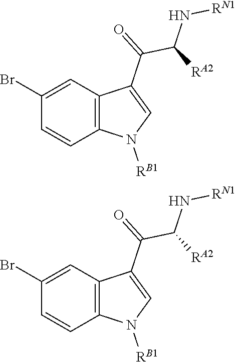

In further embodiments, the enantiomers of the present invention include compounds of Formula V:

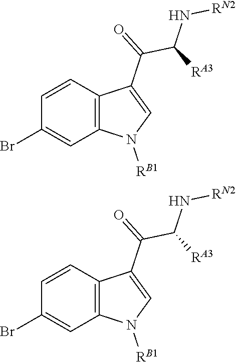

wherein R^{A3} is not —H.

In yet more embodiments, the enantiomers of the present invention include compounds of Formula VI:

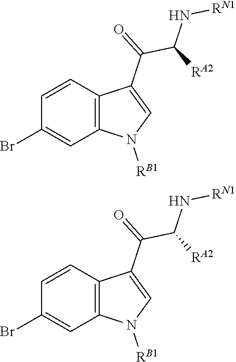

In some embodiments, the enantiomers of the present invention include compounds of Formula VII:

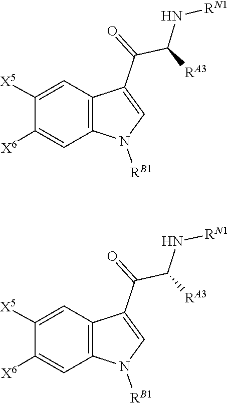

In certain embodiments, the enantiomers of the present invention include compounds of Formula VIII:

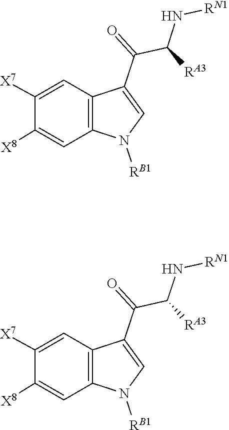

wherein R^{A3} is not —H.

In other embodiments, the enantiomers of the present invention include compounds of Formula IX:

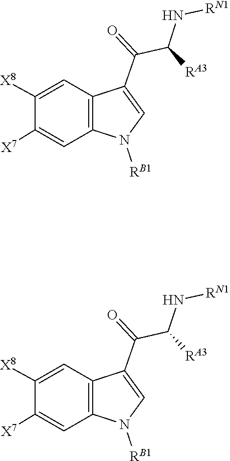

wherein R^{A3} is not —H.

In further embodiments, the enantiomers of the present invention include compounds of Formula X:

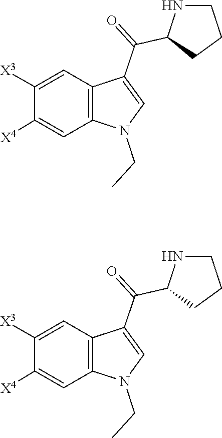

In some embodiments, the enantiomers of the present invention include compounds of Formula XI:

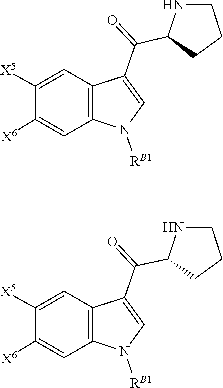

In certain embodiments, the enantiomers of the present invention include compounds of Formula XII:

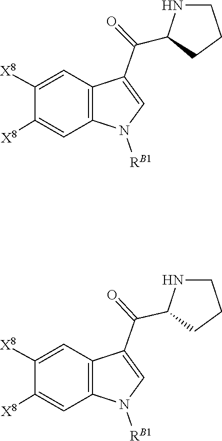

In further embodiments, the enantiomers of the present invention include compounds of Formula XIII:

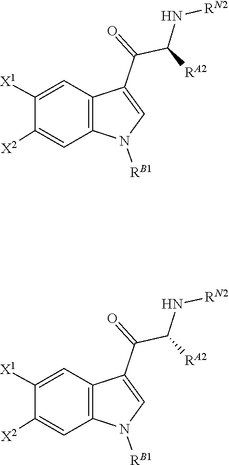

In other embodiments, the enantiomers of the present invention include compounds of Formula XIV:

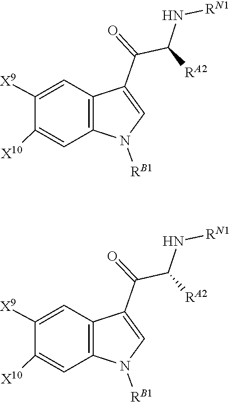

In some embodiments, the enantiomers of the present invention include compounds of Formula XV:

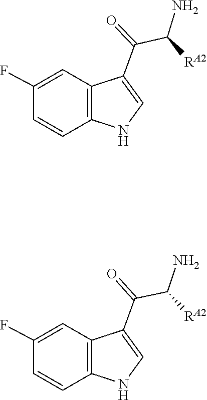

In further embodiments, the enantiomers of the present invention include compounds of Formula XVI:

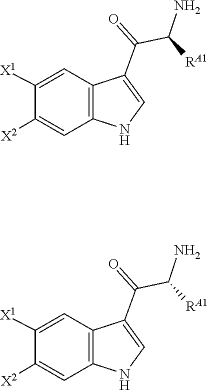

In certain embodiments, the enantiomers of the present invention include compounds of Formula XVII:

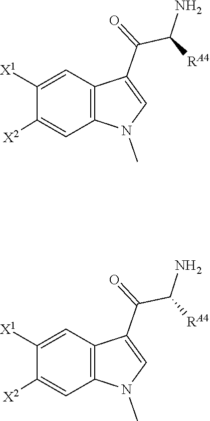

In other embodiments, the enantiomers of the present invention include compounds of Formula XVIII:

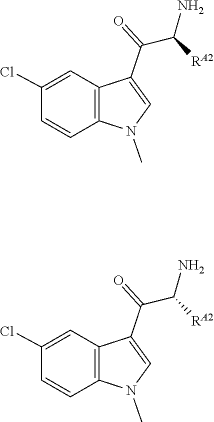

In some embodiments, the enantiomers of the present invention include compounds of Formula XIX:

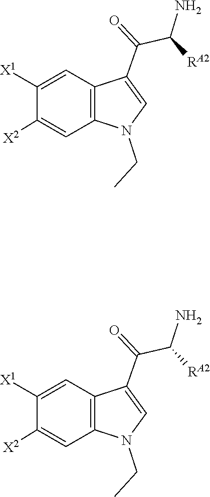

In further embodiments, the enantiomers of the present invention include compounds of Formula XX:

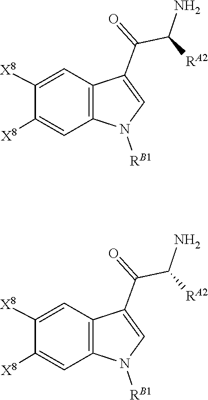

In certain embodiments, the enantiomers of the present invention include compounds of Formula XXI:

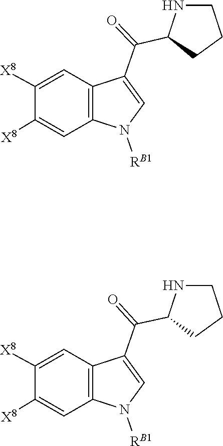

In other embodiments, the enantiomers of the present invention include compounds of Formula XXII:

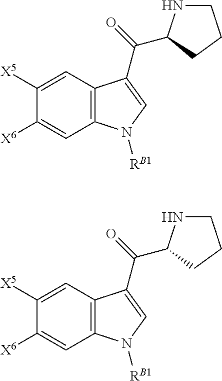

In some embodiments, the enantiomers of the present invention include compounds of Formula XXIII:

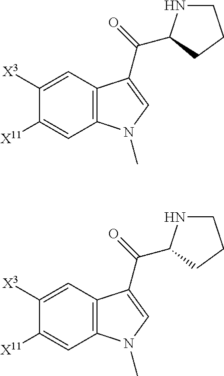

In further embodiments, the enantiomers of the present invention include compounds of Formula XXIV:

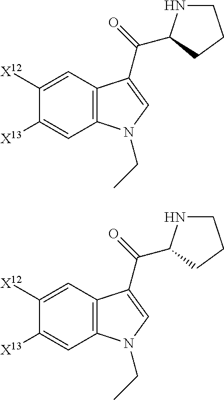

In certain embodiments, the enantiomers of the present invention include compounds of Formula XXV:

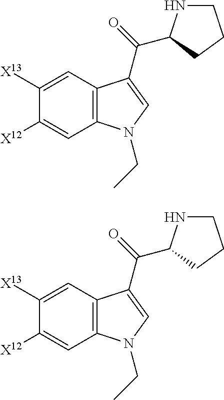

In other embodiments, the enantiomerically enriched mixtures of the present invention are enriched with compounds of Formula XXVI:

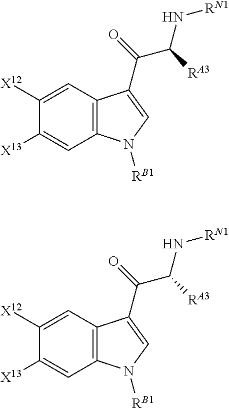

wherein R^{A3} is not —H.

In some embodiments, the enantiomerically enriched mixtures of the present invention are enriched with compounds of Formula XXVII:

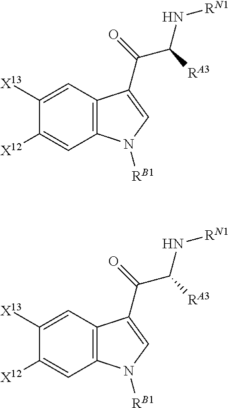

wherein R^{A3} is not —H.

In further embodiments, the enantiomerically enriched mixtures of the present invention are enriched with compounds of Formula XXVIII:

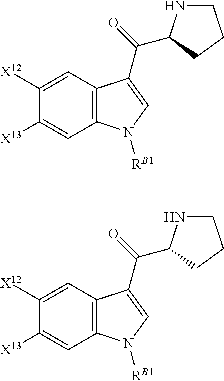

In certain embodiments, the enantiomerically enriched mixtures of the present invention are enriched with compounds of Formula XXIX:

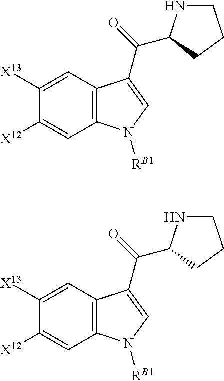

Certain compounds of the invention may also exist in several tautomeric forms including the enol form, the keto form, and mixtures thereof. The chemical structures depicted herein encompass all possible tautomeric forms of the illustrated compounds. Keto-enol tautomerism, for example, is the reversible transfer of a hydrogen from the alpha carbon adjacent to a carbonyl group followed by a double bond transfer. In solution, compounds will spontaneously undergo a kinetic transformation from one tautomer to the other until equilibrium is reached, generally strongly favoring the keto tautomer over the enol tautomer, but dependent on factors such as solvent, pH, and temperature. Keto and enol tautomers may have distinguishable physicochemical properties; however, because they will interconvert in solution, reference to a compound in its keto form (for example, where Q¹ is

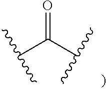 [SMILES: *C(*)=O]

will be understood to refer to and include the compound in its enol form (for example, where Q¹ is

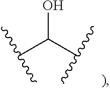 [SMILES: *C(*)O]

unless context clearly indicates otherwise. The compounds may also exist as ring-chain tautomers, as discussed below.

### Preparation of Enantiomeric Compounds

Various methods are known in the art for preparing optically active forms and determining activity. Such methods include standard processes described herein and other similar assays which are well known in the art. Examples of methods that can be used to obtain optical isomers of the compounds according to the present disclosure include but are not limited to the following:
a) physical separation of crystals whereby macroscopic crystals of the individual enantiomers are manually separated. This technique may particularly be used if crystals of the separate enantiomers exist (i.e., the material is a conglomerate), and the crystals are visually distinct;
b) simultaneous crystallization whereby the individual enantiomers are separately crystallized from a solution of the racemate, possible only if the latter is a conglomerate in the solid state;
c) enzymatic resolutions whereby partial or complete separation of a racemate by virtue of differing rates of reaction for the enantiomers with an enzyme;
d) enzymatic asymmetric synthesis, a synthetic technique whereby at least one step of the synthesis uses an enzymatic reaction to obtain an enantiomerically pure or enriched synthetic precursor of the desired enantiomer;
e) chemical asymmetric synthesis whereby the desired enantiomer is synthesized from an achiral precursor under conditions that produce asymmetry (i.e., chirality) in the product, which may be achieved using chiral catalysts or chiral auxiliaries;
f) diastereomer separations whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
g) first- and second-order asymmetric transformations whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomers;
h) kinetic resolutions comprising partial or complete resolution of a racemate (or of a further resolution of a partially resolved compound) by virtue of unequal reaction rates of the enantiomers with a chiral, enantiomerically enriched reagent or catalyst under kinetic conditions;
i) enantiospecific synthesis from enantiomerically enriched precursors whereby the desired enantiomer is obtained from non-chiral starting materials and where the stereochemical integrity is not or is only minimally compromised over the course of the synthesis;
j) chiral liquid chromatography whereby the enantiomers of a racemate are separated in a liquid mobile phase by virtue of their differing interactions with a stationary phase. The stationary phase can be made of chiral material, or the mobile phase can contain an additional chiral material to provoke the differing interactions;
k) chiral gas chromatography whereby the racemate is volatilized and enantiomers are separated by virtue of their differing interactions in the gaseous mobile phase with a column containing a fixed enantiomerically enriched chiral adsorbent phase;
l) extraction with chiral solvents whereby the enantiomers are separated by virtue of preferential dissolution of one enantiomer into a particular chiral solvent; and
m) transport across chiral membranes whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the enantiomerically enriched chiral nature of the membrane, which allows only one enantiomer of the racemate to pass through.

### Enantiomerically Enriched Pharmaceutical Compositions

Chiral compounds of the invention may be prepared by chiral chromatography from the racemic or enantiomerically enriched free amine. Pharmaceutically acceptable salts of chiral compounds may be prepared from fractional crystallization of salts from a racemic or an enantiomerically enriched free amine and a chiral acid. Alternatively, the free amine may be reacted with a chiral auxiliary and the enantiomers separated by chromatography followed by removal of the chiral auxiliary to regenerate the free amine. Furthermore, separation of enantiomers may be performed at any convenient point in the synthesis of the compounds of the invention. The compounds of the invention may also be prepared using a chiral synthesis.

An enantiomerically enriched mixture is a mixture that contains one enantiomer in a greater amount than the other. An enantiomerically enriched mixture of an S-enantiomer contains at least 55% of the S-enantiomer, and more typically at least about 60%, 65%, 70%, 75%, 80%, 85%, 90%, 95% of the S-enantiomer. An enantiomerically enriched mixture of an R-enantiomer contains at least 55% of the R-enantiomer, more typically at least about 55%, 60%, 65%, 70%, 75%, 80%, 85%, 90%, 95% of the R-enantiomer.

In one embodiment, enantiomerically enriched mixtures are created that have a greater amount of the serotonin-receptor-dependent therapeutic effects.

In one embodiment, enantiomerically enriched mixtures are created that have a greater amount of the dopaminergic effects. In one embodiment, enantiomerically enriched mixtures are created that have a greater amount of the serotonin-receptor-dependent therapeutic effects.

Non-limiting examples of unwanted effects that can be minimized include psychoactive effects (such as excess stimulation or sedation), physiological effects (such as transient hypertension or appetite suppression), toxic effects (such as to the brain or liver), effects contributing to abuse liability (such as euphoria or dopamine release), and other side effects.

One aspect of the present invention is a balanced mixture of S-BK-5F-NM-AMT and R-BK-5F-NM-AMT, a balanced mixture of S-BK-5Cl-NM-AMT and R-BK-5Cl-NM-AMT, or a balanced mixture of S-BK-5Br-NM-AMT and R-BK-5Br-NM-AMT that achieves a predetermined combination of serotonin-receptor-dependent therapeutic effects and dopaminergic effects.

In certain embodiments, pharmaceutical compositions of enantiomerically enriched preparations of BK-5F-NM-AMT, BK-5Cl-NM-AMT, or BK-5Br-NM-AMT are provided. In one embodiment, the pharmaceutical composition is enriched with S-BK-5F-NM-AMT. In one embodiment, the pharmaceutical composition is enriched with R-BK-5F-NM-AMT. In one embodiment, the pharmaceutical composition is enriched with S-BK-5Cl-NM-AMT. In one embodiment, the pharmaceutical composition is enriched with R-BK-5Cl-NM-AMT. In one embodiment, the pharmaceutical composition is enriched with S-BK-5Br-NM-AMT. In one embodiment, the pharmaceutical composition is enriched with R-BK-5Br-NM-AMT.

Example 2 provides non-limiting examples for the preparation of certain enantiomerically enriched preparations of BK-5F-NM-AMT, BK-5Cl-NM-AMT, and BK-5Br-NM-AMT (i.e., comprising the S- and R-enantiomer). Enantiomerically enriched preparations of other compounds of the present invention can be similarly produced using racemic mixtures of the same compound.

Particular embodiments for pharmaceutical compositions, including enantiomerically enriched pharmaceutical compositions, of the present invention include:
a) S-BK-5F-NM-AMT
b) R-BK-5F-NM-AMT
c) S-BK-5Cl-NM-AMT
d) R-BK-5Cl-NM-AMT
e) S-BK-5Br-NM-AMT
f) R-BK-5Br-NM-AMT
g) Embodiments (a)-(f) wherein the compound is a free base;
h) Embodiments (a)-(f) wherein the compound is a salt;
i) Embodiment (h) wherein the compound is the hydrochloride salt;
j) A mixture of S-BK-5F-NM-AMT and R-BK-5F-NM-AMT and there is more S-enantiomer than R-enantiomer;
k) A mixture of S-BK-5F-NM-AMT and R-BK-5F-NM-AMT and there is less S-enantiomer than R-enantiomer;
l) A mixture of S-BK-5Cl-NM-AMT and R-BK-5Cl-NM-AMT and there is more S-enantiomer than R-enantiomer;
m) A mixture of S-BK-5Cl-NM-AMT and R-BK-5Cl-NM-AMT and there is less S-enantiomer than R-enantiomer;
n) A mixture of S-BK-5Br-NM-AMT and R-BK-5Br-NM-AMT and there is more S-enantiomer than R-enantiomer;
o) A mixture of S-BK-5Br-NM-AMT and R-BK-5Br-NM-AMT and there is less S-enantiomer than R-enantiomer;
p) A mixture of S-BK-5F-NM-AMT and R-BK-5F-NM-AMT and at least about 65% is the S-enantiomer while no more than 35% is the R-enantiomer;
q) A mixture of S-BK-5F-NM-AMT and R-BK-5F-NM-AMT and greater than 65% is the S-enantiomer while less than 35% is the R-enantiomer;
r) A mixture of S-BK-5F-NM-AMT and R-BK-5F-NM-AMT and greater than 90% is the S-enantiomer while less than 10% is the R-enantiomer;
s) A mixture of S-BK-5F-NM-AMT and R-BK-5F-NM-AMT and at least about 35% is the S-enantiomer while not more than 65% is the R-enantiomer;
t) A mixture of S-BK-5F-NM-AMT and R-BK-5F-NM-AMT and less than 35% is the S-enantiomer while greater than 65% is the R-enantiomer;
u) A mixture of S-BK-5F-NM-AMT and R-BK-5F-NM-AMT and less than 10% is the S-enantiomer while greater than 90% is the R-enantiomer;
v) A mixture of S-BK-5Cl-NM-AMT and R-BK-5Cl-NM-AMT and at least about 65% is the S-enantiomer while no more than 35% is the R-enantiomer;
w) A mixture of S-BK-5Cl-NM-AMT and R-BK-5Cl-NM-AMT and greater than 65% is the S-enantiomer while less than 35% is the R-enantiomer;
x) A mixture of S-BK-5Cl-NM-AMT and R-BK-5Cl-NM-AMT and greater than 90% is the S-enantiomer while less than 10% is the R-enantiomer;
y) A mixture of S-BK-5Cl-NM-AMT and R-BK-5Cl-NM-AMT and at least about 35% is the S-enantiomer while not more than 65% is the R-enantiomer;
z) A mixture of S-BK-5Cl-NM-AMT and R-BK-5Cl-NM-AMT and less than 35% is the S-enantiomer while greater than 65% is the R-enantiomer;
aa) A mixture of S-BK-5Cl-NM-AMT and R-BK-5Cl-NM-AMT and less than 10% is the S-enantiomer while greater than 90% is the R-enantiomer;
bb) A mixture of S-BK-5Br-NM-AMT and R-BK-5Br-NM-AMT and at least about 65% is the S-enantiomer while no more than 35% is the R-enantiomer;
cc) A mixture of S-BK-5Br-NM-AMT and R-BK-5Br-NM-AMT and greater than 65% is the S-enantiomer while less than 35% is the R-enantiomer;
dd) A mixture of S-BK-5Br-NM-AMT and R-BK-5Br-NM-AMT and greater than 90% is the S-enantiomer while less than 10% is the R-enantiomer;
ee) A mixture of S-BK-5Br-NM-AMT and R-BK-5Br-NM-AMT and at least about 35% is the S-enantiomer while not more than 65% is the R-enantiomer;
ff) A mixture of S-BK-5Br-NM-AMT and R-BK-5Br-NM-AMT and less than 35% is the S-enantiomer while greater than 65% is the R-enantiomer;
gg) A mixture of S-BK-5Br-NM-AMT and R-BK-5Br-NM-AMT and less than 10% is the S-enantiomer while greater than 90% is the R-enantiomer;

It will be understood that the above embodiments and classes of embodiments can be combined to form additional embodiments.

### III. Methods to Treat CNS Disorders Including Mental Disorders and for Mental Enhancement

The present invention provides methods and uses for the treatment of CNS disorders, including, but not limited to, mental disorders as described herein, including post-traumatic stress and adjustment disorders, and other disorders described in the Background, Summary or Description herein, comprising administering the tryptamine compounds or composition or a pharmaceutically acceptable salt or salt mixture thereof as described herein. These compounds display a number of pharmacological properties that are beneficial to their use as therapeutics and represent an improvement over existing therapeutics.

The present invention provides, for example, methods for the treatment of disorders, including, but not limited to depression, dysthymia, anxiety and phobia disorders (including generalized anxiety, social anxiety, panic, post-traumatic stress and adjustment disorders), feeding and eating disorders (including binge eating, bulimia, and anorexia nervosa), other binge behaviors, body dysmorphic syndromes, alcoholism, tobacco abuse, drug abuse or dependence disorders, disruptive behavior disorders, impulse control disorders, gaming disorders, gambling disorders, memory loss, dementia of aging, attention deficit hyperactivity disorder, personality disorders (including antisocial, avoidant, borderline, histrionic, narcissistic, obsessive compulsive, paranoid, schizoid and schizotypal personality disorders), attachment disorders, autism, and dissociative disorders.

In addition to treating various diseases and disorders, the employed methods of modulating activity of the serotonergic system in particular can be used to improve CNS functioning in non-disease states, such as reducing neuroticism and psychological defensiveness, increasing openness to experience, increasing creativity, and aiding decision-making.

In other embodiments, a compound or composition of the present invention is provided in an effective amount to treat a host, typically a human, with a CNS disorder that can be either a neurological condition (one that is typically treated by a neurologist) or a psychiatric condition (one that is typically treated by a psychiatrist). Neurological disorders are typically those affecting the structure, biochemistry or cause electrical abnormalities of the brain, spinal cord or other nerves. Psychiatric conditions are more typically thought of as mental disorders, which are primarily abnormalities of thought, feeling or behavior that cause significant distress or impairment of personal functioning.

Thus, the disclosed compounds can be used in an effective amount to improve neurological or psychiatric functioning in a patient in need thereof. Neurological indications include, but are not limited to improved neuroplasticity, including treatment of stroke, brain trauma, dementia, and neurodegenerative diseases. MDMA has been reported to have an EC50 of 7.41 nM for promoting neuritogenesis and an Eₘₐₓ approximately twice that of ketamine, which has fast acting psychiatric benefits that are thought to be mediated by its ability to promote neuroplasticity, including the growth of dendritic spines, increased synthesis of synaptic proteins, and strengthening synaptic responses (Figure S3. in Ly et al. Cell reports 23, no. 11 (2018): 3170-3182). The compounds of the current invention can similarly be considered psychoplastogens, that is, small molecules that are able to induce rapid neuroplasticity (Olson, 2018, Journal of experimental neuroscience, 12, 1179069518800508). For example, in certain embodiments, the disclosed compounds and compositions can be used to improve stuttering and other dyspraxias or to treat Parkinson's disease or schizophrenia.

The term “improving psychiatric function” is intended to include mental health and life conditions that are not traditionally treated by neurologists but sometimes treated by psychiatrists and can also be treated by psychotherapists, life coaches, personal fitness trainers, meditation teachers, counselors, and the like. For example, it is contemplated that the disclosed compounds will allow individuals to effectively contemplate actual or possible experiences that would normally be upsetting or even overwhelming. This includes individuals with fatal illnesses planning their last days and the disposition of their estate. This also includes couples discussing difficulties in their relationship and how to address them. This also includes individuals who wish to more effectively plan their career.

In other embodiments, the tryptamine compounds and compositions of the present invention may be used in an effective amount to treat a host, typically a human, to modulate an immune or inflammatory response. The compounds disclosed herein alter extracellular serotonin, which is known to alter immune functioning. MDMA produces acute time-dependent increases and decreases in immune response.

The following nonlimiting examples are relevant to any of the disorders, indications, methods of use or dosing regimes described herein.

In certain embodiments, a host, for example a human, is treated with an effective amount of an enantiomerically enriched mixture of enantiomers of compounds of Formulas I-XXIX or a pharmaceutically acceptable salt or salt mixture, isotopic derivative, or prodrug thereof, wherein the percent of S enantiomer is greater than about 95 percent.

In certain embodiments, a host, for example a human, is treated with an effective amount of an enantiomerically enriched mixture of enantiomers of compounds of Formulas I-XXIX or a pharmaceutically acceptable salt or salt mixture, isotopic derivative, or prodrug thereof, wherein the percent of S enantiomer is greater than about 90 percent.

In certain embodiments, a host, for example a human, is treated with an effective amount of an enantiomerically enriched mixture of enantiomers of compounds of Formulas I-XXIX or a pharmaceutically acceptable salt or salt mixture, isotopic derivative, or prodrug thereof, wherein the percent of S enantiomer is greater than about 85 percent.

In certain embodiments, a host, for example a human, is treated with an effective amount of an enantiomerically enriched mixture of enantiomers of compounds of Formulas I-XXIX or a pharmaceutically acceptable salt or salt mixture, isotopic derivative, or prodrug thereof, wherein the percent of S enantiomer is greater than about 80 percent.

In certain embodiments, a host, for example a human, is treated with an effective amount of an enantiomerically enriched mixture of enantiomers of compounds of Formulas I-XXIX or a pharmaceutically acceptable salt or salt mixture, isotopic derivative, or prodrug thereof, wherein the percent of S enantiomer is greater than about 75 percent.

In certain embodiments, a host, for example a human, is treated with an effective amount of an enantiomerically enriched mixture of enantiomers of compounds of Formulas I-XXIX or a pharmaceutically acceptable salt or salt mixture, isotopic derivative, or prodrug thereof, wherein the percent of S enantiomer is greater than about 70 percent.

In certain embodiments, a host, for example a human, is treated with an effective amount of an enantiomerically enriched mixture of enantiomers of compounds of Formulas I-XXIX or a pharmaceutically acceptable salt or salt mixture, isotopic derivative, or prodrug thereof, wherein the percent of S enantiomer is greater than about 65 percent.

In certain embodiments, a host, for example a human, is treated with an effective amount of an enantiomerically enriched mixture of enantiomers of compounds of Formulas I-XXIX or a pharmaceutically acceptable salt or salt mixture, isotopic derivative, or prodrug thereof, wherein the percent of S enantiomer is greater than about 60 percent.

In certain embodiments, a host, for example a human, is treated with an effective amount of an enantiomerically enriched mixture of enantiomers of compounds of Formulas I-XXIX or a pharmaceutically acceptable salt or salt mixture, isotopic derivative, or prodrug thereof, wherein the percent of S enantiomer is greater than about 55 percent.

In certain embodiments, a host, for example a human, is treated with an effective amount of an enantiomerically enriched mixture of enantiomers of compounds of Formulas I-XXIX or a pharmaceutically acceptable salt or salt mixture, isotopic derivative, or prodrug thereof, wherein the percent of S enantiomer is greater than about 55 or 60 percent.

In certain embodiments, a host, for example a human, is treated with an effective amount of an enantiomerically enriched mixture of enantiomers of compounds of Formulas I-XXIX or a pharmaceutically acceptable salt or salt mixture, isotopic derivative, or prodrug thereof, wherein the percent of R enantiomer is greater than about 95 percent.

In certain embodiments, a host, for example a human, is treated with an effective amount of an enantiomerically enriched mixture of enantiomers of compounds of Formulas I-XXIX or a pharmaceutically acceptable salt or salt mixture, isotopic derivative, or prodrug thereof, wherein the percent of R enantiomer is greater than about 90 percent.

In certain embodiments, a host, for example a human, is treated with an effective amount of an enantiomerically enriched mixture of enantiomers of compounds of Formulas I-XXIX or a pharmaceutically acceptable salt or salt mixture, isotopic derivative, or prodrug thereof, wherein the percent of R enantiomer is greater than about 85 percent.

In certain embodiments, a host, for example a human, is treated with an effective amount of an enantiomerically enriched mixture of enantiomers of compounds of Formulas I-XXIX or a pharmaceutically acceptable salt or salt mixture, isotopic derivative, or prodrug thereof, wherein the percent of R enantiomer is greater than about 80 percent.

In certain embodiments, a host, for example a human, is treated with an effective amount of an enantiomerically enriched mixture of enantiomers of compounds of Formulas I-XXIX or a pharmaceutically acceptable salt or salt mixture, isotopic derivative, or prodrug thereof, wherein the percent of R enantiomer is greater than about 75 percent.

In certain embodiments, a host, for example a human, is treated with an effective amount of an enantiomerically enriched mixture of enantiomers of compounds of Formulas I-XXIX or a pharmaceutically acceptable salt or salt mixture, isotopic derivative, or prodrug thereof, wherein the percent of R enantiomer is greater than about 70 percent.

In certain embodiments, a host, for example a human, is treated with an effective amount of an enantiomerically enriched mixture of enantiomers of compounds of Formulas I-XXIX or a pharmaceutically acceptable salt or salt mixture, isotopic derivative, or prodrug thereof, wherein the percent of R enantiomer is greater than about 65 percent.

In certain embodiments, a host, for example a human, is treated with an effective amount of an enantiomerically enriched mixture of enantiomers of compounds of Formulas I-XXIX or a pharmaceutically acceptable salt or salt mixture, isotopic derivative, or prodrug thereof, wherein the percent of R enantiomer is greater than about 60 percent.

In certain embodiments, a host, for example a human, is treated with an effective amount of an enantiomerically enriched mixture of enantiomers of compounds of Formulas I-XXIX or a pharmaceutically acceptable salt or salt mixture, isotopic derivative, or prodrug thereof, wherein the percent of R enantiomer is greater than about 55 percent.

In certain embodiments, a host, for example a human, is treated with an effective amount of an enantiomerically enriched mixture of enantiomers of compounds of Formulas I-XXIX or a pharmaceutically acceptable salt or salt mixture, isotopic derivative, or prodrug thereof, wherein the percent of R enantiomer is greater than about 55 or 60 percent.

In certain embodiments, a host, for example a human, is treated with an effective amount of an enantiomerically enriched mixture of enantiomers of BK-5F-NM-AMT, BK-5Cl-NM-AMT, or BK-5Br-NM-AMT, or a pharmaceutically acceptable salt, salt mixture, isotopic derivative, or prodrug thereof, wherein the percent of R enantiomer is greater than about 95 percent.

In certain embodiments, a host, for example a human, is treated with an effective amount of an enantiomerically enriched mixture of enantiomers of BK-5F-NM-AMT, BK-5Cl-NM-AMT, or BK-5Br-NM-AMT, or a pharmaceutically acceptable salt, salt mixture, isotopic derivative, or prodrug thereof, wherein the percent of R enantiomer is greater than about 90 percent.

In certain embodiments, a host, for example a human, is treated with an effective amount of an enantiomerically enriched mixture of enantiomers of BK-5F-NM-AMT, BK-5Cl-NM-AMT, or BK-5Br-NM-AMT, or a pharmaceutically acceptable salt, salt mixture, isotopic derivative, or prodrug thereof, wherein the percent of R enantiomer is greater than about 85 percent.

In certain embodiments, a host, for example a human, is treated with an effective amount of an enantiomerically enriched mixture of enantiomers of BK-5F-NM-AMT, BK-5Cl-NM-AMT, or BK-5Br-NM-AMT, or a pharmaceutically acceptable salt, salt mixture, isotopic derivative, or prodrug thereof, wherein the percent of R enantiomer is greater than about 80 percent.

In certain embodiments, a host, for example a human, is treated with an effective amount of an enantiomerically enriched mixture of enantiomers of BK-5F-NM-AMT, BK-5Cl-NM-AMT, or BK-5Br-NM-AMT, or a pharmaceutically acceptable salt, salt mixture, isotopic derivative, or prodrug thereof, wherein the percent of R enantiomer is greater than about 75 percent.

In certain embodiments, a host, for example a human, is treated with an effective amount of an enantiomerically enriched mixture of enantiomers of BK-5F-NM-AMT, BK-5Cl-NM-AMT, or BK-5Br-NM-AMT, or a pharmaceutically acceptable salt, salt mixture, isotopic derivative, or prodrug thereof, wherein the percent of R enantiomer is greater than about 70 percent.

In certain embodiments, a host, for example a human, is treated with an effective amount of an enantiomerically enriched mixture of enantiomers of BK-5F-NM-AMT, BK-5Cl-NM-AMT, or BK-5Br-NM-AMT, or a pharmaceutically acceptable salt, salt mixture, isotopic derivative, or prodrug thereof, wherein the percent of R enantiomer is greater than about 65 percent.

In certain embodiments, a host, for example a human, is treated with an effective amount of an enantiomerically enriched mixture of enantiomers of BK-5F-NM-AMT, BK-5Cl-NM-AMT, or BK-5Br-NM-AMT, or a pharmaceutically acceptable salt, salt mixture, isotopic derivative, or prodrug thereof, wherein the percent of R enantiomer is greater than about 60 percent.

In certain embodiments, a host, for example a human, is treated with an effective amount of an enantiomerically enriched mixture of enantiomers of BK-5F-NM-AMT, BK-5Cl-NM-AMT, or BK-5Br-NM-AMT, or a pharmaceutically acceptable salt, salt mixture, isotopic derivative, or prodrug thereof, wherein the percent of R enantiomer is greater than about 55 percent.

In certain embodiments, a host, for example a human, is treated with an effective amount of an enantiomerically enriched mixture of enantiomers of BK-5F-NM-AMT, BK-5Cl-NM-AMT, or BK-5Br-NM-AMT, or a pharmaceutically acceptable salt, salt mixture, isotopic derivative, or prodrug thereof, wherein the percent of R enantiomer is greater than about 55 or 60 percent.

In certain embodiments, a host, for example a human, is treated with an effective amount of an enantiomerically enriched mixture of enantiomers of BK-5F-NM-AMT, BK-5Cl-NM-AMT, or BK-5Br-NM-AMT, or a pharmaceutically acceptable salt, salt mixture, isotopic derivative, or prodrug thereof, wherein the percent of S enantiomer is greater than about 95 percent.

In certain embodiments, a host, for example a human, is treated with an effective amount of an enantiomerically enriched mixture of enantiomers of BK-5F-NM-AMT, BK-5Cl-NM-AMT, or BK-5Br-NM-AMT, or a pharmaceutically acceptable salt, salt mixture, isotopic derivative, or prodrug thereof, wherein the percent of S enantiomer is greater than about 90 percent.

In certain embodiments, a host, for example a human, is treated with an effective amount of an enantiomerically enriched mixture of enantiomers of BK-5F-NM-AMT, BK-5Cl-NM-AMT, or BK-5Br-NM-AMT, or a pharmaceutically acceptable salt, salt mixture, isotopic derivative, or prodrug thereof, wherein the percent of S enantiomer is greater than about 85 percent.

In certain embodiments, a host, for example a human, is treated with an effective amount of an enantiomerically enriched mixture of enantiomers of BK-5F-NM-AMT, BK-5Cl-NM-AMT, or BK-5Br-NM-AMT, or a pharmaceutically acceptable salt, salt mixture, isotopic derivative, or prodrug thereof, wherein the percent of S enantiomer is greater than about 80 percent.

In certain embodiments, a host, for example a human, is treated with an effective amount of an enantiomerically enriched mixture of enantiomers of BK-5F-NM-AMT, BK-5Cl-NM-AMT, or BK-5Br-NM-AMT, or a pharmaceutically acceptable salt, salt mixture, isotopic derivative, or prodrug thereof, wherein the percent of S enantiomer is greater than about 75 percent.

In certain embodiments, a host, for example a human, is treated with an effective amount of an enantiomerically enriched mixture of enantiomers of BK-5F-NM-AMT, BK-5Cl-NM-AMT, or BK-5Br-NM-AMT, or a pharmaceutically acceptable salt, salt mixture, isotopic derivative, or prodrug thereof, wherein the percent of S enantiomer is greater than about 70 percent.

In certain embodiments, a host, for example a human, is treated with an effective amount of an enantiomerically enriched mixture of enantiomers of BK-5F-NM-AMT, BK-5Cl-NM-AMT, or BK-5Br-NM-AMT, or a pharmaceutically acceptable salt, salt mixture, isotopic derivative, or prodrug thereof, wherein the percent of S enantiomer is greater than about 65 percent.

In certain embodiments, a host, for example a human, is treated with an effective amount of an enantiomerically enriched mixture of enantiomers of BK-5F-NM-AMT, BK-5Cl-NM-AMT, or BK-5Br-NM-AMT, or a pharmaceutically acceptable salt, salt mixture, isotopic derivative, or prodrug thereof, wherein the percent of S enantiomer is greater than about 60 percent.

In certain embodiments, a host, for example a human, is treated with an effective amount of an enantiomerically enriched mixture of enantiomers of BK-5F-NM-AMT, BK-5Cl-NM-AMT, or BK-5Br-NM-AMT, or a pharmaceutically acceptable salt, salt mixture, isotopic derivative, or prodrug thereof, wherein the percent of S enantiomer is greater than about 55 percent.

In certain embodiments, a host, for example a human, is treated with an effective amount of an enantiomerically enriched mixture of enantiomers of BK-5F-NM-AMT, BK-5Cl-NM-AMT, or BK-5Br-NM-AMT, or a pharmaceutically acceptable salt, salt mixture, isotopic derivative, or prodrug thereof, wherein the percent of S enantiomer is greater than about 55 or 60 percent.

The present invention also provides methods for modulating the CNS in a mammal in need thereof, including a human, by administering a pharmaceutically effective amount of a compound of the present invention, including S-BK-5F-NM-AMT, R-BK-5F-NM-AMT, S-BK-5Cl-NM-AMT, R-BK-5Cl-NM-AMT, S-BK-5Br-NM-AMT, and/or R-BK-5Br-NM-AMT or a pharmaceutically acceptable salt or salt mixture thereof.

In some embodiments, a method is provided for modulating the CNS in a mammal in need thereof, including a human, by administering a pharmaceutically effective amount of BK-5F-NM-AMT, BK-5Cl-NM-AMT, and/or BK-5Br-NM-AMT or a pharmaceutically acceptable salt thereof. In one embodiment, a method is provided for modulating the CNS in a mammal in need thereof, including a human, by administering a pharmaceutically effective amount of a compound of Formula I-XXIX or a pharmaceutically acceptable salt thereof.

In one embodiment, a method is provided to treat diseases or disorders linked to inadequate functioning of neurotransmission in the CNS comprising administering BK-5F-NM-AMT, BK-5Cl-NM-AMT, and/or BK-5Br-NM-AMT or a pharmaceutically acceptable salt thereof in a host in need thereof.

In one embodiment, a method is provided to treat diseases or disorders linked to inadequate functioning of neurotransmission in the CNS comprising administering a compound of Formula I-XXIX or a pharmaceutically acceptable salt thereof in a host in need thereof.

This invention also provides the use of BK-5F-NM-AMT, BK-5Cl-NM-AMT, or BK-5Br-NM-AMT for the manufacture of a medicament for the treatment of maladaptive responses to perceived psychological threats. Additionally, this invention provides a pharmaceutical formulation adapted for the treatment of maladaptive response to perceived psychological threats containing a BK-5F-NM-AMT, BK-5Cl-NM-AMT, or BK-5Br-NM-AMT. Furthermore, this invention includes a method for the treatment of maladaptive response to perceived psychological threats that comprises administering an effective amount of BK-5F-NM-AMT, BK-5Cl-NM-AMT, or BK-5Br-NM-AMT, given either in the context of psychotherapy or as a stand-alone treatment.

This invention also provides the use of compounds of Formulas I-XXIX for the manufacture of a medicament for the treatment of maladaptive response to perceived psychological threats. Additionally, this invention provides a pharmaceutical formulation adapted for the treatment of maladaptive response to perceived psychological threats containing a compound of any of Formulas I-XXIX. Furthermore, this invention includes a method for the treatment of maladaptive response to perceived psychological threats that comprises administering an effective amount of a compound of any of Formulas I-XXIX, given either in the context of psychotherapy or as a stand-alone treatment.

This invention also provides the use S-BK-5F-NM-AMT, R-BK-5F-NM-AMT, S-BK-5Cl-NM-AMT, R-BK-5Cl-NM-AMT, S-BK-5Br-NM-AMT, or R-BK-5Br-NM-AMT or a pharmaceutically acceptable salt or composition to treat a maladaptive response to perceived psychological threats. In one embodiment, S-BK-5F-NM-AMT, R-BK-5F-NM-AMT, S-BK-5Cl-NM-AMT, R-BK-5Cl-NM-AMT, S-BK-5Br-NM-AMT, or R-BK-5Br-NM-AMT or a pharmaceutically acceptable salt or composition is administered in the context of psychotherapy. In one embodiment, S-BK-5F-NM-AMT, R-BK-5F-NM-AMT, S-BK-5Cl-NM-AMT, R-BK-5Cl-NM-AMT, S-BK-5Br-NM-AMT, or R-BK-5Br-NM-AMT or a pharmaceutically acceptable salt or composition is administered as a stand-alone treatment.

This invention also provides the use of a compound of Formula I-XXIX or a pharmaceutically acceptable salt or composition in an effective amount to treat a maladaptive response to perceived psychological threats. In one embodiment, a compound of Formula I-XXIX or a pharmaceutically acceptable salt or composition is administered in the context of psychotherapy. In one embodiment, a compound of Formula I-XXIX or a pharmaceutically acceptable salt or composition is administered as a stand-alone treatment.

### Non-Limiting Examples of Pharmacotherapeutic Counseling Use

Psychotherapy, cognitive enhancement, or life coaching conducted with the compounds or pharmaceutically acceptable salts as described herein employed as an adjunct (hereafter, “pharmacotherapy” or “pharmacotherapy counseling”) is typically conducted in widely spaced sessions with one, two, or rarely three or more administrations of an entactogen per session. These sessions can be as frequent as weekly but are more often approximately monthly or even less frequently. In most cases, a small number of pharmacotherapy counseling sessions, on the order of one to three, is needed for the patient to experience significant clinical progress, as indicated, for example, by a reduction in signs and symptoms of mental distress, by improvement in functioning in some domain of life, by arrival at a satisfactory solution to some problem, or by increased feelings of closeness to and understanding of some other person. In some embodiments, the psychotherapy, cognitive enhancement, or life coaching is conducted with an effective amount of racemic, enantiomerically pure, or enantiomerically enriched S-BK-5F-NM-AMT, R-BK-5F-NM-AMT, S-BK-5Cl-NM-AMT, R-BK-5Cl-NM-AMT, S-BK-5Br-NM-AMT, and/or R-BK-5Br-NM-AMT or a pharmaceutically acceptable salt thereof.

In one embodiment, the psychotherapy, cognitive enhancement, or life coaching is conducted with an effective amount a compound of any of Formulas I-XXIX or a pharmaceutically acceptable salt thereof.

The following sections provide detailed examples of pharmacotherapy counseling. While common procedures are described, these are intended as illustrative, non-limiting examples. It is anticipated that the prescribing physician and therapy team may wish to specify different procedures than those described here based on their clinical judgment concerning the needs of the patient.

The example methods of treatment can also be modified with minor changes to treat multiple patients at once, including couples or families. Hence, “patient” should be understood to mean one or more individuals.

Use of a Tryptamine Compound or Composition of the Present Invention in Conjunction with Conventional Psychotherapy or Coaching

In one embodiment, the use of a described tryptamine compound or composition of the present invention as pharmacotherapy is integrated into the patient's ongoing psychotherapy or coaching (hereafter abbreviated as “psychotherapy”). If a patient in need of the pharmacotherapy counseling is not in ongoing psychotherapy, then psychotherapy may be initiated and the pharmacotherapy counseling added later, after the prescribing physician and treating psychotherapist, physician, coach, member of the clergy, or other similar professional or someone acting under the supervision of such a professional (hereafter, “therapist”) agree that the pharmacotherapy counseling is indicated and that there have been sufficient meetings between the patient and therapist to establish an effective therapeutic alliance.

If the patient is not experienced with the pharmacotherapy, a conversation typically occurs in which the therapist or other members of the therapy team addresses the patient's questions and concerns about the medicine and familiarizes the patient with the logistics of pharmacotherapy-assisted session. The therapist describes the kinds of experience that can be expected during the pharmacotherapy counseling session. Optionally, parts of this conversation employ written, recorded, or interactive digital explanations, as might be used in the informed consent process in a clinical trial. The therapist may additionally make commitments to support the participant's healthcare and wellness process. In turn, the patient may be asked to make commitments of their own (such as not to hurt themselves or others and to abstain from contraindicated medicines or drugs for an adequate period before and after the pharmacotherapy counseling).

The compounds and compositions of the invention (or alternately herein for convenience, the “medicine”) is administered shortly before or during a scheduled psychotherapy session, with timing optionally selected so that therapeutic effects begin by the time the psychotherapy session begins. It is to be understood that references to administering the medicine “during” a psychotherapeutic or other session are intended to refer to timing the administration of the medicine such that the therapeutic effects of the medicine at least partly temporally overlap with the therapeutic effects of the session. Either shortly before or after administration of the medicine, it is common for the therapist to provide some reminder of their mutual commitments and expected events during the session.

The psychotherapy session is carried out by the therapist, who, optionally, may be remote and in communication with the patient using a communication means suitable for telehealth or telemedicine, such as a phone, video, or other remote two-way communication method. Optionally, video or other monitoring of the patient's response or behavior is used to document or measure the session. The therapist uses their clinical judgment and available data to adjust the session to the needs of the patient. Many therapists view their responsibility as being to facilitate rather than direct the patient's experience. This may sometimes involve silent empathic listening, while other times it may include more active support to help the patient arrive at new perspectives on their life.

It is anticipated that the therapeutic effects of the medicine will allow the patient to make more rapid therapeutic progress than would normally be possible. These effects include decreased neuroticism and increased feelings of authenticity. Patients are often able to calmly contemplate actual or possible experiences that would normally be upsetting or even overwhelming. This can facilitate decision making and creativity in addition to mental wellness.

Optionally, the prescribing physician may allow a second or even third administration of the medicine or another psychotherapeutic agent in order to extend the therapeutic effects. Optionally, a pharmaceutical preparation with modified release is employed to make this unnecessary.

Because the duration of the scheduled psychotherapy session may be shorter than the therapeutic effects of the medicine, the therapist may suggest to the patient activities to support further psychotherapeutic progress after the psychotherapy session has ended. Alternatively, the therapist may continue to work with the patient until the therapeutic effects of the medicine have become clinically minimal.

In a subsequent non-pharmacological psychotherapy session, the therapist and patient will typically discuss the patient's experiences from the pharmacotherapy counseling session and the therapist will often aid the patient in recalling the therapeutic effects and help them to incorporate the experiences into their everyday lives.

Pharmacotherapy counseling sessions may be repeated as needed, based on the judgment of the treating physician and therapy team regarding the needs of the patient.

### Use of a Tryptamine Compound or Composition of the Present Invention Outside of Conventional Psychotherapy

In one embodiment, a compound or composition of the present invention is administered outside of a conventional psychotherapy. This example method is a broader, more flexible approach to pharmacotherapy that is not centered on supervision by a therapist. These pharmacotherapy counseling sessions can take place in many different quiet and safe settings, including the patient's home. The setting is typically chosen to offer a quiet setting, with minimal disruptions, where the patient feels psychologically safe and emotionally relaxed. The setting may be the patient's home but may alternatively be a clinic, retreat center, or hotel room.

In one alternative embodiment, the medicine is taken by the patient regularly to maintain therapeutic concentrations of the active compound in the blood. In another alternative embodiment, the medicine is taken, as needed, for defined psychotherapy sessions.

Optionally, a checklist may be followed to prepare the immediate environment to minimize distractions and maximize therapeutic or decision-making benefits. This checklist can include items such as silencing phones and other communications devices, cleaning and tidying the environment, preparing light refreshments, preparing playlists of appropriate music, and pre-arranging end-of-session transportation if the patient is not undergoing pharmacotherapy counseling at home.

Before the pharmacotherapy counseling session, there may be an initial determination of the therapeutic or other life-related goals (for example, decision-making, increasing creativity, or simply appreciation of life) that will be a focus of the session. These goals can optionally be determined in advance with support from a therapist.

Optionally, the therapist may help the patient select stimuli, such as photographs, videos, augmented or virtual reality scenes, or small objects such as personal possessions, that will help focus the patient's attention on the goals of the session or on the patient's broader life journey. As examples that are intended to be illustrative and not restrictive, these stimuli can include photographs of the patient from when they were young, which can increase self-compassion, or can include stimuli relating to traumatic events or phobias experienced by the patient, which can help the patient reevaluate and change their response to such stimuli. Optionally, the patient selects these stimuli without assistance (for example, without the involvement of the therapist) or does not employ any stimuli. Optionally, stimuli are selected in real time by the therapist, or an algorithm based on the events of the session with the goal of maximizing benefits to the patient.

If the patient is not experienced with the pharmacotherapy, a conversation occurs in which the therapist addresses the patient's questions and concerns about the medicine and familiarizes the patient with the logistics of a pharmacotherapy-assisted counseling session. The therapist describes the kinds of experience that can be expected during the pharmacotherapy-assisted counseling session. Optionally, parts of this conversation employ written, recorded, or interactive digital explanations, as might be used in the informed consent process in a clinical trial. The therapist may additionally make commitments to support the participant's healthcare and wellness process. In turn, the patient may be asked to make commitments of their own (such as not to hurt themselves or others and to abstain from contraindicated medicines or drugs for an adequate period before and after the pharmacotherapy counseling).

Selected session goals and any commitments or other agreements regarding conduct between the patient and therapy team are reviewed immediately before administration of the medicine. Depending on the pharmaceutical preparation and route of administration, the therapeutic effects of the medicine usually begin within one hour. Typical therapeutic effects include decreased neuroticism and increased feelings of authenticity. Patients are often able to calmly contemplate experiences or possible experiences that would normally be upsetting or even overwhelming. This can facilitate decision making and creativity in addition to mental wellness.

Optionally, sleep shades and earphones with music or soothing noise may be used to reduce distractions from the environment. Optionally, a virtual reality or immersive reality system may be used to provide stimuli that support the therapeutic process. Optionally, these stimuli are preselected; optionally, they are selected in real time by a person, or an algorithm based on events in the session with the goal of maximizing benefits to the patient. Optionally, a therapist or other person well-known to the patient is present or available nearby or via phone, video, or other communication method in case the patient wishes to talk, however the patient may optionally undergo a session without the assistance of a therapist. Optionally, the patient may write or create artwork relevant to the selected session goals. Optionally, the patient may practice stretches or other beneficial body movements, such as yoga (“movement activity”).

Optionally, in other embodiments the patient may practice movement activity that includes more vigorous body movements, such as dance or other aerobic activity. Movement activity also may make use of exercise equipment such as a treadmill or bicycle.

In some additional embodiments, the patient may be presented with music, video, auditory messages, or other perceptual stimuli. Optionally, these stimuli may be adjusted based on the movements or other measurable aspects of the patient. Such adjustment may be done by the therapist with or without the aid of a computer, or by a computer alone in response to the patient aspects, including by an algorithm or artificial intelligence, and “computer” broadly meaning any electronic tool suitable for such purposes, whether worn or attached to a patient (for example, watches, fitness trackers, “wearables,” and other personal devices; biosensors or medical sensors; medical devices), whether directly coupled or wired to a patient or wirelessly connected (and including desktop, laptop, and notebook computers; tablets, smartphones, and other mobile devices; and the like), and whether within the therapy room or remote (for example, cloud-based systems).

For example, measurable aspects of a patient (for example, facial expression, eye movements, respiration rate, pulse rate, skin color change, patient voice quality or content, patient responses to questions) collected from these tools may be individually transformed into scores on standardized scales by subtracting a typical value and then multiplying by a constant and these scores may be further multiplied by constants and added together to create an overall score that can optionally be transformed by multiplication with a link function, such as the logit function, to create an overall score. This score may be used to select or adjust stimuli such as selecting music with higher or lower beats-per-minute or with faster or slower notes, selecting images, audio, or videos with different emotionality or autobiographical meaning, or selecting activities for the patient to engage in (such as specific movements, journaling prompts, or meditation mantras).

It should be readily appreciated that a patient can participate in numerous therapeutically beneficial activities, where such participation follows or is in conjunction with the administration of a compound or composition of the invention, including writing about a preselected topic, engaging in yoga or other movement activity, meditating, creating art, viewing of photographs or videos or emotionally evocative objects, using a virtual reality or augmented reality system, talking with a person, and thinking about a preselected problem or topic, and it should be understood that such participation can occur with or without the participation or guidance of a therapist.

Optionally, the prescribing physician may allow a second or even third administration of the medicine or another psychotherapeutic agent in order to extend the therapeutic effects. Optionally, a pharmaceutical preparation with modified release is employed to make this unnecessary.

The patient typically remains in the immediate environment until the acute therapeutic effects of the medicine are clinically minimal, usually within eight hours. After this point, the session is considered finished.

The treatment plan will often include a follow-up session with a therapist. This follow-up session occurs after the pharmacotherapy counseling session has ended, often the next day but sometimes several days later. In this session, the patient discusses their experiences from the pharmacotherapy counseling session with the therapist, who can aid them in recalling the therapeutic effects and help them to incorporate the experiences into their everyday lives.

Pharmacotherapy counseling sessions may be repeated as needed, based on the judgment of the treating physician and therapy team regarding the needs of the patient.

### IV. Pharmaceutical Compositions and Salts

The tryptamine compounds and compositions described herein can be administered in an effective amount as the neat chemical but are more typically administered as a pharmaceutical composition for a host, typically a human, in need of such treatment in an effective amount for any of the disorders described herein. The pharmaceutical composition typically comprises a pharmaceutically acceptable carrier, diluent, or excipient, and at least one compound, pure enantiomer, or enantiomerically enriched mixture of the present invention. The compounds or compositions disclosed herein may be administered orally, topically, systemically, parenterally, by inhalation, insufflation, or spray, mucosally (for example, buccal, sublingual), sublingually, transdermally, rectally, intravenous, intra-aortal, intracranial, subdermal, intraperitoneal, intramuscularly, inhaled, intranasal, subcutaneous, transnasal, or by other means, in dosage unit formulations containing conventional pharmaceutically acceptable carriers. Such compositions are prepared in a manner well known in the pharmaceutical art and comprise at least one active compound. (See, for example, Remington, 2005, Remington: The science and practice of pharmacy, 21st ed., Lippincott Williams & Wilkins.)

The pharmaceutical composition may be formulated as any pharmaceutically useful form, for example, as an aerosol, a cream, a gel, a pill, an injection or infusion solution, a capsule, a tablet, a syrup, a transdermal patch, a subcutaneous patch, a dry powder, an inhalation formulation, a suppository, a buccal or sublingual formulation, a parenteral formulation, an ophthalmic solution, or in a medical device. Some dosage forms, such as tablets and capsules, are subdivided into suitably sized unit doses containing appropriate quantities of the active components, for example, an effective amount to achieve the desired purpose.

A “pharmaceutically acceptable composition” thus refers to at least one compound (which may be a mixture of enantiomers or diastereomers, as fully described herein) of the invention and a pharmaceutically acceptable vehicle, excipient, diluent or other carrier in an effective amount to treat a host, typically a human, who may be a patient.

In certain nonlimiting embodiments the pharmaceutical composition is a dosage form that contains from about 0.1 mg to about 1500 mg, from about 10 mg to about 1000 mg, from about 100 mg to about 800 mg, or from about 200 mg to about 600 mg of the active compound and optionally from about 0.1 mg to about 1500 mg, from about 10 mg to about 1000 mg, from about 100 mg to about 800 mg, or from about 200 mg to about 600 mg of an additional active agent in a unit dosage form. Examples are dosage forms with at least 0.1, 1, 5, 10, 20, 25, 40, 50, 100, 125, 150, 200, 250, 300, 400, 500, 600, 700, or 750 mg of active compound, or its salt or salt mixture.

The pharmaceutical compositions described herein can be formulated into any suitable dosage form, including tablets, capsules, gelcaps, aqueous oral dispersions, aqueous oral suspensions, solid dosage forms including oral solid dosage forms, aerosols, controlled release formulations, fast melt formulations, effervescent formulations, self-emulsifying dispersions, solid solutions, liposomal dispersions, lyophilized formulations, pills, powders, delayed-release formulations, immediate-release formulations, modified release formulations, extended-release formulations, pulsatile release formulations, multi particulate formulations, and mixed immediate release and controlled release formulations. Generally speaking, the composition should be administered in an effective amount to administer an amount of the active agents of the present invention achieves a plasma level commensurate with the concentrations found to be effective in vivo for a period of time effective to elicit a desired therapeutic effect without abuse liability.

In making the compositions employed in the present invention the active ingredient is usually mixed with an excipient, diluted by an excipient, or enclosed within such a carrier which can be in the form of a capsule, sachet, paper or other container. When the excipient serves as a diluent, it can be a solid, semi-solid, or liquid material, which acts as a vehicle, carrier, or medium for the active ingredient. Thus, the compositions can be in the form of tablets (including orally disintegrating, swallowable, sublingual, buccal, and chewable tablets), pills, powders, lozenges, troches, oral films, thin strips, sachets, cachets, elixirs, suspensions, emulsions, solutions, slurries, syrups, aerosols (as a solid or in a liquid medium), ointments containing for example up to 10% by weight of the active compound, soft and hard gelatin capsules, suppositories, dry powders for inhalation, liquid preparations for vaporization and inhalation, topical preparations, transdermal patches, sterile injectable solutions, and sterile packaged powders. Compositions may be formulated as immediate release, controlled release, sustained (extended) release or modified release formulations.

The compositions of the present invention can be administered by multiple routes, which may differ in different patients according to their preference, co-morbidities, side effect profile, and other factors (IV, PO, transdermal, etc.). In one embodiment, the pharmaceutical composition includes the presence of other substances with the active drugs, known to those skilled in the art, such as fillers, carriers, gels, skin patches, lozenges, or other modifications in the preparation to facilitate absorption through various routes (such as, but not limited to, gastrointestinal, transdermal, etc.) and/or to extend the effect of the drugs, and/or to attain higher or more stable serum levels or to enhance the therapeutic effect of the active drugs in the combination.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, for example, about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include, but are not limited to, lubricating agents such as talc, magnesium stearate, and mineral oil; wetting agents; emulsifying and suspending agents; preserving agents such as methyl- and propylhydroxybenzoates; sweetening agents; and flavoring agents. The compositions of the invention can be formulated so as to provide quick, sustained or delayed release of the active ingredient after administration to the patient by employing procedures known in the art.

The compositions are in certain embodiments formulated in a unit dosage form, each dosage containing from at least about 0.05 to about 350 mg or less, more typically at least about 0.1 to about 280 mg or less, of the active ingredients. The term “unit dosage form” refers to physically discrete units suitable as unitary dosages for human subjects and other mammals, each unit containing a predetermined quantity of active material calculated to produce the desired therapeutic effect, in association with a suitable pharmaceutical carrier, diluent, or excipient.

The active compounds are effective over a wide dosage range. For example, as-needed dosages normally fall within the range of at least about 0.0007 to about 5 mg/kg or less. In the treatment of adult humans, the range of at least about 0.001 to about 4 mg/kg or less, in single dose may be useful.

It will be understood that the amount of the compound actually administered will be determined by a physician, in light of the relevant circumstances, including the condition to be treated, the chosen route of administration, the actual compound or compounds administered, the age, weight, and response of the individual patient, and the severity of the patient's symptoms, and therefore the above dosage ranges are not intended to limit the scope of the invention in any way.

In some instances, dosage levels below the lower limit of the aforesaid range may be more than adequate, while in other cases still larger doses may be employed without causing any harmful side effects, provided for instance that such larger doses may be first divided into several smaller doses for administration.

Generally, the pharmaceutical compositions of the invention may be administered and dosed in accordance with good medical practice, taking into account the method and scheduling of administration, prior and concomitant medications and medical supplements, the clinical condition of the individual patient and the severity of the underlying disease, the patient's age, sex, body weight, and other such factors relevant to medical practitioners, and knowledge of the particular compound(s) used. Starting and maintenance dosage levels thus may differ from patient to patient, for individual patients across time, and for different pharmaceutical compositions, but shall be able to be determined with ordinary skill.

In one embodiment, a powder comprising the active agents of the present invention described herein may be formulated to comprise one or more pharmaceutical excipients and flavors. Such a powder may be prepared, for example, by mixing the active agents of the present invention and optional pharmaceutical excipients to form a bulk blend composition. Additional embodiments also comprise a suspending agent and/or a wetting agent. This bulk blend is uniformly subdivided into unit dosage packaging or multi-dosage packaging units. The term “uniform” means the homogeneity of the bulk blend is substantially maintained during the packaging process.

### Oral Formulations

In certain embodiments, any selected compound(s) of the present invention is formulated in an effective amount in a pharmaceutically acceptable oral dosage form. In one embodiment, the compound(s) is S-BK-5F-NM-AMT, R-BK-5F-NM-AMT, S-BK-5Cl-NM-AMT, R-BK-5Cl-NM-AMT, S-BK-5Br-NM-AMT, or R-BK-5Br-NM-AMT or a pharmaceutically acceptable salt thereof. In one embodiment, the compound(s) is a compound of any of Formulas I-XXIX or a pharmaceutically acceptable salt thereof. Oral dosage forms may include, but are not limited to, oral solid dosage forms and oral liquid dosage forms. Oral solid dosage forms may include but are not limited to, tablets, capsules, caplets, powders, pellets, multiparticulates, beads, spheres and/or any combinations thereof. The oral solid dosage forms may be formulated as immediate release, controlled release, sustained (extended) release or modified release formulations.

The oral solid dosage forms of the present invention may also contain pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof.

In some embodiments, the solid dosage forms of the present invention may be in the form of a tablet (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder), a capsule (including both soft or hard capsules, for example, capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including a fast-melt tablet. Additionally, pharmaceutical formulations of the present invention may be administered as a single capsule or in multiple capsule dosage form. In some embodiments, the pharmaceutical formulation is administered in two, or three, or four, capsules or tablets.

The pharmaceutical solid dosage forms described herein can comprise the active agent of the present invention compositions described herein and one or more pharmaceutically acceptable additives such as a compatible carrier, binder, complexing agent, ionic dispersion modulator, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

Alternatively, the pharmaceutical solid dosage forms described herein can comprise the active agent or agents of the present invention (i.e., the “active agent(s)”; but for convenience herein, both “active agent” and “active agents” shall mean “active agent(s)” unless context clearly indicates that what is intended or would be suitable is only one agent or only two or more agents) and one or more pharmaceutically acceptable additives such as a compatible carrier, binder, complexing agent, ionic dispersion modulator, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

In still other aspects, using standard coating procedures, such as those described in Remington's Pharmaceutical Sciences, 20th Edition (2000), a film coating is provided around the active agent of the present invention formulation. In one embodiment, some or all of the active agent of the present invention particles are coated. In another embodiment, some or all of the active agent of the present invention particles are microencapsulated. In yet another embodiment, some or all of the active agent of the present invention is amorphous material coated and/or microencapsulated with inert excipients. In still another embodiment, the active agent of the present invention particles are not microencapsulated and are uncoated.

Suitable carriers for use in the solid dosage forms described herein include acacia, gelatin, colloidal silicon dioxide, calcium glycerophosphate, calcium lactate, maltodextrin, glycerin, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include lactose, calcium carbonate, calcium phosphate, dibasic calcium phosphate, calcium sulfate, microcrystalline cellulose (for example, Avicel®, Avicel® PH101, Avicel® PH102, Avicel® PH105, etc.), cellulose powder, dextrose, dextrates, dextrose, dextran, starches, pregelatinized starch, hydroxypropylmethylcellulose (HPMC), hydroxypropylmethylcellulose phthalate, hydroxypropylmethylcellulose acetate stearate (HPMCAS), sucrose, xylitol, lactitol, mannitol, sorbitol, sodium chloride, polyethylene glycol, and the like.

If needed, suitable disintegrants for use in the solid dosage forms described herein include natural starch such as corn starch or potato starch, a pregelatinized starch such as National 1551 or Amijel®, or a sodium starch glycolate such as Promogel® or Explotab®, a cellulose such as a wood product, microcrystalline cellulose, for example, Avicel®, Avicel® PH101, Avicel® PH102, Avicel® PH105, Elcema® P100, Emcocel®, Vivacel®, Ming Tia®, and Solka-Floc®, Ac-Di-Sol, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Binders impart cohesiveness to solid oral dosage form formulations: for powder-filled capsule formulation, they aid in plug formation that can be filled into soft- or hard-shell capsules and in tablet formulation, binders ensure that the tablet remains intact after compression and help assure blend uniformity prior to a compression or fill step. Materials suitable for use as binders in the solid dosage forms described herein include carboxymethylcellulose, methylcellulose (for example, Methocel®), hydroxypropylmethylcellulose (for example, Hypromellose USP Pharmacoat-603, hydroxypropylmethylcellulose acetate stearate (Agoate HS-LF and HS), hydroxyethylcellulose, hydroxypropylcellulose (for example, Klucel®), ethylcellulose (for example, Ethocel®), and microcrystalline cellulose (for example, Avicel®), microcrystalline dextrose, amylose, magnesium aluminum silicate, polysaccharide acids, bentonites, gelatin, polyvinylpyrrolidone/vinyl acetate copolymer, crosspovidone, povidone, starch, pregelatinized starch, tragacanth, dextrin, a sugar, such as sucrose (for example, Dipac®), glucose, dextrose, molasses, mannitol, sorbitol, xylitol (for example, Xylitab®), lactose, a natural or synthetic gum such as acacia, tragacanth, ghatti gum, mucilage of isapol husks, starch, polyvinylpyrrolidone (for example, Povidone® CL, Kollidon® CL, Polyplasdone® XL-10, and Povidone® K-12), larch arabogalactan, Veegum®, polyethylene glycol, waxes, sodium alginate, and the like. In general, binder levels of 20-70% are typically used in powder-filled gelatin capsule formulations. Binder usage level in tablet formulations is a function of whether direct compression, wet granulation, roller compaction, or usage of other excipients such as fillers which itself can act as moderate binders are used. Formulators skilled in the art can determine the binder level for the formulations, but binder usage level of up to 70% in tablet formulations is common.

Suitable lubricants or glidants for use in the solid dosage forms described herein include stearic acid, calcium hydroxide, talc, corn starch, sodium stearyl fumarate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include sugars (including lactose, sucrose, and dextrose), polysaccharides (including dextrates and maltodextrin), polyols (including mannitol, xylitol, and sorbitol), cyclodextrins and the like.

Non-water-soluble diluents are compounds typically used in the formulation of pharmaceuticals, such as calcium phosphate, calcium sulfate, starches, modified starches and microcrystalline cellulose, and micro cellulose (for example, having a density of about 0.45 g/cm³, for example Avicel®, powdered cellulose), and talc.

Suitable wetting agents for use in the solid dosage forms described herein include oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (for example, Polyquat 10®), sodium oleate, sodium lauryl sulfate, magnesium stearate, sodium docusate, triacetin, vitamin E TPGS and the like. Wetting agents include surfactants.

Suitable surfactants for use in the solid dosage forms described herein include docusate and its pharmaceutically acceptable salts, sodium lauryl sulfate, sorbitan monooleate, polyoxyethylene sorbitan monooleate, polysorbates, poloxamers, bile salts, glyceryl monostearate, copolymers of ethylene oxide and propylene oxide, for example, Pluronic® (BASF), and the like.

Suitable suspending agents for use in the solid dosage forms described here include polyvinylpyrrolidone, for example, polyvinylpyrrolidone K12, polyvinylpyrrolidone K17, polyvinylpyrrolidone K25, or polyvinylpyrrolidone K30, polyethylene glycol, for example, the polyethylene glycol can have a molecular weight of about 300 to about 6000, or about 3350 to about 4000, or about 7000 to about 18000, vinylpyrrolidone/vinyl acetate copolymer (S630), sodium alginate, gums, such as, for example, gum tragacanth and gum acacia, guar gum, xanthans, including xanthan gum, sugars, cellulosic, such as, for example, sodium carboxymethylcellulose, methylcellulose, sodium carboxymethylcellulose, hydroxypropylmethylcellulose, hydroxyethylcellulose, polysorbate-80, polyethoxylated sorbitan monolaurate, polyethoxylated sorbitan monolaurate, povidone and the like.

Suitable antioxidants for use in the solid dosage forms described herein include, for example, butylated hydroxytoluene (BHT), butyl hydroxyanisole (BHA), sodium ascorbate, Vitamin E TPGS, ascorbic acid, sorbic acid and tocopherol.

Immediate-release formulations may be prepared by combining superdisintegrants such as Croscarmellose sodium and different grades of microcrystalline cellulose in different ratios. To aid disintegration, sodium starch glycolate will be added.

The above-listed additives should be taken as merely examples and not limiting, of the types of additives that can be included in solid dosage forms of the present invention. The amounts of such additives can be readily determined by one skilled in the art, according to the particular properties desired.

Oral liquid dosage forms include solutions, emulsions, suspensions, and syrups. These oral liquid dosage forms may be formulated with any pharmaceutically acceptable excipient known to those of skill in the art for the preparation of liquid dosage forms. For example, water, glycerin, simple syrup, alcohol, and combinations thereof.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as but not limited to, an oil, water, an alcohol, and combinations of these pharmaceutically suitable surfactants, suspending agents, emulsifying agents, may be added for oral or parenteral administration. Suspensions may include oils. Such oils include peanut oil, sesame oil, cottonseed oil, corn oil, and olive oil. Suspension preparation may also contain esters of fatty acids such as ethyl oleate, isopropyl myristate, fatty acid glycerides, and acetylated fatty acid glycerides. Suspension formulations may include alcohols, such as ethanol, isopropyl alcohol, hexadecyl alcohol, glycerol, and propylene glycol. Ethers, such as poly(ethylene glycol), petroleum hydrocarbons such as mineral oil and petrolatum, and water may also be used in suspension formulations.

In some embodiments, formulations are provided comprising particles of S-BK-5F-NM-AMT, R-BK-5F-NM-AMT, S-BK-5Cl-NM-AMT, R-BK-5Cl-NM-AMT, S-BK-5Br-NM-AMT, and/or R-BK-5Br-NM-AMT and at least one dispersing agent or suspending agent for oral administration to a subject in need thereof. In some embodiments, formulations are provided comprising particles of compounds of any of Formula I-XXIX and at least one dispersing agent or suspending agent for oral administration to a subject in need thereof.

The formulation may be a powder and/or granules for suspension, and upon admixture with water, a substantially uniform suspension is obtained. As described herein, the aqueous dispersion can comprise amorphous and non-amorphous particles consisting of multiple effective particle sizes such that the drug is absorbed in a controlled manner over time. In certain embodiments, the aqueous dispersion or suspension is an immediate-release formulation. In another embodiment, an aqueous dispersion comprising amorphous particles is formulated such that a portion of the particles of the present invention are absorbed within, for example, about 0.75 hours after administration and the remaining particles are absorbed 2 to 4 hours after absorption of the earlier particles.

In other embodiments, addition of a complexing agent to the aqueous dispersion results in a larger span of the particles to extend the drug absorption phase of the active agent such that 50-80% of the particles are absorbed in the first hour and about 90% are absorbed by about 4 hours. Dosage forms for oral administration can be aqueous suspensions selected from the group including pharmaceutically acceptable aqueous oral dispersions, emulsions, solutions, and syrups. See, for example, Singh et al., Encyclopedia of Pharm. Tech., 2nd Ed., 754-757 (2002). In addition to the active agents of the present invention particles, the liquid dosage forms may comprise additives, such as (a) disintegrating agents; (b) dispersing agents; (c) wetting agents; (d) at least one preservative; (e) viscosity enhancing agents; (f) at least one sweetening agent; and (g) at least one flavoring agent.

Examples of disintegrating agents for use in the aqueous suspensions and dispersions include a starch, for example, a natural starch such as corn starch or potato starch, a pregelatinized starch such as National 1551 or Amijel®, or sodium starch glycolate such as Promogel® or Explotab®; a cellulose such as a wood product, microcrystalline cellulose, for example, Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crosspovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropylcellulose ethers (for example, HPC, HPC-SL, and HPC-L), hydroxypropylmethylcellulose and hydroxypropylmethylcellulose ethers (for example HPMC K100, HPMC K4M, HPMC K15M, and HPMC K100M), carboxymethylcellulose sodium, methylcellulose, hydroxyethylcellulose, hydroxypropylmethylcellulose phthalate, hydroxypropylmethylcellulose acetate stearate, noncrystalline cellulose, magnesium aluminum silicate, triethanolamine, polyvinyl alcohol (PVA), polyvinylpyrrolidone/vinyl acetate copolymer (Plasdone®, for example, S-630), 4-(1,1,3,3-tetramethylbutyl)-phenol polymer with ethylene oxide and formaldehyde (also known as tyloxapol), poloxamers (for example, Pluronics F68®, F88®, and F108®, which are block copolymers of ethylene oxide and propylene oxide); and poloxamines (for example, Tetronic 908®, also known as Poloxamine 908®, which is a tetrafunctional block copolymer derived from sequential addition of propylene oxide and ethylene oxide to ethylenediamine (BASF Corp., Parsippany, N.J.)).

In other embodiments, the dispersing agent is selected from a group not comprising one of the following agents: hydrophilic polymers; electrolytes; Tween® 60 or 80; PEG; polyvinylpyrrolidone (PVP); hydroxypropyl cellulose and hydroxypropyl cellulose ethers (for example, HPC, HPC-SL, and HPC-L); hydroxypropyl methylcellulose and hydroxypropyl methylcellulose ethers (for example HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethylcellulose phthalate; hydroxypropylmethylcellulose acetate stearate; non-crystalline cellulose; magnesium aluminum silicate; triethanolamine; polyvinyl alcohol (PVA); 4-(1,1,3,3-tetramethyl butyl)-phenol polymer with ethylene oxide and formaldehyde; poloxamers (for example, Pluronics F68®, F88®, and F108®, which are block copolymers of ethylene oxide and propylene oxide); or poloxamines (for example, Tetronic 908® or Poloxamine 908®).

Wetting agents (including surfactants) suitable for the aqueous suspensions and dispersions described herein are known in the art and include cetyl alcohol, glycerol monostearate, polyoxyethylene sorbitan fatty acid esters (for example, the commercially available Tweens® such as for example, Tween 20® and Tween 80® (ICI Specialty Chemicals)), and polyethylene glycols (for example, Carbowaxs 3350® and 1450®, and Carpool 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphatidylcholine and the like.

Suitable preservatives for the aqueous suspensions or dispersions described herein include potassium sorbate, parabens (for example, methylparaben and propylparaben) and their salts, benzoic acid and its salts, other esters of para hydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

In one embodiment, the aqueous liquid dispersion can comprise methylparaben and propylparaben in a concentration ranging from at least about 0.01% to about 0.3% or less methylparaben by weight to the weight of the aqueous dispersion and at least about 0.005% to about 0.03% or less propylparaben by weight to the total aqueous dispersion weight. In yet another embodiment, the aqueous liquid dispersion can comprise methylparaben from at least about 0.05 to about 0.1 or less weight % and propylparaben from at least about 0.01 to about 0.02 or less weight % of the aqueous dispersion.

Suitable viscosity enhancing agents for the aqueous suspensions or dispersions described herein include methyl cellulose, xanthan gum, carboxymethylcellulose, hydroxypropyl cellulose, hydroxypropylmethyl cellulose, Plasdone® S-630, carbomer, polyvinyl alcohol, alginates, acacia, chitosans and combinations thereof. The concentration of the viscosity-enhancing agent will depend upon the agent selected and the viscosity desired.

In addition to the additives listed above, the liquid formulations of the present invention can also comprise inert diluents commonly used in the art, such as water or other solvents, solubilizing agents, emulsifiers, and/or sweeteners.

In one embodiment, the formulation for oral delivery is an effervescent powder containing S-BK-5F-NM-AMT, R-BK-5F-NM-AMT, S-BK-5Cl-NM-AMT, R-BK-5Cl-NM-AMT, S-BK-5Br-NM-AMT, and/or R-BK-5Br-NM-AMT or a pharmaceutically acceptable salt thereof. In one embodiment, the formulation for oral delivery is an effervescent powder containing a compound of any of Formulas I-XXIX or a pharmaceutically acceptable salt thereof. Effervescent salts have been used to disperse medicines in water for oral administration. Effervescent salts have been used to disperse medicines in water for oral administration. Effervescent salts are granules or coarse powders containing a medicinal agent in a dry mixture, usually composed of sodium bicarbonate, citric acid and/or tartaric acid. When salts of the present invention are added to water, the acids and the base react to liberate carbon dioxide gas, thereby causing “effervescence.” Examples of effervescent salts include sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

Tablets of the invention described here can be prepared by methods well known in the art. Various methods for the preparation of the immediate release, modified release, controlled release, and extended-release dosage forms (for example, as matrix tablets, tablets having one or more modified, controlled, or extended-release layers, etc.) and the vehicles therein are well known in the art. Generally recognized compendia of methods include: Remington: The Science and Practice of Pharmacy, Alfonso R. Gennaro, Editor, 20th Edition, Lippincott Williams & Wilkins, Philadelphia, Pa.; and Sheth et al. (1980), Compressed tablets, in Pharmaceutical dosage forms, Vol. 1, edited by Lieberman and Lachtman, Dekker, NY.

In certain embodiments, solid dosage forms, for example tablets, effervescent tablets, and capsules, are prepared by mixing the active agents of the present invention particles with one or more pharmaceutical excipients to form a bulk blend composition. When referring to these bulk blend compositions as homogeneous, it is meant that the active agents of the present invention particles are dispersed evenly throughout the composition so that the composition may be readily subdivided into equally effective unit dosage forms, such as tablets, pills, and capsules. The individual unit dosages may also comprise film coatings, which disintegrate upon oral ingestion or upon contact with diluents. These the active agents of the present invention formulations can be manufactured by conventional pharmaceutical techniques.

Conventional pharmaceutical techniques for preparation of solid dosage forms include, for example, one or a combination of methods: (1) dry mixing, (2) direct compression, (3) milling, (4) dry or non-aqueous granulation, (5) wet granulation, or (6) fusion. See, for example, Lachman et al., Theory and Practice of Industrial Pharmacy (1986). Other methods include, for example, spray drying, pan coating, melt granulation, granulation, fluidized bed spray drying or coating (for example, Wurster coating), tangential coating, top spraying, tableting, extruding and the like.

Compressed tablets are solid dosage forms prepared by compacting the bulk blend the active agents of the present invention formulations described above. In various embodiments, compressed tablets which are designed to dissolve in the mouth will comprise one or more flavoring agents. In other embodiments, the compressed tablets will comprise a film surrounding a final compressed tablet. In some embodiments, the film coating can provide a delayed release of the active agents of the present invention formulation. In other embodiments, the film coating aids in patient compliance (for example, Opadry® coatings or sugar coating). Film coatings comprising Opadry® typically range from about 1% to about 3% of the tablet weight. Film coatings for delayed-release usually comprise 2-6% of a tablet weight or 7-15% of a spray-layered bead weight. In other embodiments, the compressed tablets comprise one or more excipients.

A capsule may be prepared, for example, by placing the bulk blend of the active agents of the present invention formulation, described above, inside of a capsule. In some embodiments, the formulations of the present invention (non-aqueous suspensions and solutions) are placed in a soft gelatin capsule. In other embodiments, the formulations of the present invention are placed in standard gelatin capsules or non-gelatin capsules such as capsules comprising HPMC. In other embodiments, the formulations of the present invention are placed in a sprinkle capsule, wherein the capsule may be swallowed whole, or the capsule may be opened, and the contents sprinkled on food prior to eating. In some embodiments of the present invention, the therapeutic dose is split into multiple (for example, two, three, or four) capsules. In some embodiments, the entire dose of the active agents of the present invention is delivered in a capsule form.

In certain embodiments, ingredients (including or not including the active agent) of the invention are wet granulated. The individual steps in the wet granulation process of tablet preparation include milling and sieving of the ingredients, dry powder mixing, wet massing, granulation, drying, and final grinding. In various embodiments, the active agents of the present invention composition are added to the other excipients of the pharmaceutical formulation after they have been wet granulated. Alternatively, the ingredients may be subjected to dry granulation, for example, via compressing a powder mixture into a rough tablet or “slug” on a heavy-duty rotary tablet press. The slugs are then broken up into granular particles by a grinding operation, usually by passage through an oscillation granulator. The individual steps include mixing of the powders, compressing (slugging) and grinding (slug reduction or granulation). No wet binder or moisture is involved in any of the steps.

In some embodiments, the active agents of the present invention formulation are dry granulated with other excipients in the pharmaceutical formulation. In other embodiments, the active agents of the present invention formulation are added to other excipients of the pharmaceutical formulation after they have been dry granulated.

In other embodiments, the formulation of the present invention formulations described herein is a solid dispersion. Methods of producing such solid dispersions are known in the art and include U.S. Pat. Nos. 4,343,789; 5,340,591; 5,456,923; 5,700,485; 5,723,269; and U.S. Pub. No. 2004/0013734. In some embodiments, the solid dispersions of the invention comprise both amorphous and non-amorphous active agents of the present invention and can have enhanced bioavailability as compared to conventional active agents of the present invention formulations. In still other embodiments, the active agents of the present invention formulations described herein are solid solutions. Solid solutions incorporate a substance together with the active agent and other excipients such that heating the mixture results in the dissolution of the drug and the resulting composition is then cooled to provide a solid blend that can be further formulated or directly added to a capsule or compressed into a tablet.

### Non-Limiting Examples of Formulations for Oral Delivery

The examples below provide non-limiting embodiments of formulations for oral delivery, which can be used to deliver any of the compounds described herein in enantiomerically enriched form, pure form or even a racemic mixture. Therefore, while the compounds below are specified, any desired purity form or compound can be used if it achieves the desired goal of treatment.

In one non-limiting embodiment, hard gelatin capsules comprising the following ingredients are prepared by mixing the ingredients and filling into hard gelatin capsules in 340 mg quantities.

| Ingredient | Quantity (mg/capsule) |
|---|---|
| T-1 | 30.0 |
| Starch | 205.0 |
| Alpha lipoic acid | 100.0 |
| Magnesium stearate | 5.0 |

In one non-limiting embodiment, a tablet formulation is prepared comprising the ingredients below. The components are blended and compressed to form tablets, each weighing 240 mg.

| Ingredient | Quantity (mg/tablet) |
|---|---|
| T-2 | 25.0 |
| Cellulose, microcrystalline | 200.0 |
| Colloidal silicon dioxide | 10.0 |
| Stearic acid | 5.0 |

In one non-limiting embodiment, a tablet, comprising the components below, including an enantiomerically enriched mixture of compound T-4 and an enantiomerically enriched mixture of compound T-5, is prepared. The active ingredients, starch and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50-60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

| Ingredient | Quantity (mg/tablet) |
|---|---|
| T-4 | 20.0 |
| T-5 | 10.0 |
| Starch | 45.0 |
| Microcrystalline cellulose | 35.0 |
| Polyvinylpyrrolidone (as 10% | 4.0 |
| solution in water) | |
| Sodium carboxymethyl starch | 4.5 |
| Magnesium stearate | 0.5 |
| Talc | 1.0 |

In one non-limiting embodiment, a capsule, comprising the components below, including the enriched R-enantiomer of compound T-6 and the enriched R-enantiomer of compound T-5, is prepared. The active ingredients, cellulose, starch, and magnesium stearate are blended, passed through a No. 20 mesh U.S. sieve, and filled into hard gelatin capsules in 150 mg quantities.

| Ingredient | Quantity (mg/capsule) |
|---|---|
| T-6 (R-enantiomer enriched) | 10.0 |
| T-5 (R-enantiomer enriched) | 30.0 |
| Starch | 109.0 |
| Magnesium stearate | 1.0 |

In one non-limiting embodiment, a capsule, comprising 100 mg of enantiomerically enriched compound T-12, is prepared using the ingredients below. The active ingredient, cellulose, starch, and magnesium stearate are blended, passed through a No. 20 mesh U.S. sieve, and filled into hard gelatin capsules in 510 mg quantities.

| Ingredient | Amount (mg/capsule) |
|---|---|
| T-12 | 100.0 |
| Starch | 407.0 |
| Magnesium stearate | 3.0 |

### Extended-Release Formulations

Depending on the desired release profile, the pharmaceutical formulation, for example, an oral solid dosage form, may contain a suitable amount of controlled-release agents, extended-release agents, and/or modified-release agents (for example, delayed-release agents). The pharmaceutical solid oral dosage forms comprising the active agents of the present invention described herein can be further formulated to provide a modified or controlled release of the active agents of the present invention. In some embodiments, the solid dosage forms described herein can be formulated as a delayed release dosage form such as an enteric-coated delayed release oral dosage forms, i.e., as an oral dosage form of a pharmaceutical composition as described herein which uses an enteric coating to affect release in the small intestine of the gastrointestinal tract. The enteric-coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated. Enteric coatings may also be used to prepare other controlled release dosage forms including extended-release and pulsatile release dosage forms.

In other embodiments, the active agents of the formulations described herein are delivered using a pulsatile dosage form. Pulsatile dosage forms comprising the active agents of the present invention described herein may be administered using a variety of formulations known in the art. For example, such formulations include those described in U.S. Pat. Nos. 5,011,692; 5,017,381; 5,229,135; and 5,840,329. Other dosage forms suitable for use with the active agents of the present invention are described in, for example, U.S. Pat. Nos. 4,871,549; 5,260,068; 5,260,069; 5,508,040; 5,567,441; and 5,837,284.

In one embodiment, the controlled release dosage form is pulsatile release solid oral dosage form comprising at least two groups of particles, each containing active agents of the present invention as described herein. The first group of particles provides a substantially immediate dose of the active agents of the present invention upon ingestion by a subject. The first group of particles can be either uncoated or comprise a coating and/or sealant. The second group of particles comprises coated particles, which may comprise from at least about 2% to about 75% or less, typically from at least about 2.5% to about 70% or less, or from at least about 40% to about 70% or less, by weight of the total dose of the active agents of the present invention in the formulation, in admixture with one or more binders.

In one embodiment, a coating for providing a controlled, delayed, or extended-release is applied to S-BK-5F-NM-AMT, R-BK-5F-NM-AMT, S-BK-5Cl-NM-AMT, R-BK-5Cl-NM-AMT, S-BK-5Br-NM-AMT, and/or R-BK-5Br-NM-AMT or to a core containing S-BK-5F-NM-AMT, R-BK-5F-NM-AMT, S-BK-5Cl-NM-AMT, R-BK-5Cl-NM-AMT, S-BK-5Br-NM-AMT, and/or R-BK-5Br-NM-AMT. In one embodiment, a coating for providing a controlled, delayed, or extended-release is applied to a compound of any of Formulas I-XXIX or to a core containing a compound of any of Formulas I-XXIX.

The coating may comprise a pharmaceutically acceptable ingredient in an amount sufficient, for example, to provide an extended release from for example, about 1 hours to about 7 hours following ingestion before release of the active agent. Suitable coatings include one or more differentially degradable coatings such as, by way of example only, pH-sensitive coatings (enteric coatings) such as acrylic resins (for example, Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, and Eudragit® NE30D, Eudragit® NE 40D®) either alone or blended with cellulose derivatives, for example, ethylcellulose, or non-enteric coatings having variable thickness to provide differential release of the active agents of the present invention formulation.

Many other types of controlled/delayed/extended-release systems known to those of ordinary skill in the art and are suitable for use with the active agents of the present invention formulations described herein. Examples of such delivery systems include polymer-based systems, such as polylactic and polyglycolic acid, polyanhydrides and polycaprolactone, cellulose derivatives (for example, ethylcellulose), porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, for example, Liberman et al., Pharmaceutical Dosage Forms, 2 Ed., Vol. 1, pp. 209-214 (1990); Singh et al., Encyclopedia of Pharmaceutical Technology, 2nd Ed., pp. 751-753 (2002); U.S. Pat. Nos. 4,327,725; 4,624,848; 4,968,509; 5,461,140; 5,456,923, 5,516,527; 5,622,721, 5,686,105; 5,700,410; 5,977,175; 6,465,014 and 6,932,983.

In certain embodiments, the controlled release systems may comprise the controlled/delayed/extended-release material incorporated with the drug(s) into a matrix, whereas in other formulations, the controlled release material may be applied to a core containing the drug(s). In certain embodiments, one drug may be incorporated into the core while the other drug is incorporated into the coating. In some embodiments, materials include shellac, acrylic polymers, cellulosic derivatives, polyvinyl acetate phthalate, and mixtures thereof. In other embodiments, materials include Eudragit® series E, L, RL, RS, NE, L, L300, S, 100-55, cellulose acetate phthalate, Aquateric, cellulose acetate trimellitate, ethyl cellulose, hydroxypropylmethylcellulose phthalate, hydroxypropylmethylcellulose acetate succinate, polyvinyl acetate phthalate, and Cotteric.

The controlled/delayed/extended-release systems may use a hydrophilic polymer, including a water-swellable polymer (for example, a natural or synthetic gum). The hydrophilic polymer may be any pharmaceutically acceptable polymer which swells and expands in the presence of water to slowly release the active agents of the present invention. These polymers include polyethylene oxide, methylcellulose, hydroxypropyl cellulose, hydroxypropylmethylcellulose, and the like.

The performance of acrylic polymers (primarily their solubility in biological fluids) can vary based on the degree and type of substitution. Examples of suitable acrylic polymers which may be used in matrix formulations or coatings include methacrylic acid copolymers and ammonia methacrylate copolymers. The Eudragit series E, L, S, RL, RS and NE (Rohm Pharma) are available as solubilized in an organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in the stomach and dissolve in the intestine; Opadry Enteric is also insoluble in the stomach and dissolves in the intestine.

Examples of suitable cellulose derivatives for use in matrix formulations or coatings include ethyl cellulose; reaction mixtures of partial acetate esters of cellulose with phthalic anhydride. The performance can vary based on the degree and type of substitution. Cellulose acetate phthalate (CAP) dissolves in pH>6. Aquateric (FMC) is an aqueous-based system and is a spray-dried CAP psuedolatex with particles <1 μm. Other components in Aquateric can include pluronic, Tweens, and acetylated monoglycerides. Other suitable cellulose derivatives include cellulose acetate trimellitate (Eastman); methylcellulose (Pharmacoat, Methocel); hydroxypropylmethylcellulose phthalate (HPMCP); hydroxypropylmethylcellulose succinate (HPMCS); and hydroxypropylmethylcellulose acetate succinate (for example, AQOAT (Shin Etsu)). The performance can vary based on the degree and type of substitution. For example, HPMCP such as, HP-50, HP-55, HP-555, HP-55F grades are suitable. The performance can vary based on the degree and type of substitution. For example, suitable grades of hydroxypropylmethylcellulose acetate succinate include AS-LG (LF), which dissolves at pH 5, AS-MG (MF), which dissolves at pH 5.5, and AS-HG (HF), which dissolves at higher pH. These polymers are offered as granules or as fine powders for aqueous dispersions. Other suitable cellulose derivatives include hydroxypropylmethylcellulose.

In some embodiments, the coating may contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate, and triacetin. Conventional coating techniques such as spray or pan coating are employed to apply coatings. The coating thickness must be sufficient to ensure that the oral dosage form remains intact until the desired site of topical delivery in the intestinal tract is reached.

Multilayer tablet delivery (for example, such as that used in the GeoMatrix™ technology) comprises a hydrophilic matrix core containing the active ingredient and one or two impermeable or semi-permeable polymeric coatings. This technology uses films or compressed polymeric barrier coatings on one or both sides of the core. The presence of polymeric coatings (for example, such as that used in the GeoMatrix™ technology) modifies the hydration/swelling rates of the core and reduces the surface area available for drug release. These partial coatings provide modulation of the drug dissolution profile: they reduce the release rate from the device and shift the typical time-dependent release rate toward constant release. This technology enables customized levels of controlled release of specific active agents and/or simultaneous release of two different active agents at different rates that can be achieved from a single tablet. The combination of layers, each with different rates of swelling, gelling and erosion, is used for the rate of drug release in the body. Exposure of the multilayer tablet as a result of the partial coating may affect the release and erosion rates, therefore, transformation of a multilayered tablet with exposure on all sides to the gastrointestinal fluids upon detachment of the barrier layer will be considered.

Multi-layered tablets containing combinations of immediate release and modified/extended release of two different active agents or dual release rate of the same drug in a single dosage form may be prepared by using hydrophilic and hydrophobic polymer matrices. Dual release repeat action multi-layered tablets may be prepared with an outer compression layer with an initial dose of rapidly disintegrating matrix in the stomach and a core inner layer tablet formulated with components that are insoluble in the gastric media but release efficiently in the intestinal environment.

In certain embodiments, the dosage form is a solid oral dosage form which is an immediate release dosage form whereby >80% of the active agents of the present invention are released within 2 hours after administration. In other embodiments, the invention provides an (for example, solid oral) dosage form that is a controlled release or pulsatile release dosage form. In such instances, the release may be, for example, 30 to 60% of the active agents of the present invention particles by weight are released from the dosage form within about 2 hours after administration and about 90% by weight of the active agents of the present invention released from the dosage form, for example, within about 4 hours after administration. In yet other embodiments, the dosage form includes at least one active agent in an immediate-release form and at least one active agent in the delayed-release form or sustained-release form. In yet other embodiments, the dosage form includes at least two active agents that are released at different rates as determined by in-vitro dissolution testing or via oral administration.

The various release dosage formulations discussed above, and others known to those skilled in the art can be characterized by their disintegration profile. A profile is characterized by the test conditions selected. Thus, the disintegration profile can be generated at a pre-selected apparatus type, shaft speed, temperature, volume, and pH of the dispersion media. Several disintegration profiles can be obtained. For example, a first disintegration profile can be measured at a pH level approximating that of the stomach (about pH 1.2); a second disintegration profile can be measured at a pH level approximating that of one point in the intestine or several pH levels approximating multiple points in the intestine (about 6.0 to about 7.5, more specifically, about 6.5 to 7.0). Another disintegration profile can be measured using distilled water. The release of formulations may also be characterized by their pharmacokinetic parameters, for example, Cₘₐₓ, Tₘₐₓ, and AUC (0-τ).

In certain embodiments, the controlled, delayed or extended-release of one or more of the active agents of the fixed-dose combinations of the invention may be in the form of a capsule having a shell comprising the material of the rate-limiting membrane, including any of the coating materials previously discussed, and filled with the active agents of the present invention particles. A particular advantage of this configuration is that the capsule may be prepared independently of the active agent of the present invention particles; thus, process conditions that would adversely affect the drug can be used to prepare the capsule.

Alternatively, the formulation may comprise a capsule having a shell made of a porous or a pH-sensitive polymer made by a thermal forming process. Another alternative is a capsule shell in the form of an asymmetric membrane, i.e., a membrane that has a thin skin on one surface and most of whose thickness is constituted of a highly permeable porous material. The asymmetric membrane capsules may be prepared by a solvent exchange phase inversion, wherein a solution of polymer, coated on a capsule-shaped mold, is induced to phase separate by exchanging the solvent with a miscible non-solvent. In another embodiment, spray layered active agents of the present invention particles are filled in a capsule.

An exemplary process for manufacturing the spray layered the active agents of the present invention is the fluidized bed spraying process. The active agents of the present invention suspensions or the active agents of the present invention complex suspensions described above may be sprayed onto sugar or microcrystalline cellulose (MCC) beads (20-35 mesh) with Wurster column insert at an inlet temperature of 50° C. to 60° C. and air temp of 30° C. to 50° C. A 15 to 20 wt % total solids content suspension containing 45 to 80 wt % the active agents of the present invention, 10 to 25 wt % hydroxymethylpropylcellulose, 0.25 to 2 wt % of SLS, 10 to 18 wt % of sucrose, 0.01 to 0.3 wt % simethicone emulsion (30% emulsion) and 0.3 to10% NaCl, based on the total weight of the solid content of the suspension, are sprayed (bottom spray) onto the beads through 1.2 mm nozzles at 10 mL/min and 1.5 bar of pressure until a layering of 400 to 700% wt % is achieved as compared to initial beads weight. The resulting spray layered the active agents of the present invention particles, or the active agents of the present invention complex particles comprise about 30 to 70 wt % of the active agents of the present invention based on the total weight of the particles.

In one embodiment the capsule is a size 0 soft gelatin capsule. In one embodiment, the capsule is a swelling plug device. In another embodiment, the swelling plug device is further coated with cellulose acetate phthalate or copolymers of methacrylic acid and methylmethacrylate. In some embodiments, the capsule includes at least 40 mg (or at least 100 mg or at least 200 mg) of the active agents of the present invention and has a total weight of less than 800 mg (or less than 700 mg). The capsule may contain a plurality of the active agents of the present invention-containing beads, for example, spray layered beads. In some embodiments, the beads are 12-25% the active agents of the present invention by weight. In some embodiments, some or all of the active agents of the present invention containing beads are coated with a coating comprising 6 to 15% (or 8 to 12%) of the total bead weight. Optimization work typically involves lower loading levels, and the beads constitute 30 to 60% of the finished bead weight. The capsule may contain a granulated composition, wherein the granulated composition comprises the active agents of the present invention.

The capsule may provide pulsatile release of the active agents of the present invention oral dosage form. In one embodiment, the formulations comprise: (a) a first dosage unit comprising BK-5F-NM-AMT, BK-5Cl-NM-AMT, and/or BK-5Br-NM-AMT that is released substantially immediately following oral administration of the dosage form to a patient; (b) a second dosage unit comprising BK-5F-NM-AMT, BK-5Cl-NM-AMT, and/or BK-5Br-NM-AMT that is released approximately 2 to 6 hours following administration of the dosage form to a patient.

In one embodiment, the formulation comprises: (a) a first dosage unit comprising compounds of any of Formulas I-XXIX that is released substantially immediately following oral administration of the dosage form to a patient; (b) a second dosage unit comprising compounds of any of Formulas I-XXIX that is released approximately 2 to 6 hours following administration of the dosage form to a patient.

For pulsatile release capsules containing beads, the beads can be coated with a coating comprising 6 to 15% (or 8 to 12%) of the total bead weight. In some embodiments, the coating is a coating that is insoluble at pH 1 to 2 and soluble at pH greater than 5.5. In other embodiments, the pulsatile release capsule contains a plurality of beads formulated for modified release and the at least one agent of the present invention is, for example, spray granulated for immediate release.

In some embodiments, the release of the active agents of the present invention particles can be modified with a modified release coating, such as an enteric coating using cellulose acetate phthalate or a sustained release coating comprising copolymers of methacrylic acid and methylmethacrylate. In one embodiment, the enteric coating may be present in an amount of about 0.5 to about 15 wt %, more specifically, about 8 to about 12 wt %, based on the weight of, for example, the spray layered particles. In one embodiment, the spray layered particles coated with the delayed and/or sustained release coatings can be filled in a modified release capsule in which both enteric-coated particles and immediate release particles of the present invention beads are filled into a soft gelatin capsule. Additional suitable excipients may also be filled with the coated particles in the capsule. The uncoated particles release the active agent of the present invention immediately upon administration while the coated particles do not release the active agent of the present invention until these particles reach the intestine. By controlling the ratios of the coated and uncoated particles, desirable pulsatile release profiles also may be obtained. In some embodiments, the ratios between the uncoated and the coated particles are for example, 20/80, or 30/70, or 40/60, or 50/50, w/w to obtain desirable release.

In certain embodiments, spray layered active agents of the present invention can be compressed into tablets with commonly used pharmaceutical excipients. Any appropriate apparatus for forming the coating can be used to make the enteric coated tablets, for example, fluidized bed coating using a Wurster column, powder layering in coating pans or rotary coaters; dry coating by double compression technique; tablet coating by film coating technique, and the like. See, for example, U.S. Pat. No. 5,322,655; Remington's Pharmaceutical Sciences Handbook: Chapter 90 “Coating of Pharmaceutical Dosage Forms,” 1990.

In certain embodiments, the spray layered active agents of the present invention described above and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the active agents of the present invention formulation into the gastrointestinal fluid. In other embodiments, the spray layered active agents of the present invention particles or spray layered active agents complex particles with enteric coatings described above and one or more excipients are dry blended and compressed into a mass, such as a tablet.

In certain embodiments, a pulsatile release of the active agent of the present invention formulation comprises a first dosage unit comprising a formulation made from the active agent of the present invention containing granules made from a spray drying or spray granulated procedure or a formulation made from the active agent of the present invention complex containing granules made from a spray drying or spray granulated procedure without enteric or sustained-release coatings and a second dosage unit comprising spray layered the active agent of the present invention particles or spray layered the active agent of the present invention complex particles with enteric or sustained-release coatings. In one embodiment, the active agent is wet or dry blended and compressed into a mass to make a pulsatile release tablet.

In certain embodiments, binding, lubricating and disintegrating agents are blended (wet or dry) to the spray layered active agent of the present invention to make a compressible blend. In one embodiment, the dosage unit containing BK-5F-NM-AMT, BK-5Cl-NM-AMT, and/or BK-5Br-NM-AMT and the dosage unit containing the other pharmacological agent are compressed separately and then compressed together to form a bilayer tablet. In yet another embodiment, the dosage unit containing the other pharmacological agent is in the form of an overcoat and completely covers the second dosage unit containing BK-5F-NM-AMT, BK-5Cl-NM-AMT, and/or BK-5Br-NM-AMT. In yet another embodiment, the dosage unit containing BK-5F-NM-AMT, BK-5Cl-NM-AMT, and/or BK-5Br-NM-AMT is in the form of an overcoat and completely covers the second dosage unit containing the other pharmacological agent.

In one embodiment, the dosage unit containing a compound of any of Formulas I-XXIX and the dosage unit containing the other pharmacological agent are compressed separately and then compressed together to form a bilayer tablet. In yet another embodiment, the dosage unit containing the other pharmacological agent is in the form of an overcoat and completely covers the second dosage unit containing a compound of any of Formulas I-XXIX. In yet another embodiment, the dosage unit containing a compound of any of Formulas I-XXIX is in the form of an overcoat and completely covers the second dosage unit containing the other pharmacological agent.

### Systemic Formulations

The formulations of the present invention can include any selected compound of the present invention for any of the disclosed indications in a form suitable for intramuscular, subcutaneous, or intravenous injection may comprise physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propylene glycol, polyethylene-glycol, glycerol, cremophor and the like), suitable mixtures thereof, vegetable oils (such as olive oil) and injectable organic esters such as ethyl oleate. Additionally, the active agents of the present invention can be dissolved at concentrations of greater than about 1 mg/ml using water-soluble beta cyclodextrins (for example, beta-sulfobutyl-cyclodextrin and 2-hydroxypropyl-beta-cyclodextrin. Proper fluidity can be maintained, for example, by the use of a coating such as a lecithin, by the maintenance of the required particle size in the case of dispersions, and by the use of surfactants.

The formulations of the present invention suitable for subcutaneous injection may also contain additives such as preserving, wetting, emulsifying, and dispensing agents. Prevention of the growth of microorganisms can be ensured by various antibacterial and antifungal agents, such as parabens, benzoic acid, benzyl alcohol, chlorobutanol, phenol, sorbic acid, and the like. It may also be desirable to include isotonic agents, such as sugars, sodium chloride, and the like. Prolonged drug absorption of the injectable pharmaceutical form can be brought about by the use of agents delaying absorption, such as aluminum monostearate and gelatin. The formulations of the present invention designed for extended-release via subcutaneous or intramuscular injection can avoid first-pass metabolism and lower dosages of the active agents of the present invention will be necessary to maintain plasma levels of about 50 ng/ml. In such formulations, the particle size of the active agents of the present invention and the range of the particle sizes of the active agents of the present invention particles can be used to control the release of the drug by controlling the rate of dissolution in fat or muscle.

In one embodiment, a pharmaceutical composition containing BK-5F-NM-AMT, BK-5Cl-NM-AMT, and/or BK-5Br-NM-AMT or a pharmaceutically acceptable salt thereof is formulated into a dosage form suitable for parenteral use. In one embodiment, pharmaceutical compositions containing compounds of any of Formulas I-XXIX or a pharmaceutically acceptable salt thereof is formulated into a dosage form suitable for parenteral use. The dosage form may be selected from, but not limited to, a lyophilized powder, a solution, or a suspension (for example, a depot suspension).

In one embodiment, a pharmaceutical composition containing BK-5F-NM-AMT, BK-5Cl-NM-AMT, and/or BK-5Br-NM-AMT or a pharmaceutically acceptable salt thereof is formulated into a topical dosage form. In one embodiment, a pharmaceutical composition containing a compound of any of Formulas I-XXIX or a pharmaceutically acceptable salt thereof is formulated into a topical dosage form. The topical dosage form is selected from, but not limited to, a patch, a gel, a paste, a cream, an emulsion, a liniment, a balm, a lotion, and an ointment.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. Such patches may be constructed for continuous, pulsatile, or on demand delivery of pharmaceutical agents.

Frequently, it will be desirable or necessary to introduce the pharmaceutical composition to the brain, either directly or indirectly. Direct techniques usually involve placement of a drug delivery catheter into the host's ventricular system to bypass the blood-brain barrier. Indirect techniques, which are generally useful, usually involve formulating the compositions to provide for drug latentiation by the conversion of hydrophilic drugs into lipid-soluble drugs or prodrugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions which can transiently open the blood-brain barrier.

### Non-Limiting Examples of Formulations for Systemic Delivery

The examples below provide non-limiting embodiments of formulations, which can be used to deliver any of the compounds described herein in enantiomerically enriched form, pure form or even a racemic mixture. Therefore, while the compounds below are specified, any desired purity form or compound can be used if it achieves the desired goal of treatment.

In one non-limiting embodiment, a suppository, comprising 25 mg of the R-enantiomer enriched compound T-5, is prepared. The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

| Ingredient | Quantity (mg) |
|---|---|
| T-5 (R-enantiomer enriched) | 25.0 |
| Saturated fatty acid glycerides | 2000.0 |

In one non-limiting embodiment, a suspension comprising 50 mg of enriched R-enantiomer of compound T-10 per 5.0 ml dose is prepared using the ingredients below. The active ingredient, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

| Ingredient | Amount |
|---|---|
| T-10 (R-enantiomer enriched) | 50.0 mg |
| Xanthan gum | 4.0 mg |
| Sodium carboxymethyl cellulose | 50.0 mg |
| (11%) | |
| Microcrystalline cellulose (89%) | 50 mg |
| Sucrose | 1.75 g |
| Sodium benzoate | 10.0 mg |
| Flavor and Color | q.v. |
| Purified water | To 5.0 ml |

In one non-limiting embodiment, an intravenous formulation is prepared using the following ingredients:

| Ingredient | Amount |
|---|---|
| T-14 | 250.0 mg |
| Isotonic saline | 1000 ml |

In one non-limiting embodiment, a topical formulation is prepared using the ingredients below. The white soft paraffin is heated until molten. The liquid paraffin and emulsifying wax are incorporated and stirred until dissolved. The active ingredient is added and stirring is continued until dispersed. The mixture is then cooled until solid.

| Ingredient | Amount (g) |
|---|---|
| T-6 | 10.0 |
| Emulsifying Wax | 30.0 |
| Liquid Paraffin | 20.0 |
| White Soft Paraffin | To 100 |

In one embodiment, a sublingual or buccal tablet, comprising 10 mg of the enantiomerically enriched compound T-18, is prepared using the following ingredients. The glycerol, water, sodium citrate, polyvinyl alcohol, and polyvinylpyrrolidone are admixed together by continuous stirring and maintaining the temperature at about 90° C. When the polymers have gone into solution, the solution is cooled to about 50-55′ C. and the medicament is slowly admixed. The homogenous mixture is poured into forms made of an inert material to produce a drug-containing diffusion matrix having a thickness of about 2-4 mm. This diffusion matrix is then cut to form individual tablets having the appropriate size.

| Ingredient | Amount (mg/tablet) |
|---|---|
| T-18 | 10.0 |
| Glycerol | 210.5 |
| Water | 143.0 |
| Sodium Citrate | 4.5 |
| Polyvinyl Alcohol | 26.5 |
| Polyvinylpyrrolidone | 15.5 |

In one non-limiting embodiment, a liquid formulation for vaporization comprising R-enantiomer enriched mixture of compound T-13 is prepared using the ingredients below. The active mixture is mixed and added to a liquid vaporization appliance.

| Ingredient | Quantity (units) |
|---|---|
| T-13 (R-enantiomer) | 500 mg |
| Propylene Glycol | 2 ml |
| Glycerin | 2 ml |

In one non-limiting embodiment, a formulation of dry powder for insufflation is prepared comprising the components below. The active mixture is mixed with the lactose and the mixture is added to a dry powder inhaling appliance.

| Ingredient | Weight % |
|---|---|
| T-3 | 5 |
| Lactose | 95 |

### Pharmaceutically Acceptable Salts

The compounds described herein, including enantiomerically enriched mixtures, can be administered if desired as a pharmaceutically acceptable salt or a salt mixture. A salt mixture may be useful to increase solubility of the active substances, to alter pharmacokinetics, or for controlled release or other objective. A salt mixture may comprise 2, 3, 4, 5, 6, or more pharmaceutically acceptable salts together to form a single composition.

The compounds of the present invention are amines and thus basic, and therefore, react with inorganic and organic acids to form pharmaceutically acceptable acid addition salts. In some embodiments, the compounds of the present invention as free amines are oily and have decreased stability at room temperature. In this case it may be beneficial to convert the free amines to their pharmaceutically acceptable acid addition salts for ease of handling and administration because in some embodiments, the pharmaceutically acceptable salt is solid at room temperature.

Acids commonly employed to form such salts are inorganic acids such as hydrochloric acid, hydrobromic acid, hydroiodic acid, sulfuric acid, nitric acid, phosphoric acid, and the like, and organic acids, such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. In one embodiment, the compounds of the present invention are administered as oxalate salts. In one embodiment of the present invention, the compounds are administered as phosphate salts.

Exemplary salts include, but are not limited to, 2-hydroxyethanesulfonate, 2-naphthalenesulfonate, 3-hydroxy-2-naphthoate, 3-phenylpropionate, acetate, adipate, alginate, amsonate, aspartate, benzenesulfonate, benzoate, besylate, bicarbonate, bisulfate, bitartrate, borate, butyrate, calcium edetate, camphorate, camphorsulfonate, camsylate, carbonate, citrate, clavulariate, cyclopentanepropionate, digluconate, dodecylsulfate, edetate, edisylate, estolate, esylate, ethanesulfonate, finnarate, gluceptate, glucoheptanoate, gluconate, glutamate, glycerophosphate, glycollylarsanilate, hemisulfate, heptanoate, hexafluorophosphate, hexanoate, hexylresorcinate, hydrabamine, hydrobromide, hydrochloride, hydroiodide, hydroxynaphthoate, iodide, sethionate, lactate, lactobionate, laurate, laurylsulphonate, malate, maleate, mandelate, mesylate, methanesulfonate, methylbromide, methylnitrate, methylsulfate, mucate, naphthylate, napsylate, nicotinate, nitrate, N-methylglucamine ammonium salt, oleate, oxalate, palmitate, pamoate, pantothenate, pectinate, persulfate, phosphate, phosphateldiphosphate, picrate, pivalate, polygalacturonate, propionate, p-toluenesulfonate, saccharate, salicylate, stearate, subacetate, succinate, sulfate, sulfosaliculate, suramate, tannate, tartrate, teoclate, thiocyanate, tosylate, triethiodide, undecanoate, and valerate salts, and the like.

Alternatively, exemplary salts include 2-hydroxyethanesulfonate, 2-naphthalenesulfonate, 2-napsylate, 3-hydroxy-2-naphthoate, 3-phenylpropionate, 4-acetamidobenzoate, acefyllinate, acetate, aceturate, adipate, alginate, aminosalicylate, ammonium, amsonate, ascorbate, aspartate, benzenesulfonate, benzoate, besylate, bicarbonate, bisulfate, bitartrate, borate, butyrate, calcium edetate, calcium, camphocarbonate, camphorate, camphorsulfonate, camsylate, carbonate, cholate, citrate, clavulariate, cyclopentanepropionate, cypionate, d-aspartate, d-camsylate, d-lactate, decanoate, dichloroacetate, digluconate, dodecylsulfate, edentate, edetate, edisylate, estolate, esylate, ethanesulfonate, ethyl sulfate, fumarate, furate, fusidate, galactarate (mucate), galacturonate, gallate, gentisate, gluceptate, glucoheptanoate, gluconate, glucuronate, glutamate, glutarate, glycerophosphate, glycolate, glycollylarsanilate, hemisulfate, heptanoate (enanthate), heptanoate, hexafluorophosphate, hexanoate, hexylresorcinate, hippurate, hybenzate, hydrabamine, hydrobromide, hydrobromide/bromide, hydrochloride, hydroiodide, hydroxide, hydroxybenzoate, hydroxynaphthoate, iodide, isethionate, isothionate, 1-aspartate, 1-camsylate, 1-lactate, lactate, lactobionate, laurate, laurylsulphonate, lithium, magnesium, malate, maleate, malonate, mandelate, meso-tartrate, mesylate, methanesulfonate, methylbromide, methylnitrate, methylsulfate, mucate, myristate, N-methylglucamine ammonium salt, napadisilate, naphthylate, napsylate, nicotinate, nitrate, octanoate, oleate, orotate, oxalate, p-toluenesulfonate, palmitate, pamoate, pantothenate, pectinate, persulfate, phenylpropionate, phosphate, phosphateldiphosphate, picrate, pivalate, polygalacturonate, potassium, propionate, pyrophosphate, saccharate, salicylate, salicylsulfate, sodium, stearate, subacetate, succinate, sulfate, sulfosaliculate, sulfosalicylate, suramate, tannate, tartrate, teoclate, terephthalate, thiocyanate, thiosalicylate, tosylate, tribrophenate, triethiodide, undecanoate, undecylenate, valerate, valproate, xinafoate, zinc, and the like. (See Berge et al. (1977) “Pharmaceutical Salts,” J. Pharm. Sci. 66:1-19.) Pharmaceutically acceptable salts include those employing a hydrochloride anion.

While salts of BK-5F-NM-AMT, BK-5Cl-NM-AMT, or BK-5Br-NM-AMT are illustrated, any of the compounds described herein can be substituted, including but not limited to a compound of any of Formulas I-XXIX. The compounds can be used as salts or salt mixtures as racemates, in enantiomerically enriched form, or in pure enantiomeric form. Nonlimiting examples are the oxalate and phosphate salts:

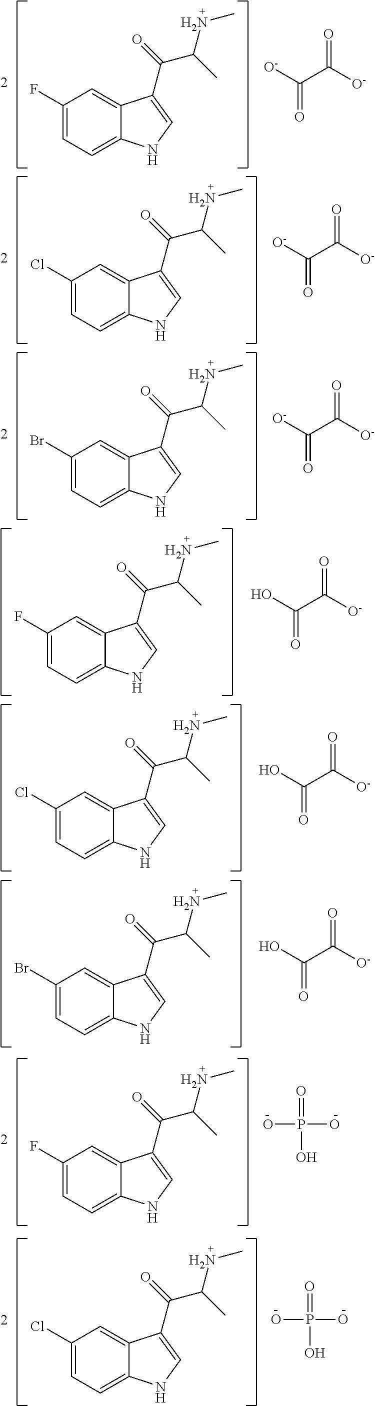

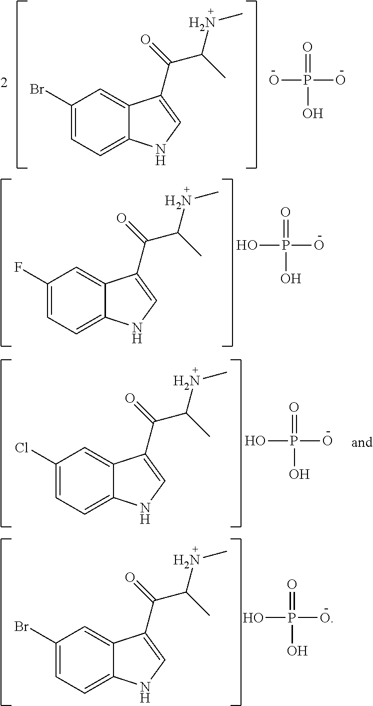

In certain illustrative nonlimiting embodiments, the pharmaceutically acceptable salt of BK-5F-NM-AMT, BK-5Cl-NM-AMT, or BK-5Br-NM-AMT, including racemic, enantiomerically pure, or enantiomerically enriched BK-5F-NM-AMT, BK-5Cl-NM-AMT, or BK-5Br-NM-AMT, is selected from:

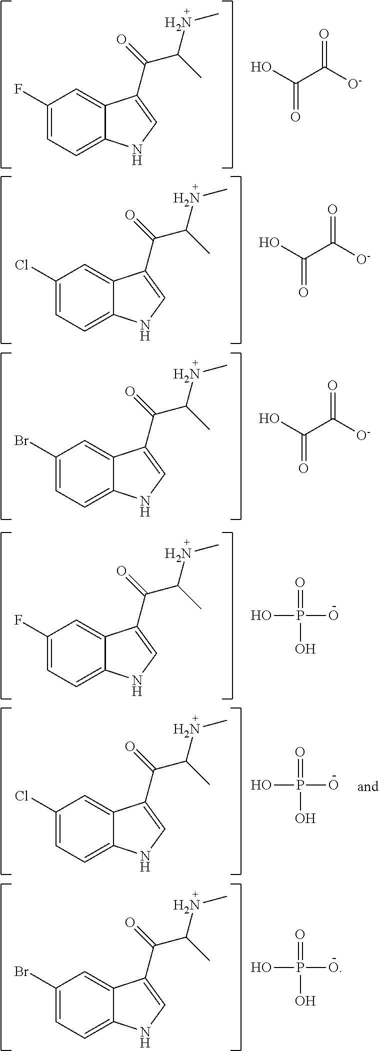

In certain illustrative nonlimiting embodiments, the pharmaceutically acceptable salt of BK-5F-NM-AMT, BK-5Cl-NM-AMT, or BK-5Br-NM-AMT, including enantiomerically pure or enantiomerically enriched BK-5F-NM-AMT, BK-5Cl-NM-AMT, or BK-5Br-NM-AMT, is selected from:

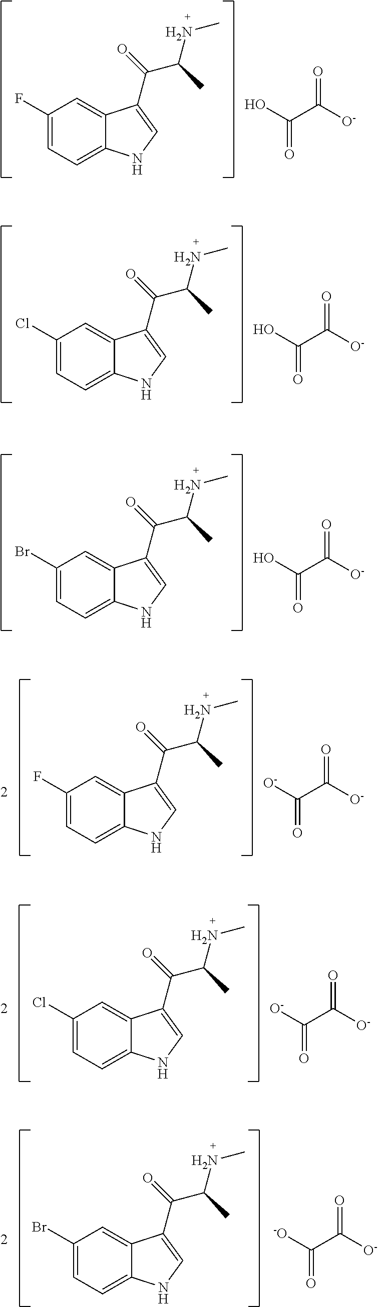

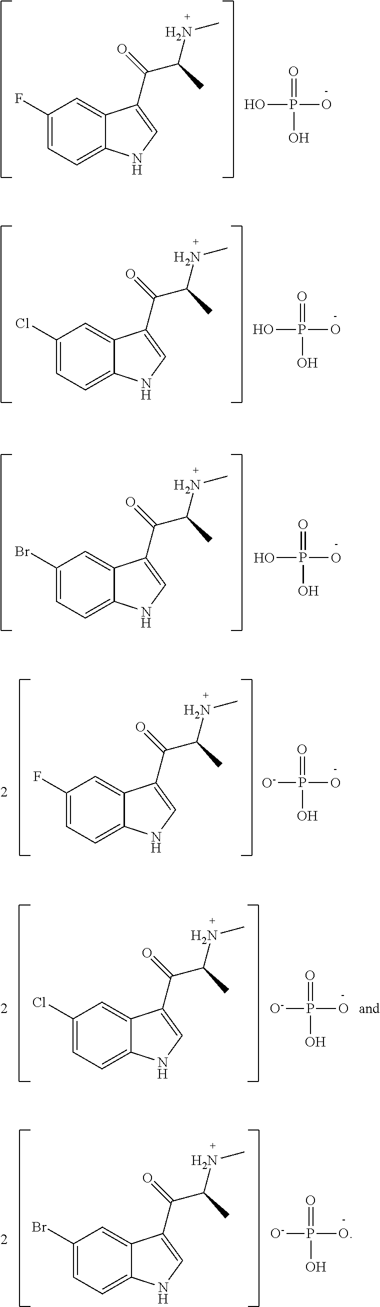

In certain illustrative nonlimiting embodiments, the pharmaceutically acceptable salt of BK-5F-NM-AMT, BK-5Cl-NM-AMT, or BK-5Br-NM-AMT, including enantiomerically pure or enantiomerically enriched BK-5F-NM-AMT, BK-5Cl-NM-AMT, or BK-5Br-NM-AMT, is selected from:

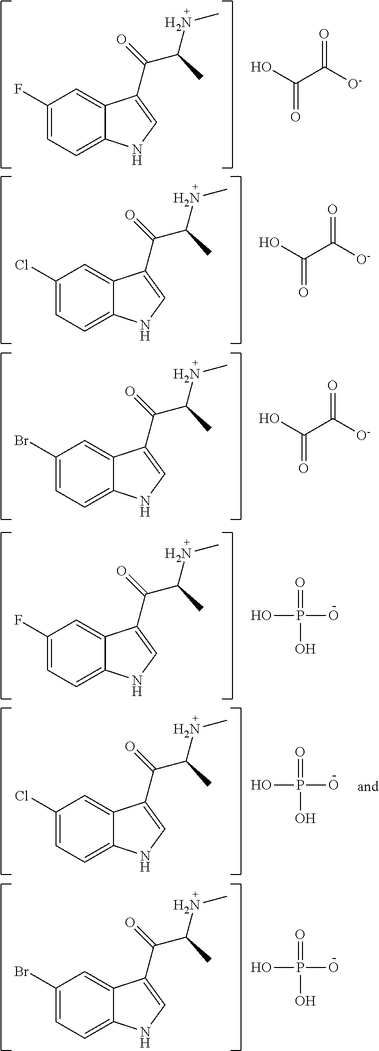

In certain illustrative nonlimiting embodiments, the pharmaceutically acceptable salt of BK-5F-NM-AMT, BK-5Cl-NM-AMT, or BK-5Br-NM-AMT, including enantiomerically pure or enantiomerically enriched BK-5F-NM-AMT, BK-5Cl-NM-AMT, or BK-5Br-NM-AMT, is selected from:

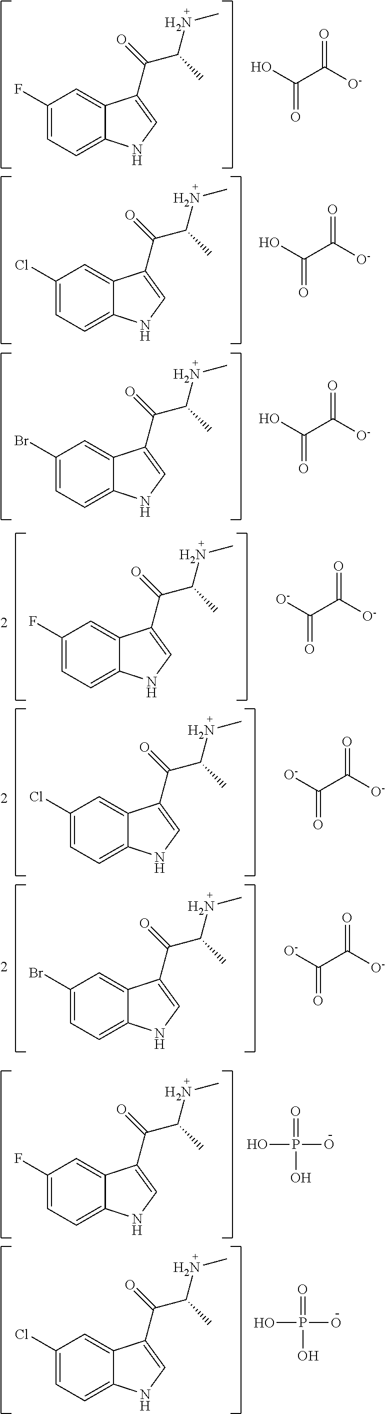

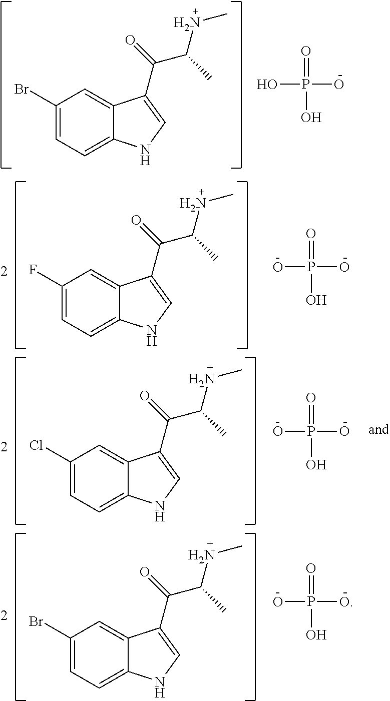

In certain illustrative nonlimiting embodiments, the pharmaceutically acceptable salt of BK-5F-NM-AMT, BK-5Cl-NM-AMT, or BK-5Br-NM-AMT, including enantiomerically pure or enantiomerically enriched BK-5F-NM-AMT, BK-5Cl-NM-AMT, or BK-5Br-NM-AMT, is selected from:

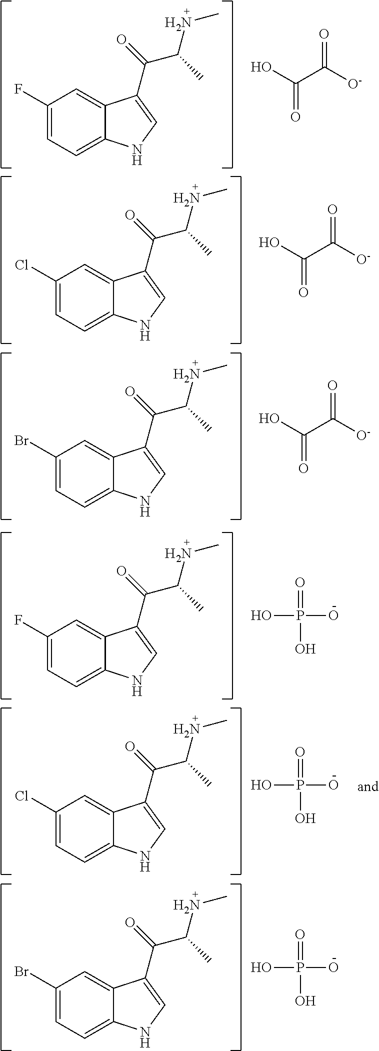

### Prodrugs

In certain aspects, the compounds of the present invention are administered as prodrugs. Prodrugs are compounds that are metabolized or otherwise transformed inside the body to the active pharmacologic agent(s) of interest. Thus, prodrug will contain the “active” component (for example, BK-5F-NM-AMT, BK-5Cl-NM-AMT, BK-5Br-NM-AMT, or a compound of any of Formulas I-XXIX and a prodrug moiety). Examples include N-alpha-acyloxyalkoxycarbonyl derivatives or addition of amino acids to the amine, which can be removed within the body by esterases or similar enzymes, and reactions at the keto-group to form enol ethers, enol esters, and imines. Prodrugs are frequently (though not necessarily) pharmacologically less active or inactive until converted to the parent drug. This is done in the body by a chemical or biological reaction. In some cases, the moiety or chemicals formed from it may also have beneficial effects, including increasing therapeutic effects, decreasing undesirable side effects, or otherwise altering the pharmacokinetics or pharmacodynamics of the active drug. When the chemical formed from the prodrug moiety has beneficial effects that contribute to the overall beneficial effects of administering the prodrug, then the formed chemical is considered a “codrug.”

Types of prodrugs contemplated to be within the scope of the invention include compounds that are transformed in various organs or locations in the body (for example, liver, kidney, G.I., lung, tissue) to release the active compound. For example, liver prodrugs will include active compounds conjugated with a polymer or chemical moiety that is not released until acted upon by liver cytochrome enzymes and CYP metabolism includes dealkylation, dehydrogenation, reduction, hydrolysis, oxidation, and the breakdown of aromatic rings. Kidney prodrugs will include active compounds conjugated to L-gamma-glutamyl or N-acetyl-L-gamma glutamic moieties so that they are metabolized by gamma-glutamyl transpeptidase before they are bioactive. Alternatively, the compounds may be conjugated to alkylglucoside moieties to create glycosylation-based prodrugs. Digestive or G.I. prodrugs will include those where an active compound is, for example, formulated into microspheres or nanospheres that do not degrade until the spheres are subjected to an acidic pH; formulated with an amide that will resist biochemical degradation until colonic pH is achieved; or, conjugated with a linear polysaccharide such as pectin that will delay activation until the combination reaches the bacteria in the colon. Besides these exemplary prodrug forms, many others will be known to those of ordinary skill.

Among derivatives of a compound are included its “physiologically functional derivatives,” which refers to physiologically tolerated chemical derivatives of the compound having the same physiological function thereof, for example, by being convertible in the body thereto, and which on administration to a mammal such as a human is able to form (directly or indirectly) the compound or an active metabolite thereof (acting therefore, like a prodrug), or by otherwise having the same physiological function, despite one or more structural differences. According to the present invention, examples of physiologically functional derivatives include esters, amides, carbamates, ureas, and heterocycles.

In yet other embodiments, the present invention includes enantiomerically enriched mixtures, or their pharmaceutically acceptable salts, salt mixtures, or prodrugs, of the R- or S-enantiomer of the racemic structure selected from:

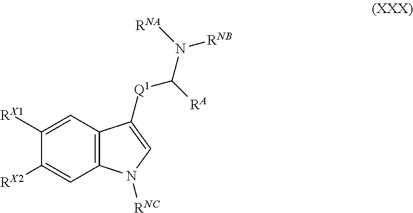

wherein:

R^{NA} and R^{NB} are independently selected from —H, optionally substituted —(C₁-C₆)alkyl, —OH, —Y, and —CH₂Y, and R^{NA} and R^{NB} can be taken together to form ═CHY;

R^{A} is selected from —H, —CH₃, —CH₂X, —CHX₂, —CX₃, —CH₂CH₃, —CH₂CH₂X, —CH₂CHX₂, —CH₂CX₃, —CH₂OH and —CH₂CH₂OH;

Q¹ is selected from

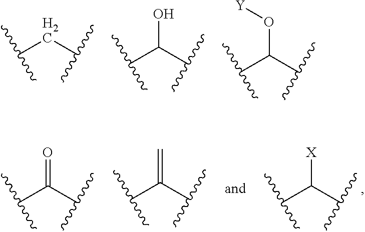

alternatively,
Q¹ and R^{NA} can be taken together to form

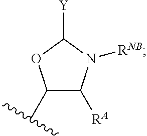

R^{NC} is selected from —H, —CH₃, —CH₂CH₃, and Y;

R^{X1} and R^{X2} are independently selected from —H, —X, —OH, —N(R^{c})₂, —(C₁-C₆)alkyl, —(C₁-C₄)alkenyl, —(C₂-C₈)alkynyl, —(C₁-C₆)alkoxy, —CF₃, —S(O)ₘR^{D}, —OS(O)₂CH₃, —OS(O)₂CF₃, —OP(O)(OH)₂, and —OP(O)(OR^{C})₂;

R^{C} is independently selected in each instance from —H, —CF₃, —(C₁-C₆)alkyl, —(C₂-C₈)alkenyl, (C₃-C₈)cycloalkyl, and aryl, wherein each —(C₁-C₆)alkyl, —(C₂-C₈)alkenyl, —(C₃-C₈)cycloalkyl, and aryl is optionally substituted as allowed by valence;

R^{D} is selected from —R^{C}, and —OR^{C};

m is selected from 0, 1, and 2;

X is independently in each instance selected from —F, —Cl, —Br, and —I;

Y is independently in each occurrence selected from —R^{P}, —R^{E}, —R^{BA}, —C(O)(CH₂)₀₋₈R^{P}, and —OC(O)(CH₂)₀₋₈R^{P};

R^{P} is an optionally substituted amino acid, dipeptide, tripeptide, or tetrapeptide selected from Table 2 in any combination that achieves the desired effects, wherein the R^{P} group is bound to the rest of the molecule at the N-terminal, C-terminal, or side chain of any amino acid contained therein;

R^{E} is independently selected in each instance from —H, alkyl, aryl, arylalkyl, cycloalkyl, cycloheteroalkyl, heteroaryl, heteroarylalkyl, and the prodrug structures given in Table 3 or its derivative, wherein each alkyl, aryl, arylalkyl, cycloalkyl, cycloheteroalkyl, heteroaryl, and heteroarylalkyl is optionally substituted as allowed by valence and at least one R^{E} is selected from Table 3; and

R^{BA} is a bile acid group, typically selected from:

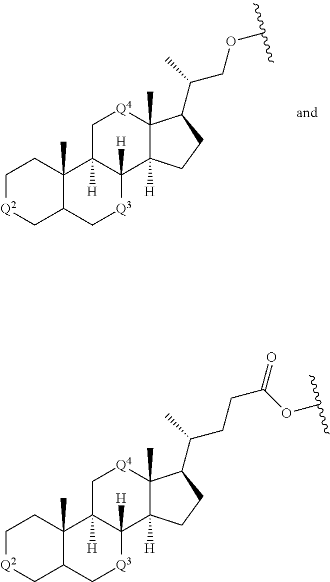

wherein:

Q², Q³, and Q⁴ are independently selected in each instance from

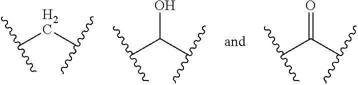

Some embodiments of Formula XXX include rings containing the nitrogen and oxygen, as in the following example, and in the structures of Table 4. Such ring structures act as prodrugs for the enol form of a beta-keto-tryptamine and will readily form from the enol when R^{NA} and R^{NB} together are ═CHY. A five-membered ring such as the one below can be formed, and such ring structures are also considered in this invention.

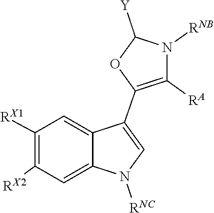

Exemplary R^{P} and R^{E} Groups

In some embodiments, an entactogen prodrug is provided. In some embodiments, the entactogen prodrug comprises at least one amino acid directly bonded to the entactogen. In some embodiments, the at least one amino acid is selected from Table 1. In some embodiments, the at least one amino acid comprises at least two amino acids as a peptide. In some embodiments, the at least two amino acids are a valine bonded to a valine via a peptide bond. In some embodiments, the at least two amino acids are three glycines bonded via peptide bonds.

The following examples provide non-exhaustive illustrations of R^{P} contemplated in some embodiments. However, this table is used for illustrative purposes and other possibilities inherent in the definition of R are contemplated. Similarly, geometric and other isomers are also contemplated.

TABLE 2
Exemplary R^{P} Amino Acids and Peptides

| Example Number | Example R^{P} |
|---|---|
| R^{P}-1 | Gly |
| R^{P}-2 | Gly-Gly |
| R^{P}-3 | Gly-Gly-Gly |
| R^{P}-4 | Phe |
| R^{P}-5 | Trp |
| R^{P}-6 | Trp-Trp |
| R^{P}-7 | Met |
| R^{P}-8 | Met-Met |
| R^{P}-9 | Met-Met-Met |
| R^{P}-10 | N-acetyl-Met |
| R^{P}-11 | Cys |
| R^{P}-12 | Cys-Cys |
| R^{P}-13 | N-acetyl-Cys |
| R^{P}-14 | D-ribose-Cys |
| R^{P}-15 | His |
| R^{P}-16 | His-His |
| R^{P}-17 | Ala-His |
| R^{P}-18 | Arg |
| R^{P}-19 | Arg-Arg |
| R^{P}-20 | Tyr |
| R^{P}-21 | Tyr-Tyr |
| R^{P}-22 | Val |
| R^{P}-23 | Val-Val |

In some embodiments, a tryptamine of the present invention has one or more R^{E} moieties conjugated either directly to the tryptamine or to an R^{P} group that is directly bonded to the tryptamine. Table 3 provides non-limiting illustrations of R^{E} contemplated in some embodiments. However, these are intended for illustrative purposes and other possibilities inherent in the definition of R^{E} are contemplated. Similarly, enantiomers and other stereoisomers are also contemplated.

TABLE 3
Example R^{E} Prodrug Groups

| R^{E} | |
|---|---|
| Number | R^{E} Prodrug Group |
| R^{E}-1 | 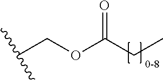 |
| R^{E}-2 | 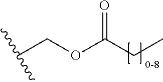 |
| R^{E}-3 | 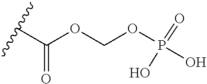 [SMILES: *C(=O)OCOP(=O)(O)O] |
| R^{E}-4 | 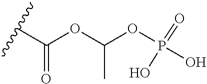 [SMILES: *C(=O)OC(C)OP(=O)(O)O] |
| R^{E}-5 | 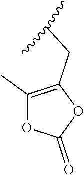 [SMILES: *Cc1oc(=O)oc1C] |
| R^{E}-6 | 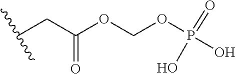 [SMILES: *CC(=O)OCOP(=O)(O)O] |
| R^{E}-7 | 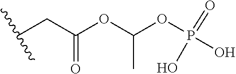 [SMILES: *CC(=O)OC(C)OP(=O)(O)O] |
| R^{E}-8 | 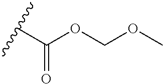 [SMILES: *C(=O)OCOC] |
| R^{E}-9 | 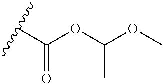 [SMILES: *C(=O)OC(C)OC] |
| R^{E}-10 | 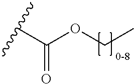 |
| R^{E}-11 | 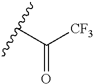 [SMILES: *C(=O)C(F)(F)F] |
| R^{E}-12 | 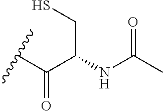 [SMILES: *C(=O)[C@H](CS)NC(C)=O] |
| R^{E}-13 | 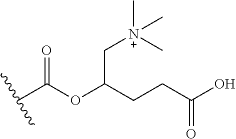 [SMILES: *C(=O)OC(CCC(=O)O)C[N+](C)(C)C] |
| R^{E}-14 | 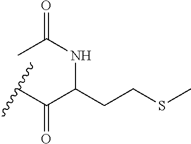 [SMILES: *C(=O)C(CCSC)NC(C)=O] |
| R^{E}-15 | 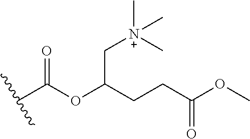 [SMILES: *C(=O)OC(CCC(=O)OC)C[N+](C)(C)C] |
| R^{E}-16 | 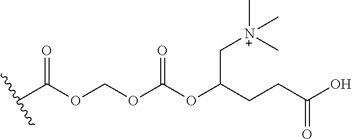 [SMILES: *C(=O)OCOC(=O)OC(CCC(=O)O)C[N+](C)(C)C] |
| R^{E}-17 | 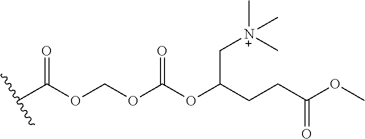 [SMILES: *C(=O)OCOC(=O)OC(CCC(=O)OC)C[N+](C)(C)C] |
| R^{E}-18 | 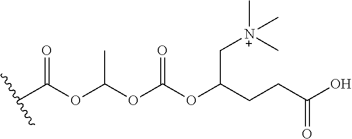 [SMILES: *C(=O)OC(C)OC(=O)OC(CCC(=O)O)C[N+](C)(C)C] |
| R^{E}-19 | 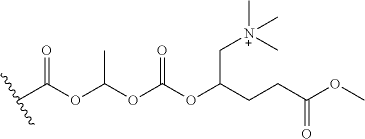 [SMILES: *C(=O)OC(C)OC(=O)OC(CCC(=O)OC)C[N+](C)(C)C] |
| R^{E}-20 | 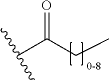 |
| R^{E}-21 | 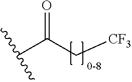 |
| R^{E}-22 | 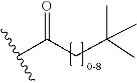 |
| R^{E}-23 | 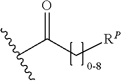 |
| R^{E}-24 | 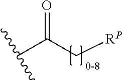 |

In some embodiments, the entactogen prodrug is selected from the non-limiting structures shown below (wherein the R^{E}-n substituents refer to the examples R^{E}-1 through R^{E}-24 in Table 3 above).

TABLE 4
Example Enantiomerically Enriched
Prodrugs of the Present Invention

| | |
|---|---|
| 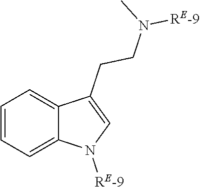 | P-1 |
| 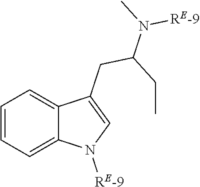 | P-2 |
| 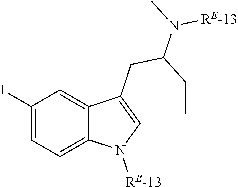 | P-3 |
| 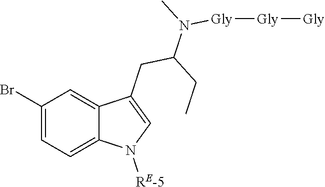 | P-4 |
| 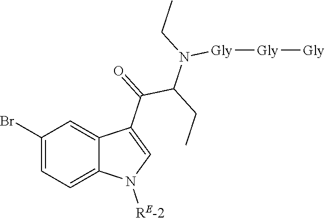 | P-5 |
| 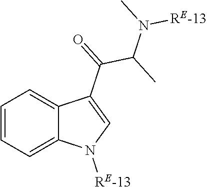 | P-6 |
| 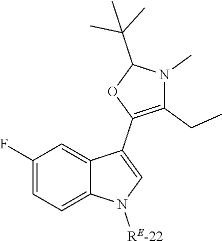 | P-7 |
| 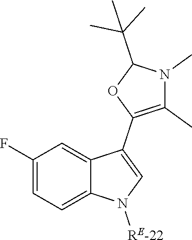 | P-8 |
| 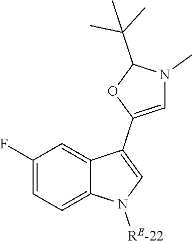 | P-9 |
| 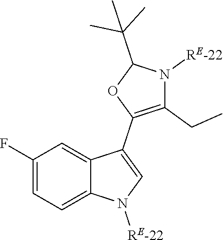 | P-10 |
| 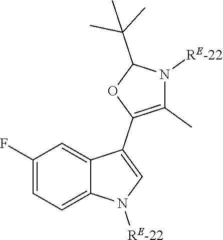 | P-11 |
| 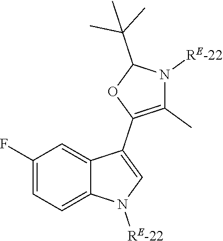 | P-12 |
| 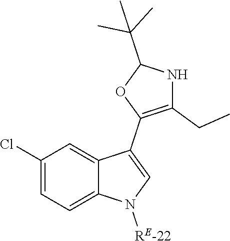 | P-13 |
| 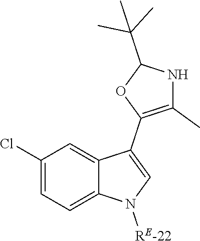 | P-14 |
| 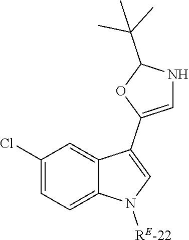 | P-15 |
| 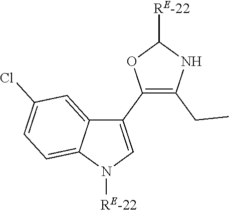 | P-16 |
| 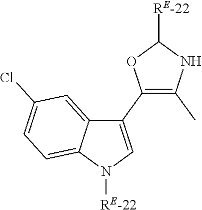 | P-17 |
| 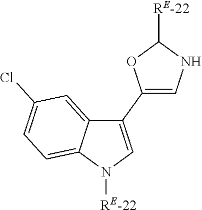 | P-18 |
| 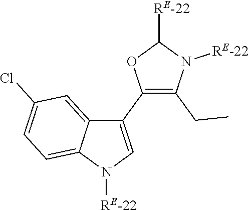 | P-19 |
| 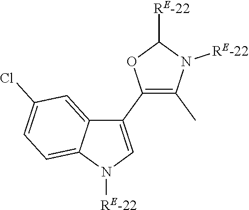 | P-20 |
| 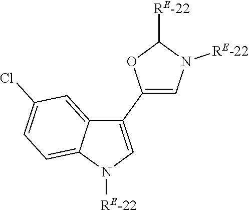 | P-21 |
| 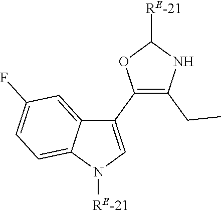 | P-22 |
| 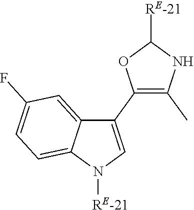 | P-23 |
| 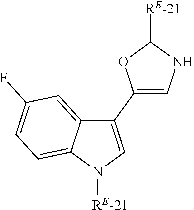 | P-24 |
| 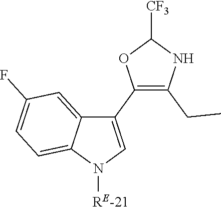 | P-25 |
| 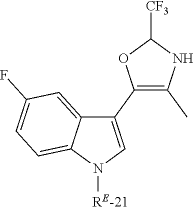 | P-26 |
| 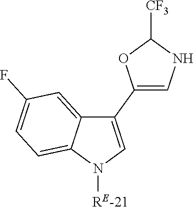 | P-27 |
| 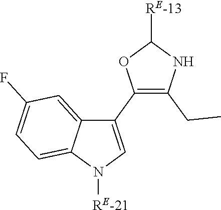 | P-28 |
| 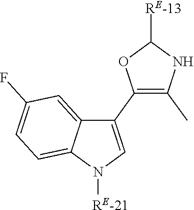 | P-29 |
| 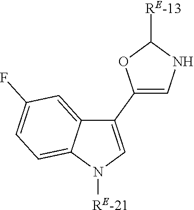 | P-30 |

In table 5 below, Q indicates either oxygen or sulfur.

TABLE 5
Embodiments of Enantiomerically Enriched Mixtures
of Formula XXX

| Com- | | | | | | | |
|---|---|---|---|---|---|---|---|
| pound | R^{NA} | R^{NB} | R^{A} | Q¹ | R^{NC} | RX¹ | R^{X2} |
| T-1 | CH₂CH₃ | CH₂CH₃ | CH₂CH₃ | Oxo | CH₃ | H | Halo |
| T-2 | CH₂CH₃ | CH₂CH₃ | CH₂CH₃ | Oxo | CH₃ | Halo | H |
| T-3 | CH₂CH₃ | CH₂CH₃ | CH₂CH₃ | Oxo | H | H | Halo |
| T-4 | CH₂CH₃ | CH₂CH₃ | CH₂CH₃ | Oxo | H | Halo | H |
| T-5 | CH₂CH₃ | CH₂CH₃ | CH₂CH₃ | Oxo | Y | H | Halo |
| T-6 | CH₂CH₃ | CH₂CH₃ | CH₂CH₃ | Oxo | Y | Halo | H |
| T-7 | CH₂CH₃ | CH₂CH₃ | CH₂CH₃ | H | CH₃ | H | H |
| T-8 | CH₂CH₃ | CH₂CH₃ | CH₂CH₃ | H | CH₃ | H | Halo |
| T-9 | CH₂CH₃ | CH₂CH₃ | CH₂CH₃ | H | CH₃ | H | QCH₂CH₃ |
| T-10 | CH₂CH₃ | CH₂CH₃ | CH₂CH₃ | H | CH₃ | H | QCH₃ |
| T-11 | CH₂CH₃ | CH₂CH₃ | CH₂CH₃ | H | CH₃ | Halo | H |
| T-12 | CH₂CH₃ | CH₂CH₃ | CH₂CH₃ | H | CH₃ | QCH₂CH₃ | H |
| T-13 | CH₂CH₃ | CH₂CH₃ | CH₂CH₃ | H | CH₃ | QCH₃ | H |
| T-14 | CH₂CH₃ | CH₂CH₃ | CH₃ | Oxo | CH₃ | H | Halo |
| T-15 | CH₂CH₃ | CH₂CH₃ | CH₃ | Oxo | CH₃ | Halo | H |
| T-16 | CH₂CH₃ | CH₂CH₃ | CH₃ | Oxo | H | H | Halo |
| T-17 | CH₂CH₃ | CH₂CH₃ | CH₃ | Oxo | H | Halo | H |
| T-18 | CH₂CH₃ | CH₂CH₃ | CH₃ | Oxo | Y | H | Halo |
| T-19 | CH₂CH₃ | CH₂CH₃ | CH₃ | Oxo | Y | Halo | H |
| T-20 | CH₂CH₃ | CH₂CH₃ | CH₃ | H | CH₃ | H | H |
| T-21 | CH₂CH₃ | CH₂CH₃ | CH₃ | H | CH₃ | H | Halo |
| T-22 | CH₂CH₃ | CH₂CH₃ | CH₃ | H | CH₃ | H | QCH₂CH₃ |
| T-23 | CH₂CH₃ | CH₂CH₃ | CH₃ | H | CH₃ | H | QCH₃ |
| T-24 | CH₂CH₃ | CH₂CH₃ | CH₃ | H | CH₃ | Halo | H |
| T-25 | CH₂CH₃ | CH₂CH₃ | CH₃ | H | CH₃ | QCH₂CH₃ | H |
| T-26 | CH₂CH₃ | CH₂CH₃ | CH₃ | H | CH₃ | QCH₃ | H |
| T-27 | CH₂CH₃ | CH₂CH₃ | H | Oxo | CH₃ | H | Halo |
| T-28 | CH₂CH₃ | CH₂CH₃ | H | Oxo | CH₃ | Halo | H |
| T-29 | CH₂CH₃ | CH₂CH₃ | H | Oxo | H | H | Halo |
| T-30 | CH₂CH₃ | CH₂CH₃ | H | Oxo | H | Halo | H |
| T-31 | CH₂CH₃ | CH₂CH₃ | H | Oxo | Y | H | Halo |
| T-32 | CH₂CH₃ | CH₂CH₃ | H | Oxo | Y | Halo | H |
| T-33 | CH₂CH₃ | CH₂CH₃ | H | H | CH₃ | H | H |
| T-34 | CH₂CH₃ | CH₂CH₃ | H | H | CH₃ | H | Halo |
| T-35 | CH₂CH₃ | CH₂CH₃ | H | H | CH₃ | H | QCH₂CH₃ |
| T-36 | CH₂CH₃ | CH₂CH₃ | H | H | CH₃ | H | QCH₃ |
| T-37 | CH₂CH₃ | CH₂CH₃ | H | H | CH₃ | Halo | H |
| T-38 | CH₂CH₃ | CH₂CH₃ | H | H | CH₃ | QCH₂CH₃ | H |
| T-39 | CH₂CH₃ | CH₂CH₃ | H | H | CH₃ | QCH₃ | H |
| T-40 | CH₃ | CH₃ | CH₂CH₃ | Oxo | CH₃ | H | Halo |
| T-41 | CH₃ | CH₃ | CH₂CH₃ | Oxo | CH₃ | Halo | H |
| T-42 | CH₃ | CH₃ | CH₂CH₃ | Oxo | H | H | Halo |
| T-43 | CH₃ | CH₃ | CH₂CH₃ | Oxo | H | Halo | H |
| T-44 | CH₃ | CH₃ | CH₂CH₃ | Oxo | Y | H | Halo |
| T-45 | CH₃ | CH₃ | CH₂CH₃ | Oxo | Y | Halo | H |
| T-46 | CH₃ | CH₃ | CH₂CH₃ | H | CH₃ | H | H |
| T-47 | CH₃ | CH₃ | CH₂CH₃ | H | CH₃ | H | Halo |
| T-48 | CH₃ | CH₃ | CH₂CH₃ | H | CH₃ | H | QCH₂CH₃ |
| T-49 | CH₃ | CH₃ | CH₂CH₃ | H | CH₃ | H | QCH₃ |
| T-50 | CH₃ | CH: | CH₂CH₃ | H | CH₃ | Halo | H |
| T-51 | CH₃ | CH₃ | CH₂CH₃ | H | CH₃ | QCH₂CH₃ | H |
| T-52 | CH₃ | CH₃ | CH₂CH₃ | H | CH₃ | QCH₃ | H |
| T-53 | CH₃ | CH₃ | CH₃ | Oxo | CH₃ | H | Halo |
| T-54 | CH₃ | CH₃ | CH₃ | Oxo | CH₃ | Halo | H |
| T-55 | CH₃ | CH₃ | CH₃ | Oxo | H | H | Halo |
| T-56 | CH₃ | CH₃ | CH₃ | Oxo | H | Halo | H |
| T-57 | CH₃ | CH₃ | CH₃ | Oxo | Y | H | Halo |
| T-58 | CH₃ | CH₃ | CH₃ | Oxo | Y | Halo | H |
| T-59 | CH₃ | CH₃ | CH₃ | H | CH₃ | H | H |
| T-60 | CH₃ | CH₃ | CH₃ | H | CH₃ | H | Halo |
| T-61 | CH₃ | CH₃ | CH₃ | H | CH₃ | H | QCH₂CH₃ |
| T-62 | CH₃ | CH₃ | CH₃ | H | CH₃ | H | QCH₃ |
| T-63 | CH₃ | CH₃ | CH₃ | H | CH₃ | Halo | H |
| T-64 | CH₃ | CH₃ | CH₃ | H | CH₃ | QCH₂CH₃ | H |
| T-65 | CH₃ | CH₃ | CH₃ | H | CH₃ | QCH₃ | H |
| T-66 | CH₃ | CH₃ | H | Oxo | CH₃ | H | Halo |
| T-67 | CH₃ | CH₃ | H | Oxo | CH₃ | Halo | H |
| T-68 | CH₃ | CH₃ | H | Oxo | H | H | Halo |
| T-69 | CH₃ | CH₃ | H | Oxo | H | Halo | H |
| T-70 | CH₃ | CH₃ | H | Oxo | Y | H | Halo |
| T-71 | CH₃ | CH₃ | H | Oxo | Y | Halo | H |
| T-72 | H | CH₂CH₃ | CH₂CH₃ | Oxo | CH₃ | H | Halo |
| T-73 | H | CH₂CH₃ | CH₂CH₃ | Oxo | CH₃ | Halo | H |
| T-74 | H | CH₂CH₃ | CH₂CH₃ | Oxo | H | H | Halo |
| T-75 | H | CH₂CH₃ | CH₂CH₃ | Oxo | H | Halo | H |
| T-76 | H | CH₂CH₃ | CH₂CH₃ | Oxo | Y | H | H |
| T-77 | H | CH₂CH₃ | CH₂CH₃ | Oxo | Y | H | Halo |
| T-78 | H | CH₂CH₃ | CH₂CH₃ | Oxo | Y | H | QCH₂CH₃ |
| T-79 | H | CH₂CH₃ | CH₂CH₃ | Oxo | Y | H | QCH₃ |
| T-80 | H | CH₂CH₃ | CH₂CH₃ | Oxo | Y | Halo | H |
| T-81 | H | CH₂CH₃ | CH₂CH₃ | Oxo | Y | QCH₂CH₃ | H |
| T-82 | H | CH₂CH₃ | CH₂CH₃ | Oxo | Y | QCH₃ | H |
| T-83 | H | CH₂CH₃ | CH₂CH₃ | H | CH₃ | H | H |
| T-84 | H | CH₂CH₃ | CH₂CH₃ | H | CH₃ | H | Halo |
| T-85 | H | CH₂CH₃ | CH₂CH₃ | H | CH₃ | H | QCH₂CH₃ |
| T-86 | H | CH₂CH₃ | CH₂CH₃ | H | CH₃ | H | QCH₃ |
| T-87 | H | CH₂CH₃ | CH₂CH₃ | H | CH₃ | Halo | H |
| T-88 | H | CH₂CH₃ | CH₂CH₃ | H | CH₃ | QCH₂CH₃ | H |
| T-89 | H | CH₂CH₃ | CH₂CH₃ | H | CH₃ | QCH₃ | H |
| T-90 | H | CH₂CH₃ | CH₂CH₃ | H | Y | H | H |
| T-91 | H | CH₂CH₃ | CH₂CH₃ | H | Y | H | Halo |
| T-92 | H | CH₂CH₃ | CH₂CH₃ | H | Y | H | QCH₂CH₃ |
| T-93 | H | CH₂CH₃ | CH₂CH₃ | H | Y | H | QCH₃ |
| T-94 | H | CH₂CH₃ | CH₂CH₃ | H | Y | Halo | H |
| T-95 | H | CH₂CH₃ | CH₂CH₃ | H | Y | QCH₂CH₃ | H |
| T-96 | H | CH₂CH₃ | CH₂CH₃ | H | Y | QCH₃ | H |
| T-97 | H | CH₂CH₃ | CH₃ | Oxo | CH₃ | H | Halo |
| T-98 | H | CH₂CH₃ | CH₃ | Oxo | CH₃ | Halo | H |
| T-99 | H | CH₂CH₃ | CH₃ | Oxo | H | H | Halo |
| T-100 | H | CH₂CH₃ | CH₃ | Oxo | H | Halo | H |
| T-101 | H | CH₂CH₃ | CH₃ | Oxo | Y | H | H |
| T-102 | H | CH₂CH₃ | CH₃ | Oxo | Y | H | Halo |
| T-103 | H | CH₂CH₃ | CH₃ | Oxo | Y | H | QCH₂CH₃ |
| T-104 | H | CH₂CH₃ | CH₃ | Oxo | Y | H | QCH₃ |
| T-105 | H | CH₂CH₃ | CH₃ | Oxo | Y | Halo | H |
| T-106 | H | CH₂CH₃ | CH₃ | Oxo | Y | QCH₂CH₃ | H |
| T-107 | H | CH₂CH₃ | CH₃ | Oxo | Y | QCH₃ | H |
| T-108 | H | CH₂CH₃ | CH₃ | H | CH₃ | H | H |
| T-109 | H | CH₂CH₃ | CH₃ | H | CH₃ | H | Halo |
| T-110 | H | CH₂CH₃ | CH₃ | H | CH₃ | H | QCH₂CH₃ |
| T-111 | H | CH₂CH₃ | CH₃ | H | CH₃ | H | QCH₃ |
| T-112 | H | CH₂CH₃ | CH₃ | H | CH₃ | Halo | H |
| T-113 | H | CH₂CH₃ | CH₃ | H | CH₃ | QCH₂CH₃ | H |
| T-114 | H | CH₂CH₃ | CH₃ | H | CH₃ | QCH₃ | H |
| T-115 | H | CH₂CH₃ | CH₃ | H | Y | H | H |
| T-116 | H | CH₂CH₃ | CH₃ | H | Y | H | Halo |
| T-117 | H | CH₂CH₃ | CH₃ | H | Y | H | QCH₂CH₃ |
| T-118 | H | CH₂CH₃ | CH₃ | H | Y | H | QCH₃ |
| T-119 | H | CH₂CH₃ | CH₃ | H | Y | Halo | H |
| T-120 | H | CH₂CH₃ | CH₃ | H | Y | QCH₂CH₃ | H |
| T-121 | H | CH₂CH₃ | CH₃ | H | Y | QCH₃ | H |
| T-122 | H | CH₂CH₃ | H | Oxo | CH₃ | H | Halo |
| T-123 | H | CH₂CH₃ | H | Oxo | CH₃ | Halo | H |
| T-124 | H | CH₂CH₃ | H | Oxo | H | H | Halo |
| T-125 | H | CH₂CH₃ | H | Oxo | H | Halo | H |
| T-126 | H | CH₂CH₃ | H | Oxo | Y | H | H |
| T-127 | H | CH₂CH₃ | H | Oxo | Y | H | Halo |
| T-128 | H | CH₂CH₃ | H | Oxo | Y | H | QCH₂CH₃ |
| T-129 | H | CH₂CH₃ | H | Oxo | Y | H | QCH₃ |
| T-130 | H | CH₂CH₃ | H | Oxo | Y | Halo | H |
| T-131 | H | CH₂CH₃ | H | Oxo | Y | QCH₂CH₃ | H |
| T-132 | H | CH₂CH₃ | H | Oxo | Y | QCH₃ | H |
| T-133 | H | CH₂CH₃ | H | H | CH₃ | H | H |
| T-134 | H | CH₂CH₃ | H | H | CH₃ | H | Halo |
| T-135 | H | CH₂CH₃ | H | H | CH₃ | H | QCH₂CH₃ |
| T-136 | H | CH₂CH₃ | H | H | CH₃ | H | QCH₃ |
| T-137 | H | CH₂CH₃ | H | H | CH₃ | Halo | H |
| T-138 | H | CH₂CH₃ | H | H | CH₃ | QCH₂CH₃ | H |
| T-139 | H | CH₂CH₃ | H | H | CH₃ | QCH₃ | H |
| T-140 | H | CH₂CH₃ | H | H | Y | H | H |
| T-141 | H | CH₂CH₃ | H | H | Y | H | QCH₂CH₃ |
| T-142 | H | CH₂CH₃ | H | H | Y | H | QCH₃ |
| T-143 | H | CH₂CH₃ | H | H | Y | QCH₂CH₃ | H |
| T-144 | H | CH₂CH₃ | H | H | Y | QCH₃ | H |
| T-145 | H | CH₃ | CH₂CH₃ | Oxo | CH₃ | H | Halo |
| T-146 | H | CH₃ | CH₂CH₃ | Oxo | CH₃ | Halo | H |
| T-147 | H | CH₃ | CH₂CH₃ | Oxo | H | H | Halo |
| T-148 | H | CH₃ | CH₂CH₃ | Oxo | H | Halo | H |
| T-149 | H | CH₃ | CH₂CH₃ | Oxo | Y | H | H |
| T-150 | H | CH₃ | CH₂CH₃ | Oxo | Y | H | Halo |
| T-151 | H | CH₃ | CH₂CH₃ | Oxo | Y | H | QCH₂CH₃ |
| T-152 | H | CH₃ | CH₂CH₃ | Oxo | Y | H | QCH₃ |
| T-153 | H | CH₃ | CH₂CH₃ | Oxo | Y | Halo | H |
| T-154 | H | CH₃ | CH₂CH₃ | Oxo | Y | QCH₂CH₃ | H |
| T-155 | H | CH₃ | CH₂CH₃ | Oxo | Y | QCH₃ | H |
| T-156 | H | CH₃ | CH₂CH₃ | H | CH₃ | H | H |
| T-157 | H | CH₃ | CH₂CH₃ | H | CH₃ | H | Halo |
| T-158 | H | CH₃ | CH₂CH₃ | H | CH₃ | H | QCH₂CH₃ |
| T-159 | H | CH₃ | CH₂CH₃ | H | CH₃ | H | QCH₃ |
| T-160 | H | CH₃ | CH₂CH₃ | H | CH₃ | Halo | H |
| T-161 | H | CH₃ | CH₂CH₃ | H | CH₃ | QCH₂CH₃ | H |
| T-162 | H | CH₃ | CH₂CH₃ | H | CH₃ | QCH₃ | H |
| T-163 | H | CH₃ | CH₂CH₃ | H | Y | H | H |
| T-164 | H | CH₃ | CH₂CH₃ | H | Y | H | Halo |
| T-165 | H | CH₃ | CH₂CH₃ | H | Y | H | QCH₂CH₃ |
| T-166 | H | CH₃ | CH₂CH₃ | H | Y | H | QCH₃ |
| T-167 | H | CH₃ | CH₂CH₃ | H | Y | Halo | H |
| T-168 | H | CH₃ | CH₂CH₃ | H | Y | QCH₂CH₃ | H |
| T-169 | H | CH₃ | CH₂CH₃ | H | Y | QCH₃ | H |
| T-170 | H | CH₃ | CH₃ | Oxo | CH₃ | H | Halo |
| T-171 | H | CH₃ | CH₃ | Oxo | CH₃ | Halo | H |
| T-172 | H | CH₃ | CH₃ | Oxo | H | H | Halo |
| T-173 | H | CH₃ | CH₃ | Oxo | H | Halo | H |
| T-174 | H | CH₃ | CH₃ | Oxo | Y | H | H |
| T-175 | H | CH₃ | CH₃ | Oxo | Y | H | Halo |
| T-176 | H | CH₃ | CH₃ | Oxo | Y | H | QCH₂CH₃ |
| T-177 | H | CH₃ | CH₃ | Oxo | Y | H | QCH₃ |
| T-178 | H | CH₃ | CH₃ | Oxo | Y | Halo | H |
| T-179 | H | CH₃ | CH₃ | Oxo | Y | QCH₂CH₃ | H |
| T-180 | H | CH₃ | CH₃ | Oxo | Y | QCH₃ | H |
| T-181 | H | CH₃ | CH₃ | H | CH₃ | H | H |
| T-182 | H | CH₃ | CH₃ | H | CH₃ | H | Halo |
| T-183 | H | CH₃ | CH₃ | H | CH₃ | H | QCH₂CH₃ |
| T-184 | H | CH₃ | CH₃ | H | CH₃ | H | QCH₃ |
| T-185 | H | CH₃ | CH₃ | H | CH₃ | Halo | H |
| T-186 | H | CH₃ | CH₃ | H | CH₃ | QCH₂CH₃ | H |
| T-187 | H | CH₃ | CH₃ | H | CH₃ | QCH₃ | H |
| T-188 | H | CH₃ | CH₃ | H | Y | H | H |
| T-189 | H | CH₃ | CH₃ | H | Y | H | Halo |
| T-190 | H | CH₃ | CH₃ | H | Y | H | QCH₂CH₃ |
| T-191 | H | CH₃ | CH₃ | H | Y | H | QCH₃ |
| T-192 | H | CH₃ | CH₃ | H | Y | Halo | H |
| T-193 | H | CH₃ | CH₃ | H | Y | QCH₂CH₃ | H |
| T-194 | H | CH₃ | CH₃ | H | Y | QCH₃ | H |
| T-195 | H | CH₃ | H | Oxo | CH₃ | H | Halo |
| T-196 | H | CH₃ | H | Oxo | CH₃ | Halo | H |
| T-197 | H | CH₃ | H | Oxo | H | H | Halo |
| T-198 | H | CH₃ | H | Oxo | H | Halo | H |
| T-199 | H | CH₃ | H | Oxo | Y | H | H |
| T-200 | H | CH₃ | H | Oxo | Y | H | Halo |
| T-201 | H | CH₃ | H | Oxo | Y | H | QCH₂CH₃ |
| T-202 | H | CH₃ | H | Oxo | Y | H | QCH₃ |
| T-203 | H | CH₃ | H | Oxo | Y | Halo | H |
| T-204 | H | CH₃ | H | Oxo | Y | QCH₂CH₃ | H |
| T-205 | H | CH₃ | H | Oxo | Y | QCH₃ | H |
| T-206 | H | CH₃ | H | H | CH₃ | H | H |
| T-207 | H | CH₃ | H | H | CH₃ | H | Halo |
| T-208 | H | CH₃ | H | H | CH₃ | H | QCH₂CH₃ |
| T-209 | H | CH₃ | H | H | CH₃ | H | QCH₃ |
| T-210 | H | CH₃ | H | H | CH₃ | Halo | H |
| T-211 | H | CH₃ | H | H | CH₃ | QCH₂CH₃ | H |
| T-212 | H | CH₃ | H | H | CH₃ | QCH₃ | H |
| T-213 | H | CH₃ | H | H | Y | H | H |
| T-214 | H | CH₃ | H | H | Y | H | QCH₂CH₃ |
| T-215 | H | CH₃ | H | H | Y | H | QCH₃ |
| T-216 | H | CH₃ | H | H | Y | QCH₂CH₃ | H |
| T-217 | H | CH₃ | H | H | Y | QCH₃ | H |
| T-218 | H | H | CH₂CH₃ | Oxo | CH₃ | H | Halo |
| T-219 | H | H | CH₂CH₃ | Oxo | CH₃ | H | QCH₃ |
| T-220 | H | H | CH₂CH₃ | Oxo | CH₃ | Halo | H |
| T-221 | H | H | CH₂CH₃ | Oxo | CH₃ | QCH₃ | H |
| T-222 | H | H | CH₂CH₃ | Oxo | H | H | Halo |
| T-223 | H | H | CH₂CH₃ | Oxo | H | Halo | H |
| T-224 | H | H | CH₂CH₃ | Oxo | Y | H | H |
| T-225 | H | H | CH₂CH₃ | Oxo | Y | H | Halo |
| T-226 | H | H | CH₂CH₃ | Oxo | Y | H | QCH₂CH₃ |
| T-227 | H | H | CH₂CH₃ | Oxo | Y | H | QCH₃ |
| T-228 | H | H | CH₂CH₃ | Oxo | Y | Halo | H |
| T-229 | H | H | CH₂CH₃ | Oxo | Y | QCH₂CH₃ | H |
| T-230 | H | H | CH₂CH₃ | Oxo | Y | QCH₃ | H |
| T-231 | H | H | CH₂CH₃ | H | CH₃ | H | H |
| T-232 | H | H | CH₂CH₃ | H | CH₃ | H | Halo |
| T-233 | H | H | CH₂CH₃ | H | CH₃ | H | QCH₂CH₃ |
| T-234 | H | H | CH₂CH₃ | H | CH₃ | H | QCH₃ |
| T-235 | H | H | CH₂CH₃ | H | CH₃ | Halo | H |
| T-236 | H | H | CH₂CH₃ | H | CH₃ | QCH₂CH₃ | H |
| T-237 | H | H | CH₂CH₃ | H | CH₃ | QCH₃ | H |
| T-238 | H | H | CH₂CH₃ | H | H | H | QCH₂CH₃ |
| T-239 | H | H | CH₂CH₃ | H | H | H | QCH₃ |
| T-240 | H | H | CH₂CH₃ | H | H | QCH₂CH₃ | H |
| T-241 | H | H | CH₂CH₃ | H | H | QCH₃ | H |
| T-242 | H | H | CH₂CH₃ | H | Y | H | H |
| T-243 | H | H | CH₂CH₃ | H | Y | H | QCH₂CH₃ |
| T-244 | H | H | CH₂CH₃ | H | Y | H | QCH₃ |
| T-245 | H | H | CH₂CH₃ | H | Y | QCH₂CH₃ | H |
| T-246 | H | H | CH₂CH₃ | H | Y | QCH₃ | H |
| T-247 | H | H | CH₃ | Oxo | CH₃ | H | Halo |
| T-248 | H | H | CH₃ | Oxo | CH₃ | H | QCH₃ |
| T-249 | H | H | CH₃ | Oxo | CH₃ | Halo | H |
| T-250 | H | H | CH₃ | Oxo | CH₃ | QCH₃ | H |
| T-251 | H | H | CH₃ | Oxo | H | H | Halo |
| T-252 | H | H | CH₃ | Oxo | H | Halo | H |
| T-253 | H | H | CH₃ | Oxo | Y | H | H |
| T-254 | H | H | CH₃ | Oxo | Y | H | Halo |
| T-255 | H | H | CH₃ | Oxo | Y | H | QCH₂CH₃ |
| T-256 | H | H | CH₃ | Oxo | Y | H | QCH₃ |
| T-257 | H | H | CH₃ | Oxo | Y | Halo | H |
| T-258 | H | H | CH₃ | Oxo | Y | QCH₂CH₃ | H |
| T-259 | H | H | CH₃ | Oxo | Y | QCH₃ | H |
| T-260 | H | H | CH₃ | H | CH₃ | H | H |
| T-261 | H | H | CH₃ | H | CH₃ | H | Halo |
| T-262 | H | H | CH₃ | H | CH₃ | H | QCH₂CH₃ |
| T-263 | H | H | CH₃ | H | CH₃ | H | QCH₃ |
| T-264 | H | H | CH₃ | H | CH₃ | Halo | H |
| T-265 | H | H | CH₃ | H | CH₃ | QCH₂CH₃ | H |
| T-266 | H | H | CH₃ | H | CH | QCH₃ | H |
| T-267 | H | H | CH₃ | H | H | H | QCH₂CH₃ |
| T-268 | H | H | CH₃ | H | H | QCH₂CH₃ | H |
| T-269 | H | H | CH₃ | H | Y | H | H |
| T-270 | H | H | CH₃ | H | Y | H | QCH₂CH₃ |
| T-271 | H | H | CH₃ | H | Y | H | QCH₃ |
| T-272 | H | H | CH₃ | H | Y | QCH₂CH₃ | H |
| T-273 | H | H | CH₃ | H | Y | QCH₃ | H |
| T-274 | H | H | H | Oxo | CH₃ | H | Halo |
| T-275 | H | H | H | Oxo | CH₃ | H | QCH₃ |
| T-276 | H | H | H | Oxo | CH₃ | Halo | H |
| T-277 | H | H | H | Oxo | CH₃ | QCH₃ | H |
| T-278 | H | H | H | Oxo | H | H | Halo |
| T-279 | H | H | H | Oxo | H | Halo | H |
| T-280 | H | H | H | Oxo | Y | H | H |
| T-281 | H | H | H | Oxo | Y | H | Halo |
| T-282 | H | H | H | Oxo | Y | H | QCH₂CH₃ |
| T-283 | H | H | H | Oxo | Y | H | QCH₃ |
| T-284 | H | H | H | Oxo | Y | Halo | H |
| T-285 | H | H | H | Oxo | Y | QCH₂CH₃ | H |
| T-286 | H | H | H | Oxo | Y | QCH₃ | H |
| T-287 | H | H | H | H | CH₃ | H | Halo |
| T-288 | H | H | H | H | CH₃ | Halo | H |
| T-289 | H | H | H | H | H | H | QCH₂CH₃ |
| T-290 | H | H | H | H | H | QCH₂CH₃ | H |
| T-291 | H | H | H | H | Y | H | H |
| T-292 | H | H | H | H | Y | H | QCH₂CH₃ |
| T-293 | H | H | H | H | Y | H | QCH₃ |
| T-294 | H | H | H | H | Y | QCH₂CH₃ | H |
| T-295 | H | H | H | H | Y | QCH₃ | H |
| T-296 | Y | CH₂CH₃ | CH₂CH₃ | Oxo | CH₃ | H | H |
| T-297 | Y | CH₂CH₃ | CH₂CH₃ | Oxo | CH₃ | H | Halo |
| T-298 | Y | CH₂CH₃ | CH₂CH₃ | Oxo | CH₃ | H | QCH₂CH₃ |
| T-299 | Y | CH₂CH₃ | CH₂CH₃ | Oxo | CH₃ | H | QCH₃ |
| T-300 | Y | CH₂CH₃ | CH₂CH₃ | Oxo | CH₃ | Halo | H |
| T-301 | Y | CH₂CH₃ | CH₂CH₃ | Oxo | CH₃ | QCH₂CH₃ | H |
| T-302 | Y | CH₂CH₃ | CH₂CH₃ | Oxo | CH₃ | QCH₃ | H |
| T-303 | Y | CH₂CH₃ | CH₂CH₃ | Oxo | H | H | H |
| T-304 | Y | CH₂CH₃ | CH₂CH₃ | Oxo | H | H | Halo |
| T-305 | Y | CH₂CH₃ | CH₂CH₃ | Oxo | H | H | QCH₂CH₃ |
| T-306 | Y | CH₂CH₃ | CH₂CH₃ | Oxo | H | H | QCH₃ |
| T-307 | Y | CH₂CH₃ | CH₂CH₃ | Oxo | H | Halo | H |
| T-308 | Y | CH₂CH₃ | CH₂CH₃ | Oxo | H | QCH₂CH₃ | H |
| T-309 | Y | CH₂CH₃ | CH₂CH₃ | Oxo | H | QCH₃ | H |
| T-310 | Y | CH₂CH₃ | CH₂CH₃ | Oxo | Y | H | H |
| T-311 | Y | CH₂CH₃ | CH₂CH₃ | Oxo | Y | H | Halo |
| T-312 | Y | CH₂CH₃ | CH₂CH₃ | Oxo | Y | H | QCH₂CH₃ |
| T-313 | Y | CH₂CH₃ | CH₂CH₃ | Oxo | Y | H | QCH₃ |
| T-314 | Y | CH₂CH₃ | CH₂CH₃ | Oxo | Y | Halo | H |
| T-315 | Y | CH₂CH₃ | CH₂CH₃ | Oxo | Y | QCH₂CH₃ | H |
| T-316 | Y | CH₂CH₃ | CH₂CH₃ | Oxo | Y | QCH₃ | H |
| T-317 | Y | CH₂CH₃ | CH₂CH₃ | H | CH₃ | H | H |
| T-318 | Y | CH₂CH₃ | CH₂CH₃ | H | CH₃ | H | Halo |
| T-319 | Y | CH₂CH₃ | CH₂CH₃ | H | CH₃ | H | QCH₂CH₃ |
| T-320 | Y | CH₂CH₃ | CH₂CH₃ | H | CH₃ | H | QCH₃ |
| T-321 | Y | CH₂CH₃ | CH₂CH₃ | H | CH₃ | Halo | H |
| T-322 | Y | CH₂CH₃ | CH₂CH₃ | H | CH₃ | QCH₂CH₃ | H |
| T-323 | Y | CH₂CH₃ | CH₂CH₃ | H | CH₃ | QCH₃ | H |
| T-324 | Y | CH₂CH₃ | CH₂CH₃ | H | H | H | H |
| T-325 | Y | CH₂CH₃ | CH₂CH₃ | H | H | H | Halo |
| T-326 | Y | CH₂CH: | CH₂CH₃ | H | H | H | QCH₂CH₃ |
| T-327 | Y | CH₂CH₃ | CH₂CH₃ | H | H | H | QCH₃ |
| T-328 | Y | CH₂CH₃ | CH₂CH₃ | H | H | Halo | H |
| T-329 | Y | CH₂CH₃ | CH₂CH₃ | H | H | QCH₂CH₃ | H |
| T-330 | Y | CH₂CH₃ | CH₂CH₃ | H | H | QCH₃ | H |
| T-331 | Y | CH₂CH₃ | CH₂CH₃ | H | Y | H | H |
| T-332 | Y | CH₂CH₃ | CH₂CH₃ | H | Y | H | Halo |
| T-333 | Y | CH₂CH₃ | CH₂CH₃ | H | Y | H | QCH₂CH₃ |
| T-334 | Y | CH₂CH₃ | CH₂CH₃ | H | Y | H | QCH₃ |
| T-335 | Y | CH₂CH₃ | CH₂CH₃ | H | Y | Halo | H |
| T-336 | Y | CH₂CH₃ | CH₂CH₃ | H | Y | QCH₂CH₃ | H |
| T-337 | Y | CH₂CH₃ | CH₂CH₃ | H | Y | QCH₃ | H |
| T-338 | Y | CH₂CH₃ | CH₃ | Oxo | CH₃ | H | H |
| T-339 | Y | CH₂CH₃ | CH₃ | Oxo | CH₃ | H | Halo |
| T-340 | Y | CH₂CH₃ | CH₃ | Oxo | CH₃ | H | QCH₂CH₃ |
| T-341 | Y | CH₂CH₃ | CH₃ | Oxo | CH₃ | H | QCH₃ |
| T-342 | Y | CH₂CH₃ | CH₃ | Oxo | CH₃ | Halo | H |
| T-343 | Y | CH₂CH₃ | CH₃ | Oxo | CH₃ | QCH₂CH₃ | H |
| T-344 | Y | CH₂CH₃ | CH₃ | Oxo | CH₃ | QCH₃ | H |
| T-345 | Y | CH₂CH₃ | CH₃ | Oxo | H | H | H |
| T-346 | Y | CH₂CH₃ | CH₃ | Oxo | H | H | Halo |
| T-347 | Y | CH₂CH₃ | CH₃ | Oxo | H | H | QCH₂CH₃ |
| T-348 | Y | CH₂CH₃ | CH₃ | Oxo | H | H | QCH₃ |
| T-349 | Y | CH₂CH₃ | CH₃ | Oxo | H | Halo | H |
| T-350 | Y | CH₂CH₃ | CH₃ | Oxo | H | QCH₂CH₃ | H |
| T-351 | Y | CH₂CH₃ | CH₃ | Oxo | H | QCH₃ | H |
| T-352 | Y | CH₂CH₃ | CH₃ | Oxo | Y | H | H |
| T-353 | Y | CH₂CH₃ | CH₃ | Oxo | Y | H | Halo |
| T-354 | Y | CH₂CH₃ | CH₃ | Oxo | Y | H | QCH₂CH₃ |
| T-355 | Y | CH₂CH₃ | CH₃ | Oxo | Y | H | QCH₃ |
| T-356 | Y | CH₂CH₃ | CH₃ | Oxo | Y | Halo | H |
| T-357 | Y | CH₂CH₃ | CH₃ | Oxo | Y | QCH₂CH₃ | H |
| T-358 | Y | CH₂CH₃ | CH₃ | Oxo | Y | QCH₃ | H |
| T-359 | Y | CH₂CH₃ | CH₃ | H | CH₃ | H | H |
| T-360 | Y | CH₂CH₃ | CH₃ | H | CH₃ | H | Halo |
| T-361 | Y | CH₂CH₃ | CH₃ | H | CH₃ | H | QCH₂CH₃ |
| T-362 | Y | CH₂CH₃ | CH₃ | H | CH₃ | H | QCH₃ |
| T-363 | Y | CH₂CH₃ | CH₃ | H | CH₃ | Halo | H |
| T-364 | Y | CH₂CH₃ | CH₃ | H | CH₃ | QCH₂CH₃ | H |
| T-365 | Y | CH₂CH₃ | CH₃ | H | CH₃ | QCH₃ | H |
| T-366 | Y | CH₂CH₃ | CH₃ | H | H | H | H |
| T-367 | Y | CH₂CH₃ | CH₃ | H | H | H | Halo |
| T-368 | Y | CH₂CH₃ | CH₃ | H | H | H | QCH₂CH₃ |
| T-369 | Y | CH₂CH₃ | CH₃ | H | H | H | QCH₃ |
| T-370 | Y | CH₂CH₃ | CH₃ | H | H | Halo | H |
| T-371 | Y | CH₂CH₃ | CH₃ | H | H | QCH₂CH₃ | H |
| T-372 | Y | CH₂CH₃ | CH₃ | H | H | QCH₃ | H |
| T-373 | Y | CH₂CH₃ | CH₃ | H | Y | H | H |
| T-374 | Y | CH₂CH₃ | CH₃ | H | Y | H | Halo |
| T-375 | Y | CH₂CH₃ | CH₃ | H | Y | H | QCH₂CH₃ |
| T-376 | Y | CH₂CH₃ | CH₃ | H | Y | H | QCH₃ |
| T-377 | Y | CH₂CH₃ | CH₃ | H | Y | Halo | H |
| T-378 | Y | CH₂CH₃ | CH₃ | H | Y | QCH₂CH₃ | H |
| T-379 | Y | CH₂CH₃ | CH₃ | H | Y | QCH₃ | H |
| T-380 | Y | CH₂CH₃ | H | Oxo | CH₃ | H | H |
| T-381 | Y | CH₂CH₃ | H | Oxo | CH₃ | H | Halo |
| T-382 | Y | CH₂CH₃ | H | Oxo | CH₃ | H | QCH₂CH₃ |
| T-383 | Y | CH₂CH₃ | H | Oxo | CH₃ | H | QCH₃ |
| T-384 | Y | CH₂CH₃ | H | Oxo | CH₃ | Halo | H |
| T-385 | Y | CH₂CH₃ | H | Oxo | CH₃ | QCH₂CH₃ | H |
| T-386 | Y | CH₂CH₃ | H | Oxo | CH₃ | QCH₃ | H |
| T-387 | Y | CH₂CH₃ | H | Oxo | H | H | H |
| T-388 | Y | CH₂CH₃ | H | Oxo | H | H | Halo |
| T-389 | Y | CH₂CH₃ | H | Oxo | H | H | QCH₂CH₃ |
| T-390 | Y | CH₂CH₃ | H | Oxo | H | H | QCH₃ |
| T-391 | Y | CH₂CH₃ | H | Oxo | H | Halo | H |
| T-392 | Y | CH₂CH₃ | H | Oxo | H | QCH₂CH₃ | H |
| T-393 | Y | CH₂CH₃ | H | Oxo | H | QCH₃ | H |
| T-394 | Y | CH₂CH₃ | H | Oxo | Y | H | H |
| T-395 | Y | CH₂CH₃ | H | Oxo | Y | H | Halo |
| T-396 | Y | CH₂CH₃ | H | Oxo | Y | H | QCH₂CH₃ |
| T-397 | Y | CH₂CH₃ | H | Oxo | Y | H | QCH₃ |
| T-398 | Y | CH₂CH₃ | H | Oxo | Y | Halo | H |
| T-399 | Y | CH₂CH₃ | H | Oxo | Y | QCH₂CH₃ | H |
| T-400 | Y | CH₂CH₃ | H | Oxo | Y | QCH₃ | H |
| T-401 | Y | CH₂CH₃ | H | H | CH₃ | H | H |
| T-402 | Y | CH₂CH₃ | H | H | CH₃ | H | Halo |
| T-403 | Y | CH₂CH₃ | H | H | CH₃ | H | QCH₂CH₃ |
| T-404 | Y | CH₂CH₃ | H | H | CH₃ | H | QCH₃ |
| T-405 | Y | CH₂CH₃ | H | H | CH₃ | Halo | H |
| T-406 | Y | CH₂CH₃ | H | H | CH₃ | QCH₂CH₃ | H |
| T-407 | Y | CH₂CH₃ | H | H | CH₃ | QCH₃ | H |
| T-408 | Y | CH₂CH₃ | H | H | H | H | H |
| T-409 | Y | CH₂CH₃ | H | H | H | H | QCH₂CH₃ |
| T-410 | Y | CH₂CH₃ | H | H | H | H | QCH₃ |
| T-411 | Y | CH₂CH₃ | H | H | H | QCH₂CH₃ | H |
| T-412 | Y | CH₂CH₃ | H | H | H | QCH₃ | H |
| T-413 | Y | CH₂CH₃ | H | H | Y | H | H |
| T-414 | Y | CH₂CH₃ | H | H | Y | H | QCH₂CH₃ |
| T-415 | Y | CH₂CH₃ | H | H | Y | H | QCH₃ |
| T-416 | Y | CH₂CH₃ | H | H | Y | QCH₂CH₃ | H |
| T-417 | Y | CH₂CH₃ | H | H | Y | QCH₃ | H |
| T-418 | Y | CH₃ | CH₂CH₃ | Oxo | CH₃ | H | H |
| T-419 | Y | CH₃ | CH₂CH₃ | Oxo | CH₃ | H | Halo |
| T-420 | Y | CH₃ | CH₂CH₃ | Oxo | CH₃ | H | QCH₂CH₃ |
| T-421 | Y | CH₃ | CH₂CH₃ | Oxo | CH₃ | H | QCH₃ |
| T-422 | Y | CH₃ | CH₂CH₃ | Oxo | CH₃ | Halo | H |
| T-423 | Y | CH₃ | CH₂CH₃ | Oxo | CH₃ | QCH₂CH₃ | H |
| T-424 | Y | CH₃ | CH₂CH₃ | Oxo | CH₃ | QCH₃ | H |
| T-425 | Y | CH₃ | CH₂CH₃ | Oxo | H | H | H |
| T-426 | Y | CH₃ | CH₂CH₃ | Oxo | H | H | Halo |
| T-427 | Y | CH₃ | CH₂CH₃ | Oxo | H | H | QCH₂CH₃ |
| T-428 | Y | CH₃ | CH₂CH₃ | Oxo | H | H | QCH₃ |
| T-429 | Y | CH₃ | CH₂CH₃ | Oxo | H | Halo | H |
| T-430 | Y | CH₃ | CH₂CH₃ | Oxo | H | QCH₂CH₃ | H |
| T-431 | Y | CH₃ | CH₂CH₃ | Oxo | H | QCH₃ | H |
| T-432 | Y | CH₃ | CH₂CH₃ | Oxo | Y | H | H |
| T-433 | Y | CH₃ | CH₂CH₃ | Oxo | Y | H | Halo |
| T-434 | Y | CH₃ | CH₂CH₃ | Oxo | Y | H | QCH₂CH₃ |
| T-435 | Y | CH₃ | CH₂CH₃ | Oxo | Y | H | QCH₃ |
| T-436 | Y | CH₃ | CH₂CH₃ | Oxo | Y | Halo | H |
| T-437 | Y | CH₃ | CH₂CH₃ | Oxo | Y | QCH₂CH₃ | H |
| T-438 | Y | CH₃ | CH₂CH₃ | Oxo | Y | QCH₃ | H |
| T-439 | Y | CH₃ | CH₂CH₃ | H | CH₃ | H | H |
| T-440 | Y | CH₃ | CH₂CH₃ | H | CH₃ | H | Halo |
| T-441 | Y | CH₃ | CH₂CH₃ | H | CH₃ | H | QCH₂CH₃ |
| T-442 | Y | CH₃ | CH₂CH₃ | H | CH₃ | H | QCH₃ |
| T-443 | Y | CH₃ | CH₂CH₃ | H | CH₃ | Halo | H |
| T-444 | Y | CH₃ | CH₂CH₃ | H | CH₃ | QCH₂CH₃ | H |
| T-445 | Y | CH₃ | CH₂CH₃ | H | CH₃ | QCH₃ | H |
| T-446 | Y | CH₃ | CH₂CH₃ | H | H | H | H |
| T-447 | Y | CH₃ | CH₂CH₃ | H | H | H | Halo |
| T-448 | Y | CH₃ | CH₂CH₃ | H | H | H | QCH₂CH₃ |
| T-449 | Y | CH₃ | CH₂CH₃ | H | H | H | QCH₃ |
| T-450 | Y | CH₃ | CH₂CH₃ | H | H | Halo | H |
| T-451 | Y | CH₃ | CH₂CH₃ | H | H | QCH₂CH₃ | H |
| T-452 | Y | CH₃ | CH₂CH₃ | H | H | QCH₃ | H |
| T-453 | Y | CH₃ | CH₂CH₃ | H | Y | H | H |
| T-454 | Y | CH₃ | CH₂CH₃ | H | Y | H | Halo |
| T-455 | Y | CH₃ | CH₂CH₃ | H | Y | H | QCH₂CH₃ |
| T-456 | Y | CH₃ | CH₂CH₃ | H | Y | H | QCH₃ |
| T-457 | Y | CH₃ | CH₂CH₃ | H | Y | Halo | H |
| T-458 | Y | CH₃ | CH₂CH₃ | H | Y | QCH₂CH₃ | H |
| T-459 | Y | CH₃ | CH₂CH₃ | H | Y | QCH₃ | H |
| T-460 | Y | CH₃ | CH₃ | Oxo | CH₃ | H | H |
| T-461 | Y | CH₃ | CH₃ | Oxo | CH₃ | H | Halo |
| T-462 | Y | CH₃ | CH₃ | Oxo | CH₃ | H | QCH₂CH₃ |
| T-463 | Y | CH₃ | CH₃ | Oxo | CH₃ | H | QCH₃ |
| T-464 | Y | CH₃ | CH₃ | Oxo | CH₃ | Halo | H |
| T-465 | Y | CH₃ | CH₃ | Oxo | CH₃ | QCH₂CH₃ | H |
| T-466 | Y | CH₃ | CH₃ | Oxo | CH₃ | QCH₃ | H |
| T-467 | Y | CH₃ | CH₃ | Oxo | H | H | H |
| T-468 | Y | CH₃ | CH₃ | Oxo | H | H | Halo |
| T-469 | Y | CH₃ | CH₃ | Oxo | H | H | QCH₂CH₃ |
| T-470 | Y | CH₃ | CH₃ | Oxo | H | H | QCH₃ |
| T-471 | Y | CH₃ | CH₃ | Oxo | H | Halo | H |
| T-472 | Y | CH₃ | CH₃ | Oxo | H | QCH₂CH₃ | H |
| T-473 | Y | CH₃ | CH₃ | Oxo | H | QCH₃ | H |
| T-474 | Y | CH₃ | CH₃ | Oxo | Y | H | H |
| T-475 | Y | CH₃ | CH₃ | Oxo | Y | H | Halo |
| T-476 | Y | CH₃ | CH₃ | Oxo | Y | H | QCH₂CH₃ |
| T-477 | Y | CH₃ | CH₃ | Oxo | Y | H | QCH₃ |
| T-478 | Y | CH₃ | CH₃ | Oxo | Y | Halo | H |
| T-479 | Y | CH₃ | CH₃ | Oxo | Y | QCH₂CH₃ | H |
| T-480 | Y | CH₃ | CH₃ | Oxo | Y | QCH₃ | H |
| T-481 | Y | CH₃ | CH₃ | H | CH₃ | H | H |
| T-482 | Y | CH₃ | CH₃ | H | CH₃ | H | Halo |
| T-483 | Y | CH₃ | CH₃ | H | CH₃ | H | QCH₂CH₃ |
| T-484 | Y | CH₃ | CH₃ | H | CH₃ | H | QCH₃ |
| T-485 | Y | CH₃ | CH₃ | H | CH₃ | Halo | H |
| T-486 | Y | CH₃ | CH₃ | H | CH₃ | QCH₂CH₃ | H |
| T-487 | Y | CH₃ | CH₃ | H | CH₃ | QCH₃ | H |
| T-488 | Y | CH₃ | CH₃ | H | H | H | H |
| T-489 | Y | CH₃ | CH₃ | H | H | H | Halo |
| T-490 | Y | CH₃ | CH₃ | H | H | H | QCH₂CH₃ |
| T-491 | Y | CH₃ | CH₃ | H | H | H | QCH₃ |
| T-492 | Y | CH₃ | CH₃ | H | H | Halo | H |
| T-493 | Y | CH₃ | CH₃ | H | H | QCH₂CH₃ | H |
| T-494 | Y | CH₃ | CH₃ | H | H | QCH₃ | H |
| T-495 | Y | CH₃ | CH₃ | H | Y | H | H |
| T-496 | Y | CH₃ | CH₃ | H | Y | H | Halo |
| T-497 | Y | CH₃ | CH₃ | H | Y | H | QCH₂CH₃ |
| T-498 | Y | CH₃ | CH₃ | H | Y | H | QCH₃ |
| T-499 | Y | CH₃ | CH₃ | H | Y | Halo | H |
| T-500 | Y | CH₃ | CH₃ | H | Y | QCH₂CH₃ | H |
| T-501 | Y | CH₃ | CH₃ | H | Y | QCH₃ | H |
| T-502 | Y | CH₃ | H | Oxo | CH₃ | H | H |
| T-503 | Y | CH₃ | H | Oxo | CH₃ | H | Halo |
| T-504 | Y | CH₃ | H | Oxo | CH₃ | H | QCH₂CH₃ |
| T-505 | Y | CH₃ | H | Oxo | CH₃ | H | QCH₃ |
| T-506 | Y | CH₃ | H | Oxo | CH₃ | Halo | H |
| T-507 | Y | CH₃ | H | Oxo | CH₃ | QCH₂CH₃ | H |
| T-508 | Y | CH₃ | H | Oxo | CH₃ | QCH₃ | H |
| T-509 | Y | CH₃ | H | Oxo | H | H | H |
| T-510 | Y | CH₃ | H | Oxo | H | H | Halo |
| T-511 | Y | CH₃ | H | Oxo | H | H | QCH₂CH₃ |
| T-512 | Y | CH₃ | H | Oxo | H | H | QCH₃ |
| T-513 | Y | CH₃ | H | Oxo | H | Halo | H |
| T-514 | Y | CH₃ | H | Oxo | H | QCH₂CH₃ | H |
| T-515 | Y | CH₃ | H | Oxo | H | QCH₃ | H |
| T-516 | Y | CH₃ | H | Oxo | Y | H | H |
| T-517 | Y | CH₃ | H | Oxo | Y | H | Halo |
| T-518 | Y | CH₃ | H | Oxo | Y | H | QCH₂CH₃ |
| T-519 | Y | CH₃ | H | Oxo | Y | H | QCH₃ |
| T-520 | Y | CH₃ | H | Oxo | Y | Halo | H |
| T-521 | Y | CH₃ | H | Oxo | Y | QCH₂CH₃ | H |
| T-522 | Y | CH₃ | H | Oxo | Y | QCH₃ | H |
| T-523 | Y | CH₃ | H | H | CH₃ | H | H |
| T-524 | Y | CH₃ | H | H | CH₃ | H | Halo |
| T-525 | Y | CH₃ | H | H | CH₃ | H | QCH₂CH₃ |
| T-526 | Y | CH₃ | H | H | CH₃ | H | QCH₃ |
| T-527 | Y | CH₃ | H | H | CH₃ | Halo | H |
| T-528 | Y | CH₃ | H | H | CH₃ | QCH₂CH₃ | H |
| T-529 | Y | CH₃ | H | H | CH₃ | QCH₃ | H |
| T-530 | Y | CH₃ | H | H | H | H | H |
| T-531 | Y | CH₃ | H | H | H | H | QCH₂CH₃ |
| T-532 | Y | CH₃ | H | H | H | H | QCH₃ |
| T-533 | Y | CH₃ | H | H | H | QCH₂CH₃ | H |
| T-534 | Y | CH₃ | H | H | H | QCH₃ | H |
| T-535 | Y | CH₃ | H | H | Y | H | H |
| T-536 | Y | CH₃ | H | H | Y | H | QCH₂CH₃ |
| T-537 | Y | CH₃ | H | H | Y | H | QCH₃ |
| T-538 | Y | CH₃ | H | H | Y | QCH₂CH₃ | H |
| T-539 | Y | CH₃ | H | H | Y | QCH₃ | H |
| T-540 | Y | H | CH₂CH₃ | Oxo | CH₃ | H | H |
| T-541 | Y | H | CH₂CH₃ | Oxo | CH₃ | H | Halo |
| T-542 | Y | H | CH₂CH₃ | Oxo | CH₃ | H | QCH₂CH₃ |
| T-543 | Y | H | CH₂CH₃ | Oxo | CH₃ | H | QCH₃ |
| T-544 | Y | H | CH₂CH₃ | Oxo | CH₃ | Halo | H |
| T-545 | Y | H | CH₂CH₃ | Oxo | CH₃ | QCH₂CH₃ | H |
| T-546 | Y | H | CH₂CH₃ | Oxo | CH₃ | QCH₃ | H |
| T-547 | Y | H | CH₂CH₃ | Oxo | H | H | H |
| T-548 | Y | H | CH₂CH₃ | Oxo | H | H | Halo |
| T-549 | Y | H | CH₂CH | Oxo | H | H | QCH₂CH₃ |
| T-550 | Y | H | CH₂CH₃ | Oxo | H | H | QCH₃ |
| T-551 | Y | H | CH₂CH₃ | Oxo | H | Halo | H |
| T-552 | Y | H | CH₂CH₃ | Oxo | H | QCH₂CH₃ | H |
| T-553 | Y | H | CH₂CH₃ | Oxo | H | QCH₃ | H |
| T-554 | Y | H | CH₂CH₃ | Oxo | Y | H | H |
| T-555 | Y | H | CH₂CH₃ | Oxo | Y | H | Halo |
| T-556 | Y | H | CH₂CH₃ | Oxo | Y | H | QCH₂CH₃ |
| T-557 | Y | H | CH₂CH₃ | Oxo | Y | H | QCH₃ |
| T-558 | Y | H | CH₂CH₃ | Oxo | Y | Halo | H |
| T-559 | Y | H | CH₂CH₃ | Oxo | Y | QCH₂CH₃ | H |
| T-560 | Y | H | CH₂CH₃ | Oxo | Y | QCH₃ | H |
| T-561 | Y | H | CH₂CH₃ | H | CH₃ | H | H |
| T-562 | Y | H | CH₂CH₃ | H | CH₃ | H | Halo |
| T-563 | Y | H | CH₂CH₃ | H | CH₃ | H | QCH₂CH₃ |
| T-564 | Y | H | CH₂CH₃ | H | CH₃ | H | QCH₃ |
| T-565 | Y | H | CH₂CH₃ | H | CH₃ | Halo | H |
| T-566 | Y | H | CH₂CH₃ | H | CH₃ | QCH₂CH₃ | H |
| T-567 | Y | H | CH₂CH₃ | H | CH₃ | QCH₃ | H |
| T-568 | Y | H | CH₂CH₃ | H | H | H | H |
| T-569 | Y | H | CH₂CH₃ | H | H | H | QCH₂CH₃ |
| T-570 | Y | H | CH₂CH₃ | H | H | H | QCH₃ |
| T-571 | Y | H | CH₂CH₃ | H | H | QCH₂CH₃ | H |
| T-572 | Y | H | CH₂CH₃ | H | H | QCH₃ | H |
| T-573 | Y | H | CH₂CH₃ | H | Y | H | H |
| T-574 | Y | H | CH₂CH₃ | H | Y | H | QCH₂CH₃ |
| T-575 | Y | H | CH₂CH₃ | H | Y | H | QCH₃ |
| T-576 | Y | H | CH₂CH₃ | H | Y | QCH₂CH₃ | H |
| T-577 | Y | H | CH₂CH₃ | H | Y | QCH₃ | H |
| T-578 | Y | H | CH₃ | Oxo | CH₃ | H | H |
| T-579 | Y | H | CH₃ | Oxo | CH₃ | H | Halo |
| T-580 | Y | H | CH₃ | Oxo | CH₃ | H | QCH2CH₃ |
| T-581 | Y | H | CH₃ | Oxo | CH₃ | H | QCH₃ |
| T-582 | Y | H | CH₃ | Oxo | CH₃ | Halo | H |
| T-583 | Y | H | CH₃ | Oxo | CH₃ | QCH₂CH₃ | H |
| T-584 | Y | H | CH₃ | Oxo | CH₃ | QCH₃ | H |
| T-585 | Y | H | CH₃ | Oxo | H | H | H |
| T-586 | Y | H | CH₃ | Oxo | H | H | Halo |
| T-587 | Y | H | CH₃ | Oxo | H | H | QCH₂CH₃ |
| T-588 | Y | H | CH₃ | Oxo | H | H | QCH₃ |
| T-589 | Y | H | CH₃ | Oxo | H | Halo | H |
| T-590 | Y | H | CH₃ | Oxo | H | QCH₂CH₃ | H |
| T-591 | Y | H | CH₃ | Oxo | H | QCH₃ | H |
| T-592 | Y | H | CH₃ | Oxo | Y | H | H |
| T-593 | Y | H | CH₃ | Oxo | Y | H | Halo |
| T-594 | Y | H | CH₃ | Oxo | Y | H | QCH₂CH₃ |
| T-595 | Y | H | CH₃ | Oxo | Y | H | QCH₃ |
| T-596 | Y | H | CH₃ | Oxo | Y | Halo | H |
| T-597 | Y | H | CH₃ | Oxo | Y | QCH₂CH₃ | H |
| T-598 | Y | H | CH₃ | Oxo | Y | QCH₃ | H |
| T-599 | Y | H | CH₃ | H | CH₃ | H | H |
| T-600 | Y | H | CH₃ | H | CH₃ | H | Halo |
| T-601 | Y | H | CH₃ | H | CH₃ | H | QCH₂CH₃ |
| T-602 | Y | H | CH₃ | H | CH₃ | H | QCH₃ |
| T-603 | Y | H | CH₃ | H | CH₃ | Halo | H |
| T-604 | Y | H | CH₃ | H | CH₃ | QCH₂CH₃ | H |
| T-605 | Y | H | CH₃ | H | CH₃ | QCH₃ | H |
| T-606 | Y | H | CH₃ | H | H | H | H |
| T-607 | Y | H | CH₃ | H | H | H | QCH₂CH₃ |
| T-608 | Y | H | CH₃ | H | H | H | QCH₃ |
| T-609 | Y | H | CH₃ | H | H | QCH₂CH₃ | H |
| T-610 | Y | H | CH₃ | H | H | QCH₃ | H |
| T-611 | Y | H | CH₃ | H | Y | H | H |
| T-612 | Y | H | CH₃ | H | Y | H | QCH₂CH₃ |
| T-613 | Y | H | CH₃ | H | Y | H | QCH₃ |
| T-614 | Y | H | CH₃ | H | Y | QCH₂CH₃ | H |
| T-615 | Y | H | CH₃ | H | Y | QCH₃ | H |
| T-616 | Y | H | H | Oxo | CH₃ | H | H |
| T-617 | Y | H | H | Oxo | CH₃ | H | Halo |
| T-618 | Y | H | H | Oxo | CH₃ | H | QCH₂CH₃ |
| T-619 | Y | H | H | Oxo | CH₃ | H | QCH₃ |
| T-620 | Y | H | H | Oxo | CH₃ | Halo | H |
| T-621 | Y | H | H | Oxo | CH₃ | QCH₂CH₃ | H |
| T-622 | Y | H | H | Oxo | CH₃ | QCH₃ | H |
| T-623 | Y | H | H | Oxo | H | H | H |
| T-624 | Y | H | H | Oxo | H | H | Halo |
| T-625 | Y | H | H | Oxo | H | H | QCH₂CH₃ |
| T-626 | Y | H | H | Oxo | H | H | QCH₃ |
| T-627 | Y | H | H | Oxo | H | Halo | H |
| T-628 | Y | H | H | Oxo | H | QCH₂CH₃ | H |
| T-629 | Y | H | H | Oxo | H | QCH₃ | H |
| T-630 | Y | H | H | Oxo | Y | H | H |
| T-631 | Y | H | H | Oxo | Y | H | Halo |
| T-632 | Y | H | H | Oxo | Y | H | QCH₂CH₃ |
| T-633 | Y | H | H | Oxo | Y | H | QCH₃ |
| T-634 | Y | H | H | Oxo | Y | Halo | H |
| T-635 | Y | H | H | Oxo | Y | QCH₂CH₃ | H |
| T-636 | Y | H | H | Oxo | Y | QCH₃ | H |
| T-637 | Y | H | H | H | CH₃ | H | H |
| T-638 | Y | H | H | H | CH₃ | H | Halo |
| T-639 | Y | H | H | H | CH₃ | H | QCH₂CH₃ |
| T-640 | Y | H | H | H | CH₃ | H | QCH₃ |
| T-641 | Y | H | H | H | CH₃ | Halo | H |
| T-642 | Y | H | H | H | CH₃ | QCH₂CH₃ | H |
| T-643 | Y | H | H | H | CH₃ | QCH₃ | H |
| T-644 | Y | H | H | H | H | H | H |
| T-645 | Y | H | H | H | H | H | QCH₂CH₃ |
| T-646 | Y | H | H | H | H | H | QCH₃ |
| T-647 | Y | H | H | H | H | QCH₂CH₃ | H |
| T-648 | Y | H | H | H | H | QCH₃ | H |
| T-649 | Y | H | H | H | Y | H | H |
| T-650 | Y | H | H | H | Y | H | QCH₂CH₃ |
| T-651 | Y | H | H | H | Y | H | QCH₃ |
| T-652 | Y | H | H | H | Y | QCH₂CH₃ | H |
| T-653 | Y | H | H | H | Y | QCH₃ | H |
| T-654 | Y | Y | CH₂CH₃ | Oxo | CH₃ | H | H |
| T-655 | Y | Y | CH₂CH₃ | Oxo | CH₃ | H | Halo |
| T-656 | Y | Y | CH₂CH₃ | Oxo | CH₃ | H | QCH₂CH₃ |
| T-657 | Y | Y | CH₂CH₃ | Oxo | CH₃ | H | QCH₃ |
| T-658 | Y | Y | CH₂CH₃ | Oxo | CH₃ | Halo | H |
| T-659 | Y | Y | CH₂CH₃ | Oxo | CH₃ | QCH₂CH₃ | H |
| T-660 | Y | Y | CH₂CH₃ | Oxo | CH₃ | QCH₃ | H |
| T-661 | Y | Y | CH₂CH₃ | Oxo | H | H | H |
| T-662 | Y | Y | CH₂CH₃ | Oxo | H | H | Halo |
| T-663 | Y | Y | CH₂CH₃ | Oxo | H | H | QCH₂CH₃ |
| T-664 | Y | Y | CH₂CH₃ | Oxo | H | H | QCH₃ |
| T-665 | Y | Y | CH₂CH₃ | Oxo | H | Halo | H |
| T-666 | Y | Y | CH₂CH₃ | Oxo | H | QCH₂CH₃ | H |
| T-667 | Y | Y | CH₂CH₃ | Oxo | H | QCH₃ | H |
| T-668 | Y | Y | CH₂CH₃ | Oxo | Y | H | H |
| T-669 | Y | Y | CH₂CH₃ | Oxo | Y | H | Halo |
| T-670 | Y | Y | CH₂CH₃ | Oxo | Y | H | QCH₂CH₃ |
| T-671 | Y | Y | CH₂CH₃ | Oxo | Y | H | QCH₃ |
| T-672 | Y | Y | CH₂CH₃ | Oxo | Y | Halo | H |
| T-673 | Y | Y | CH₂CH₃ | Oxo | Y | QCH₂CH₃ | H |
| T-674 | Y | Y | CH₂CH₃ | Oxo | Y | QCH₃ | H |
| T-675 | Y | Y | CH₂CH₃ | H | CH₃ | H | H |
| T-676 | Y | Y | CH₂CH₃ | H | CH₃ | H | Halo |
| T-677 | Y | Y | CH₂CH₃ | H | CH₃ | H | QCH₂CH₃ |
| T-678 | Y | Y | CH₂CH₃ | H | CH₃ | H | QCH₃ |
| T-679 | Y | Y | CH₂CH₃ | H | CH₃ | Halo | H |
| T-680 | Y | Y | CH₂CH₃ | H | CH₃ | QCH₂CH₃ | H |
| T-681 | Y | Y | CH₂CH₃ | H | CH₃ | QCH₃ | H |
| T-682 | Y | Y | CH₂CH₃ | H | H | H | H |
| T-683 | Y | Y | CH₂CH₃ | H | H | H | QCH₂CH₃ |
| T-684 | Y | Y | CH₂CH₃ | H | H | H | QCH₃ |
| T-685 | Y | Y | CH₂CH₃ | H | H | QCH₂CH₃ | H |
| T-686 | Y | Y | CH₂CH₃ | H | H | QCH₃ | H |
| T-687 | Y | Y | CH₂CH₃ | H | Y | H | H |
| T-688 | Y | Y | CH₂CH₃ | H | Y | H | QCH₂CH₃ |
| T-689 | Y | Y | CH₂CH₃ | H | Y | H | QCH₃ |
| T-690 | Y | Y | CH₂CH₃ | H | Y | QCH₂CH₃ | H |
| T-691 | Y | Y | CH₂CH₃ | H | Y | QCH₃ | H |
| T-692 | Y | Y | CH | Oxo | CH₃ | H | H |
| T-693 | Y | Y | CH₃ | Oxo | CH₃ | H | Halo |
| T-694 | Y | Y | CH₃ | Oxo | CH₃ | H | QCH₂CH₃ |
| T-695 | Y | Y | CH₃ | Oxo | CH₃ | H | QCH₃ |
| T-696 | Y | Y | CH₃ | Oxo | CH₃ | Halo | H |
| T-697 | Y | Y | CH₃ | Oxo | CH₃ | QCH₂CH₃ | H |
| T-698 | Y | Y | CH₃ | Oxo | CH₃ | QCH₃ | H |
| T-699 | Y | Y | CH₃ | Oxo | H | H | H |
| T-700 | Y | Y | CH₃ | Oxo | H | H | Halo |
| T-701 | Y | Y | CH₃ | Oxo | H | H | QCH₂CH₃ |
| T-702 | Y | Y | CH₃ | Oxo | H | H | QCH₃ |
| T-703 | Y | Y | CH₃ | Oxo | H | Halo | H |
| T-704 | Y | Y | CH₃ | Oxo | H | QCH₂CH₃ | H |
| T-705 | Y | Y | CH₃ | Oxo | H | QCH₃ | H |
| T-706 | Y | Y | CH₃ | Oxo | Y | H | H |
| T-707 | Y | Y | CH₃ | Oxo | Y | H | Halo |
| T-708 | Y | Y | CH₃ | Oxo | Y | H | OCH₂CH₃ |
| T-709 | Y | Y | CH₃ | Oxo | Y | H | OCH₃ |
| T-710 | Y | Y | CH₃ | Oxo | Y | Halo | H |
| T-711 | Y | Y | CH₃ | Oxo | Y | QCH₂CH₃ | H |
| T-712 | Y | Y | CH₃ | Oxo | Y | QCH₃ | H |
| T-713 | Y | Y | CH₃ | H | CH₃ | H | H |
| T-714 | Y | Y | CH₃ | H | CH₃ | H | Halo |
| T-715 | Y | Y | CH₃ | H | CH₃ | H | QCH₂CH₃ |
| T-716 | Y | Y | CH₃ | H | CH₃ | H | QCH₃ |
| T-717 | Y | Y | CH₃ | H | CH₃ | Halo | H |
| T-718 | Y | Y | CH₃ | H | CH₃ | QCH₂CH₃ | H |
| T-719 | Y | Y | CH₃ | H | CH₃ | QCH₃ | H |
| T-720 | Y | Y | CH₃ | H | H | H | H |
| T-721 | Y | Y | CH₃ | H | H | H | QCH₂CH₃ |
| T-722 | Y | Y | CH₃ | H | H | H | QCH₃ |
| T-723 | Y | Y | CH₃ | H | H | QCH₂CH₃ | H |
| T-724 | Y | Y | CH₃ | H | H | QCH₃ | H |
| T-725 | Y | Y | CH₃ | H | Y | H | H |
| T-726 | Y | Y | CH₃ | H | Y | H | QCH₂CH₃ |
| T-727 | Y | Y | CH₃ | H | Y | H | QCH₃ |
| T-728 | Y | Y | CH₃ | H | Y | QCH₂CH₃ | H |
| T-729 | Y | Y | CH₃ | H | Y | QCH₃ | H |
| T-730 | Y | Y | H | Oxo | CH₃ | H | H |
| T-731 | Y | Y | H | Oxo | CH₃ | H | Halo |
| T-732 | Y | Y | H | Oxo | CH₃ | H | QCH₂CH₃ |
| T-733 | Y | Y | H | Oxo | CH₃ | H | QCH₃ |
| T-734 | Y | Y | H | Oxo | CH₃ | Halo | H |
| T-735 | Y | Y | H | Oxo | CH₃ | QCH₂CH₃ | H |
| T-736 | Y | Y | H | Oxo | CH₃ | QCH₃ | H |
| T-737 | Y | Y | H | Oxo | H | H | H |
| T-738 | Y | Y | H | Oxo | H | H | Halo |
| T-739 | Y | Y | H | Oxo | H | H | QCH₂CH₃ |
| T-740 | Y | Y | H | Oxo | H | H | QCH₃ |
| T-741 | Y | Y | H | Oxo | H | Halo | H |
| T-742 | Y | Y | H | Oxo | H | QCH₂CH₃ | H |
| T-743 | Y | Y | H | Oxo | H | QCH₃ | H |
| T-744 | Y | Y | H | Oxo | Y | H | H |
| T-745 | Y | Y | H | Oxo | Y | H | Halo |
| T-746 | Y | Y | H | Oxo | Y | H | QCH₂CH₃ |
| T-747 | Y | Y | H | Oxo | Y | H | QCH₃ |
| T-748 | Y | Y | H | Oxo | Y | Halo | H |
| T-749 | Y | Y | H | Oxo | Y | QCH₂CH₃ | H |
| T-750 | Y | Y | H | Oxo | Y | QCH₃ | H |
| T-751 | Y | Y | H | H | CH₃ | H | H |
| T-752 | Y | Y | H | H | CH₃ | H | Halo |
| T-753 | Y | Y | H | H | CH₃ | H | QCH₂CH₃ |
| T-754 | Y | Y | H | H | CH₃ | H | QCH₃ |
| T-755 | Y | Y | H | H | CH₃ | Halo | H |
| T-756 | Y | Y | H | H | CH₃ | QCH₂CH₃ | H |
| T-757 | Y | Y | H | H | CH₃ | QCH₃ | H |
| T-758 | Y | Y | H | H | H | H | H |
| T-759 | Y | Y | H | H | H | H | QCH₂CH₃ |
| T-760 | Y | Y | H | H | H | H | QCH₃ |
| T-761 | Y | Y | H | H | H | QCH₂CH₃ | H |
| T-762 | Y | Y | H | H | H | QCH₃ | H |
| T-763 | Y | Y | H | H | Y | H | H |
| T-764 | Y | Y | H | H | Y | H | QCH₂CH₃ |
| T-765 | Y | Y | H | H | Y | H | QCH₃ |
| T-766 | Y | Y | H | H | Y | QCH₂CH₃ | H |
| T-767 | Y | Y | H | H | Y | QCH₃ | H |

In certain embodiments, pharmaceutical compositions are disclosed which comprise a compound of Formula XXX, either racemic, as pure enantiomers, or in some combination of enantiomers, and which may be in association with another active agent, as well as with a pharmaceutically acceptable carrier, diluent, or excipient.

### V. Combination Therapy

In certain embodiments the compositions of the invention are not limited to combinations of a single compound, and a single carrier, diluent, or excipient alone, but also include combinations of multiple such compounds, and/or multiple carriers, diluents, and excipients. In certain embodiments, a pharmaceutical composition can be provided to the host, for example a human who can be a patient, with an effective amount of one or more other compounds either of the present invention or other active compounds, in combination, together with one or more other active compounds, and one or more pharmaceutically acceptable carriers, diluents, and/or excipients.

In some aspects, a compound of the present invention is formulated in a pharmaceutical preparation with other active compounds to increase therapeutic efficacy, decrease unwanted effects, increase stability/shelf-life, and/or alter pharmacokinetics. Such other active compounds include: antioxidants (such alpha-lipoate in acid or salt form, ascorbate in acid or salt form, selenium, or N-acetylcysteine), H2-receptor agonists or antagonists (such as famotidine), stimulants (such as dextroamphetamine, lisdextroamphetamine, or methamphetamine), entactogens (such as MDMA), antiinflammatories (such as ibuprofen or ketoprofen), matrix metalloproteinase inhibitors (such as doxycycline), NOS inhibitors (such as S-methyl-L-thiocitrulline), proton pump inhibitors (such as omeprazole), phosphodiesterase 5 inhibitors (such as sildenafil), drugs with cardiovascular effects (beta antagonists such as propranolol, mixed alpha and beta antagonists such as carvedilol, alpha antagonists such as prazosin, imidazoline receptor agonists such as rilmenidine or moxonidine, serotonin antagonists such as ketanserin or lisuride), norepinephrine transporter blockers (such as reboxetine), acetylcholine nicotinic receptor modulators (such as bupropion, hydroxybupropion, methyllycaconitine, memantine, or mecamylamine), gastrointestinal acidifying agents (such as ascorbic acid or glutamic acid hydrochloride) or alkalinizing agents (such as sodium bicarbonate), NMDA receptor antagonists (such as ketamine), TrkB agonists (such as 7,8-dihydroxyflavone, 7,8,3′-trihydroxyflavone, or N-acetylserotonin), and serotonin receptor agonists (such as 5-methoxy-N-methyl-N-isopropyltryptamine, N,N-Dimethyl-2-(2-methyl-1H-indol-1-yl)ethan-1-amine, psilocin, or psilocybin). These ingredients may be in ion, freebase, or salt form and may be isomers or prodrugs.

The pharmacological agents that make up the combination therapy disclosed herein may be a combined dosage form or in separate dosage forms intended for substantially simultaneous administration. The pharmacological agents that make up the combination therapy may also be administered sequentially, with either therapeutic compound being administered by a regimen calling for two-step administration. The two-step administration regimen may call for sequential administration of the active agents or spaced-apart administration of the separate active agents.

The time period between the multiple administration steps may range from a few minutes to several hours, depending upon the properties of each pharmacological agent, such as potency, solubility, bioavailability, plasma half-life and kinetic profile of the pharmacological agent. Circadian variation of the target molecule concentration may also determine the optimal dose interval. For example, a compound of the present invention may be administered while the other pharmacological agent is being administered (concurrent administration) or may be administered before or after other pharmacological agent is administered (sequential administration).

In cases where the two (or more) drugs are included in the fixed-dose combinations of the present invention are incompatible, cross-contamination can be avoided, for example, by incorporation of the drugs in different drug layers in the oral dosage form with the inclusion of a barrier layer(s) between the different drug layers, wherein the barrier layer(s) comprise one or more inert/non-functional materials.

In certain embodiments, the formulations of the present invention are fixed-dose combinations of a compound of the present invention or a pharmaceutically acceptable salt thereof and at least one other pharmacological agent. Fixed-dose combination formulations may contain, but are not limited to, the following combinations in the form of single-layer monolithic tablet or multi-layered monolithic tablet or in the form of a core tablet-in-tablet or multi-layered multi-disk tablet or beads inside a capsule or tablets inside a capsule.

In one embodiment, the fixed-dose combination is a therapeutically efficacious fixed-dose combinations of immediate-release formulations of BK-5F-NM-AMT, BK-5Cl-NM-AMT, and/or BK-5Br-NM-AMT and other pharmacological agents.

In one embodiment, the fixed-dose combination is a therapeutically efficacious fixed-dose combinations of extended-release formulations of BK-5F-NM-AMT, BK-5Cl-NM-AMT, and/or BK-5Br-NM-AMT and delayed and/or extended-release other pharmacological agents contained in a single dosage form.

In one embodiment, the fixed-dose combination is a therapeutically efficacious fixed-dose combinations of immediate-release formulations of compounds of any of Formulas I-XXIX and other pharmacological agents.

In one embodiment, the fixed-dose combination is a therapeutically efficacious fixed-dose combinations of extended-release formulations of compounds of any of Formulas I-XXIX and delayed and/or extended-release other pharmacological agents contained in a single dosage form.

In certain embodiments, the invention includes pharmaceutically acceptable complex derivatives of the compound or composition, including solvates, salts, esters, enantiomers, isomers (stereoisomers and/or constitutional, including ones based on substituting deuterium for hydrogen), derivatives or prodrugs of BK-5F-NM-AMT, BK-5Cl-NM-AMT, and/or BK-5Br-NM-AMT.

In certain embodiments, the invention includes pharmaceutically acceptable complex derivatives of the compound or composition, including solvates, salts, esters, enantiomers, isomers (stereoisomers and/or constitutional, including ones based on substituting deuterium for hydrogen), derivatives or prodrugs of compounds of any of Formulas I-XXIX.

In one embodiment, extended-release multi-layered matrix tablets are prepared using fixed-dose combinations of BK-5F-NM-AMT, BK-5Cl-NM-AMT, and/or BK-5Br-NM-AMT with another pharmacological agent. In one embodiment, extended-release multi-layered matrix tablets are prepared using fixed-dose combinations of compounds of any one of Formulas I-XXIX, or a pharmaceutically acceptable salt thereof, with another pharmacological agent. For example, a hydrophilic polymer may comprise guar gum, hydroxypropylmethylcellulose, and xanthan gum as matrix formers. Lubricated formulations may be compressed by a wet granulation method.

Another embodiment of the invention includes multiple variations in the pharmaceutical dosages of each drug in the combination as further outlined below. Another embodiment of the invention includes various forms of preparations including using solids, liquids, immediate or delayed or extended-release forms. Many types of variations are possible as known to those skilled in the art.

Pharmaceutical Combinations with Dextroamphetamine

In certain embodiments, BK-5F-NM-AMT, BK-5Cl-NM-AMT, and/or BK-5Br-NM-AMT, either an enantiomer or a mixture of enantiomers, with zero to five or zero to seven hydrogens replaced with deuterium, is formulated in a pharmaceutical composition that contains a pharmaceutically acceptable salt of dextroamphetamine in the amount of 2 mg, 4 mg, 5 mg, 7 mg, 10 mg, 15 mg, 20 mg, or 25 mg. The required amount of dextroamphetamine will vary depending on the needs of the patient.

In other embodiments, BK-5F-NM-AMT, BK-5Cl-NM-AMT, and/or BK-5Br-NM-AMT, with zero to five or zero to seven hydrogens replaced with deuterium, is formulated in a pharmaceutical composition that contains a pharmaceutically acceptable salt of dextroamphetamine with dextroamphetamine in a ratio by weight of 1:2, 1:3, 1:4, or 1:5 to the BK-5F-NM-AMT, BK-5Cl-NM-AMT, and/or BK-5Br-NM-AMT. The required amount of dextroamphetamine will vary depending on the needs of the patient. The BK-5F-NM-AMT, BK-5Cl-NM-AMT, and/or BK-5Br-NM-AMT can be a racemic compound, an R- or S-enantiomer, or an enantiomerically enriched mixture of R- or S-enantiomers.

In one embodiment, the ratio of dextroamphetamine (with or without salt) to the BK-5F-NM-AMT, BK-5Cl-NM-AMT, and/or BK-5Br-NM-AMT (with or without salt) is about 1:2, about 1:3, about 1:4, or about 1:5 by weight, about 1:6, about 1:7, about 1:8, about 1:9, or about 1:10 by weight.

In certain embodiments, a compound of any of Formulas I-XXIX, with zero to five or zero to seven hydrogens replaced with deuterium, is formulated in a pharmaceutical composition that contains a pharmaceutically acceptable salt of dextroamphetamine in the amount of 2 mg, 4 mg, 5 mg, 7 mg, 10 mg, 15 mg, 20 mg, or 25 mg. The required amount of dextroamphetamine will vary depending on the needs of the patient. The compound of any of Formulas I-XXIX can be a racemic compound, an R- or S-enantiomer, or an enantiomerically enriched mixture of R- or S-enantiomers.

In another embodiments, a compound of any of Formulas I-XXIX, with zero to five or zero to seven hydrogens replaced with deuterium, is formulated in a pharmaceutical composition that contains a pharmaceutically acceptable salt of dextroamphetamine with dextroamphetamine in a ratio by weight of 1:2, 1:3, 1:4, or 1:5 to the compound of any of Formulas I-XXIX. The required amount of dextroamphetamine will vary depending on the needs of the patient.

In one embodiment, the ratio of dextroamphetamine (with or without salt) to the compound of any of Formulas I-XXIX (with or without salt) is about 1:2, about 1:3, about 1:4, or about 1:5 by weight, about 1:6, about 1:7, about 1:8, about 1:9, or about 1:10 by weight.

Pharmaceutical Combinations with MDMA

In one embodiment, BK-5F-NM-AMT, BK-5Cl-NM-AMT, and/or BK-5Br-NM-AMT is formulated in a pharmaceutical composition that contains MDMA or a pharmaceutically acceptable salt thereof. In one embodiment, the composition comprises between about at least 5 and about 180 mg or less of MDMA or a pharmaceutically acceptable salt thereof. In one embodiment, the composition comprises between about 15-60 mg of MDMA or a pharmaceutically acceptable salt thereof. The required amount of MDMA will vary depending on the needs of the patient. The BK-5F-NM-AMT, BK-5Cl-NM-AMT, and/or BK-5Br-NM-AMT can be a racemic compound, an R- or S-enantiomer, or an enantiomerically enriched mixture of R- or S-enantiomers. In one embodiment, the BK-5F-NM-AMT, BK-5Cl-NM-AMT, and/or BK-5Br-NM-AMT is deuterated wherein one to five hydrogens have been replaced with deuterium.

In one embodiment, the ratio of MDMA (with or without salt) to BK-5F-NM-AMT, BK-5Cl-NM-AMT, and/or BK-5Br-NM-AMT (with or without salt) is at least about 3:1, about 2:1, about 1:1, about 1:2, about 1:3, about 1:4, or about 1:5 by weight.

In one embodiment, a compound of any of Formulas I-XXIX is formulated in a pharmaceutical composition that contains MDMA or a pharmaceutically acceptable salt thereof. In one embodiment, the composition comprises between about at least 5 and about 180 mg or less of MDMA or a pharmaceutically acceptable salt thereof. In one embodiment, the composition comprises between about 15-60 mg of MDMA or a pharmaceutically acceptable salt thereof. The compound of any of Formulas I-XXIX can be a racemic compound, an R- or S-enantiomer, or an enantiomerically enriched mixture of R- or S-enantiomers. In one embodiment, the compound of any of Formulas I-XXIX is deuterated wherein one to five hydrogens have been replaced with deuterium.

In one embodiment, the ratio of MDMA (with or without salt) to the compound of any of Formulas I-XXIX (with or without salt) is at least about 3:1, about 2:1, about 1:1, about 1:2, about 1:3, about 1:4, or about 1:5 by weight.

Pharmaceutical Combinations with Psilocybin

In one embodiment, BK-5F-NM-AMT, BK-5Cl-NM-AMT, and/or BK-5Br-NM-AMT or a pharmaceutically acceptable salt thereof is formulated in a pharmaceutical composition that also contains psilocybin or a pharmaceutically acceptable salt thereof in the amount of at least about 0.01 mg, 0.1 mg, 0.5 mg, 1 mg, 2 mg, 3 mg, 4 mg, 5 mg, 10 mg, 15 mg, 20 mg, 25 mg, or 30 mg. The required amount of psilocybin will vary depending on the needs of the patient. The BK-5F-NM-AMT, BK-5Cl-NM-AMT, and/or BK-5Br-NM-AMT can be a racemic compound, an R- or S-enantiomer, or an enantiomerically enriched mixture of R- or S-enantiomers. In one embodiment, the BK-5F-NM-AMT, BK-5Cl-NM-AMT, and/or BK-5Br-NM-AMT is deuterated wherein one to five hydrogens have been replaced with deuterium.

In one embodiment, a compound of any of Formulas I-XXIX or a pharmaceutically acceptable salt thereof is formulated in a pharmaceutical composition that also contains psilocybin or a pharmaceutically acceptable salt thereof in the amount of at least about 0.01 mg, 0.1 mg, 0.5 mg, 1 mg, 2 mg, 3 mg, 4 mg, 5 mg, 10 mg, 15 mg, 20 mg, 25 mg, or 30 mg. The required amount of psilocybin will vary depending on the needs of the patient. The compound of any of Formulas I-XXIX can be a racemic compound, an R- or S-enantiomer, or an enantiomerically enriched mixture of R- or S-enantiomers. In one embodiment, the compound of any of Formulas I-XXIX is deuterated wherein one to five hydrogens have been replaced with deuterium.

Non-Limiting Examples of Combination Formulations

The examples below provide non-limiting embodiments of combination formulations, which can be used to deliver any of the compounds described herein in enantiomerically enriched form, pure form or even a racemic mixture. Therefore, while the compounds below are specified, any desired purity form or compound can be used if it achieves the desired goal of treatment.

In one non-limiting embodiment, a capsule comprising an enantiomerically enriched mixture of compound T-1, an enantiomerically enriched mixture of compound T-12, and amphetamine sulfate is prepared using the ingredients below. The active ingredients, cellulose, starch, and magnesium stearate are blended, passed through a No. 20 mesh U.S. sieve, and filled into hard gelatin capsules in 155 mg quantities.

| Ingredient | Quantity (mg/capsule) |
|---|---|
| T-1 | 30.0 |
| T-12 | 10.0 |
| Amphetamine sulfate | 5.0 |
| Starch | 109.0 |
| Magnesium stearate | 1.0 |

In one non-limiting embodiment, a capsule, comprising the enriched R-enantiomer of compound T-2, the enriched S-enantiomer of compound T-1, and psilocybin hydrochloride, is prepared using the ingredients below. The active ingredients, cellulose, starch, and magnesium stearate are blended, passed through a No. 20 mesh U.S. sieve, and filled into hard gelatin capsules in 155 mg quantities.

| Ingredient | Quantity (mg/capsule) |
|---|---|
| T-2 (70% R-enantiomer) | 30.0 |
| T-1 (70% S-enantiomer) | 10.0 |
| Psilocybin hydrochloride | 2.0 |
| Alpha lipoic acid | 40.0 |
| Starch | 72.0 |
| Magnesium stearate | 1.0 |

It should be readily appreciated that the above formulation examples are illustrative only. It should be understood that reference to particular compounds(s) is likewise illustrative, and the compounds(s) in any of the non-limiting examples of combination formulations may be substituted by other compounds(s) of the invention. Likewise, any of the other active compounds (for example, amphetamine sulfate or psilocybin hydrochloride as described above) may be substituted by a different other active compound, as may the inactive compounds.

Moreover, for any of BK-5F-NM-AMT, BK-5Cl-NM-AMT, BK-5Br-NM-AMT, or a compound of any of Formulas I-XXIX or for any other active compounds of the invention, substitution of the compound by its prodrug, free base, salt, or hydrochloride salt shall be understood to provide merely an alternative embodiment still within the scope of the invention. Further, compositions within the scope of the invention should be understood to be open-ended and may include additional active or inactive compounds and ingredients.

The type of formulation employed for the administration of the compounds employed in the methods of the present invention generally may be dictated by the compound(s) employed, the type of pharmacokinetic profile desired from the route of administration and the compound(s), and the state of the patient.

### VI. Dosage Regimes

The compounds or pharmaceutically acceptable formulations of the present invention can be administered to the host in any amount, and with any frequency, that achieves the goals of the invention as used by the healthcare provider, or otherwise by the host in need thereof, typically a human, as necessary or desired.

In certain embodiments, the composition as described herein is provided only in a controlled counseling session, and administered only once, or perhaps 2, 3, 4, or 5 or more times in repeated counseling sessions to address a mental disorder as described herein.

In other embodiments, the composition as described herein is provided outside of a controlled counseling session, and perhaps self-administered, as needed to perhaps 2, 3, 4, or 5 or more times in to address a mental disorder as described herein.

In other embodiments, the composition of the present invention may be administered on a routine basis for mental wellbeing or for entactogenic treatment.

The compounds of the current invention can be administered in a variety of doses, routes of administration, and dosing regimens, based on the indication and needs of the patient. Non-limiting examples of therapeutic use include discrete psychotherapeutic sessions, ad libitum use for treatment of episodic disorders, and ongoing use for treatment of subchronic and chronic disorders.

Psychotherapeutic Sessions

For some indications, the medicine is taken in discrete psychotherapy or other beneficial sessions. It is anticipated that these sessions will typically be separated by more than 5 half-lives of the medicine and, for most patients, will typically occur only 1 to 5 times each year.

For these sessions, it will typically be desirable to induce clearly perceptible entactogenic effects that will facilitate fast therapeutic progress. Non-exhaustive examples of oral doses of medicine that produce clearly perceptible entactogenic effects for exemplary purposes for any of the compounds described herein include (using compounds for illustrative purposes only): about 75 to about 800 mg of BK-5F-NM-AMT, about 75 to about 800 mg of BK-5Cl-NM-AMT, about 75 to about 800 mg of BK-5Br-NM-AMT.

It is anticipated that the medicine would be taken once or, more rarely, two or three times in a single therapeutic session. In these cases, it is common for each subsequent dose to be half of the previous dose or lower. Multiple doses within a session typically occur because either the patient's sensitivity to the medicine was unknown and too low of an initial dose was employed or because the patient is experiencing a productive session and it is desirable to extend the duration of therapeutic effects. Controlled release preparations may be used to lengthen the duration of therapeutic effects from a single administration of the medicine. In cases where multiple administrations are used in a session, it is anticipated that individual doses will be lower so that plasma concentrations remain within a desired therapeutic range.

Non-limiting, non-exhaustive examples of indications that may benefit from psychotherapeutic sessions include post-traumatic stress disorder, depression, dysthymia, anxiety and phobia disorders, feeding, eating, and binge disorders, body dysmorphic syndromes, alcoholism, tobacco abuse, drug abuse or dependence disorders, disruptive behavior disorders, impulse control disorders, gaming disorders, gambling disorders, personality disorders, attachment disorders, autism, and dissociative disorders. Also included as exemplary situations where an individual would benefit from a psychotherapeutic session are situations from a reduction of neuroticism or psychological defensiveness, an increase in openness to experience, an increase in creativity, or an increase in decision-making ability.

### Ad Libitum Use for Treatment of Episodic Disorders

For some indications, such as social anxiety, where the patient has need for relief from episodic occurrence of a disorder, it is anticipated that the medicine would be taken as needed but that uses should be separated by more than 5 half-lives of the medicine to avoid bioaccumulation and formation of tolerance.

For treating episodic disorders, clearly perceptible entactogenic effects are often not desirable, as they can impair some aspects of functioning. Non-exhaustive examples of oral doses of medicine for any of the compounds described herein include (using compounds for illustrative purposes only) that produce subtle, barely perceptible therapeutic effects include: about 20 to about 200 mg of BK-5F-NM-AMT, about 20 to about 200 mg of BK-5Cl-NM-AMT, and about 20 to about 200 mg of BK-5Br-NM-AMT.

Non-limiting, non-exhaustive examples of indications that may benefit from episodic treatment include post-traumatic stress disorder, depression, dysthymia, anxiety and phobia disorders, feeding, eating, and binge disorders, body dysmorphic syndromes, alcoholism, tobacco abuse, drug abuse or dependence disorders, disruptive behavior disorders, impulse control disorders, gaming disorders, gambling disorders, personality disorders, attachment disorders, autism, and dissociative disorders, provided that clinically significant signs and symptoms worsen episodically or in predictable contexts.

### Ongoing Use for Treatment of Subchronic and Chronic Disorders

For some indications, such as substance use disorders, inflammatory conditions, and neurological indications, including treatment of stroke, brain trauma, dementia, and neurodegenerative diseases, where the patient has need for ongoing treatment, it is anticipated that the medicine would be taken daily, twice daily, or three times per day. With some indications (subchronic disorders), such as treatment of stroke or traumatic brain injury, it is anticipated that treatment duration will be time-limited and dosing will be tapered when the patient has recovered. An example dose taper regimen is a reduction in dose of 10% of the original dose per week for nine weeks. With other, chronic disorders, such as dementia, it is anticipated that treatment will be continued as long as the patient continues to receive clinically significant benefits.

For treating subchronic and chronic disorders, clearly perceptible entactogenic effects are often not desirable. Non-exhaustive examples of oral doses of medicine for any of the compounds described herein include (using compounds for illustrative purposes only) that produce subtle, barely perceptible therapeutic effects with ongoing dosing include: about 10 to about 200 mg of BK-5F-NM-AMT, about 10 to about 200 mg of BK-5Cl-NM-AMT, and about 10 to about 200 mg of BK-5Br-NM-AMT.

Non-limiting, non-exhaustive examples of subchronic and chronic disorders that may benefit from regular treatment include migraine, headaches (for example, cluster headache), neurodegenerative disorders, Alzheimer's disease, Parkinson's disease, schizophrenia, stroke, traumatic brain injury, phantom limb syndrome, and other conditions where increasing neuronal plasticity is desirable.

### VII. Examples

### Example 1: Synthesis of Select Compounds of the Present Invention

Methods for synthesis of the compounds described herein and/or starting materials are either described in the art or will be readily apparent to the skilled artisan in view of general references well-known in the art (see, for example, Green et al., “Protective Groups in Organic Chemistry,” (Wiley, 2nd ed. 1991); Harrison et al., “Compendium of Synthetic Organic Methods,” Vols. 1-8 (John Wiley and Sons, 1971-1996); “Beilstein Handbook of Organic Chemistry,” Beilstein Institute of Organic Chemistry, Frankfurt, Germany; Feiser et al, “Reagents for Organic Synthesis,” Volumes 1-17, Wiley Interscience; Trost et al., “Comprehensive Organic Synthesis,” Pergamon Press, 1991; “Theilheimer's Synthetic Methods of Organic Chemistry,” Volumes 1-45, Karger, 1991; March, “Advanced Organic Chemistry,” Wiley Interscience, 1991; Larock “Comprehensive Organic Transformations,” VCH Publishers, 1989; Paquette, “Encyclopedia of Reagents for Organic Synthesis,” John Wiley & Sons, 1995) and may be used to synthesize the compounds of the invention. In general, the approaches used by Blough and colleagues may be used with only minor adaptation (Schemes 1, 2, and 3 in Blough et al. Bioorg. Med. Chem. Lett., 2014, 24(19), 4754-4758), such adaptation being that known and understood to those of ordinary skill.

In general, the approaches used for similar compounds (Shulgin & Shulgin. 1992. PiHKAL. A chemical love story, Transform Press, Berkeley Calif.; Glennon et al. 1986. Journal of medicinal chemistry, 29(2), 194-199; Nichols et al. 1991. Journal of medicinal chemistry, 34(1), 276-281; Kedrowski et al. 2007. Organic Letters, 9(17), 3205-3207; Heravi & Zadsirjan. 2016. Current Organic Synthesis, 13(6), 780-833; Keri et al. 2017. European journal of medicinal chemistry, 138, 1002-1033; Perez-Silanes et al. 2001. Journal of Heterocyclic Chemistry, 38(5), 1025-1030; and references therein), such adaptation being that known and understood to those of ordinary skill.

### Synthesis of 1-(5-Fluoro-1H-indol-3-yl)-2-(methylamino) propan-1-one hydrochloride (BK-5F-NM-AMT)

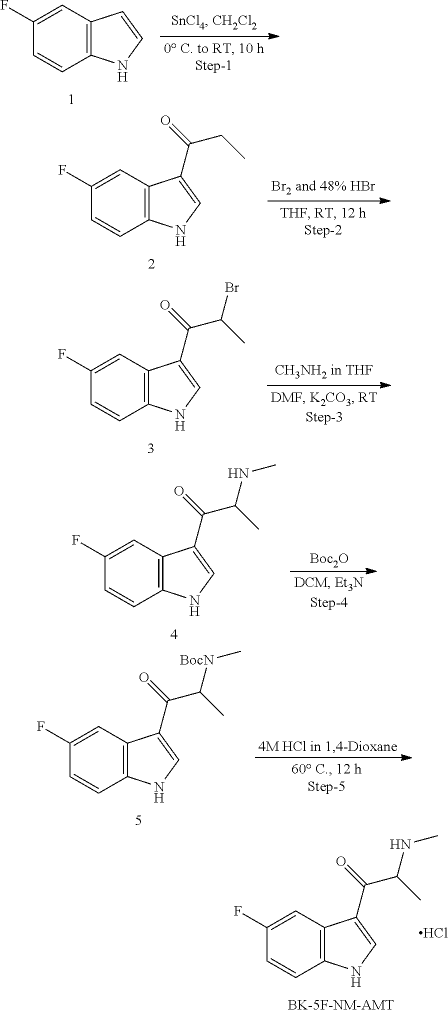

### Step 1: Synthesis of 1-(5-Fluoro-1H-indol-3-yl) propan-1-one (2)

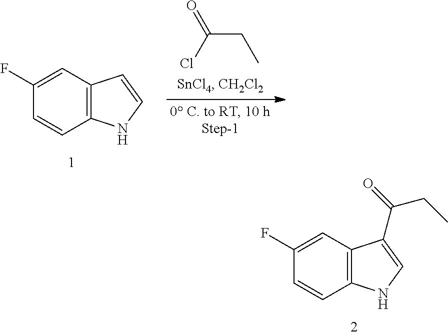

To a stirred solution of 5-Fluoro-1H-indole (1) (10 g, 73.99 mmol, 1 eq.) in dry DCM (200 mL) was added SnCl₄ (10.39 mL, 88.79 mmol, 1.2 eq.) at 0° C. under argon atmosphere. The resulting reaction mixture was allowed to stir at 0° C. for 30 min, then continue to stir at room temperature and Propionyl chloride (6.46 mL, 73.99 mmol, 1 eq.) and Nitromethane (140 mL) were added to the reaction mixture and continue to stir at room temperature for 10 h. Upon completion, monitored by TLC (30% EA in Hexane), the reaction mixture was quenched with water, extracted with ethyl acetate (2×200 mL), washed with water, followed by brine solution. Combined organic layer was dried over anhydrous sodium sulphate. Solvent was removed under vacuum and purified by silica gel column chromatography using ethyl acetate/hexane (20:80 v/v) as eluent to afford 1-(5-Fluoro-1H-indol-3-yl)propan-1-one (2) as light yellow solid (6 g, 42.41%). ¹H NMR (400 MHz, DMSO-d₆) δ 11.99 (s, 1H), 8.37 (d, J=2.64 Hz, 1H), 7.86-7.83 (dd, J=2.52 Hz, 10.08 Hz, 1H), 7.48-7.45 (dd, J=4.64 Hz, 8.84 Hz, 1H), 7.08-7.02 (m, 1H), 2.89-2.83 (q, 2H), 1.12-1.08 (t, J=7.36 Hz, 7.44 Hz, 3H). MS (ES) C₁₁H₁₀FNO requires 191, found 192 [M+H]⁺.

### Step 2: Synthesis of 2-bromo-1-(5-Fluoro-1H-indol-3-yl) propan-1-one (3)

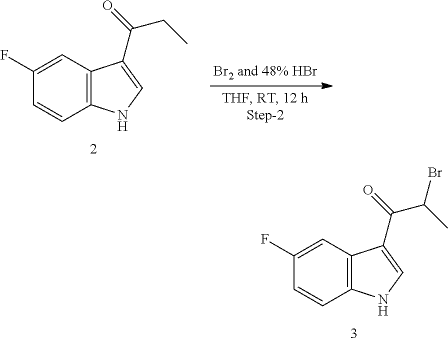

To a stirred solution of 1-(5-Fluoro-1H-indol-3-yl)propan-1-one (2) (3.5 g, 18.31 mmol, 1 eq.) in dry THF (50 mL) was added Hydrobromic acid 48% in Water (31.83 mL, 586.17 mmol, 32 eq.) and Bromine (1.03 mL, 20.15 mmol, 1.1 eq.) at 0° C. and the resulting reaction mixture was allowed to stir at room temperature for 12 h. Upon completion, monitored by TLC (20% EA in Hexane), the reaction mixture was basified with saturated sodium carbonate solution up to pH-8 and was extracted with ethyl acetate (2×150 mL), washed with water, followed by brine solution. Combined organic layer was dried over anhydrous sodium sulphate, solvent was removed under vacuum and purified by silica gel column chromatography using ethyl acetate/hexane (10:90 v/v) as eluent to afford 2-bromo-1-(5-Fluoro-1H-indol-3-yl)propan-1-one (3) as light yellow solid (2.2 g, 44.65%). ¹H NMR (400 MHz, DMSO-d₆) δ 12.27 (s, 1H), 8.58 (d, J=3.08 Hz, 1H), 7.86-7.82 (dd, J=2.56 Hz, 9.84 Hz, 1H), 7.53-7.50 (q, 1H), 7.13-7.08 (m, 1H), 5.65-5.60 (q, 1H), 1.77 (d, J=6.56 Hz, 3H). MS (ES) C₁₁H₉BrFNO requires 269, found 272 [M+H]⁺.

### Step 3: Synthesis of 1-(5-Fluoro-1H-indol-3-yl)-2-(methylamino) propan-1-one (4)

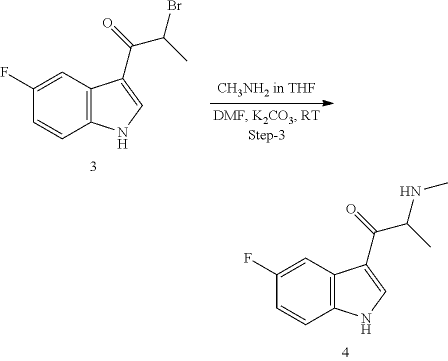 [SMILES: CC(Br)C(=O)c1c[nH]c2ccc(F)cc12 ; CNC(C)C(=O)c1c[nH]c2ccc(F)cc12]

To a stirred solution of 2-bromo-1-(5-Fluoro-1H-indol-3-yl)propan-1-one (3) (4.4 g, 16.35 mmol, 1 eq.) in dry DMF (50 mL) was added potassium carbonate (3.39 g, 24.53 mmol, 1.5 eq.) and methyl amine 2M in THF (49 mL, 98.14 mmol, 6 eq.) and the resulting reaction mixture was allowed to stir at room temperature for 12 h. Upon completion, monitored by TLC (10% EA in Hexane), the volatiles were evaporated and the reaction mixture was extracted with ethyl acetate (2×100 mL), washed with cold water (twice), followed by brine solution. Combined organic layer was dried over anhydrous sodium sulphate, solvent was removed under vacuum to afford crude 1-(5-Fluoro-1H-indol-3-yl)-2-(methylamino) propan-1-one (4) as yellow sticky solid (2.3 g, 63.84%). ¹H NMR crude data (400 MHz, DMSO-d₆) δ 12.12 (bs, 1H), 8.54 (s, 1H), 7.90 (d, J=9.72 Hz, 1H), 7.49-7.46 (m, 1H), 7.09-7.05 (m, 1H), 3.98-3.93 (m, 1H), 2.21 (s, 3H), 1.18 (d, J=6.72 Hz, 3H), MS (ES) C₁₂H₁₃FN₂O requires 220, found 221 [M+H]⁺.

### Step 4: Synthesis of tert-butyl 3-(N-(tert-butoxycarbonyl)-N-methylalanyl)-5-fluoro-1H-indole-1-carboxylate (5)

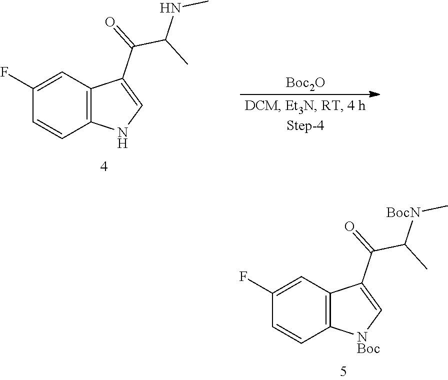 [SMILES: "Boc2O" ; CNC(C)C(=O)c1c[nH]c2ccc(F)cc12 ; CC(C(=O)c1cn(C(=O)OC(C)(C)C)c2ccc(F)cc12)N(C)C(=O)OC(C)(C)C]

To a stirred solution of crude 1-(5-fluoro-1H-indol-3-yl)-2-(methylamino)propan-1-one (4) (2.3 g, 9.74 mmol, 1 eq.) in dry DCM (40 mL) was added triethylamine (2.71 mL, 19.49 mmol, 2 eq.) and Boc anhydride (5.59 mL, 24.34 mmol, 2.5 eq.) and the resulting reaction mixture was allowed to stir at room temperature for 4 h. Upon completion (monitored by TLC, 10% EA in Hexane), the reaction mixture was extracted with DCM (2×100 mL), washed with water, followed by brine solution. Combined organic layer was dried over anhydrous sodium sulphate, solvent was evaporated under vacuum and purified by silica gel column chromatography using ethyl acetate/hexane (10:90 v/v) as eluent to afford tert-butyl 3-(N-(tert-butoxycarbonyl)-N-methylalanyl)-5-fluoro-1H-indole-1-carboxylate (5) as yellow sticky gum (3.0 g, 73%). ¹H NMR (400 MHz, DMSO-d₆) δ 8.65-8.47 (s, 1H), 8.10 (bs, 1H), 7.92-7.90 (m, 1H), 7.32-7.30 (m, 1H), 5.44-5.08 (s, 1H), 2.87 (m, 1H), 1.65 (s, 9H), 1.40-1.25 (m, 12H). MS (ES) C₂₂H₂₉FN₂O₅ requires 420, found 421 [M+H]⁺, 321 [M−100+2], rotamers observed.

### Step 5: Synthesis of 1-(5-Fluoro-1H-indol-3-yl)-2-(methylamino) propan-1-one hydrochloride (BK-5F-NM-AMT)

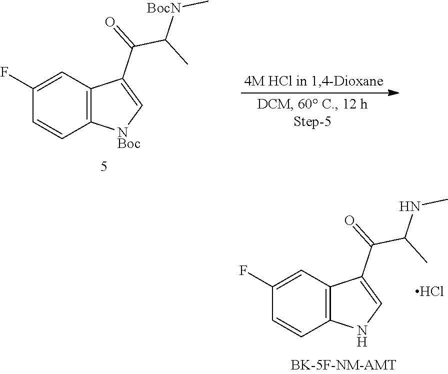 [SMILES: CC(C(=O)c1cn(C(=O)OC(C)(C)C)c2ccc(F)cc12)N(C)C(=O)OC(C)(C)C ; CNC(C)C(=O)c1c[nH]c2ccc(F)cc12 ; Cl]

To a stirred solution of tert-butyl 3-(N-(tert-butoxycarbonyl)-N-methylalanyl)-5-fluoro-1H-indole-1-carboxylate (5) (3.0 g, 9.36 mmol, 1 eq.) in dry DCM (40 mL) was added 4M HCl in 1,4 dioxane (30 mL) at 0° C. and the resulting reaction mixture was allowed to stir at 60° C. for 12 h. Upon completion of reaction (monitored by TLC, 10% EA in Hexane), the solvent were evaporated and the crude was washed twice with diethyl ether (2×50 mL) and pentane (1×50 mL) then dried under vacuum to afford 1-(5-fluoro-1H-indol-3-yl)-2-(methylamino)propan-1-one hydrochloride (BK-5F-NM-AMT) as off white solid (1.5 g, 72.7%). ¹H NMR (400 MHz, DMSO-d₆) δ 12.77 (s, 1H), 9.42 (bs, 2H), 8.64 (s, 1H), 7.85 (d, J=9.36 Hz, 1H), 7.57-7.54 (m, 1H), 7.14-7.10 (t, J=8.28 Hz, 8.76 Hz, 1 Hz), 4.86 (d, J=6.84 Hz, 1H), 2.54 (s, 3H), 1.53 (d, J=6.68 Hz, 3H). MS (ES) C₁₂H₁₃FN₂O requires 220, found 221 [M+H]⁺. HPLC: Purity (λ 210 nm): 99.83%.

### Synthesis of 1-(5-chloro-1H-indol-3-yl)-2-(methylamino) propan-1-one hydrochloride (BK-5Cl-NM-AMT)

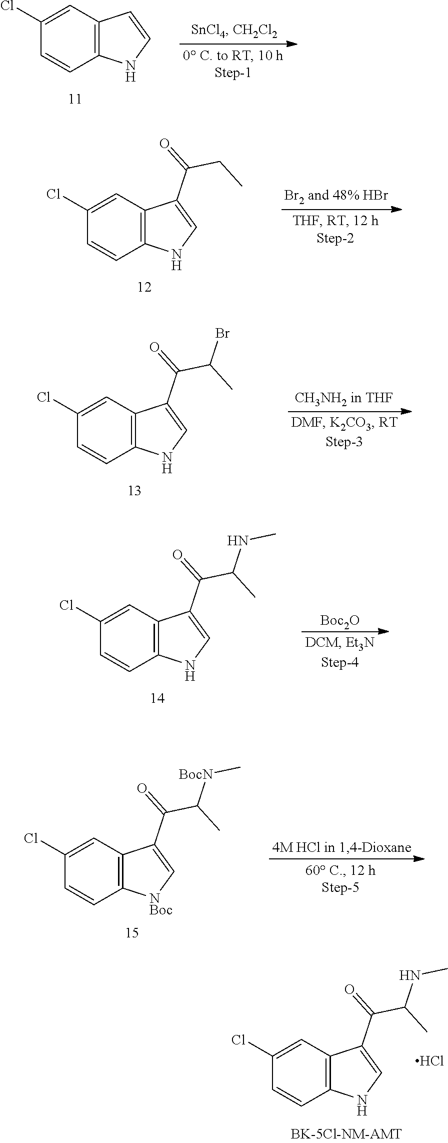

### Step 1: Synthesis of 1-(5-chloro-1H-indol-3-yl) propan-1-one (12)

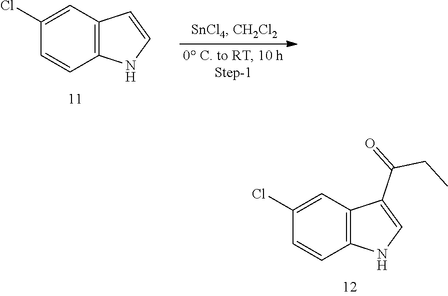

To a stirred solution of 5-chloro-1H-indole (11) (10 g, 65.96 mmol, 1 eq.) in dry DCM (200 mL) was added SnCl₄ (9.26 mL, 79.16 mmol, 1.2 eq.) at 0° C. under argon atmosphere. The resulting reaction mixture was allowed to stir at 0° C. for 30 min, then continue to stir at room temperature and Propionyl chloride (5.26 mL, 65.96 mmol, 1 eq.) and Nitromethane (140 mL) were added to the reaction mixture and continue to stir at room temperature for 10 h. Upon completion, monitored by TLC (30% EA in Hexane), the reaction mixture was quenched with water, extracted with ethyl acetate (2×200 mL), washed with water, followed by brine solution. Combined organic layer was dried over anhydrous sodium sulphate. Solvent was removed under vacuum and purified by silica gel column chromatography using ethyl acetate/hexane (20:80 v/v) as eluent to afford 1-(5-chloro-1H-indol-3-yl)propan-1-one (12) as light yellow solid (6 g, 44%). ¹H NMR (400 MHz, DMSO-d₆) δ 12.06 (s, 1H), 8.38 (d, J=2.88 Hz, 1H), 8.17 (d, J=1.56 Hz, 1H), 7.49 (d, J=8.6 Hz, 1H), 7.22-7.20 (dd, J=1.84 Hz, 8.6 Hz, 1H), 2.89-2.84 (q, 2H), 1.12-1.08 (t, J=7.36 Hz, 7.40 Hz, 3H). MS (ES) C₁₁H₁₀ClNO requires 207, found 208 [M+H]⁺.

### Step 2: Synthesis of 2-bromo-1-(5-chloro-1H-indol-3-yl) propan-1-one (13)

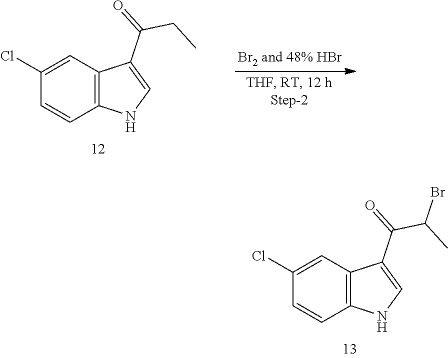

To a stirred solution of 1-(5-chloro-1H-indol-3-yl)propan-1-one (12) (3.5 g, 16.85 mmol, 1 eq.) in dry THE (50 mL) was added Hydrobromic Acid 48% in Water (29.28 mL, 539.369 mmol, 32 eq.) and Bromine (0.95 mL, 18.54 mmol, 1.1 eq.) at 0° C. and the resulting reaction mixture was allowed to stir at room temperature for 12 h. Upon completion, monitored by TLC (20% EA in Hexane), the reaction mixture was basified with saturated Sodium Carbonate solution up to pH-8 and was extracted with ethyl acetate (2×150 mL), washed with water, followed by brine solution. Combined organic layer was dried over anhydrous sodium sulphate, solvent was removed under vacuum and purified by silica gel column chromatography using ethyl acetate/hexane (10:90 v/v) as eluent to afford 2-bromo-1-(5-chloro-1H-indol-3-yl)propan-1-one (13) as light yellow solid (2.2 g, 45.5%). ¹H NMR (400 MHz, DMSO-d₆) δ 12.32 (s, 1H), 8.58 (d, J=3.2 Hz, 1H), 8.15 (d, J=2 Hz, 1H), 7.54 (d, J=8.64 Hz, 1H), 7.28 (dd, J=2.16 Hz, 8.68 Hz, 1H), 5.65-5.60 (q, 1H), 1.69 (d, J=6.92 Hz, 3H). MS (ES) C₁₁H₉BrClNO requires 285, found 286 [M+H]⁺.

### Step 3: Synthesis of 1-(5-chloro-1H-indol-3-yl)-2-(methylamino) propan-1-one (14)

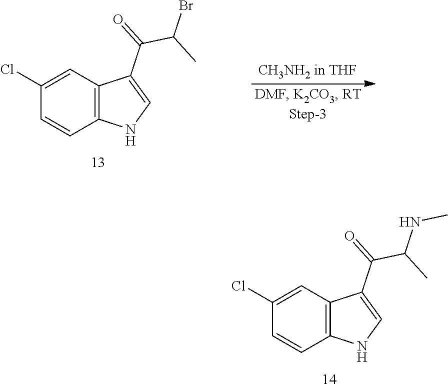 [SMILES: CC(Br)C(=O)c1c[nH]c2ccc(Cl)cc12 ; CNC(C)C(=O)c1c[nH]c2ccc(Cl)cc12]

To a stirred solution of 2-bromo-1-(5-chloro-1H-indol-3-yl)propan-1-one (13) (2.1 g, 7.32 mmol, 1 eq.) in dry DMF (30 mL) was added Potassium Carbonate (1.52 g, 10.99 mmol, 1.5 eq.) and methyl amine 2M in THE (22 mL, 43.97 mmol, 6 eq.), then the resulting reaction mixture was allowed to stir at room temperature for 12 h. Upon completion, monitored by TLC (10% EA in Hexane), the volatiles were evaporated and the reaction mixture was extracted with ethyl acetate (2×100 mL), washed with cold water (twice), followed by brine solution. Combined organic layer was dried over anhydrous sodium sulphate, solvent was removed under vacuum to afford crude 1-(5-chloro-1H-indol-3-yl)-2-(methylamino) propan-1-one (14) as yellow sticky solid (1.7 g, 98%). Proceeded for next step without further purification.

### Step 4: Synthesis of tert-butyl 3-(N-(tert-butoxycarbonyl)-N-methylalanyl)-5-chloro-1H-indole-1-carboxylate (15)

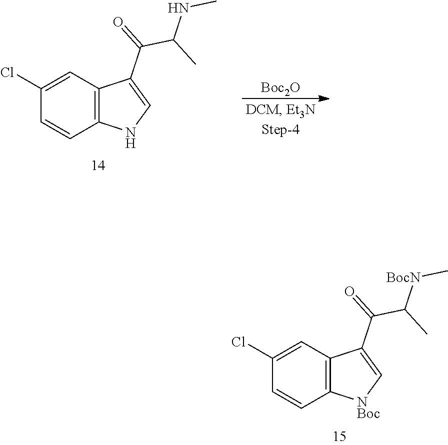 [SMILES: "Boc2O" ; CNC(C)C(=O)c1c[nH]c2ccc(Cl)cc12 ; CC(C(=O)c1cn(C(=O)OC(C)(C)C)c2ccc(Cl)cc12)N(C)C(=O)OC(C)(C)C]

To a stirred solution of crude 1-(5-chloro-1H-indol-3-yl)-2-(methylamino)propan-1-one (14) (1.8 g, 7.62 mmol, 1 eq.) in dry DCM (30 mL) was added triethylamine (2.12 mL, 15.25 mmol, 2 eq.) and Boc anhydride (3.5 mL, 15.25 mmol, 2 eq.), then the resulting reaction mixture was allowed to stir at room temperature for 4 h. Upon completion (monitored by TLC, 10% EA in Hexane), the reaction mixture was extracted with DCM (2×100 mL), washed with water, followed by brine solution. Combined organic layer was dried over anhydrous sodium sulphate, solvent was evaporated under vacuum and purified by silica gel column chromatography using ethyl acetate/hexane (10:90 v/v) as eluent to afford tert-butyl 3-(N-(tert-butoxycarbonyl)-N-methylalanyl)-5-chloro-1H-indole-1-carboxylate (15) as yellow sticky gum (1.3 g, 50.6%). ¹H NMR (400 MHz, DMSO-d₆) δ 8.63-8.45 (s, 1H), 8.20 (d, J=2 Hz, 1H), 8.11-8.09 (d, J=8.28 Hz, 1H), 7.48-7.45 (dd, J=1.48 Hz, 8.80 Hz, 1H), 5.43-5.08 (m, 1H), 2.83-2.66 (s, 3H), 1.65 (s, 9H), 1.40-1.26 (s, 12H). MS (ES) C₂₂H₂₉ClN₂O₅ requires 436, found 436.8, 335 [M−100+2], rotamer observed.

### Step 5: Synthesis of 1-(5-chloro-1H-indol-3-yl)-2-(methylamino) propan-1-one hydrochloride (BK-5Cl-NM-AMT)

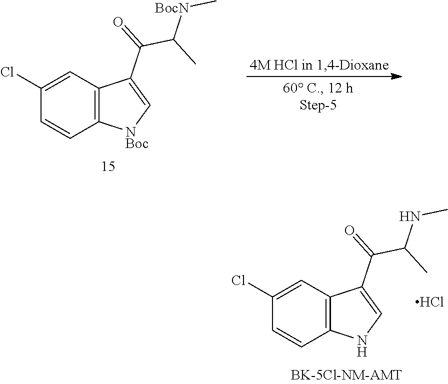 [SMILES: CC(C(=O)c1cn(C(=O)OC(C)(C)C)c2ccc(Cl)cc12)N(C)C(=O)OC(C)(C)C ; CNC(C)C(=O)c1c[nH]c2ccc(Cl)cc12 ; Cl]

To a stirred solution of tert-butyl 3-(N-(tert-butoxycarbonyl)-N-methylalanyl)-5-chloro-1H-indole-1-carboxylate (15) (2.2 g, 6.53 mmol, 1 eq.) in dry DCM (20 mL) was added 4M HCl in 1,4 dioxane (30 mL) at 0° C. and the resulting reaction mixture was allowed to stir at 60° C. for 12 h. Upon completion of reaction (monitored by TLC, 10% EA in Hexane), the solvent were evaporated and the crude was washed twice with diethyl ether (2×50 mL) and pentane (1×50 mL), then dried under vacuum to afford 1-(5-chloro-1H-indol-3-yl)-2-(methylamino)propan-1-one hydrochloride (BK-5Cl-NM-AMT) as off white solid (1.3 g, 72.8%). ¹H NMR (400 MHz, DMSO-d₆) δ 12.68 (s, 1H), 9.22 (bs, 1H), 8.63 (s, 1H), 8.15 (d, J=1.76 Hz, 1H), 7.58 (d, J=8.64 Hz, 1H), 7.32-7.29 (dd, J=1.84 Hz, 8.64 Hz, 1 Hz), 4.86-4.81 (q, 1H), 2.56 (s, 3H), 1.52 (d, J=6.96 Hz, 3H). MS (ES) C₁₂H₁₃ClN₂O requires 236, found 237 [M+H]⁺. HPLC: Purity (λ 220 nm): 99.43%.

### Synthesis of 1-(5-bromo-1H-indol-3-yl)-2-(methylamino) propan-1-one hydrochloride (BK-5Br-NM-AMT)

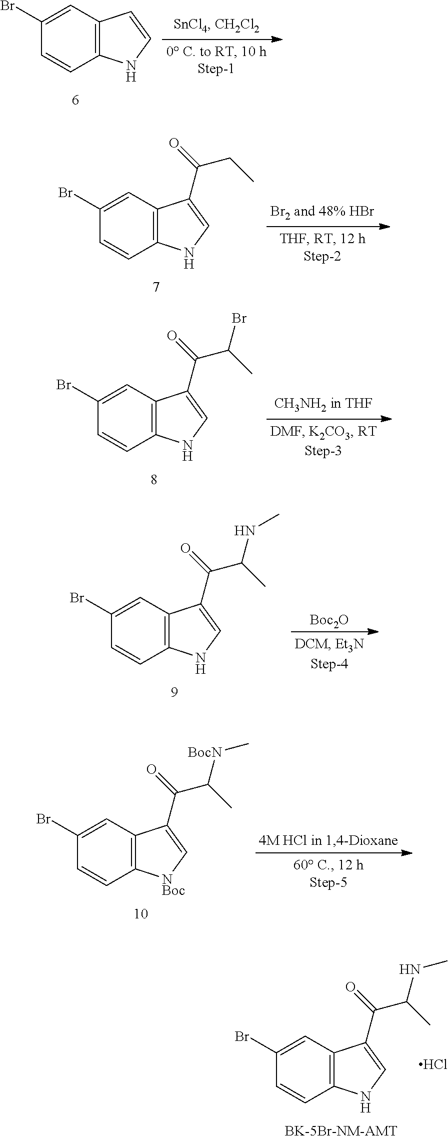

### Step 1: Synthesis of 1-(5-bromo-1H-indol-3-yl) propan-1-one (7)

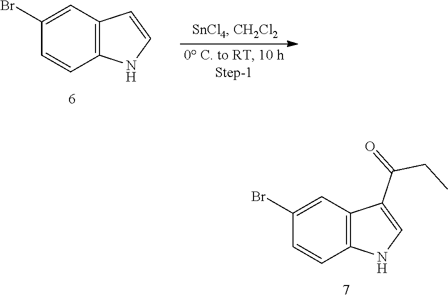

To a stirred solution of 5-bromo-1H-indole (6) (10 g, 51.02 mmol, 1 eq.) in dry DCM (200 mL) was added SnCl₄ (7.18 mL, 61.22 mmol, 1.2 eq.) at 0° C. under argon atmosphere. The resulting reaction mixture was allowed to stir at 0° C. for 30 min, then continue to stir at room temperature and Propionyl chloride (4.49 mL, 51.02 mmol, 1 eq.) and Nitromethane (140 mL) were added to the reaction mixture and the resulting reaction mixture was allowed to stir at room temperature for 10 h. Upon completion, monitored by TLC (30% EA in Hexane), the reaction mixture was quenched with water, extracted with ethyl acetate (2×200 mL), washed with water, followed by brine solution. Combined organic layer was dried over anhydrous sodium sulphate. Solvent was removed under vacuum and purified by silica gel column chromatography using ethyl acetate/hexane (20:80 v/v) as eluent to afford 1-(5-bromo-1H-indol-3-yl)propan-1-one (7) as light yellow solid (5 g, 39%). ¹H NMR (400 MHz, DMSO-d₆) δ 12.07 (s, 1H), 8.36 (s, 1H), 8.32 (d, J=1.64 Hz, 1H), 7.45 (d, J=8.6 Hz, 1H), 7.34-7.31 (dd, J=1.84 Hz, 8.64 Hz, 1H), 2.89-2.84 (q, 2H), 1.12-1.08 (t, J=7.36 Hz, 3H). MS (ES) C₁₁H₁₀BrNO requires 251, found 252 [M+H]⁺.

### Step 2: Synthesis of 2-bromo-1-(5-bromo-1H-indol-3-yl) propan-1-one (8)

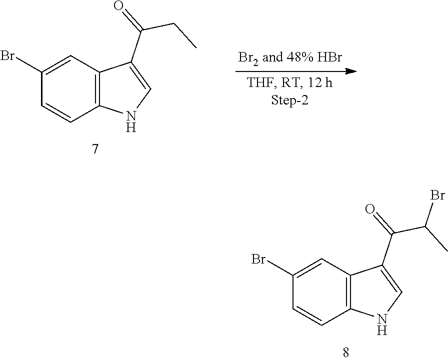

To a stirred solution of 1-(5-bromo-1H-indol-3-yl)propan-1-one (7) (5 g, 19.84 mmol, 1 eq.) in dry THE (50 mL) was added Hydrobromic Acid 48% in Water (51.37 mL, 634.92 mmol, 32 eq.) and Bromine (1.11 mL, 21.82 mmol, 1.1 eq.) at 0° C. and the resulting reaction mixture was allowed to stir at room temperature for 12 h. Upon completion, monitored by TLC (20% EA in Hexane), the reaction mixture was basified with saturated Sodium Carbonate solution up to pH-8 and was extracted with ethyl acetate (2×150 mL), washed with water, followed by brine solution. Combined organic layer was dried over anhydrous sodium sulphate, solvent was removed under vacuum and purified by silica gel column chromatography using ethyl acetate/hexane (10:90 v/v) as eluent to afford 2-bromo-1-(5-bromo-1H-indol-3-yl)propan-1-one (8) as light yellow solid (4 g, 60%). ¹H NMR (400 MHz, DMSO-d₆) δ 12.33 (s, 1H), 8.56 (d, J=3 Hz, 1H), 8.31 (d, J=1.4 Hz, 1H), 7.49 (d, J=8.64 Hz, 1H), 7.39-7.37 (dd, J=1.68 Hz, 8.60 Hz, 1H), 5.65-5.60 (q, 1H), 1.77 (d, J=6.6 Hz, 3H). MS (ES) C₁₁H₉Br₂NO requires 329, found 330 [M+H]⁺.

### Step 3: Synthesis of 1-(5-bromo-1H-indol-3-yl)-2-(methylamino)propan-1-one (9)

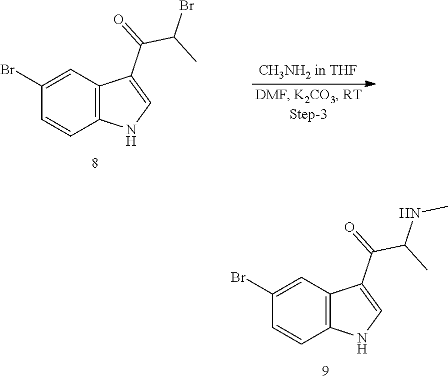 [SMILES: CC(Br)C(=O)c1c[nH]c2ccc(Br)cc12 ; CNC(C)C(=O)c1c[nH]c2ccc(Br)cc12]

To a stirred solution of 2-bromo-1-(5-bromo-1H-indol-3-yl)propan-1-one (8) (4 g, 12.08 mmol, 1 eq.) in dry DMF (40 mL) was added Potassium Carbonate (2.5 g, 18.12 mmol, 1.5 eq.) and methyl amine 2M in THE (36.25 mL, 72.50 mmol, 6 eq.) and the resulting reaction mixture was allowed to stir at room temperature for 12 h. Upon completion, monitored by TLC (10% EA in Hexane), the volatiles were evaporated and the reaction mixture was extracted with ethyl acetate (2×100 mL), washed with cold water (twice), followed by brine solution. Combined organic layer was dried over anhydrous sodium sulphate, solvent was removed under vacuum to afford crude 1-(5-bromo-1H-indol-3-yl)-2-(methylamino) propan-1-one (9) as yellow sticky solid (3.2 g, 94%). ¹H NMR crude data (400 MHz, DMSO-d₆) δ 8.54 (s, 1H), 8.36 (s, 1H), 7.46 (d, J=8.52 Hz, 1H), 7.35 (d, J=8.56 Hz, 1H), 3.98-3.93 (q, 1H), 2.20 (s, 3H), 1.18 (d, J=6.76 Hz, 3H). MS (ES) C₁₂H₁₃BrN₂O requires 280, found 281 [M+H]⁺.

### Step 4: Synthesis of tert-butyl 5-bromo-3-(N-(tert-butoxycarbonyl)-N-methylalanyl)-1H-indole-1-carboxylate (10)

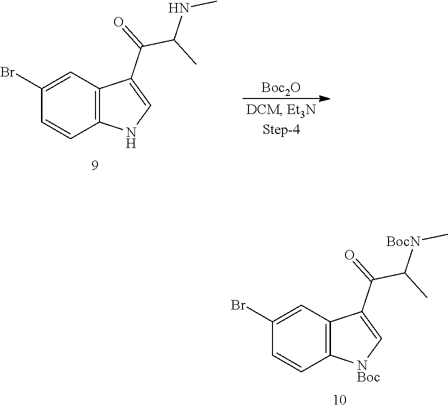 [SMILES: "Boc2O" ; CNC(C)C(=O)c1c[nH]c2ccc(Br)cc12 ; CC(C(=O)c1cn(C(=O)OC(C)(C)C)c2ccc(Br)cc12)N(C)C(=O)OC(C)(C)C]

To a stirred solution of crude 1-(5-bromo-1H-indol-3-yl)-2-(methylamino)propan-1-one (9) (3.2 g, 11.38 mmol, 1 eq.) in dry DCM (30 mL) was added triethylamine (3.28 mL, 22.77 mmol, 2 eq.) and Boc anhydride (5.22 mL, 22.77 mmol, 2 eq.) and the resulting reaction mixture was allowed to stir at room temperature for 4 h. Upon completion (monitored by TLC, 10% EA in Hexane), the reaction mixture was extracted with DCM (2×100 mL), washed with water, followed by brine solution. Combined organic layer was dried over anhydrous sodium sulphate, solvent was evaporated under vacuum and purified by silica gel column chromatography using ethyl acetate/hexane (10:90 v/v) as eluent to afford tert-butyl 5-bromo-3-(N-(tert-butoxycarbonyl)-N-methylalanyl)-1H-indole-1-carboxylate (10) as yellow sticky gum (3 g, 54%). ¹H NMR (400 MHz, DMSO-d₆) δ 8.59 & 8.41 (s, 1H), 8.33 (d, J=1.92 Hz, 1H), 8.04-8.00 (t, J=8.08 Hz, 7.76 Hz, 1H), 7.58-7.55 (dd, J=1.56 Hz, 8.92 Hz, 1H), 5.41 & 5.06 (s, 1H), 2.81 & 2.63 (s, 3H), 1.62 (s, 9H), 1.37-1.23 (m, 12H). MS (ES) C₂₂H₂₉BrN₂O₅ requires 480, found 383 [M−100+2], rotamers observed.

### Step 5: Synthesis of 1-(5-bromo-1H-indol-3-yl)-2-(methylamino) propan-1-one hydrochloride BK-5Br-NM-AMT)

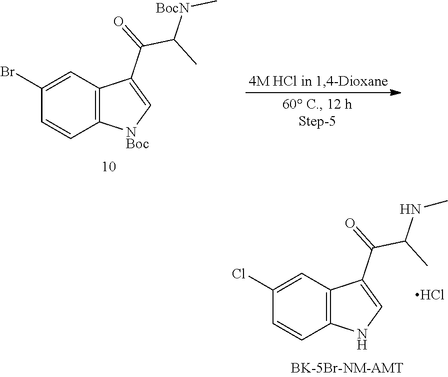 [SMILES: CC(C(=O)c1cn(C(=O)OC(C)(C)C)c2ccc(Br)cc12)N(C)C(=O)OC(C)(C)C ; CNC(C)C(=O)c1c[nH]c2ccc(Cl)cc12 ; Cl]

To a stirred solution of tert-butyl 5-bromo-3-(N-(tert-butoxycarbonyl)-N-methylalanyl)-1H-indole-1-carboxylate (10) (3 g, 6.23 mmol, 1 eq.) in dry DCM (20 ml) was added 4M HCl in 1,4 dioxane (30 ml) at 0° C. and the resulting reaction mixture was allowed to stir at 60° C. for 12 hrs. Upon completion of reaction (monitored by TLC, 10% EA in Hexane), the solvent were evaporated and the crude was washed twice with diethyl ether (2×50 ml) and pentane (1×50 ml) and dried under vacuum to afford 1-(5-bromo-1H-indol-3-yl)-2-(methylamino) propan-1-one hydrochloride (BK-5Br-NM-AMT) as off white solid (1.8 g, 81%). ¹H NMR (400 MHz, DMSO-d₆) δ 12.69 (s, 1H), 9.39-9.07 (bs, 2H), 8.61 (s, 1H), 8.30 (d, J=1.4 Hz, 1H), 7.53 (d, J=8.6 Hz, 1H), 7.43-7.40 (dd, J=1.68 Hz, 8.6 Hz, 1 Hz), 4.86-4.81 (q, 1H), 2.55 (s, 3H), 1.51 (d, J=6.92 Hz, 3H). MS (ES) C₁₂H₁₃BrN₂O requires 280, found 281 [M+H]⁺. HPLC: Purity (λ 220 nm): 98.46%.

### Additional Synthetic Examples

Starting with known starting materials the skilled artisan can synthesize compounds of the present invention with conventional methods and the teachings of this patent application. For example, pyrrolidine containing compounds of the present invention can be prepared from commercially available protected starting materials.

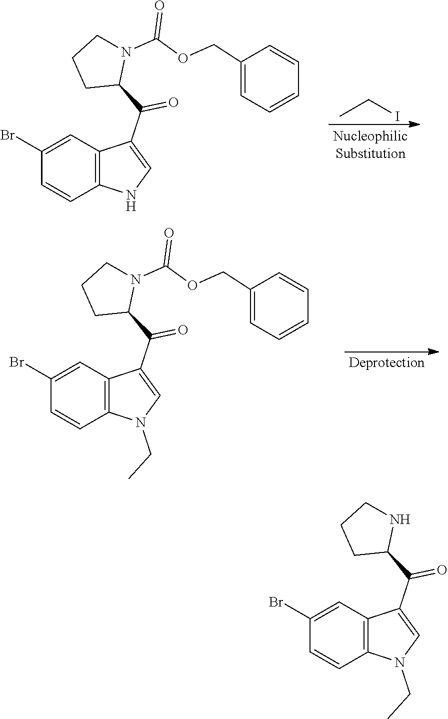

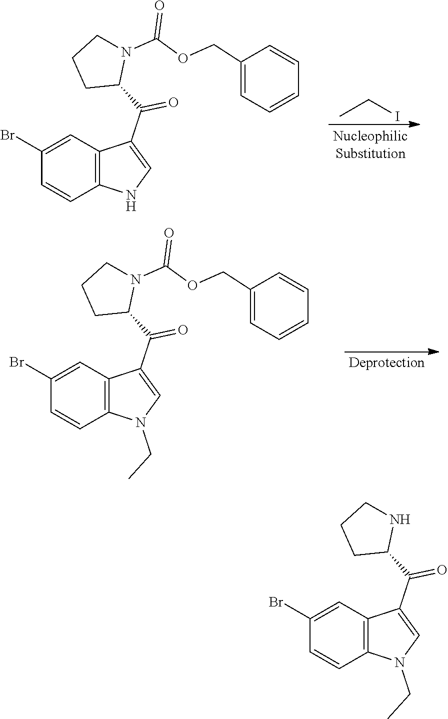

Additional compounds of the present invention can by synthesized by changing the order of reactions and if necessary switching protecting groups.

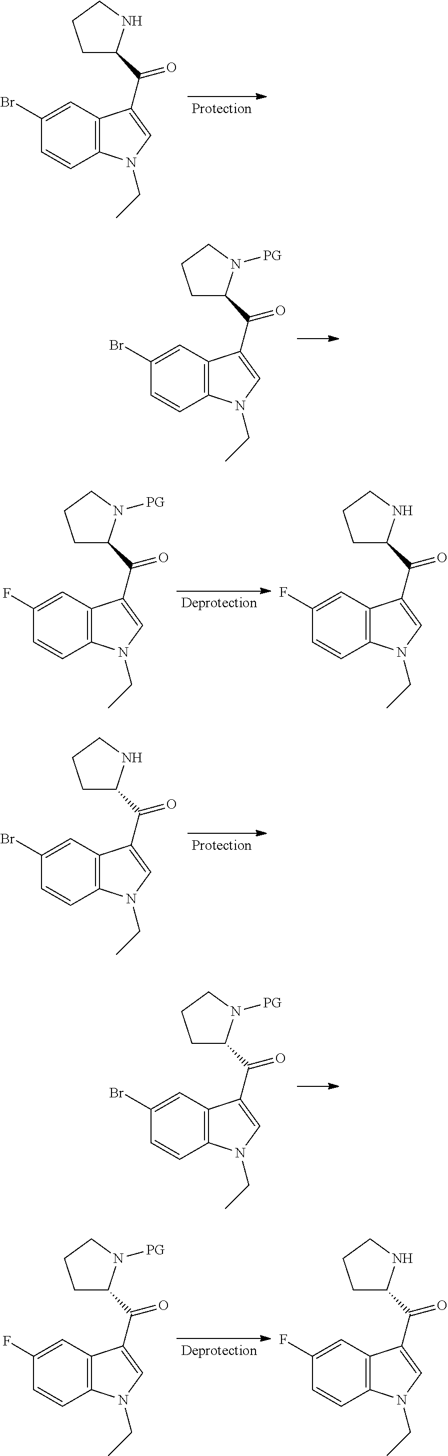

These techniques can be further modified by using different indoles to provide additional compounds of the present invention.

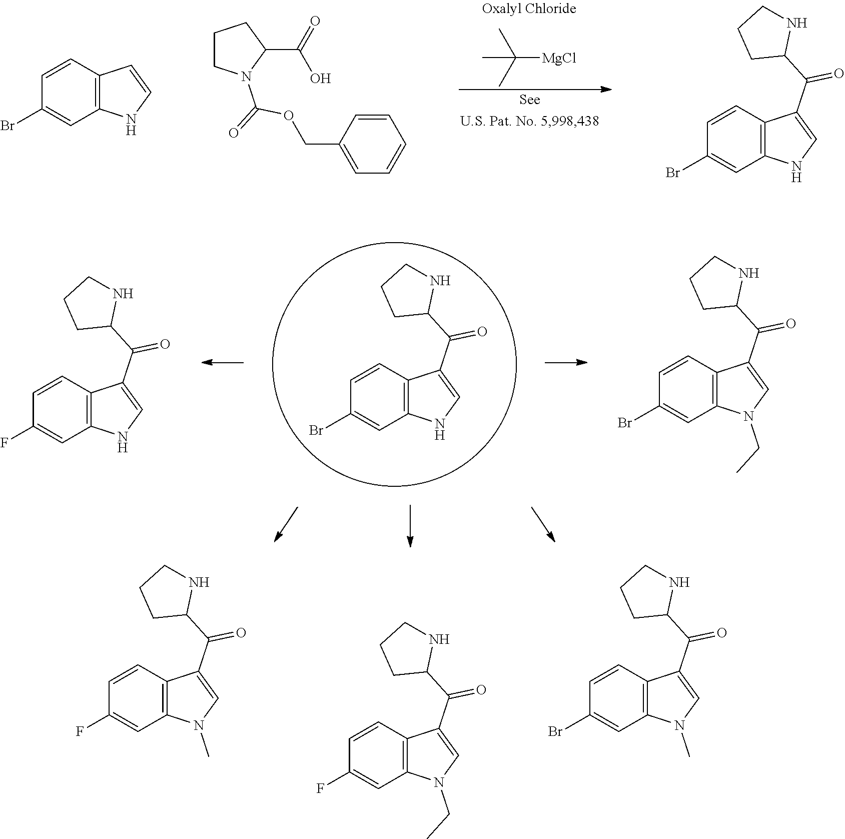

### Example 2: Production of Enantiomerically Enriched Preparations

Racemic compounds of the present invention are separated into pure enantiomers using the methods described herein or otherwise known to one of skill in the art. Exemplary synthetic transformations and supercritical fluid chiral chromatography conditions are given here as illustrative examples. Although there is variance in the chemical properties of the compounds of the present invention, it is routine to one skilled in the art to determine the exact conditions necessary to achieve separation in each case.

Enantiomeric Separation of BK-5F-NM-AMT:

Isomer separation of BK-5F-NM-AMT was done by normal phase chiral Prep-HPLC using the method given below.

Column: CHIRALPAK IE (250×21 mm) 5 μm

Flow rate—21 mL/min

Mobile phase—hexane/EtOH/IPA: 93/07/0.1

Solubility—MeOH

Wavelength—254 nm

Run time—30 min

Stack time—15 min

Loading per injection—2.5 mg

130 mg of BK-5F-NM-AMT was submitted and after separation about 25 mg of BK-5F-NM-AMT_Peak-1 and about 10 mg of BK-5F-NM-AMT_Peak-2 was obtained. about 60 mg of mixture was recovered. Peak 1 was obtained at −7.34 min. Peak 2 was obtained at −8.04 min.

BK-5F-NM-AMT_Peak-1: ¹H NMR (400 MHz, DMSO-d₆) δ 12.59 (s, 1H), 9.39 (bs, 1H), 9.00 (bs, 1H), 8.61 (d, J=3 Hz, 1H), 7.86-7.83 (dd, J=2 Hz, 9.68 Hz, 1H), 7.57-7.54 (m, 1H), 7.17-7.11 (m, 1 Hz), 4.85-4.80 (m, 1H), 2.56 (s, 3H), 1.52 (d, J=7.2 Hz, 3H). LCMS: Rt 1.79 min. MS (ES) C₁₂H₁₃FN₂O requires 220, found 221 [M+H]⁺. HPLC Purity (λ 210 nm): 99.11%, ee: 99.48%.

BK-5F-NM-AMT_Peak-2: ¹H NMR (400 MHz, DMSO-d₆) δ 12.53 (s, 1H), 9.20 (bs, 1H), 8.90 (bs, 1H), 8.59 (s, 1H), 7.86 (d, J=9.64 Hz, 1H), 7.58-7.54 (m, 1H), 7.17-7.13 (t, J=9.36 Hz, 8.04 Hz, 1 Hz), 4.82 (d, J=6.4 Hz, 1H), 2.57 (s, 3H), 1.51 (d, J=6.8 Hz, 3H). LCMS: Rt 1.76 min. MS (ES) C₁₂H₁₃FN₂O requires 220, found 221 [M+H]⁺. HPLC Purity (λ 210 nm): 99.80%, ee: 98.48%.

Enantiomeric Separation of BK-5Cl-NM-AMT:

Isomer separation of BK-5Cl-NM-AMT was done by normal phase Chiral Prep-HPLC and the

method was given below

Column—CHIRALPAK IE (250×21 mm) 5 μm

Flow rate—21 mL/min

Mobile phase—hexane/EtOH/IPA-93/07/0.1

Solubility—MeOH

Wavelength—296 nm

Run time—30 min

Stack time—15 min

Loading per injection—2.0 mg

100 mg of BK-5Cl-NM-AMT was submitted and after separation about 20 mg of BK-5Cl-NM-AMT_Peak-1 and about 12 mg of BK-5Cl-NM-AMT_Peak-2 was obtained. about 30 mg of mixture was recovered. Peak 1 was obtained at −6.82 min. Peak 2 was obtained at −7.24 min.

BK-5Cl-NM-AMT_Peak-1: ¹H NMR (400 MHz, DMSO-d₆) δ 12.61 (s, 1H), 9.24 (s, 1H), 8.96 (s, 1H), 8.61 (d, J=2.80 Hz, 1H), 8.15 (s, 1H), 7.58 (d, J=8.32 Hz, 1H), 7.32-7.30 (dd, J=1.72 Hz, 8.60 Hz, 1H), 4.83 (d, J=6.80 Hz, 1H), 2.57 (s, 3H), 1.51 (d, J=6.80 Hz, 3H). LCMS: Rt 1.97 min. MS (ES) C₁₂H₁₃ClN₂O requires 236, found 237 [M+H]⁺. HPLC: Purity (λ 220 nm): 95.65%, ee: 100%.

BK-5Cl-NM-AMT_Peak-2: ¹H NMR (400 MHz, DMSO-d₆) δ 12.63 (s, 1H), 9.30 (s, 1H), 8.97 (s, 1H), 8.62 (d, J=2.8 Hz, 1H), 8.15 (s, 1H), 7.58 (d, J=8.40 Hz, 1H), 7.32 (d, J=8.40 Hz, 1H), 4.83 (d, J=5.60 Hz, 1H), 2.57 (s, 3H), 1.51 (d, J=7.20 Hz, 3H). LCMS: Rt 1.97 min. MS (ES) C₁₂H₁₃ClN₂O requires 236, found 237 [M+H]⁺. HPLC: Purity (λ 210 nm): 99.85%, ee: 93.36%.

Enantiomeric Separation of BK-5Br-NM-AMT:

Isomer separation of BK-5Br-NM-AMT was done by normal phase chiral Prep-HPLC using the method given below.

Column—CHIRALPAK IE (250×21 mm) 5 μm

Flow rate—21 mL/min

Mobile phase—hexane/EtOH/IPA-92/08/0.1

Solubility—MeOH

Wavelength—296 nm

Run time—27 min

Stack time—13.5 min

Loading per injection—2.0 mg

100 mg of BK-5Br-NM-AMT was submitted and after separation about 19 mg of BK-5Br-NM-AMT_Peak-1 and about 11 mg of BK-5Br-NM-AMT_Peak-2 was obtained. about 26 mg of mixture was recovered. Peak 1 was obtained at −6.89 min. Peak 2 was obtained at −7.43 min

BK-5Br-NM-AMT_Peak-1: ¹H NMR (400 MHz, DMSO-d₆) δ 12.58 (s, 1H), 9.00 (bs, 2H), 8.59 (d, J=2.76 Hz, 1H), 8.31 (d, J=1.6 Hz, 1H), 7.53 (d, J=8.6 Hz, 1H), 7.44-7.41 (dd, J=1.84 Hz, 8.64 Hz, 1H), 4.82-4.81 (d, J=7.12 Hz, 1H), 2.58 (s, 3H), 1.51 (d, J=7 Hz, 3H). LCMS: Rt 1.99 min. MS (ES) C₁₂H₁₃BrN₂O requires 280, found 281 [M+H]⁺. HPLC: Purity (λ 220 nm): 96.41%, ee: 100%.

BK-5Br-NM-AMT_Peak-2: ¹H NMR (400 MHz, DMSO-d6) δ 12.57 (s, 1H), 9.00 (bs, 2H), 8.59 (d, J=3 Hz, 1H), 8.31 (d, J=1.84 Hz, 1H), 7.53 (d, J=8.64 Hz, 1H), 7.44-7.41 (dd, J=1.92 Hz, 8.64 Hz, 1H), 4.82 (d, J=7.16 Hz, 1H), 2.58 (s, 3H), 1.51 (d, J=6.96 Hz, 3H). LCMS: Rt 1.99 min. MS (ES) C₁₂H₁₃BrN₂O requires 280, found 281 [M+H]⁺. HPLC: Purity (λ 220 nm): 97.57%, ee: 95.12%.

### Example 3: Human 5-HT_{2A} Serotonin Receptor Agonist Activity

Stimulation of 5-HT_{2A} receptors can be measured in several well-known methods. One method employs a calcium flux assay and a cloned human 5-HT_{2A}-expressing cell line. In this example method of measuring agonist activity, 5-HT_{2A}-expressing Chem-1 cells are seeded at 50,000 cells per well into a 96-well plate, and the following day the cells are loaded with a fluorescent calcium indicator. Calcium flux in response to the indicated ligand with a final concentration of 0.5% DMSO is determined on a Molecular Devices FLIPRTETRA® with ICCD camera or equivalent. Parental control cells are tested to determine the specificity of the resulting signal. In addition to measuring calcium flux, it is also common to measure intracellular inositol phosphates or arachidonic acid.

Potential agonist effects of BK-5F-NM-AMT, BK-5F-NM-AMT, and BK-5F-NM-AMT at 5-HT_{2A} receptors was measured by HTRF as changes in IPl. The effects were measured using human 5-HT_{2A} expressed in HEK-293 cells, based on methods reported in Porter et al. British Journal of Pharmacology, 1999. 128(1), 13-20. Incubation was 30 min at 37° C. 10 μM serotonin was used as a positive control. The tryptamine derivatives were each tested at six concentrations from 3.0E-10 to 3.0E-05 M using log steps. All assays were performed in duplicate with results averaged.

Signal changes at the highest tested concentration were −0.8, −1.1, and 1.3% of control for BK-5F-NM-AMT, BK-5Br-NM-AMT, and BK-5Cl-NM-AMT, respectively, indicating that these molecules have no relevant agonist activity at 5-HT_{2A} receptors.

### Example 4: Marble Burying Measure of Decreased Anxiety and Neuroticism

The marble burying test is a model of neophobia, anxiety, and obsessive-compulsive behavior that has been proposed to have predictive validity for the screening of novel antidepressants and anxiolytics. Rodents use bedding material to bury noxious as well as harmless objects. It is well established to be sensitive to the effects of SSRIs as well as serotonin releasers such as fenfluramine and MDMA (De Brouwer et al., Cognitive, Affective, and Behavioral Neuroscience, 2019, 19(1), 1-39). The test involves the placement of a standardized number of marbles gently onto the surface of a layer of bedding material within a testing arena. Mice are then introduced into the arena for a standardized amount of time and allowed to explore the environment. The outcome measure of the test is the number of marbles covered, as scored by automatic scoring software or blinded observers. Compounds that attenuate anxiety, neuroticism, or obsessive-compulsive behavior decrease marble burying.

### Example 5: In Vitro Activity Studies

BK-5F-NM-AMT, BK-5Br-NM-AMT, and BK-5Cl-NM-AMT were evaluated for activity at 47 target sites at ten concentrations up to 10 μM, with EC₅₀ or IC₅₀ determined whenever possible.

In general, the three tryptamine derivatives showed few interactions with these potential binding sites, with antagonism of 5-HT_{2B} receptors and blocking of 5-HT_{3A} receptors being the only interactions with sub-micromolar potency.

TABLE 6
Results from Screening Against Potential Binding Sites

| | | BK-5F-NM-AMT | | BK-5Cl-NM-AMT | | BK-5Br-NM-AMT | |
|---|---|---|---|---|---|---|---|
| | | | IC₅₀ | | IC₅₀ | | IC₅₀ |
| | | EC₅₀ | (μM) | EC₅₀ | (μM) | EC₅₀ | (μM) |
| | | (μM) | Antag./ | (μM) | Antag./ | (μM) | Antag./ |
| | | Agonist/ | Blocker/ | Agonist/ | Blocker/ | Agonist/ | Blocker/ |
| Assay Target | Assay Type | Opener | Inhibitor | Opener | Inhibitor | Opener | Inhibitor |

Serotonergic Receptors

| | | | | | | | |
|---|---|---|---|---|---|---|---|
| 5-HT_{1B} | cAMP | >10 | >10 | >10 | >10 | >10 | >10 |
| 5-HT_{2A} | Calcium | >10 | >10 | >10 | >10 | >10 | 8.63237 |
| | Flux | | | | | | |
| 5-HT_{2B} | Calcium | >10 | 0.5865 | >10 | 0.0921 | >10 | 0.04469 |
| | Flux | | | | | | |
| 5-HT_{3A} | Ion | >10 | >30 | >10 | 0.96518 | >10 | 0.67278 |
| | Channel | | | | | | |
| 5-HT_{1A} | cAMP | >10 | >10 | >10 | >10 | >10 | >10 |

Other GPCRs

| | | | | | | | |
|---|---|---|---|---|---|---|---|
| ADRA1A | Calcium | >10 | >10 | >10 | >10 | 8.23569 | >10 |
| | Flux | | | | | | |
| ADRA2A | cAMP | >10 | >10 | >10 | >10 | >10 | >10 |
| ADRB2 | cAMP | >10 | >10 | >10 | 8.6794 | >10 | 5.44427 |
| HRH1 | Calcium | >10 | >10 | >10 | >10 | >10 | >10 |
| | Flux | | | | | | |
| ADORA2A | Calcium | >10 | >10 | >10 | >10 | >10 | >10 |
| | Flux | | | | | | |
| ADRB1 | cAMP | >10 | >10 | >10 | >10 | >10 | >10 |
| AVPR1A | Calcium | >10 | >10 | >10 | >10 | >10 | >10 |
| | Flux | | | | | | |
| CCKAR | Calcium | >10 | >10 | >10 | >10 | >10 | >10 |
| | Flux | | | | | | |
| CHRM1 | Calcium | >10 | >10 | >10 | >10 | >10 | >10 |
| | Flux | | | | | | |
| CHRM2 | cAMP | >10 | >10 | >10 | >10 | >10 | >10 |
| CHRM3 | Calcium | >10 | >10 | >10 | >10 | >10 | >10 |
| | Flux | | | | | | |
| CNR1 | cAMP | >10 | >10 | >10 | >10 | >10 | >10 |
| CNR2 | cAMP | >10 | >10 | >10 | >10 | >10 | >10 |
| DRD1 | cAMP | >10 | >10 | >10 | >10 | >10 | 9.69818 |
| DRD2S | cAMP | >10 | 5.20367 | >10 | 6.22272 | >10 | 4.25902 |
| EDNRA | Calcium | >10 | >10 | >10 | >10 | >10 | >10 |
| | Flux | | | | | | |
| HRH2 | cAMP | >10 | >10 | >10 | >10 | >10 | >10 |
| OPRD1 | cAMP | >10 | >10 | >10 | >10 | >10 | >10 |
| OPRK1 | cAMP | >10 | >10 | >10 | >10 | >10 | >10 |
| OPRM1 | cAMP | >10 | >10 | >10 | >10 | >10 | >10 |

Other Ion Channels

| | | | | | | | |
|---|---|---|---|---|---|---|---|
| nAChR(a4/b2) | Ion | >10 | >10 | >10 | >10 | >10 | >10 |
| | Channel | | | | | | |
| NAV1.5 | Ion | | >10 | | >10 | | >10 |
| | Channel | | | | | | |
| CAV1.2 | Ion | | >10 | | >10 | | >10 |
| | Channel | | | | | | |
| GABAA | Ion | >10 | >10 | >10 | >10 | >10 | >10 |
| | Channel | | | | | | |
| hERG | Ion | | >10 | | >10 | | >10 |
| | Channel | | | | | | |
| KvLQT1/minK | Ion | >10 | >10 | >10 | >10 | >10 | >10 |
| | Channel | | | | | | |
| NMDAR | Ion | >10 | >10 | >10 | >10 | >10 | >10 |
| (1A/2B) | Channel | | | | | | |

Monoamine Transporters

| | | | | |
|---|---|---|---|---|
| DAT | Transporter | >10 | 6.07645 | 4.23506 |
| NET | Transporter | >10 | >10 | >10 |
| SERT | Transporter | >10 | >10 | >10 |

Enzymes

| | | | | |
|---|---|---|---|---|
| MAOA | Enzymatic | >10 | >10 | >10 |
| AChE | Enzymatic | >10 | >10 | >10 |
| COX1 | Enzymatic | >10 | >10 | >10 |
| COX2 | Enzymatic | >10 | >10 | >10 |
| PDE3A | Enzymatic | >10 | >10 | >10 |
| PDE4D2 | Enzymatic | >10 | >10 | >10 |

Kinases

| | | | | | | | |
|---|---|---|---|---|---|---|---|
| ROCK1 | Binding | | >10 | | >10 | | >10 |
| INSR | Binding | | >10 | | >10 | | >10 |
| AR | NHR | >10 | >10 | >10 | >10 | >10 | >10 |
| | Nuclear | | | | | | |
| | Trans- | | | | | | |
| | location | | | | | | |
| GR | NHR | >10 | >10 | >10 | >10 | >10 | >10 |
| | Protein | | | | | | |
| | Interaction | | | | | | |
| LCK | Binding | | >10 | | >10 | | >10 |
| VEGFR2 | Binding | | >10 | | >10 | | >10 |

### In Vitro Assay Methods

Concentrations of test compounds were 0.00050806, 0.00152416, 0.0045724, 0.0137174, 0.041152, 0.123456, 0.37038, 1.11112, 3.3334, and 10 μM.

### CAMP Secondary Messenger Assays

CAMP secondary messenger assays used cell lines that stably expressed non-tagged GPCRs. Hit Hunter® CAMP assays monitored the activation of a GPCR via Gi and Gs secondary messenger signaling in a homogenous, non-imaging assay format using Enzyme Fragment Complementation (EFC) with β-galactosidase (β-gal) as the functional endpoint. For the assay system, exogenously introduced Enzyme Donor (ED) fused to cAMP (ED-cAMP) competes with endogenously generated cAMP for binding to an anti-cAMP-specific antibody. Active 3-gal is formed by complementation of exogenous Enzyme Acceptor (EA) to any unbound ED-cAMP. Active enzyme can then convert a chemiluminescent substrate, generating an output signal detectable on a standard microplate reader. Specific assay steps and reference compounds are given below for each assay type.

### Calcium Flux Secondary Messenger Assays

The Calcium No WashLs assay was used to monitor GPCR activity via Gq secondary messenger signaling in a live cell, non-imaging assay format. Calcium mobilization in PathHunter® cell lines or other cell lines stably expressing Gq-coupled GPCRs was monitored using calcium-sensitive dye loaded into cells. GPCR activation by a compound resulted in the release of calcium from intracellular stores and an increase in dye fluorescence that was measured in real-time. Specific assay steps and reference compounds are given below for each assay type.

### Nuclear Hormone Receptor Assays

PathHunter® NHR Protein Interaction (NHR Pro) and Nuclear Translocation (NHR NT) assays monitored the activation of specific nuclear hormone receptors in a homogenous, non-imaging assay format using Enzyme Fragment Complementation (EFC). The NHR Pro assay is based on detection of protein-protein interactions between an activated, full length NHR protein and a nuclear fusion protein containing Steroid Receptor Co-activator Peptide (SRCP) domains with one or more canonical LXXLL interaction motifs.

The NHR was tagged with the ProLink™ (PK) component of the DiscoverX EFC assay system, and the SRCP domain was fused to the Enzyme Acceptor component (EA) expressed in the nucleus. When bound by ligand, the NHR migrates to the nucleus and recruits the SRCP domain, whereby complementation occurs, generating a unit of active β-galactosidase (β-gal) and production of chemiluminescent signal upon the addition of PathHunter detection reagents.

The NHR NT assay monitored movement of an NHR between the cytoplasmic and nuclear compartments. The receptor was tagged with the ProLabel™ (PL) component of the EFC assay system, and EA was fused to a nuclear location sequence that restricted the expression of EA to the nucleus. Migration of the NHR to the nucleus resulted in complementation with EA generating a unit of active B-gal and production of a chemiluminescent signal upon the addition of Path Hunter detection reagents. Specific assay steps and reference compounds are given below for each assay type.

### KINOMEscan® Assays

Kinase activity was measured using the KINOMEscan screening platform, which employs a site-directed competition binding assay to quantitatively measure interactions between test compounds and the kinases. Compounds that bind the kinase active site and directly (sterically) or indirectly (allosterically) prevent kinase binding to the immobilized ligand, will reduce the amount of kinase captured on the solid support (A and B). Conversely, test molecules that do not bind the kinase have no effect on the amount of kinase captured on the solid support (C). Screening “hits” were identified by measuring the amount of kinase captured in test versus control samples by using a quantitative, precise and ultra-sensitive qPCR method that detects the associated DNA label (D). In a similar manner, dissociation constants (Kds) for test compound-kinase interactions were calculated by measuring the amount of kinase captured on the solid support as a function of the test compound concentration. Specific assay steps and reference compounds are given below for each assay type.

### Monoamine Transporter Uptake Assays

The Neurotransmitter Transporter Uptake Assay Kit from Molecular Devices was used as a homogeneous fluorescence-based assay for the detection of dopamine, norepinephrine or serotonin transporter activity in cells expressing these transporters. The kit employs a fluorescent substrate that mimics the biogenic amine neurotransmitters that are taken into the cell through these specific transporters, resulting in increased intracellular fluorescence intensity.

It should be noted that fluorescence-based assays for the detection of dopamine, norepinephrine or serotonin transporter activity have poor sensitivity for compounds that are substrates for these monoamine transporters. Interactions with these transporters were therefore separately measured using two additional types of assays: an antagonist radioligand assay of inhibition of the human 5-HT transporter (hSERT) expressed in CHO cells (Tatsumi, M. et al. (1999), Eur. J. Pharmacol., 368: 277-283) and an assay measuring release of [³H] Serotonin or [³H] dopamine, respectively, from cells stably expressing SERT or DAT. While the former is sensitive to classic reuptake inhibition, the latter can detect the effects of substrates, which also induce release. Specific assay steps and reference compounds are given below for each assay type.

### Potassium Assay

The FLIPR Potassium Assay Kit from Molecular Devices was used for ion channel assays. This approach exploited the permeability of thallium ions (TI+) through both voltage and ligand-gated potassium (K+) channels. A highly sensitive Tl+ indicator dye produced a bright fluorescent signal upon the binding to Tl+ conducted through potassium channels. The intensity of the Tl+ signal was proportional to the number of potassium channels in the open state and therefore provided a functional indication of the potassium channel activities. In addition, a masking dye was included to reduce background fluorescence for improved signal/noise ratio. Specific assay steps and reference compounds are given below for each assay type.

### Membrane Potential Assay

The FLIPR® Membrane Potential Assay Kit was used which employs a fluorescent indicator dye in combination with a quencher to reflect real-time membrane potential changes associated with ion channel activation and ion transporter proteins. Unlike traditional dyes such as DiBAC, the FLIPR Membrane Potential Assay Kit detects bidirectional ion fluxes so both variable and control conditions can be monitored within a single experiment. Specific assay steps and reference compounds are given below for each assay type.

### Calcium Assays

The DiscoveRx Calcium NWPLUS Assay Kit was used for detection of changes in intracellular calcium. Cells expressing a receptor of interest that signals through calcium were pre-loaded with a calcium sensitive dye and then treated with compound. Upon stimulation, the receptor signaled release of intracellular calcium, which resulted in an increase of dye fluorescence. Signal was measured on a fluorescent plate reader equipped with fluidic handling capable of detecting rapid changes in fluorescence upon compound stimulation. Specific assay steps and reference compounds are given below for each assay type.

### Enzymatic Assays

Enzymatic assays determined enzymatic activity by measuring either the consumption of substrate or production of product over time. Different detection methods were used in each enzymatic assay to measure the concentrations of substrates and products, including spectrophotometric, fluorometric, and luminescent readouts. Specific assay steps and reference compounds are given below for each assay type.

Assay Design: GPCR cAMP Modulation

### Cell Handling

1. cAMP Hunter cell lines were expanded from freezer stocks according to standard procedures.
2. Cells were seeded in a total volume of 20 μL into white walled, 384-well microplates and incubated at 37° C. for the appropriate time prior to testing.
3. cAMP modulation was determined using the DiscoverX HitHunter cAMP XS+ assay.

### Gs Agonist Format

1. For agonist determination, cells were incubated with sample to induce response.
2. Media was aspirated from cells and replaced with 15 μL 2:1 HBSS/10 mM Hepes: cAMP XS+Ab reagent.
3. Intermediate dilution of sample stocks was performed to generate 4× sample in assay buffer.
4. 5 μL of 4× sample was added to cells and incubated at 37° C. or room temperature for 30 or 60 minutes.

### Gi Agonist Format

1. For agonist determination, cells were incubated with sample in the presence of EC₅₀ forskolin to induce response.
2. Media was aspirated from cells and replaced with 15 μL 2:1 HBSS/10 MM Hepes: cAMP XS+Ab reagent.
3. Intermediate dilution of sample stocks was performed to generate 4× sample in assay buffer containing 4× EC₅₀ forskolin.
4. 5 μL of 4× sample was added to cells and incubated at 37° C. or room temperature for 30 or 60 minutes.

### Antagonist Format

1. For antagonist determination, cells were pre-incubated with sample followed by agonist challenge at the EC₅₀ concentration.
2. Media was aspirated from cells and replaced with 10 μL 1:1 HBSS/Hepes: cAMP XS+Ab reagent.
3. 5 μL of 4× compound was added to the cells and incubated at 37° C. or room temperature for 30 minutes.
4. 5 μL of 4× EC₅₀ agonist was added to cells and incubated at 37° C. or room temperature for 30 or 60 minutes. For Gi coupled GPCRs, EC₅₀ forskolin was included.

### Signal Detection

1. After appropriate compound incubation, assay signal was generated through incubation with 20 L cAMP XS+ED/CL lysis cocktail for one hour followed by incubation with 20 μL cAMP XS+EA reagent for three hours at room temperature.
2. Microplates were read following signal generation with PerkinElmer Envision instrument for chemiluminescent signal detection.

### Data Analysis

1. Compound activity was analyzed using CBIS data analysis suite (ChemInnovation, CA).
2. For Gs agonist mode assays, percentage activity was calculated using the following formula: % Activity=100%×(mean RLU of test sample−mean RLU of vehicle control)/(mean RLU of MAX control−mean RLU of vehicle control).
3. For Gs antagonist mode assays, percentage inhibition was calculated using the following formula: % Inhibition=100%×(1−(mean RLU of test sample−mean RLU of vehicle control)/(mean RLU of EC₅₀ control−mean RLU of vehicle control)).
4. For Gi agonist mode assays, percentage activity was calculated using the following formula: % Activity=100%×(1−(mean RLU of test sample−mean RLU of MAX control)/(mean RLU of vehicle control−mean RLU of MAX control)).
5. For Gi antagonist or negative allosteric mode assays, percentage inhibition was calculated using the following formula: % Inhibition=100%×(mean RLU of test sample−mean RLU of EC₅₀ control)/(mean RLU of forskolin positive control−mean RLU of EC₅₀ control).

For Primary screens, percent response was capped at 0% or 100% where calculated percent response returned a negative value or a value greater than 100, respectively.

### Assay Design: Calcium Mobilization

### Cell Handling

1. Cell lines were expanded from freezer stocks according to standard procedures.
2. Cells (10,000 cells/well) were seeded in a total volume of 50 μL (200 cells/μL) into black-walled, clear-bottom, Poly-D-lysine coated 384-well microplates and incubated at 37° C. for the appropriate time prior to testing.

### Dye Loading

1. Assays were performed in 1× Dye Loading Buffer consisting of 1× Dye (DiscoverX, Calcium No Wash PLUS kit, Catalog No. 90-0091), 1× Additive A and 2.5 mM Probenecid in HBSS/20 mM Hepes. Probenecid was prepared fresh.
2. Cells were loaded with dye prior to testing. Media was aspirated from cells and replaced with 25 μL Dye Loading Buffer.
3. Cells were incubated for 45 minutes at 37° C. and then 20 minutes at room temperature.

### Agonist Format

1. For agonist determination, cells were incubated with sample to induce response.
2. After dye loading, cells were removed from the incubator and 25 μL of 2× compound in HBSS/20 mM Hepes was added using a FLIPR Tetra (MDS).
3. Compound agonist activity was measured on a FLIPR Tetra. Calcium mobilization was monitored for 2 minutes with a 5 second baseline read.

### Antagonist Format

1. For antagonist determination, cells were pre-incubated with sample followed by agonist challenge at the EC₅₀ concentration.
2. After dye loading, cells were removed from the incubator and 25 μL 2× sample was added.
Cells were incubated for 30 minutes at room temperature in the dark to equilibrate plate temperature.
3. After incubation, antagonist determination was initiated with addition of 25 μL 1× compound with 3× EC₅₀ agonist using FLIPR
4. Compound antagonist activity was measured on a FLIPR Tetra (MDS). Calcium mobilization was monitored for 2 minutes with a 5 second baseline read.

### Data Analysis

1. FLIPR read—Area under the curve was calculated for the entire two minute read.
2. Compound activity was analyzed using CBIS data analysis suite (ChemInnovation, CA).
3. For agonist mode assays, percentage activity was calculated as: % Activity=100%×(mean RFU of test sample−mean RFU of vehicle control)/(mean MAX RFU control ligand−mean RFU of vehicle control).
4. For antagonist mode assays, percentage inhibition was calculated as: % Inhibition=100%×(1−(mean RFU of test sample−mean RFU of vehicle control)/(mean RFU of EC₅₀ control−mean RFU of vehicle control)).

For Primary screens, percent response was capped at 0% or 100% where calculated percent response returned a negative value or a value greater than 100, respectively.

### Assay Design: Nuclear Hormone Receptor

### Cell Handling

1. PathHunter NHR cell lines were expanded from freezer stocks according to standard procedures.
2. Cells were seeded in a total volume of 20 μL into white walled, 384-well microplates and incubated at 37° C. for the appropriate time prior to testing. Assay media contained charcoal-dextran filtered serum to reduce the level of hormones present.

### Agonist Format

1. For agonist determination, cells were incubated with sample to induce response.
2. Intermediate dilution of sample stocks was performed to generate 5× sample in assay buffer.
3. 5 μL of 5× sample was added to cells and incubated at 37° C. or room temperature for 3-16 hours.

### Antagonist Format

1. For antagonist determination, cells were pre-incubated with antagonist followed by agonist challenge at the EC₅₀ concentration.
2. Intermediate dilution of sample stocks was performed to generate 5× sample in assay buffer.
3. 5 μL of 5× sample was added to cells and incubated at 37° C. or room temperature for 60 minutes.
Vehicle concentration was 1%.
4. 5 μL of 6× EC80 agonist in assay buffer was added to the cells and incubated at 37° C. or room temperature for 3-16 hours.

### Signal Detection

1. Assay signal was generated through a single addition of 12.5 or 15 μL (50% v/v) of PathHunter Detection reagent cocktail, followed by a one-hour incubation at room temperature.
2. Microplates were read following signal generation with a PerkinElmer Envision instrument for chemiluminescent signal detection.

### Data Analysis

1. Compound activity was analyzed using CBIS data analysis suite (ChemInnovation, CA).
2. For agonist mode assays, percentage activity was calculated as: % Activity=100%×(mean RLU of test sample−mean RLU of vehicle control)/(mean MAX control ligand−mean RLU of vehicle control).
3. For antagonist mode assays, percentage inhibition was calculated as:

% Inhibition=100%×(1−(mean RLU of test sample−mean RLU of vehicle control)/(mean RLU of EC₅₀ control−mean RLU of vehicle control)).

4. Note that for select assays, the ligand response produces a decrease in receptor activity (inverse agonist with a constitutively active target). For those assays inverse agonist activity was calculated as: % Inverse Agonist Activity=100%×((mean RLU of vehicle control−mean RLU of test sample)/(mean RLU of vehicle control−mean RLU of MAX control)).

For Primary screens, percent response was capped at 0% or 100% where calculated percent response returned a negative value or a value greater than 100, respectively.

### Assay Design: KINOMEscan Binding Assays

### Protein Expression

For most assays, kinase-tagged T7 phage strains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (6,000×g) and filtered (0.2 m) to remove cell debris. The remaining kinases were produced in HEK-293 cells and subsequently tagged with DNA for qPCR detection.

### Capture Ligand Production

Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific phage binding.

### Binding Reaction Assembly

Binding reactions were assembled by combining kinases, liganded affinity beads, and test compounds in 1× binding buffer (20% SeaBlock, 0.17×PBS, 0.05% Tween 20, 6 mM DTT). All reactions were performed in polypropylene 384-well plates in a final volume of 0.02 mL. The assay plates were incubated at room temperature with shaking for 1 hour and the affinity beads were washed with wash buffer (lx PBS, 0.05% Tween 20). The beads were then re-suspended in elution buffer (lx PBS, 0.05% Tween 20, 0.5 μM non-biotinylated affinity ligand) and incubated at room temperature with shaking for 30 minutes. The kinase concentration in the eluates was measured by qPCR.

### Signal Detection

The kinase concentration in the eluates was measured by qPCR. qPCR reactions were assembled by adding 2.5 μL of kinase eluate to 7.5 μL of qPCR master mix containing 0.15 μM amplicon primers and 0.15 μM amplicon probe. The qPCR protocol consisted of a 10-minute hot start at 950 C, followed by 35 cycles of 95° C. for 15 seconds, 60° C. for 1 minute.

### Data Analysis

### Percent Response Calculation

100*(test compound signal−positive control signal)/(negative compound signal−positive control signal)

Where:
Test compound=compound submitted by Customer
Negative control=DMSO (100% Ctrl)
Positive control=control compound (0% Ctrl)

Percent of Control was converted to Percent Response with the conversion: Percent Response=(100−Percent Control).

For Primary screens, percent response was capped at 0% or 100% where calculated percent response returned a negative value or a value greater than 100, respectively.

### Binding Constants (Kds)

Binding constants (Kds) were calculated with a standard dose response curve using the Hill equation with Hill Slope set to −1. Curves were fitted using a non-linear least square fit with the Levenberg-Marquardt algorithm.

### Assay Design: Ion Channel Assays

### Cell Handling

1. Cell lines were expanded from freezer stocks according to standard procedures.
2. Cells were seeded in a total volume of 20 μL into black-walled, clear-bottom, Poly-D-lysine coated 384-well microplates and incubated at 37° C. for the appropriate time prior to testing.

### Dye Loading

1. Assays were performed in 1× Dye Loading Buffer consisting of 1× Dye, and 2.5 mM Probenecid when applicable. Probenecid was prepared fresh.
2. Cells were loaded with dye prior to testing.
3. Cells were incubated for 30-60 minutes at 37° C.

### Agonist/Opener Format

1. For agonist determination, cells were incubated with sample to induce response.
2. Intermediate dilution of sample stocks was performed to generate 2-5× sample in assay buffer.
3. 10-25 μL of 2-5× sample was added to cells and incubated at 37° C. or room temperature for 30 minutes.

### Antagonist/Blocker Format

1. For antagonist determination, cells were pre-incubated with sample.
2. Intermediate dilution of sample stocks was performed to generate 2-5× sample in assay buffer.
3. After dye loading, cells were removed from the incubator and 10-25 μL 2-5× sample was added to cells in the presence of EC80 agonist when appropriate. Cells were incubated for 30 minutes at room temperature in the dark to equilibrate plate temperature.

### Signal Detection

1. Compound activity was measured on a FLIPR Tetra (MDS).

### Data Analysis

1. Compound activity was analyzed using CBIS data analysis suite (ChemInnovation, CA).
2. For agonist mode assays, percentage activity was calculated using the following formula: % Activity=100%×(mean RLU of test sample−mean RLU of vehicle control)/(mean MAX control ligand−mean RLU of vehicle control).
3. For antagonist percentage inhibition was calculated using the following formula:

% Inhibition=100%×(1−(mean RLU of test sample−mean RLU of vehicle control)/(mean RLU of EC80 control−mean RLU of vehicle control)).

For Primary screens, percent response was capped at 0% or 100% where calculated percent response returned a negative value or a value greater than 100, respectively.

### Assay Design: Transporter Assays

### Cell Handling

1. Cell lines were expanded from freezer stocks according to standard procedures.
2. Cells were seeded in a total volume of 25 μL into black-walled, clear-bottom, Poly-D-lysine coated 384-well microplates and incubated at 37° C. for the appropriate time prior to testing.

### Blocker/Antagonist Format

1. After cell plating and incubation, media was removed and 25 μL of 1× compound in 1× HBSS/0.1% BSA was added.
2. Compounds were incubated with cells at 37° C. for 30 minutes.

### Dye Loading

1. Assays were performed in IX Dye Loading Buffer consisting of IX Dye, IX HBSS/20 mM Hepes.
2. After compound incubation, 25 μL of 1× dye was added to wells.
3. Cells were incubated for 30-60 minutes at 37° C.

### Signal Detection

1. After dye incubation, microplates were transferred to a PerkinElmer Envision instrument for fluorescence signal detection.

### Data Analysis

1. Compound activity was analyzed using CBIS data analysis suite (ChemInnovation, CA).
2. For blocker mode assays, percentage inhibition was calculated using the following formula: % Inhibition=100%×(1−(mean RLU of test sample−mean RLU of vehicle control)/(mean RLU of positive control−mean RLU of vehicle control)).

For Primary screens, percent response was capped at 0% or 100% where calculated percent response returned a negative value or a value greater than 100, respectively.

### Assay Design: Enzymatic Assays

### Enzyme Preparations

Enzyme preparations were sourced from various vendors: AChE (R&D Systems), COX1 and COX2 (BPS Bioscience), MAOA (Sigma), PDE3A and PDE4D2 (Signal Chem).

### Enzyme Activity Assays

1. Enzymatic assays determine the enzymatic activity by measuring either the consumption of substrate or production of product over time. Different detection methods were used in each enzymatic assay to measure the concentrations of value greater than 100, respectively. substrates and products.
2. ACHE: Enzyme and test compound were preincubated for 15 minutes at room temp before substrate addition. Acetylthiocholine and DTNB were added and incubated at room temperature for 30 minutes. Signal was detected by measuring absorbance at 405 nm.
3. COX1 & COX2: Enzyme stocks were diluted in Assay Buffer (40 mM Tris-HCl, 1×PBS, 0.5 mM Phenol, 0.01% Tween-20+100 nM Hematin) and allowed to equilibrate with compounds at room temperature for 30 minutes (binding incubation). Arachidonic acid (1.7 μM) and Ampliflu Red (2.5 μM) were prepared and dispensed into a reaction plate. Plates were read immediately on a fluorimeter with the emission detection at 590 nm and excitation wavelength 544 nm.
4. MAOA: Enzyme and test compound were preincubated for 15 minutes at 37° C. before substrate addition. The reaction was initiated by addition of kynuramine and incubated at 37° C. for 30 minutes. The reaction was terminated by addition of NaOH. The amount of 4-hydroxyquioline formed was determined through spectrofluorimetric readout with the emission detection at 380 nm and excitation wavelength 310 nm.
5. PDE3A & PDE4D2: Enzyme and test compound were preincubated for 15 minutes at room temp before substrate addition. cAMP substrate (at a concentration equal to EC₅₀) was added and incubated at room temperature for 30 minutes. Enzyme reaction was terminated by addition of 9 mM IBMX. Signal was detected using the HitHunter® cAMP detection kit.

### Signal Detection

1. For each assay, microplates were transferred to a PerkinElmer Envision instrument and readout as described.

### Data Analysis

1. Compound activity was analyzed using CBIS data analysis suite (ChemInnovation, CA).
2. For enzyme activity assays, percentage inhibition was calculated using the following formula:

% Inhibition=100%×(1−(mean RLU of test sample−mean RLU of vehicle control)/(mean RLU of positive control−mean RLU of vehicle control)).

For Primary screens, percent response was capped at 0% or 100% where calculated percent response returned a negative value or a value greater than 100, respectively.

TABLE 7
Target Names and Reference Compound Activity (Positive Controls) in Assays

| | | | Reference | Result | RC₅₀ |
|---|---|---|---|---|---|
| Target | Abbreviation | Mode | Compound | Type | (μM) |
| 5-Hydroxytryptamine | 5-HTR_{1A} | Agonist | Serotonin | EC₅₀ | 0.00395 |
| (Serotonin) Receptor 1A | | | Hydrochloride | | |
| 5-Hydroxytryptamine | 5-HTR_{1A} | Antagonist | Spiperone | IC₅₀ | 0.10535 |
| (Serotonin) Receptor 1A | | | | | |
| 5-Hydroxytryptamine | 5-HTR_{1B} | Agonist | Serotonin | EC₅₀ | 2.00E−04 |
| (Serotonin) Receptor 1B | | | Hydrochloride | | |
| 5-Hydroxytryptamine | 5-HTR_{1B} | Antagonist | SB 224289 | IC₅₀ | 0.00606 |
| (Serotonin) Receptor 1B | | | | | |
| 15-Hydroxytryptamine | 5-HTR_{2A} | Agonist | Serotonin | EC₅₀ | 0.00257 |
| (Serotonin) Receptor 2A | | | Hydrochloride | | |
| 5-Hydroxytryptamine | 5-HTR_{2A} | Antagonist | Altanserin | IC₅₀ | 0.01553 |
| (Serotonin) Receptor 2A | | | | | |
| 5-Hydroxytryptamine | 5-HTR_{2B} | Agonist | Serotonin | EC₅₀ | 0.00396 |
| (Serotonin) Receptor 2B | | | Hydrochloride | | |
| 5-Hydroxytryptamine | 5-HTR_{2B} | Antagonist | LY 272015 | IC₅₀ | 3.00E−04 |
| (Serotonin) Receptor 2B | | | | | |
| 5-Hydroxytryptamine | 5-HTR_{3A} | Blocker | Bemesetron | IC₅₀ | 0.00305 |
| (Serotonin) Receptor 3A | | | | | |
| 5-Hydroxytryptamine | 5-HTR_{3A} | Opener | Serotonin | EC₅₀ | 0.36698 |
| (Serotonin) Receptor 3A | | | Hydrochloride | | |
| Acetylcholinesterase | ACHE | Inhibitor | Physostigmine | IC₅₀ | 0.03747 |
| Adenosine Receptor A2A | ADORA2A | Antagonist | SCH 442416 | IC₅₀ | 0.0798 |
| Adenosine Receptor A2A | ADORA2A | Agonist | NECA | EC₅₀ | 0.01783 |
| Adrenergic Receptor α₁A | ADRA1A | Agonist | A 61603 | EC₅₀ | 9.00E−05 |
| | | | Hydrobromide | | |
| Adrenergic Receptor α₁A | ADRA1A | Antagonist | Tamsulosin | IC₅₀ | 0.00115 |
| Adrenergic Receptor α₂A | ADRA2A | Agonist | UK 14304 | EC₅₀ | 6.00E−05 |
| Adrenergic Receptor α₂A | ADRA2A | Antagonist | Yohimbine | IC₅₀ | 0.00463 |
| Adrenergic Receptor β1 | ADRB1 | Agonist | (−)-Isoproterenol | EC₅₀ | 0.002 |
| Adrenergic Receptor β1 | ADRB1 | Antagonist | Betaxolol | IC₅₀ | 0.0034 |
| Adrenergic Receptor β2 | ADRB2 | Agonist | (−)-Isoproterenol | EC₅₀ | 3.00E−04 |
| Adrenergic Receptor β2 | ADRB2 | Antagonist | ICI 118,551 | IC₅₀ | 0.00056 |
| | | | hydrochloride | | |
| Nuclear Hormone | AR | Agonist | 6a- | EC₅₀ | 0.00195 |
| Androgen Receptor | | | Fluorotestosterone | | |
| Nuclear Hormone | AR | Antagonist | Geldanamycin | IC₅₀ | 0.09429 |
| Androgen Receptor | | | | | |
| Arginine Vasopressin | AVPR1A | Agonist | [Arg⁸]-Vasopressin | EC₅₀ | 0.00037 |
| Receptor 1A | | | | | |
| Arginine Vasopressin | AVPR1A | Antagonist | SR 49059 | IC₅₀ | 0.00182 |
| Receptor 1A | | | | | |
| Voltage-gated L-type | CAV1.2 | Blocker | Isradipine | IC₅₀ | 0.01691 |
| calcium channel | | | | | |
| Cholecystokinin Receptor | CCKAR | Agonist | (Tyr[SO₃H]₂₇) | EC₅₀ | 1.00E−04 |
| A | | | Cholecystokinin | | |
| | | | fragment 26-33 | | |
| | | | Amide | | |
| Cholecystokinin Receptor | CCKAR | Antagonist | SR 27897 | IC₅₀ | 0.03707 |
| A | | | | | |
| Muscarinic acetylcholine | CHRM1 | Agonist | Acetylcholine | EC₅₀ | 0.01621 |
| Receptor M1 | | | chloride | | |
| Muscarinic acetylcholine | CHRM1 | Antagonist | Atropine | IC₅₀ | 0.00306 |
| Receptor M1 | | | | | |
| Muscarinic acetylcholine | CHRM2 | Agonist | Acetylcholine | EC₅₀ | 0.02486 |
| Receptor M2 | | | chloride | | |
| Muscarinic acetylcholine | CHRM2 | Antagonist | Atropine | IC₅₀ | 0.00406 |
| Receptor M2 | | | | | |
| Muscarinic acetylcholine | CHRM3 | Agonist | Acetylcholine | EC₅₀ | 0.03952 |
| Receptor M3 | | | chloride | | |
| Muscarinic acetylcholine | CHRM3 | Antagonist | Atropine | IC₅₀ | 0.0015 |
| Receptor M3 | | | | | |
| Cannabinoid Receptor 1 | CNR1 | Agonist | CP 55940 | EC₅₀ | 4.00E−05 |
| Cannabinoid Receptor 1 | CNR1 | Antagonist | AM 251 | IC₅₀ | 0.00324 |
| Cannabinoid Receptor 2 | CNR2 | Agonist | CP 55940 | EC₅₀ | 0.00016 |
| Cannabinoid Receptor 2 | CNR2 | Antagonist | SR 144528 | IC₅₀ | 0.03516 |
| Cyclooxygenase 1 | COX1 | Inhibitor | Indomethacin | IC₅₀ | 0.0329 |
| Cyclooxygenase 2 | COX2 | Inhibitor | NS-398 | IC₅₀ | 0.02245 |
| Dopamine transporter | DAT | Blocker | GBR 12909 | IC₅₀ | 0.01456 |
| Dopamine Receptor D1 | DRD1 | Agonist | Dopamine | EC₅₀ | 0.0855 |
| Dopamine Receptor D1 | DRD1 | Antagonist | SCH 39166 | IC₅₀ | 0.00092 |
| Dopamine Receptor D2 | DRD2S | Agonist | Dopamine | EC₅₀ | 0.001 |
| Dopamine Receptor D2 | DRD2S | Antagonist | Risperidone | IC₅₀ | 0.00158 |
| Endothelin Receptor Type | EDNRA | Agonist | Endothelin 1 | EC₅₀ | 0.0011 |
| A | | | | | |
| Endothelin Receptor Type | EDNRA | Antagonist | BMS 182874 | IC₅₀ | 1.10701 |
| A | | | | | |
| Gamma-aminobutyric acid | GABAA | Blocker | Picrotoxin | IC₅₀ | 2.77847 |
| Receptor A | | | | | |
| Gamma-aminobutyric acid | GABAA | Opener | GABA | EC₅₀ | 6.35785 |
| Receptor A | | | | | |
| Nuclear Hormone | GR | Agonist | Dexamethasone | EC₅₀ | 0.04971 |
| Glucocorticoid Receptor | | | | | |
| Nuclear Hormone | GR | Antagonist | Mifepristone | IC₅₀ | 0.07236 |
| Glucocorticoid Receptor | | | | | |
| Nuclear Hormone | HERG | Blocker | Astemizole | IC₅₀ | 0.22171 |
| Glucocorticoid Receptor | | | | | |
| Histamine Receptor H1 | HRH1 | Agonist | Histamine | EC₅₀ | 0.03202 |
| Histamine Receptor H1 | HRH1 | Antagonist | Mepyramine | IC₅₀ | 0.00538 |
| Histamine Receptor H2 | HRH2 | Agonist | Histamine | EC₅₀ | 0.26388 |
| Histamine Receptor H2 | HRH2 | Antagonist | Tiotidine | IC₅₀ | 0.13915 |
| Insulin Receptor (tyrosine | INSR | Inhibitor | BMS-754807 | IC₅₀ | 0.00052 |
| kinase) | | | | | |
| Kv11.1, the alpha subunit | KvLQT1/minK | Blocker | XE 991 | IC₅₀ | 1.66819 |
| of a potassium ion channel | | | | | |
| Kv11.1, the alpha subunit | KvLQT1/mink | Opener | ML-277 | EC₅₀ | 2.30579 |
| of a potassium ion channel | | | | | |
| Lymphocyte Cell-Specific | LCK | Inhibitor | Gleevec | IC₅₀ | 13.36093 |
| Protein-Tyrosine Kinase | | | | | |
| (Src family) | | | | | |
| Monoamine oxidase type A | MAOA | Inhibitor | Clorgyline | IC₅₀ | 0.00446 |
| Nicotinic acetylcholine | nACHR(a4/b2) | Blocker | Dihydro-AY- | IC₅₀ | 0.68211 |
| Receptor α4 β2 | | | erythroidine | | |
| Nicotinic acetylcholine | nACHR(a4/b2) | Opener | (−)-Nicotine | EC₅₀ | 2.3741 |
| Receptor α4 β2 | | | | | |
| A tetrodotoxin-resistant | NAV1.5 | Blocker | Lidocaine | IC₅₀ | 32.04972 |
| voltage-gated sodium | | | | | |
| channel N-methyl-D- | | | | | |
| aspartate (NMDA) | | | | | |
| Glutamate | | | | | |
| Norepinephrine transporter | NET | Blocker | Desipramine | IC₅₀ | 0.01292 |
| N-methyl-D-aspartate | NMDAR | Blocker | (+)-MK 801 maleate | IC₅₀ | 0.03884 |
| (NMDA) Glutamate | (1A/2B) | | | | |
| Receptor 1A/2B | | | | | |
| N-methyl-D-aspartate | NMDAR | Opener | L-Glutamic Acid | EC₅₀ | 0.413 |
| (NMDA) Glutamate | (1A/2B) | | | | |
| Receptor 1A/2B | | | | | |
| Opioid Receptor Delta 1 | OPRD1 | Agonist | DADLE | EC₅₀ | 0.00012 |
| Opioid Receptor Delta 1 | OPRD1 | Antagonist | Naltriben | IC₅₀ | 0.00039 |
| Opioid Receptor Kappa 1 | OPRK1 | Agonist | Dynorphin A (1-17) | EC₅₀ | 0.01234 |
| Opioid Receptor Kappa 1 | OPRK1 | Antagonist | nor- | IC₅₀ | 0.00724 |
| | | | Binaltorphimine | | |
| Opioid Receptor Mu | OPRM1 | Agonist | DAMGO | EC₅₀ | 0.00221 |
| Opioid Receptor Mu | OPRM1 | Antagonist | Naloxone | IC₅₀ | 0.00552 |
| CGMP-inhibited cyclic | PDE3A | Inhibitor | Cilostamide | IC₅₀ | 0.05554 |
| nucleotide | | | | | |
| phosphodiesterase 3A | | | | | |
| CAMP-specific 3′,5′-cyclic | PDE4D2 | Inhibitor | Cilomilast | IC₅₀ | 0.01688 |
| phospho-diesterase | | | | | |
| Catecholamine | | | | | |
| Transporters | | | | | |
| Rho Associated Coiled- | ROCK1 | Inhibitor | Staurosporine | IC₅₀ | 8.00E−05 |
| Coil Containing Protein | | | | | |
| Kinase 1 (serine-threonine | | | | | |
| kinase) | | | | | |
| Serotonin transporter | SERT | Blocker | Clomipramine | IC₅₀ | 0.00242 |
| Vascular endothelial | VEGFR2 | Inhibitor | SU-11248 | IC₅₀ | 0.00022 |
| growth factor receptor 2 | | | | | |
| (KDR tyrosine kinase) | | | | | |

### Example 6: Human Serotonin Transporter (SERT, SLC6A4) Functional Antagonist Uptake Assay

Three tryptamine derivatives were evaluated for inhibiting the human 5-HT transporter (hSERT) as expressed in CHO cells using an antagonist radioligand assay (Tatsumi, M. et al. 1999, Eur. J. Pharmacol., 368: 277-283). Compound binding was calculated as a percent inhibition of the binding of 2 nM [³H]imipramine using a scintillation method and inhibition constants (Ki) were calculated using the Cheng Prusoff equation. Test compounds were assayed in two trials at 3.0E-10, 3.0E-9, 3.0E-8, 3.0E-7, 3.0E-6, and 3.0E-5 M.

All tested compounds showed inhibition of hSERT at the tested concentrations, as summarized in the table below.

TABLE 8
Human Serotonin Transporter Functional Antagonist Uptake Results

| Compound | IC₅₀ (μM) | Ki (μM) |
|---|---|---|
| 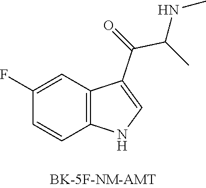 [SMILES: CNC(C)C(=O)c1c[nH]c2ccc(F)cc12] | 3.5 | 1.60 |
| 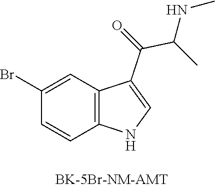 [SMILES: CNC(C)C(=O)c1c[nH]c2ccc(Br)cc12] | 5.60 | 2.60 |
| 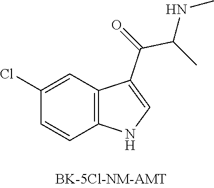 [SMILES: CNC(C)C(=O)c1c[nH]c2ccc(Cl)cc12] | 2.50 | 1.10 |

When compounds are substrates for monoamine transporters instead of solely inhibitors, it is known that IC₅₀s underestimate their potency for interacting with these transporters (for example, Ilic, M. et al. 2020, Frontiers in Pharmacology 11: 673). It is also informative to measure EC₅₀s for 5-HT release.

### Example 7: Human Monoamine Transporter (hMAT) Release

To assess the effects of tryptamine derivatives on extracellular dopamine and serotonin concentrations, in vitro measures of serotonin and dopamine release were made using Chinese hamster ovary cells that stably express human monoamine transporters, dopamine (hDAT) and serotonin (hSERT) transporter. Dextroamphetamine and norfenfluramine were used as reference releasers of dopamine and serotonin, respectively.

Assay results revealed that the disclosed tryptamine derivatives are more potent at releasing 5-HT than DA, with DA/SERT ratios suggesting MDMA-like effects and indicating reduced abuse liability compared to amphetamine and other substances with higher DAT/SERT ratios.

TABLE 9
Effects of Tryptamines on DAT and SERT

| | EC₅₀ | EC₅₀ | DAT/ |
|---|---|---|---|
| | DAT | SERT | SERT |
| | (nM) | (nM) | ratio* |
| 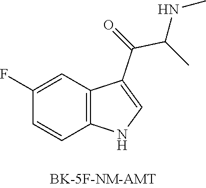 [SMILES: CNC(C)C(=O)c1c[nH]c2ccc(F)cc12] | 620 | 190 | 0.31 |
| 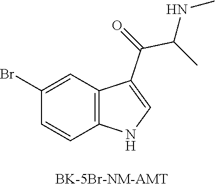 [SMILES: CNC(C)C(=O)c1c[nH]c2ccc(Br)cc12] | 2100 | 295 | 0.14 |
| 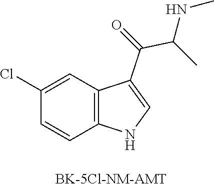 [SMILES: CNC(C)C(=O)c1c[nH]c2ccc(Cl)cc12] | 865 | 200 | 0.23 |

*DAT/SERT ratios are calculated here as (DAT EC₅₀)⁻¹/(SERT EC₅₀)⁻¹ where larger numbers indicate higher DAT selectivity

hSERT Release Measurement Methods

Chinese hamster ovary cells expressing human SERT were seeded in Cytostar™ (PerkinElmer) plate with standard culture medium the day before the experiment at a single density (5,000 cells/assay). Cells were incubated overnight with 5% CO₂ at 37° C. The day of experiment, the medium was replaced by incubation buffer (140 mM NaCl, 4.8 mM KCl, 1.2 mM MgSO₄, 0.1 mM KH₂PO₄, 10 mM HEPES, pH 7.4) with a single concentration of [³H]serotonin at 150 nM. Experiments comparing release in radioligand-free incubation buffer versus incubation buffer containing [³H]serotonin determined that the latter provided better signal stability. Therefore, this was used for experiments.

Two control conditions were used: (1) buffer only (with 1% DMSO concentration to match that in the test compound condition) to verify the background level of release; and (2) one reference SERT substrate compound, norfenfluramine, at 100 μM, to make it possible to calculate a relative Eₘₐₓ. Pilot studies varying DMSO concentration from 0.1 to 3% indicated that signal decreased at higher DMSO concentrations but that 1% DMSO retained good properties.

Cells were incubated at room temperature at different incubation times and radioactivity counted. Test compounds were measured at concentrations of 1e-10, 1e-09, 1e-08, 1e-07, 1e-06, 1e-05, and 1e-04 M. Each experiment was performed in duplicate (n=2) and results calculated at two inhibition times (60 and 90).

hDAT Release Measurement Methods

Chinese hamster ovary cells expressing human DAT were seeded in Cytostar™ plate with standard culture medium the day before experiment at one single density (2 500 cells/assay). Cells were incubated overnight with 5% CO₂ at 37° C. The day of experiment, the medium was replaced by incubation buffer (TrisHCl 5 mM, 120 mM NaCl, 5.4 mM KCl, 1.2 mM MgSO₄, 1.2 mM CaCl₂), Glucose 5 mM, 7.5 mM HEPES, pH 7.4) with a single concentration of [³H]dopamine at 300 nM. Experiments comparing release in radioligand-free incubation buffer versus incubation buffer containing [3H]dopamine determined that the latter provided better signal stability. Therefore, this was used for experiments.

For all assays, three reference conditions were employed: (1) radioligand-containing buffer only, to verify the control level of release, (2) buffer with 1% DMSO (solvent used to solubilize the test compounds), (3) 100 uM amphetamine (in 1% DMSO) to make it possible to calculate a relative Eₘₐₓ.

Cells were incubated at room temperature at different incubation times and radioactivity counted. Test compounds were measured at concentrations of 1e-10, 1e-09, 1e-08,1e-07,1e-06, 1e-05, and 1e-04 M. Each experiment was performed in duplicate (n=2) and results calculated at two inhibition times (60 and 90).

### Statistical Analysis

EC/IC₅₀s were calculated using the R packages drm (to fit the regression model) and LL.4 (to define the structure of the log-logistic regression model). Values were fit to the following function:

f(x)=c+(d−c)/(1+exp(b(log(x)−log(e)))

where b=the Hill coefficient, c=minimum value, d=maximum value, and e=EC₅₀/IC₅₀.
Values were calculated for both experimental repetitions at both stable inhibition times (60 and 90 minutes), resulting in four estimates of EC₅₀ and other parameters for each compound and transporter. These four values were averaged to produce final estimates for each compound and transporter.

### Example 8: Evaluation of Monoamine Oxidase A (MAOA) Inhibition

MAO (monoamine oxygen oxidoreductase (deaminating) (flavin-containing); EC 1.4.3.4) is the main enzyme that metabolizes biological monoamines such as serotonin, dopamine, and norepinephrine. MAO exists in two isoforms known as MAOA and MAOB. While MAOA preferentially metabolizes serotonin, MAO-B preferentially metabolizes phenethylamine and benzylamine. DA and NE are non-selective substrates of both isoforms. MAO inhibitors (MAOI) are currently used in the treatment of CNS disorders, including depression and Parkinson's disease.

Select compounds of the present invention, as well as representative previously known compounds, were assayed using the method described in Example 5 for monoamine oxidase A (MAOA) inhibition activity. This data, along with previously reported data also presented below, show that the compounds of the present invention present unexpectedly low MAOA inhibition, given the sub-micromolar MAOA IC₅₀s of many similar compounds. For example, while 5F-AMT and 5F-NM-AMT are MAOIs, BK-5F-NM-AMT is not an MAOI. Because a similar compound with a beta-position ketone but without the alpha-methyl, BK-5F-NMT, is also an MAOI, the unexpected attenuation of MAO interactions cannot be attributed entirely to the addition of the beta-keto. The affect seems to be a result of multiple modifications that do not on their own eliminate MAOA inhibition. This indicates that the compounds of the present invention pose little risk of inducing MAOI-related toxicities from overdose, in contrast to AMT and related entactogenic tryptamines.

TABLE 10
MAOA Inhibition Data for a Selection of Tryptamines

| | | MAOA | | |
|---|---|---|---|---|
| | | IC₅₀ | | |
| Compound | IUPAC Name | (uM) | Hill | Source |
| 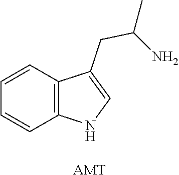 | 1-(1H-indol-3-yl)propan-2-amine | 0.38 | NA | (1) |
| 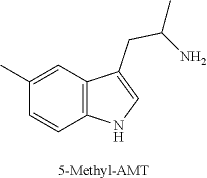 | 1-(5-methyl-1H-indol-3-yl) propan-2-amine | 1.5 | NA | (1) |
| 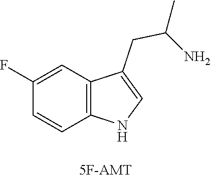 | 1-(5-fluoro-1H-indol-3-yl) propan-2-amine | 0.45 | NA | (1) |
| 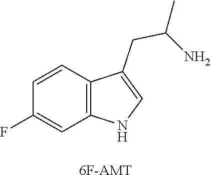 | 1-(6-fluoro-1H-indol-3-yl) propan-2-amine | 1.8 | NA | (1) |
| 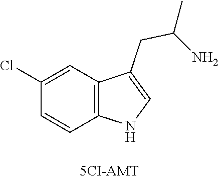 | 1-(5-chloro-1H-indol-3-yl) propan-2-amine | 0.25 | NA | (1) |
| 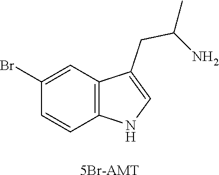 | 1-(5-bromo-1H-indol-3-yl)propan-2-amine | 1.3 | NA | (1) |
| 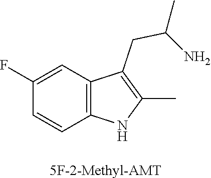 | 1-(5-fluoro-2-methyl-1H-indol- 3-yl)propan-2-amine | 4.1 | NA | (1) |
| 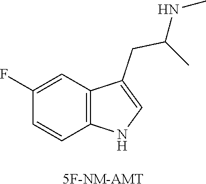 | [1-(5-fluoro-1H-indol-3-yl)propan- 2-yl](methyl)amine | 0.45 | 1.01 | Original data |
| 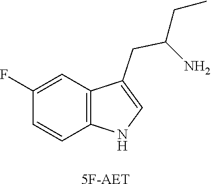 | 1-(5-fluoro-1H-indol-3-yl)butan-2-amine | 2.48 | 0.89 | Original data |
| 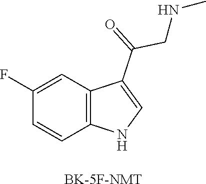 | 1-(5-fluoro-1H-indol-3-yl)-2- (methylamino)ethan-1-one | 3.43 | 1.02 | Original data |
| 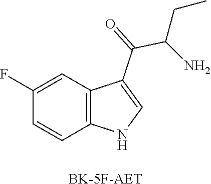 [SMILES: CCC(N)C(=O)c1c[nH]c2ccc(F)cc12 ; "BK-5F-AET"] | 2-amino-1-(5-fluoro-1H-indol-3-yl) butan-1-one | >30 | NA | Original data |
| 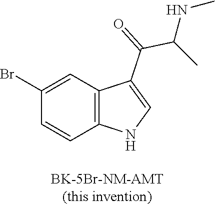 [SMILES: CNC(C)C(=O)c1c[nH]c2ccc(Br)cc12 ; "BK-5Br-NM-AMT(this invention)"] | 1-(5-bromo-1H-indol-3-yl)-2- (methylamino)propan-1-one | >10 | NA | Original data |
| 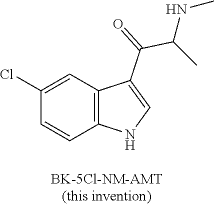 [SMILES: CNC(C)C(=O)c1c[nH]c2ccc(Cl)cc12 ; "BK-5Cl-NM-AMT(this invention)"] | 1-(5-chloro-1H-indol-3-yl)-2- (methylamino)propan-1-one | >10 | NA | Original data |
| 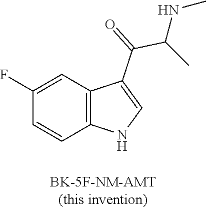 [SMILES: CNC(C)C(=O)c1c[nH]c2ccc(F)cc12] | 1-(5-fluoro-1H-indol-3-yl)-2- (methylamino)propan-1-one | >10 | NA | Original data |

(1) Wagmann et al. Toxicology letters, 2017. 272, 84-93

### Example 9: Comparison of Novel Compounds to MDMA in Rats Trained to Distinguish MDMA from Placebo

Rodent drug discrimination assays are the main methodology for understanding the interoceptive (i.e., felt experience) effects of drugs in animals (Baker, Neurobiology of Psychedelic Drugs, 2017, 201-219; Fantegrossi, Murnane & Reissig. Biochemical pharmacology, 2008, 75(1), 17-33). In a typical discrimination task, an animal is trained to emit one response (often pressing of a lever) during experimental sessions shortly after the administration of a particular drug (the “training drug”), and a different response during sessions that follow administration of the placebo drug vehicle. Once the animals are stably engaged in this task, a novel drug can be tested for similarity to the training drug.

Training and testing procedures were conducted in standard operant conditioning chambers housed in sound-attenuating shells. Dustless Precision Pellets (45 mg; Product #F0021; BioServ, Flemington, N.J.) were used as reinforcements for lever pressing.

A training dose of 1.5 mg/kg MDMA was used and training followed standard procedures (Baker 2017, Neurobiology of Psychedelic Drugs, 201-219). Briefly, rats were trained to discriminate 1.5 mg/kg MDMA from placebo (saline vehicle) under a fixed ratio (FR) 20 schedule of food reinforcement. Lever assignment to stimulus condition was counterbalanced among rats in each experiment. Drug and vehicle training sessions were alternated with the order guaranteeing that the same stimulus condition occurred no more than twice consecutively. The criterion for stimulus control was a minimum of eight out of ten consecutive discrimination training sessions with 80% or higher correct lever responses prior to delivery of the first reinforcer and for the total session.

After stimulus control was established, test sessions were conducted. The test compound was administered via intraperitoneal injection 30 min prior to starting the test sessions. Test sessions were similar to training sessions, with the exception that responses were not reinforced and sessions ended upon completion of the first FR 20 or after 20 min, whichever occurred first. Testing criteria between sessions required subjects to complete at least one drug and at least one vehicle training session consecutively with 80% or higher injection-appropriate responding. The main outcome measure was percent responses on the MDMA-associated lever, while rate of lever pressing (responses per second) was used as a control measure. Each dose level of the test compound included data from at least three animals.

BK-5F-NM-AMT was tested at 1, 2, and 4 mg/kg in four MDMA-trained rats. Results indicate that BK-5F-NM-AMT produces effects that are perceived by rats as significantly and dose-dependently different from those of MDMA. Rate of lever pressing was not altered by the test drug, indicating a lack of nonspecific effects, such as sedative or stimulant effects. This is consistent with BK-5F-NM-AMT having reduced dopaminergic and noradrenergic effects compared to MDMA. Alternatively, this could be explained by BK-5F-NM-AMT having a brief acute effect that resolved prior to testing 30 minutes after drug administration. Either of these possibilities indicate significant and unexpected utility.

### Example 10: Evaluation of Entactogenic Effect of Decreased Neuroticism

The entactogenic effect of decreased neuroticism can be measured as a decrease in social anxiety using the Brief Fear of Negative Evaluation-revised (BFNE) (Carleton et al., 2006, Depression and Anxiety, 23(5), 297-303; Leary, 1983, Personality and Social Psychology bulletin, 9(3), 371-375). This 12-item Likert scale questionnaire measures apprehension and distress due to concerns about being judged disparagingly or with hostility by others. Ratings use a five-point Likert scale with the lowest, middle, and highest values labeled with “much less than normal,” “normal,” and “much more than normal.” The BFNE can be administered before and repeatedly during therapeutic drug effects. Participants are instructed to answer how they have been feeling for the past hour, or otherwise during the effect of the drug. Baseline-subtracted responses are typically used in statistical models.

### Example 11: Evaluation of Entactogenic Effect of Authenticity

The entactogenic effect of authenticity can be measured using the Authenticity Inventory (Kernis & Goldman. 2006. Advances in experimental social psychology, 38, 283-357) as modified by Baggott et al (Journal of Psychopharmacology 2016, 30.4: 378-87). Administration and scoring of the instrument is almost identical to that of the BFNE. The Authenticity Inventory consists of the following items, which are each rated on a 1-5 scale, with select items reverse scored as specified by Kernis & Goldman:
I am confused about my feelings.
I feel that I would pretend to enjoy something when in actuality I really didn't.
For better or worse, I am aware of who I truly am.
I understand why I believe the things I do about myself
I want the people with whom I am close to understand my strengths.
I actively understand which of my self-aspects fit together to form my core or true self.
I am very uncomfortable objectively considering my limitations and shortcomings.
I feel that I would use my silence or head-nodding to convey agreement with someone else's statement or position even though I really disagreed.
I have a very good understanding of why I do the things I do.
I am willing to change myself for others if the reward is desirable enough.
I would find it easy to pretend to be something other than my true self.
I want people with whom I am close to understand my weaknesses.
I find it difficult to critically assess myself (unchanged)
I am not in touch with my deepest thoughts and feelings.
I feel that I would make it a point to express to those I am close with how much I truly care for them.
I have difficulty accepting my personal faults, so I try to cast them in a more positive way.
I feel that I idealize the people close to me rather than objectively see them as they truly are.
If asked, people I am close to could accurately describe what kind of person I am.
I prefer to ignore my darkest thoughts and feelings.
I am aware of times when I am not being my true self.
I am able to distinguish the self-aspects that are important to my core or true self from those that are unimportant.
People close to me would be shocked or surprised if they discovered what I am keeping inside me.
It is important for me to understand the needs and desires of those with whom I am close.
I want people close to me to understand the real me, rather than just my public persona or “image”.
I could act in a manner that is consistent with my personally held values, even if others criticized me or rejected me for doing so.
If a close other and I were in disagreement, I would rather ignore the issue than constructively work it out.
I feel that I would do things that I don't want to do merely to avoid disappointing people.
My behavior expresses my values.
I actively attempt to understand myself as well as possible.
I feel that I'd rather feel good about myself than objectively assess my personal limitations and shortcomings.
My behavior expresses my personal needs and desires.
I have on a “false face” for others to see.
I feel that I would spend a lot of energy pursuing goals that are very important to other people even though they are unimportant to me.
I am not in touch with what is important to me.
I try to block out any unpleasant feelings I have about myself.
I question whether I really know what I want to accomplish in my lifetime.
I am overly critical about myself.
I am in touch with my motives and desires.
I feel that I would deny the validity of any compliments that I receive.
I place a good deal of importance on people close to me understanding who I truly am.
I find it difficult to embrace and feel good about the things I have accomplished.
If someone pointed out or focused on one of my shortcomings, I would quickly try to block it out of my mind and forget it.
The people close to me could count on me being who I am, regardless of what setting we were in.
My openness and honesty in close relationships are extremely important to me.
I am willing to endure negative consequences by expressing my true beliefs about things.

### Example 12: Evaluation of Side Effects of Entactogens

Adverse effects of an entactogen include formation of tolerance to entactogens, headache, difficulty concentrating, lack of appetite, lack of energy, and decreased mood. To measure these adverse effects, patients can be asked to complete a self-report symptom questionnaire, such as the Subjective Drug Effects Questionnaire (SDEQ) or List of Complaints. The SDEQ is a 272-item self-report instrument measuring perceptual, mood, and somatic changes caused by drugs including hallucinogens like LSD (Katz et al. 1968. J Abnorm Psychology 73:1-14). It has also been used to measure the therapeutic and adverse effects of MDMA (Harris et al. 2002. Psychopharmacology, 162(4), 396-405). The List of Complaints is a 66-item questionnaire that measures physical and general discomfort and is sensitive to entactogen-related complaints (for example, Vizeli & Liechti. 2017. Journal of Psychopharmacology, 31(5), 576-588).

In addition to these mild toxicities, MDMA is associated with a number of more severe toxicities, including but not limited to acute and chronic cardiovascular changes, hepatotoxicity, hyperthermic syndromes, hyponatremia, and neurotoxicity (see the MDMA Investigator's Brochure, 13th Edition: Mar. 22, 2021, and references therein, available from the sponsor of MDMA clinical trials at MAPS.org).

Alternatively, individual items can be taken from the SDEQ or List of Complaints in order to create more focused questionnaires and reduce the burden of filling out time-consuming paperwork on participants. To measure tolerance formation, a global measure of the intensity of therapeutic effects can be used, such as the question “on a scale from 0 to 100 where 0 is no ‘good drug effect’ and 100 is the most ‘good drug effect’ you have ever felt, how would you rate this drug experience?”

In some embodiments, the questionnaire will be administered approximately 7 hours after a patient takes the tryptamine (with instructions to answer for the time since taking the entactogen) and then daily (with instructions to answer for the last 24 hours) for up to 96 hours after the tryptamine was taken. Decreases in adverse effects of a compound compared to MDMA can be shown by comparing the intensity (for the tolerance question) or prevalence (for other symptom questions) of effects that occur. Prevalence of adverse effects including formation of tolerance to entactogens, headache, difficulty concentrating, lack of appetite, lack of energy, and decreased mood may be decreased by approximately 10%, 20%, 30%, 40%, 50%, 60%, 70%, 80%, 90%, 99%, or 100%.

While the present invention is described in terms of particular embodiments and applications, it is not intended that these descriptions in any way limit its scope to any such embodiments and applications, and it will be understood that many modifications, substitutions, changes, and variations in the described embodiments, applications, and details of the invention illustrated herein can be made by those skilled in the art without departing from the spirit of the invention, or the scope of the invention as described in the appended claims.

I claim:

1. A compound of formula 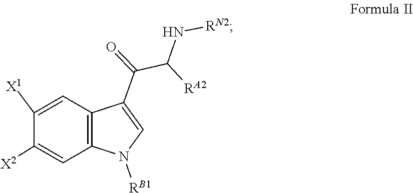
or a pharmaceutically acceptable salt or salt mixture thereof,
wherein:
R^{N2} is —CH₃ or —CH₂CH₃;
R^{A2} is —CH₃, —CH₂CH₃, —CH₂CH₂X, —CH₂CHX₂, —CH₂CX₃, —CH₂OH or —CH₂CH₂OH;
R^{B1} is —H, —CH₃, or —CH₂CH₃;
X¹ is independently selected in each instance from the group consisting of —H, —F and —Cl;
X² is selected from the group consisting of —H, —F and —Cl, wherein X¹ and X² must be different;
X is independently selected in each instance from the group consisting of —F, —Cl, and —Br.

2. The compound of claim 1 of formula: 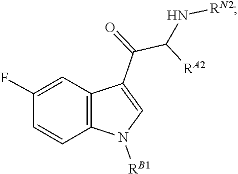
or a pharmaceutically acceptable salt or salt mixture thereof.

3. The compound of claim 1 of formula: 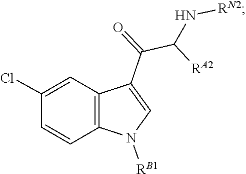
or a pharmaceutically acceptable salt or salt mixture thereof.

4. The compound of claim 1 of formula: 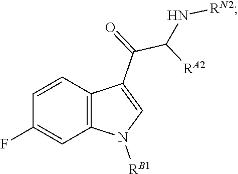
or a pharmaceutically acceptable salt or salt mixture thereof.

5. The compound of claim 1 of formula: 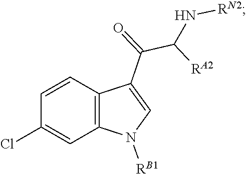
or a pharmaceutically acceptable salt or salt mixture thereof.

6. The compound of claim 1 of structure 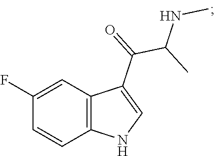 [SMILES: CNC(C)C(=O)c1c[nH]c2ccc(F)cc12]
or a pharmaceutically acceptable salt or salt mixture thereof.

7. The compound of claim 1 of structure 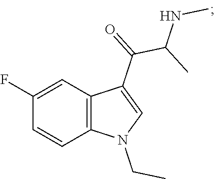 [SMILES: CCn1cc(C(=O)C(C)NC)c2cc(F)ccc21]
or a pharmaceutically acceptable salt or salt mixture thereof.

8. The compound of claim 1 of structure 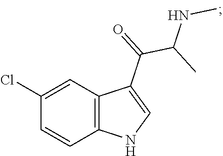 [SMILES: CNC(C)C(=O)c1c[nH]c2ccc(Cl)cc12]
or a pharmaceutically acceptable salt or salt mixture thereof.

9. The compound of claim 1 of structure 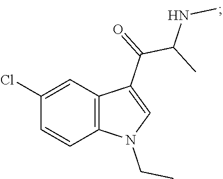 [SMILES: CCn1cc(C(=O)C(C)NC)c2cc(Cl)ccc21]
or a pharmaceutically acceptable salt or salt mixture thereof.

10. The compound of claim 1 of structure 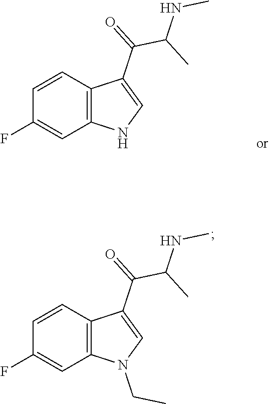 [SMILES: CNC(C)C(=O)c1c[nH]c2cc(F)ccc12 ; CCn1cc(C(=O)C(C)NC)c2ccc(F)cc21]
or a pharmaceutically acceptable salt thereof.

11. The compound of claim 1 of structure 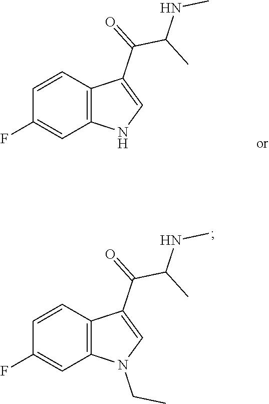 [SMILES: CNC(C)C(=O)c1c[nH]c2cc(F)ccc12 ; CCn1cc(C(=O)C(C)NC)c2ccc(F)cc21]
or a pharmaceutically acceptable salt thereof.

12. The compound of claim 1 of structure 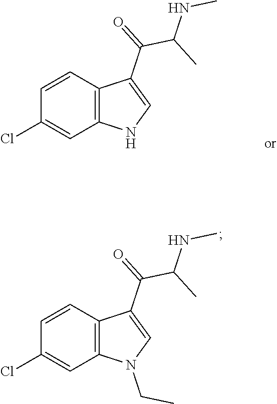 [SMILES: CNC(C)C(=O)c1c[nH]c2cc(Cl)ccc12 ; CCn1cc(C(=O)C(C)NC)c2ccc(Cl)cc21]
or a pharmaceutically acceptable salt thereof.

13. The compound of claim 1, wherein the pharmaceutically acceptable salt is selected from the group consisting of HCl, sulfate, aspartate, saccharate, phosphate, oxalate, acetate, amino acid anion, gluconate, maleate, malate, citrate, mesylate, nitrate and tartrate, or a mixture thereof.

14. The compound of claim 1 in an enantiomerically enriched form that has at least about 60% S-enantiomer.

15. The compound of claim 1 in an enantiomerically enriched form that has at least about 60% R-enantiomer.

16. A pharmaceutical composition comprising a compound of claim 1, or a pharmaceutically acceptable salt or salt mixture thereof, and a pharmaceutically acceptable carrier or excipient.

17. The pharmaceutical composition of claim 16 wherein the compound is 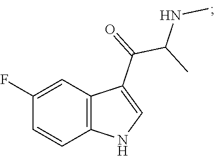 [SMILES: CNC(C)C(=O)c1c[nH]c2ccc(F)cc12]
or a pharmaceutically acceptable salt or salt mixture thereof.

18. The pharmaceutical composition of claim 16 wherein the compound is 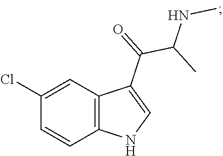 [SMILES: CNC(C)C(=O)c1c[nH]c2ccc(Cl)cc12]
or a pharmaceutically acceptable salt or salt mixture thereof.

19. The pharmaceutical composition of claim 16 wherein the pharmaceutical composition is suitable for administration systematically, orally, rectally, topically, subcutaneously, intravenously, intramuscularly, via inhalation, or to mucosal tissue.

20. The pharmaceutical composition of claim 19, wherein the pharmaceutical composition is a tablet, a gelcap, a capsule, an aqueous emulsion, an aqueous solution, or a pill.

21. The pharmaceutical composition of claim 19, wherein the pharmaceutical composition is a strip, liquid, spray, or gel.

22. The pharmaceutical composition of claim 19, wherein the pharmaceutical composition is a cream or a topical solution.
